SEQUENCE LISTING (136 sequences)

SEQ ID NO: 1 - protein, 88 aa, Mus musculus
MDRVVLGWTAVFWLTAMVEGLQVTVPDKKKVAMLFQPTVLRCHFSTSSHQPAVVQWKFKSYCQDRMGESLGMSSPRAQALSKRNLEWD

SEQ ID NO: 2 - protein, 88 aa, Mus musculus
MDRVVLGWTAVFWLTAMVEGLQVTVPDKKKVAMLFQRTVLRCHFSTSSHQPAVVQWKFKSYCQDRMGESLGMSSPRAQALSKRNLEWD

SEQ ID NO: 3 - protein, 88 aa, Mus musculus
MDRVVLGWTAVFWLTAMVEGLQVTVPDKKKVAMLFQPTVLRCHFSTSSLQPAVVQWKFKSYCQDRMGESLGMSSPRAQALSKRNLEWD

SEQ ID NO: 4 - protein, 88 aa, Mus musculus
MDRVVLGWTAVFWLTAMVEGLQVTVPDKKKVAMLFQPTVLRCHFSTSSHQPAVVQWKFKSYCLDRMGESLGMSSPRAQALSKRNLEWD

SEQ ID NO: 5 - protein, 88 aa, Mus musculus
MDRVVLGWTAVFWLTAMVEGLQVTVPDKKKVAMLFQPTVLRCHFSTSSHQPAVVQWKFKSYCQVRMGESLGMSSPRAQALSKRNLEWD

SEQ ID NO: 6 - protein, 104 aa, Artificial Sequence
  Description of Artificial Sequence Synthetic polypeptide
YRIQADKERDSMKVLYYVEKELAQFDPARRMRGRYNNTISELSSLHDDDSNFRQSYHQMRNKQFPMSGDLESNPDYWSGVMGGNSGTNRGPALEYNKEDRESFR

SEQ ID NO: 7 - protein, 165 aa, Artificial Sequence
  Description of Artificial Sequence Synthetic polypeptide
QVTVPDKKKVAMLFQPTVLRCHFSTSSHQPAVVQWKFKSYCQDRMGESLGMSSPRAQALSKRNLEWDPYLDCLDSRRTVRVVASKQGSTVTLGDFYRGREITIVHDADLQIGKLMWGDSGLYYCIITTPDDLEGKNEDSVELLVLGRTGLLADLLPSFAVEIMPE

SEQ ID NO: 8 - protein, 105 aa, Homo sapiens
YRIQADKERDSMKVLYYVEKELAQFDPARRMRGRYNNTISELSSLHEEDSNFRQSFHQMRSKQFPVSGDLESNPDYWSGVMGGSSGASRGPSAMEYNKEDRESFR

SEQ ID NO: 9 - protein, 165 aa, Homo sapiens
QVTVPDKKKVAMLFQPTVLRCHFSTSSHQPAVVQWKFKSYCQDRMGESLGMSSTRAQSLSKRNLEWDPYLDCLDSRRTVRVVASKQGSTVTLGDFYRGREITIVHDADLQIGKLMWGDSGLYYCIITTPDDLEGKNEDSVELLVLGRTGLLADLLPSFAVEIMPE

SEQ ID NO: 10 - DNA, 854 bp, Mus musculus
gtgggcatagcccatggaaatgtacgctgcaaatggagaatgtataggtgtagtctccctctccctctccctcctcctcccccctccccctctccctcccattctctctctccctctctctctctctgctcattttctgttaaaaaggccacatacgttttaacagaaaaaagatttttgcctaaagggtttctcccctatggatcctaatttggttggggcctcttggttcgttgaaccagatgcaccagccagggcacaacaaaaacaaacaaacaaacaaacaccatacaggtctaagccccaggagaagttatgccaagtccttgtagcctttctgtccctgacacccagtacaggtgcaaggaaacatcgagtcccagcctgcttggtggctcaagtagagcttgagtcgcagcccccgccacatggtccgccctctggggtggacttcgctgctaggttctgacctcccagccccgggagacggcacgtgaccgagaaactttggcgggcggtgttggcgcgggcggcggcgcggcgatcgagctcccccgcgcggcgagctctgctgcggggaggcgctcgccggtgccgcgcagcctgtgcgtgcgggaacggcccccgcagcccaatcggactctagagccagcggcagcgcgcctctcgcaggcggcggcgtccagcgcccggggccgggctgcgcggccagccccgagggctgcggcgccagggacgcgcggggcccgcgctccgccgccgccgccgcctgctgctgcgaggtcatccggatcttatcgtgccagctgatgcccgcttttcccactctggatctggatgggaagttggggaag

SEQ ID NO: 11 - DNA, 869 bp, Mus musculus
gtgggcatagcccatggaaatgtacgctgcaaatggagaatgtataggtgtagtctccctctccctccccctccccctccccctccccctccccctccccctcccattctctctctccctctctctctctctctgctcattttctgttaaaaaggccacatacgttttaacagaaaaaagatttttgcctaaagggtttctcccctatggatcctaatttggttggggcctcttggttcgttgaaccagatgcaccagccagggcacaacaaaaacaaacaaacaaacaaacaaacaaacaccacacaggtctaagccccaggagaagttatgccaagtccttgtagcctttctgtccctgacacccagtacaggtgcaaggaaacatcgagtcccagcctgcttggtggctcaagtagagcttgagtcgcagcccccgccacatggtccgccctctggggtggacttcgctgctaggctctgacctcccagccccgggagacggcacgtgaccgagaaactttggcgggcggtgttggcgcgggcggcggcgcggcgatcgagctcccccgcgcggcgagctctgctgcggggaggcgctcgccggtgccgcgcagcctgtgcgtgcgggaacggcccccgcagcccaatcggactctagagccagcggcagcgcgcctctcgcaggcggcggcgtccagcgcccggggccgggctgcgcggccagccccgagggctgcggcgccagggacgcgcggggcccgcgctccgccgccgccgccgcctgctgctgcgaggtcatccggatcttatcgtgccagctgatgcccgcttttcccactctggatctggatgggaagttggggaag

SEQ ID NO: 12 - DNA, 1941 bp, Mus musculus
atggatagggtcgtgttggggtggactgctgtcttctggttaacagccatggttgaaggccttcaggtcacagtgcctgacaagaagaaggtggccatgctcttccagcccactgtgcttcgatgccacttttccacgtcctcccatcagcctgcggtggttcagtggaagttcaaatcctactgccaggatcgcatgggagaatccttgggcatgtcttctccccgagcccaagcactcagcaagaggaacctggaatgggacccctacttggattgtttagacagcagaaggaccgtccgagtggtagcttccaaacagggctcgacggttaccctgggagatttctacaggggcagagagatcacaatagttcacgatgcagatcttcaaattggaaaactcatgtggggagacagcggactctactactgtatcatcaccaccccggatgacctggaaggcaaaaacgaagactcagtggaactgctggtgttgggcaggacagggctgcttgctgatctcttgcccagttttgctgtggagattatgccagagtgggtgtttgtcggcctggtgatcctggggattttcctcttcttcgtgctggtggggatctgctggtgccaatgctgccctcacagttgctgctgctatgtccgctgcccatgctgcccagattcctgctgctgccctcaggccttgtatgaagcagggaaagcagccaaggccgggtaccctccctctgtctccggtgtccccggcccctactccatcccctctgtccctttgggaggagccccctcttctggcatgctgatggacaagccgcatccacctcccctggcaccaagtgattccactggaggaagccacagtgttcgaaaaggttaccggatccaggctgacaaagagagagactccatgaaggtcctgtactatgtcgagaaggagctggctcagtttgatccagccaggaggatgagaggcagatataacaacaccatctcggaactcagctccctgcatgatgatgacagcaatttccgccagtcttaccaccagatgcggaataagcagttccctatgtctggagacctggaaagcaatcccgactactggtcaggtgtcatgggaggcaacagtgggaccaacagggggccagccttggagtataacaaagaggaccgtgagagcttcaggcacagccagcagcgctccaaatccgagatgctgtcgcggaagaactttgccacgggcgtgccggccgtgtcgatggacgagctggcagccttcgcagactcgtacggccagcggtcgcggcgcgccaatggcaacagccacgaggcgcgggcggggagccgcttcgagcgctcggagtcgcgggcccacggtgccttctaccaggacggctcgctggatgagtactacgggcgcggacgcagtcgcgagcccccgggagacggggagcgcggctggacctacagccccgcacccgcacgccgccggccgccggaggatgcgcctctgccgcgcctggtgagccggaccccgggcaccgcgcccaagtacgatcactcgtacctgagcagcgtgctggagcgccaggcgcggccggagagcagcagccgcgggggcagcctggagacgccgtccaagctgggcgcgcagctgggcccgcgcagcgcatcctactacgcctggtcgccgccagccacatacaaggctggggccagcgagggcgaagacgaggacgacgcggcggatgaggacgcgctgccaccctacagcgagctggagctgagccgcggagagctgagccggggcccgtcctaccgtgggcgtgacctgtccttccacagcaactcggagaagaggaggaaaaaggagcccgtcaagaaacccggtgactttccaaccaggatgtcccttgtagtctga

SEQ ID NO: 13 - DNA, 1941 bp, Mus musculus
atggatagggtcgtgttggggtggaccgctgtcttctggttaacagccatggttgaaggccttcaggtcacagtgcctgacaagaagaaggtggccatgctcttccagcccactgtgcttcgatgccacttttccacgtcctcccatcagcctgcggtggttcagtggaagttcaaatcctactgccaggatcgcatgggagaatccttgggcatgtcttctccccgagcccaagcgctcagcaagaggaacctggaatgggacccctacttggattgtttagacagcagaaggaccgtccgagtggtagcttccaaacagggctcgacggttaccctgggagatttctacaggggcagagagatcacaatagttcacgatgcagatcttcaaattggaaagctcatgtggggagacagcggactctactactgtatcatcaccaccccggatgacctggaaggcaaaaacgaagactcagtggaactgctggtgttgggcaggacagggctgcttgctgatctcttgcccagttttgctgtggagattatgccagagtgggtgtttgtcggcctggtgatcctggggattttcctcttcttcgtgctggtggggatctgctggtgccaatgctgccctcacagttgctgctgctatgtccgctgcccatgctgcccagattcctgctgctgccctcaggccttgtatgaagcagggaaagcagccaaggccgggtaccctccctctgtctccggtgtccccggcccctactccatcccctctgtccctttgggaggagccccctcttctggcatgctgatggacaagccgcatccacctcccctggcaccaagtgattccactggaggaagccacagtgttcgcaaaggttaccggatccaggctgacaaagagagagactccatgaaggtcctgtactatgtcgagaaggagctggctcagtttgatccagccaggaggatgagaggcagatataacaacaccatctcggaactcagctccctgcatgatgatgacagcaatttccgccagtcttaccaccagatgcggaataagcagttccctatgtctggagacctggaaagcaatcctgactactggtcaggtgtcatgggaggcaacagtgggaccaacagggggccagccttggagtataacaaagaggaccgtgagagcttcaggcacagccagcagcgctccaaatctgagatgctgtcgcggaagaactttgccacgggcgtgccggccgtgtcgatggacgagctggcagccttcgcagactcgtacggccagcggtcgcggcgcgccaatggcaacagccacgaggcgcgggcggggagccgcttcgagcgctcggagtcgcgggcccacggtgccttctaccaggacggctcgctggatgagtactacgggcgcggacgcagtcgcgagccgccgggagacggggagcgtggctggacctacagccccgcacccgcacgccgccggccgccagaggatgcgcctctgccgcgcctggtgagccggaccccgggcaccgcgcccaagtacgatcactcgtacctgagcagcgtgctggagcgccaggcgcggccggagagcagcagccgcgggggcagcctggagacgccgtccaagctgggcgcgcagctgggcccgcgcagcgcatcctactacgcctggtcgccgccaaccacatacaaagctggggccagcgagggcgaagacgaggacgacgcggcggatgaggacgcgctgccaccctacagcgagctggagctgagccgcggagagctgagccggggcccgtcctaccgtgggcgtgacctgtccttccacagcaactcggagaagaggaggaaaaaggagcccgccaagaaacccggcgactttccaaccaggatgtcccttgtagtctga

SEQ ID NO: 14 - protein, 646 aa, Mus musculus
  MOD_RES (572)..(572): Ala or Thr
MDRVVLGWTAVFWLTAMVEGLQVTVPDKKKVAMLFQPTVLRCHFSTSSHQPAVVQWKFKSYCQDRMGESLGMSSPRAQALSKRNLEWDPYLDCLDSRRTVRVVASKQGSTVTLGDFYRGREITIVHDADLQIGKLMWGDSGLYYCIITTPDDLEGKNEDSVELLVLGRTGLLADLLPSFAVEIMPEWVFVGLVILGIFLFFVLVGICWCQCCPHSCCCYVRCPCCPDSCCCPQALYEAGKAAKAGYPPSVSGVPGPYSIPSVPLGGAPSSGMLMDKPHPPPLAPSDSTGGSHSVRKGYRIQADKERDSMKVLYYVEKELAQFDPARRMRGRYNNTISELSSLHDDDSNFRQSYHQMRNKQFPMSGDLESNPDYWSGVMGGNSGTNRGPALEYNKEDRESFRHSQQRSKSEMLSRKNFATGVPAVSMDELAAFADSYGQRSRRANGNSHEARAGSRFERSESRAHGAFYQDGSLDEYYGRGRSREPPGDGERGWTYSPAPARRRPPEDAPLPRLVSRTPGTAPKYDHSYLSSVLERQARPESSSRGGSLETPSKLGAQLGPRSASYYAWSPPXTYKAGASEGEDEDDAADEDALPPYSELELSRGELSRGPSYRGRDLSFHSNSEKRRKKEPXKKPGDFPTRMSLVV

SEQ ID NO: 15 - DNA, 6052 bp, Mus musculus
tacttataagacacctctctggatgactggaaatcagatgcagactatggagacaagacccaaatctgagagccggcaagcctaggatcttctctggccagcagccaccttggaagctttgctgatctctgctttggcaagggatcctcctttaagaaggctgatttcaaatcttagtgcccaactatctcgagcaacttaccaagaaaacgctctgtgagaacatatcacgtaataaccgaccaagtttatcttacactcccaccccccaccccccatttccttagcagaaacaagactctgcgtccagttctgaagctggaagctttgaacccctgatctctagaaattacctatgcctgcagtatgtttttctatgagtgctgttctgtgcttagacagaggaatttactactacagttagaagaccgtctgctcacaagagagataaatggtaaaatgtaccttgtatccccttgcttccagtcactggtcaatgagtcttgttatgctaaaatcagaaggcctttagtgagcgtactggccgtgacctcctgggcaatcacagaaatggcttcaatttgctgctctgactcacaattctaagtggctgggacaaacagaggagagcattttgaaaaaccatcttaagtggtctttctttttccattcagaggacacaaactgcttttcatctttctgtcaaacagagtgacaatcctaaggttctccctgcccagcccacaccggtccctctctttcctccctctcctggtcttttcagggctggtgcctctgagggtgttccactccatgcttcagtgtgaatagcttgtcatcaggtgcctttgacagatgcttcaaacaaacatttgagagagaagaaaagcagaagtcggtgatacaaaatgaacaggaaatgacatgtaggctcattatattttgaatgtgggttgtttccccacaaacacactcagatttgtttttgtttttatttttggatttgtacttcacttaagaattatttctaccatcctgattctgcagctgttgggcaccagggaatgtggtgtccacatcttttggcctcactggcccaccactattgatgctttggggaaaagaaggacagcacttcctcttcctgccattgcaaaaaaaaaaaaatgatttttgcctgaatccctaattgaacttttgtaggtaaactgcaaaagtggccacaaactcttcccctctcatgttcctgtgaagggatttgtcctcttgctgccacaggccctgccaaatgcacctcagctatcctacatgatgagagaagagcctggtcaccaccgtcattatctgtgcccatcttatcaactttaagcagacttggaagaacatctagccacgaccaacaaaagaactgcctagctgagccgagcccaaactggagattcccgcttgagaggagacattcagcattcctgtgttcgtttaccatcgacgataaacctcccatcagaatatttgtctctggtcggttactcacccaaccttgggtgtcacacaaccttcacttttgttagcagacttttcaatctgcattattgtggtgagacacgtgactggatgaagtgactggagcaaggggatgcttgctatcccctaatccagtggtggtctacttctacttattgatctacatgtagtctctgattcactggtcagtatttccatggccatgtgactggaattccagagtccattctgttagcatccattatacttcatgagatttccagaaaaggtcctctgtgagtggtgtaagagctgctgggttagggtgggtgttggggggtggaatcattacttggaggagaactggcctgctaaaggacttcacggttgctttggcctgccctagatggatcaggaggatacttcagcccaatgctggcacttccaagggctggaagacaaaagccataaccctggtgctgagttttaggtttgctagtgtccctggcctcagaacacctaggtctgatctgtctgtttgggctctaaatcaatatggcaaaaacatcatttcttagtcaccagcttttgatttcaacttgctcaggcacttttgaagaatattggatagccgcagtagctattgttatactgagcactgtgtcaggcttcttagcaccaaagagccccatagcacaggctacagagaccaaatatattgctttatagagccaggggcgtgtatgagcttggggaaagctgagggagcgatgaatgaaagaaaaaagttaaaattggaaacataaggttctaaagacaacaagtctataggctgacaaattaaaaaaaaaatttcaatgtagagaagataacaggctttcaatataacgggggaaagtggggcacagattgttctttatagggcatgagtcacgtgggcttccagaccttcagtacagaggaaattcagttgcttctgggtccgtggataggagatgatctgaatggacaaggctaagctggccgtccttgatgcccttgacatttctttacacacccctttgtttcttctccaaatactgtgtcctgcacaggaagtgcctatgcgtattagttcctttcctgtttttctagggcataagcaaagtgtaagaggtgatctccatccactgatcccctacaatttaagaaggaagataagtcatgcccaagaaaggatgagtatattttatgcatatgataagaaatagtgctatggataaattataataaacccagagatttaaagttttctttaaaaacaaaaaccttaaatgggaatattttgatatttaagtgttgtgtgtttgtccatccattccatttttaggacatgctcagtgatctgcaaagccaggctgtagaagtctgagctgaaaggaggtgaaggagaagaaagagggatgagtggcctcagggaggagggaagagagtagaggcccgcttacaggagcttctgtctctgcctgtgactcacagctgagtcagggacaagctggaggagggagtatggaagcaggtggcaggagaggtcccctggtgctcagagctcttctctaggctatgtatagactcattaggagactcaggactgtattcagttcttccatccaagcaagcccaggggagcttgggatttagtcctcctggcacttgtatctacagcttggggtgcagtagtacctcacatgggttgggaacctcacctcccttctcatgatcctcactctgcatgtggtgtaggggtgggcaccccagggtgagagggggctggcgctacatataaaaatctggttagatccgaagcagtctttgagaggagtggagtaactaacagacaccgctttggctcatctgctctccatccatttctaaatagatggataagccatcatccacatttatggagtcacaaaccagtcagatctttagattcccaatctataggcctttcctgctggatctgtgtttttgcaaaattgcctagtcataagaattacttgcctagggactggagagagatggcttaacagtcaaaaacactgctttagccgaggacccgagtttggttcccagcactctactaatgctcacagctgtctgtaacttcaattccagggacccactgatctcatagagcatctgggagcactaaactcacatggtacgcatatataccatcaaaaaactctcaggcacacaaaataaaaataaatacattgttaaaaactgaaaaagaaaggggctggagagatggctctgtgattacgtgtgctggctactcttccagaggacccacattcatttttcagcacatggagactcccagcaccagttcagacacacacacacacacacacacacacacacacacacacacacacacacacacacgaaggcaaaatacacacctaacaaagaaataaagcatttaaaaatacttggtaaaacaattacttgcctgggggactgggatgtggctcagttggtagagcacttgattagcatgcacaaagccctggttcaatccctagcacaataaactaggggtagtggcacatgcctgtgatcccaacatctgggagagtttcaagttcaaggtcatccttggctacatagtgagttcaaggtcaccctgggctgtatgatactctgccttaaagaaccaaacactgaaaataacaatgaaaaaacacaaggattactgactgccactgtcacaaatgctgttgcactgtaccttggagaatggatgggtggatctggagtaaggatttgtattctgaacatatccttttagaatgccttgtggtagatgcatttgggtggtgctatactggatcatacctctggtgcacccctatctgctggccaggaatattgtttgtgctgtggattatttcattccaatatcactgtgaggtccctctaacttccttaggtctggcactggtgtggcatccagcagtcccagctatgacactggagaatggctcaccagagtcaggctgaaaggaaacatttaaagggagggggttggaggacctccccccgggagacttcttgacatgttccaactcccagaatagtgatatgttgtgacaggctgagatcagacaacaggaattacagacaattttcttattcccttaccatcctgaataaaacttagctcatgaatagaaaaaaaaaaagccatcagagaaaatggcaaacgtaaatcatttttaaagggtaaaaattaaaagctttgctaacataactttcatgctaggaccaaaagtgggtggagaaaaaaatagtaaaatatatattacctattccaaaactgatttaattgcagccagaatcttatggaagtttagaagtgatgtatagagtacaggaatcacccatggaaattctaaggtcttagaaagcaaaaggttccctaccaggacctacctcctagtcacttgggattacctgtgaagctcaaaggccctagtggcatcaaaggtgagtaagaagaagccgagatgctttaagcaacagcgtgaggttggcatcaacggggcacatttgttcttcacagcaagcccagtgttttcccatcttacccaatgtggagctgggtctgaaagtgtgccaagtgatcacctattgccaaatagcttttagtctttagatggccttctgactgtccaggtcctaagcctacagtaatcacgggcccagcctctagtgtgttctcttcccaagcagatggatagtggagagagccctgactcaatattcactcacacatcattggtgaggagaagctaggaaggcaggcatttgccacttcatctatccacaggaggttccttgaagtctgccctgagaaggaggtgtctttgctggggaggatcttcagcatcagcatcaagctgtgaggggaaaggctttgacaaaagggttgccactttctgaattcttctcaaagaggaatttctaagccaagctacagattcatccaggctcagaattccatggctgtgggcaggagctgtcatcttcactatattttgagatacattttttttaggtagaactcgaggtccagatctagagggggataagggagatgagaaggataaagttgtggcagttgagctaaaagtcatgttcgagttttttggtgggtctgactggacaggggaaaatgtggtccgactccttttatctaaaaggttgggaaagatacccatagcttctctcttgccatgtttattaacaaagatgttagacactactccatgagaaatttccttgtgaaaataaaaaccatgccatcaaaagagtcgggtgcaaagacgcctacttcatgagaatcacctgcccagttgtttttgtgccttgtctgtgacatcaaaactgaaacatttatatcactgtcactcatggttttattttcctgtgtcatacatacaacgtgcatttgattgtaatgatttaaagtaaataaagcatttcatctacttttgtt

SEQ ID NO: 16 - DNA, 6037 bp, Mus musculus
tacttataagacacctctctggatgactggaaatcagatgcagactatggagacaagacccaaatctgagagccggcaagcctaggatcttctctggccagcaaccaccttggaagctttgctgatctctgctttggcaagggatcctcctttaagaaggctgatttcaaatcttagtgcccatctatctcgagcaacttaccaagaaaacgctctgtgagaacatatcacgtaataaccgaccaagtttatcttacacccccacccccacccccatttccttagcagaaacaagactctgcgtccagttctgaagctggaagctttgaacccctgatctctagaaattacctatgcctgcagtatgtttttctacgagtgctattctgtgcttagacagaggaatttactactacagttagaagaccgtctgctcacaagagagataaatggtaaaatggaccttgtatccccttgcttccagtcactggtcaatgagtcttgttatgctaaaaccagaaggcctttagtgagcgtactggccgtgacctcctgggcaatcacagaaatggcttcaatttgctgctctgactcacaattctaagtggctgggacaaacagaggagagcattttgaaaaaccatcttaagtggtctttctttttccattcagaggacacaaactgcttttcatctttctgtcgaacagagtgacaatcctaaggttctccctgcccagcccacactggtccctctcttccctccctctcctggtcttttcagggctggtgcctctgagggtgttccactccatgcttcagtgtgaatagcttgtcatcaggtgcctttgacagatgcttcaaacaaacatttgagagagaagaaaagcagaagtcggtgatacaaaatgaacaggaaatgatatgtaggctcattatattttgaatgtgggttgtttccccacaaacacactcagatttgtttttgtttttatttttggatttgtacttcacttaagaattatttctaccatcctgattctgcagctgttgggcaccagggaatgtggtgtccacatcttttggcctcactggcccaccactattgatgctttggggaaaagaaggacagcacttcctcttcctgccattgcaaaaaaaaaaaattgatttttgcctgaatcccaaattgaacttttgtaggtaaactgcaaaagtggccacaaactcttcccctctcatgttcctgtgaagggatttgtcctcttgctgccacaggccctgccaaatgcacctcagctatcctacatgatgagagaagagcctggtcaccaccgtcattatctgtgcccatcttatcaactttaagcagacttggaagaacatctagccacgaccaacaaaagaactgcctagctgagccgagcccaaactggagattcccgcttgagaggagacattcagcattcctgtgttcatttatcattgacgataaacctcccgtcagaatatttgtctctggtcggttactcacccaaccttgggtgtcacacaaccttcacttttgttagcagacttttcaatctgcattattgtggtgagacacgtgactggatgaagtgactggagcaaggggatgcttgctatcccctaatccagtggtggtctacttctacttattgatctacatgtagtctctgattcactggtcagtatttccatggccacgtgactggaattccagagtccattctgttagcatccattatacttcatgagatttccagaaaaggtcctctgtgagtggtgtaagagctgctgggttagggtgggtgttggggggtggaatcattacttggaggagaactggcctgccaaaggacttcacggttgctttggcctgccctagatggatcaggaggatacttcagcccaatgctggcacttccaagggctggaagacaaaagccataacccttgtgctgagttttaggtttgttagtgtccctggcctcagaacacctaggtctgatctgtctgtttgggctctaaatcaatatggcaaaaacatcatttcttagtcaccagcttttgatttcaacttgctcaggcacttttgaagaatattggatagccgcagtagctattgttatactgagcactgtgtcaggcttcttagcaccaaagagccccatagcacaggctacagagaccaaatatattgctttatagagccaggggcgtgtatgagcttggggaaagctgagggatcgatgagtgaaagaaaaaagttaaaattggaaacataaggttctaaagacaacaagtctataggctgacaaattaaaaaaaatttcaatgtagagaagataacaggctttcaatataacgggggaaagtggggcacagattgttctttatagggcatgagtcacgtgggcttccagaccttcagtacagaggaaattcagttgcttctgggtccgtggataggagatgatctgaatggacaaggctaagctggccgtccttgatgccctcgacatttctttacacacccctttgtttcttctccaaatactgtgtcctgtacaggaagtgcctatgcgtattagttcctttcctgtttttctagggcataagcaaagtgtaagaggtgatctccatccactgatcccctacaatttaagaaggaagataagtcatgcccaagaaaggatgagtatattttatgcatatgataagaaatagtgctatggataaattataataaacccagagatttaaagttttctttaaaaacaaaaaccttaaatgggaatattttgatatttaagtgttgtgtgtttgtccatccatcccatttttaggacatgctcagtgatctgcaaagccaggctgtagaagtctgagctgaaaggaggtgaaggagaagagagagggatgagtggcctcagggaggagggaagagagtagaggcccgcttacaggagcttctgtctctgtctgtgactcacagctgagtcagggacaagctggaggagggagtatggaagcaggtggcaggagaggtcccctggtgctcagagctcttctctaggctatgtatagactcattaggagactcaggactgtattcagttcttccatccaagcaagcccaggggagcttgggatttagtcctcctggcacttgtatctacagcttggggtgcagtagcacctcacatgggttggggacctcacctcccttctcatgatcctcactctgcatgtggtgtaggggtgggcaccccagggtgagagggggctggcgctacatataaaaatctggttagatccgaagcagtctttgagaggagtggagtaactaacagacaccgctttggctcatctgctctccatccatttctaaatagatggataagccatcatccacatttatggagtcacaaaccagtcagatctttagattcccaatctataggcctttcctgctggatctgtgtttttgcaaaattgcctagtcataagaattacttgcctagggactggagagagatggcttaacagtcaaaaacactgctttagccgaggacccgagtttggttcccagcactctactaatgctcacagctgtctgtaacttcaattccagggacccactgatctcatagagcatctgggagcactaaactcacatggtacgcatatataccatcaaaaaactctcaggcacacaaaataaaaataaatacattgttaaaaactgaaaaagaaaggggctggagagatggctctgtgattacgtgtgctggctactcttccagaggacccacattcatttttcagcacatggagactcccagcaccagttcagacacacacacacacacacacacacacacacacacacacgaaggcaaaatacacacctaacaaagaaataaagcatttaaaaatacttggtaaaacaattacttgcctgggggactgggatgtggctcagttggtagagcgcttgattagcatgcacaaagccctggttcaatccctagcacaataaactaggggtagtggcacatgcctgtgatcccaacatctgggagagtttcaagttcaaggtcatccttggctacatagtgagttcaaggtcaccctgggctgtatgatactctgccttaaagaaccaaacactgaaaataacaatgaaaaaacacaaggattactgactgccactgtcacaaatgctgttggactgtaccttggagaatggatgggtggatctggagtaaggatttgtattctgaacatatccttttagaatgccttgtggtagatgcatttgggtggtgctatactggatcatacctctggtgcacccctatctgctggccaggaatattgtttgtgctgtggattatttcattccaatatcactgtgaggtccctctaacttccttaggtctggcactggtgtggcatccagcagtcccagctatgacactggagaatggctcaccagagtcaggctgaaaggaaacatttaaagggagggggttggaggacctccccccgggaggcttcttgacatgttccaactcccagaatagtgatatgttgtgacaggctgagatcagacaacaggaattacagacaattttcttattcccttaccatcctgaataaaacttagctcatgaatagaaaaaaaaaaaagccatcagagaaaatggcaaacgtaaatcatttttaaagggtaaaaattaaaagctttgctaataacataactttcatgctaggaccaaaagtgggtggagaaaaaaatagtaaaatatatattacctattccaaaactgatttaattgcagccagaatcttatggaagtttagaagtgatgtatagagtacaggaatcacccatggaaattctaaggtcttagaaagcaaaaggttccctaccaggacctaccccctagtcacttgggattacctgtgaagctcaaaggccctagtggcatcaaaggtgagtaagaagaagccgagatgctttaagcaacagcgtgaggttggcatcaacggggcacatttgttcttcacagcaagcccagtgttttcccatcttacccaatgtggagctgggtctgaaagtgtgccaagtgatcacctattgccaaatagcttttagtctttagatggccttctgactgtccaggtcctaagcctacagtaatcacgggcccagcctctagtgtgttctcttcccaagcaggtggatagtggagagagccctgactcaatattcactcacacatcattggtgaggagaagctaggaaggcaggcatttgccacttcatctatccacaggaggttccttgaagtctgccctgagaaggaggtgtctttgctggggaggatcttcatcatcagcatcaagctgtgaggggaaaggctttgacaaaagggttgccactttctgaattcttctcaaagaggaatttctaagccaagctacagattcatccaggctcagaattccatggctgtgggcaggagctgtcatcttcactatattttgagatacatttttttttaggtagaactcgaggtccagatctagagggggataaggacgatgagaaggataaagttgtggcagttgagctaaaagtcatgttcgagttttttggtgggtctgactggacaggggaaaatgtggtccgacttcttttatctaaaagtttgggaaagatacccatagcttctctcttgccatgtttattaacaaagatgttagacactactccatgagaaatgtccttgtgaaaataaaaaccatgccatcaaaagagtcgggtgcaaagatgcctacttcatgagaatcacctgcccagttgtttttgtgccttgtctgtgacatcaaaactgaaacacttatatcactgtcactcatggttttcttttcctgtgtcatacatacaaagtgcatttgattgtaatgatttaaagtaaataaagcatttcatctacttttgtt

SEQ ID NO: 17 - DNA, 8845 bp, Mus musculus
gtgggcatagcccatggaaatgtacgctgcaaatggagaatgtataggtgtagtctccctctccctctccctcctcctcccccctccccctctccctcccattctctctctccctctctctctctgctcattttctgttaaaaaggccacatacgttttaacagaaaaaagatttttgcctaaagggtttctcccctatggatcctaatttggttggggcctcttggttcgttgaaccagatgcaccagccagggcacaacaaaaacaaacaaacaaacaaacaccatacaggtctaagccccaggagaagttatgccaagtccttgtagcctttctgtccctgacacccagtacaggtgcaaggaaacatcgagtcccagcctgcttggtggctcaagtagagcttgagtcgcagcccccgccacatggtccgccctctggggtggacttcgctgctaggttctgacctcccagccccgggagacggcacgtgaccgagaaactttggcgggcggtgttggcgcgggcggcggcgcggcgatcgagctcccccgcgcggcgagctctgctgcggggaggcgctcgccggtgccgcgcagcctgtgcgtgcgggaacggcccccgcagcccaatcggactctagagccagcggcagcgcgcctctcgcaggcggcggcgtccagcgcccggggccgggctgcgcggccagccccgagggctgcggcgccagggacgcgcggggcccgcgctccgccgccgccgccgcctgctgctgcgaggtcatccggatcttatcgtgccagctgatgcccgcttttcccactctggatctggatgggaagttggggaagatggatagggtcgtgttggggtggactgctgtcttctggttaacagccatggttgaaggccttcaggtcacagtgcctgacaagaagaaggtggccatgctcttccagcccactgtgcttcgatgccacttttccacgtcctcccatcagcctgcggtggttcagtggaagttcaaatcctactgccaggatcgcatgggagaatccttgggcatgtcttctccccgagcccaagcactcagcaagaggaacctggaatgggacccctacttggattgtttagacagcagaaggaccgtccgagtggtagcttccaaacagggctcgacggttaccctgggagatttctacaggggcagagagatcacaatagttcacgatgcagatcttcaaattggaaaactcatgtggggagacagcggactctactactgtatcatcaccaccccggatgacctggaaggcaaaaacgaagactcagtggaactgctggtgttgggcaggacagggctgcttgctgatctcttgcccagttttgctgtggagattatgccagagtgggtgtttgtcggcctggtgatcctggggattttcctcttcttcgtgctggtggggatctgctggtgccaatgctgccctcacagttgctgctgctatgtccgctgcccatgctgcccagattcctgctgctgccctcaggccttgtatgaagcagggaaagcagccaaggccgggtaccctccctctgtctccggtgtccccggcccctactccatcccctctgtccctttgggaggagccccctcttctggcatgctgatggacaagccgcatccacctcccctggcaccaagtgattccactggaggaagccacagtgttcgaaaaggttaccggatccaggctgacaaagagagagactccatgaaggtcctgtactatgtcgagaaggagctggctcagtttgatccagccaggaggatgagaggcagatataacaacaccatctcggaactcagctccctgcatgatgatgacagcaatttccgccagtcttaccaccagatgcggaataagcagttccctatgtctggagacctggaaagcaatcccgactactggtcaggtgtcatgggaggcaacagtgggaccaacagggggccagccttggagtataacaaagaggaccgtgagagcttcaggcacagccagcagcgctccaaatccgagatgctgtcgcggaagaactttgccacgggcgtgccggccgtgtcgatggacgagctggcagccttcgcagactcgtacggccagcggtcgcggcgcgccaatggcaacagccacgaggcgcgggcggggagccgcttcgagcgctcggagtcgcgggcccacggtgccttctaccaggacggctcgctggatgagtactacgggcgcggacgcagtcgcgagcccccgggagacggggagcgcggctggacctacagccccgcacccgcacgccgccggccgccggaggatgcgcctctgccgcgcctggtgagccggaccccgggcaccgcgcccaagtacgatcactcgtacctgagcagcgtgctggagcgccaggcgcggccggagagcagcagccgcgggggcagcctggagacgccgtccaagctgggcgcgcagctgggcccgcgcagcgcatcctactacgcctggtcgccgccagccacatacaaggctggggccagcgagggcgaagacgaggacgacgcggcggatgaggacgcgctgccaccctacagcgagctggagctgagccgcggagagctgagccggggcccgtcctaccgtgggcgtgacctgtccttccacagcaactcggagaagaggaggaaaaaggagcccgtcaagaaacccggtgactttccaaccaggatgtcccttgtagtctgatacttataagacacctctctggatgactggaaatcagatgcagactatggagacaagacccaaatctgagagccggcaagcctaggatcttctctggccagcagccaccttggaagctttgctgatctctgctttggcaagggatcctcctttaagaaggctgatttcaaatcttagtgcccaactatctcgagcaacttaccaagaaaacgctctgtgagaacatatcacgtaataaccgaccaagtttatcttacactcccaccccccaccccccatttccttagcagaaacaagactctgcgtccagttctgaagctggaagctttgaacccctgatctctagaaattacctatgcctgcagtatgtttttctatgagtgctgttctgtgcttagacagaggaatttactactacagttagaagaccgtctgctcacaagagagataaatggtaaaatgtaccttgtatccccttgcttccagtcactggtcaatgagtcttgttatgctaaaatcagaaggcctttagtgagcgtactggccgtgacctcctgggcaatcacagaaatggcttcaatttgctgctctgactcacaattctaagtggctgggacaaacagaggagagcattttgaaaaaccatcttaagtggtctttctttttccattcagaggacacaaactgcttttcatctttctgtcaaacagagtgacaatcctaaggttctccctgcccagcccacaccggtccctctctttcctccctctcctggtcttttcagggctggtgcctctgagggtgttccactccatgcttcagtgtgaatagcttgtcatcaggtgcctttgacagatgcttcaaacaaacatttgagagagaagaaaagcagaagtcggtgatacaaaatgaacaggaaatgacatgtaggctcattatattttgaatgtgggttgtttccccacaaacacactcagatttgtttttgtttttatttttggatttgtacttcacttaagaattatttctaccatcctgattctgcagctgttgggcaccagggaatgtggtgtccacatcttttggcctcactggcccaccactattgatgctttggggaaaagaaggacagcacttcctcttcctgccattgcaaaaaaaaaaaaatgatttttgcctgaatccctaattgaacttttgtaggtaaactgcaaaagtggccacaaactcttcccctctcatgttcctgtgaagggatttgtcctcttgctgccacaggccctgccaaatgcacctcagctatcctacatgatgagagaagagcctggtcaccaccgtcattatctgtgcccatcttatcaactttaagcagacttggaagaacatctagccacgaccaacaaaagaactgcctagctgagccgagcccaaactggagattcccgcttgagaggagacattcagcattcctgtgttcgtttaccatcgacgataaacctcccatcagaatatttgtctctggtcggttactcacccaaccttgggtgtcacacaaccttcacttttgttagcagacttttcaatctgcattattgtggtgagacacgtgactggatgaagtgactggagcaaggggatgcttgctatcccctaatccagtggtggtctacttctacttattgatctacatgtagtctctgattcactggtcagtatttccatggccacgtgactggaattccagagtccattctgttagcatccattatacttcatgagatttccagaaaaggtcctctgtgagtggtgtaagagctgctgggttagggtgggtgttggggggtggaatcattacttggaggagaactggcctgctaaaggacttcacggttgctttggcctgccctagatggatcaggaggatacttcagcccaatgctggcacttccaagggctggaagacaaaagccataaccctggtgctgagttttaggtttgctagtgtccctggcctcagaacacctaggtctgatctgtctgtttgggctctaaatcaatatggcaaaaacatcatttcttagtcaccagcttttgatttcaacttgctcaggcacttttgaagaatattggatagccgcagtagctattgttatactgagcactgtgtcaggcttcttagcaccaaagagccccatagcacaggctacagagaccaaatatattgctttatagagccaggggcgtgtatgagcttggggaaagctgagggagcgatgaatgaaagaaaaaagttaaaattggaaacataaggttctaaagacaacaagtctataggctgacaaattaaaaaaaaaatttcaatgtagagaagataacaggctttcaatataacgggggaaagtggggcacagattgttctttatagggcatgagtcacgtgggcttccagaccttcagtacagaggaaattcagttgcttctgggtccgtggataggagatgatctgaatggacaaggctaagctggccgtccttgatgcccttgacatttctttacacacccctttgtttcttctccaaatactgtgtcctgcacaggaagtgcctatgcgtattagttcctttcctgtttttctagggcataagcaaagtgtaagaggtgatctccatccactgatcccctacaatttaagaaggaagataagtcatgcccaagaaaggatgagtatattttatgcatatgataagaaatagtgctatggataaattataataaacccagagatttaaagttttctttaaaaacaaaaaccttaaatgggaatattttgatatttaagtgttgtgtgtttgtccatccattccatttttaggacatgctcagtgatctgcaaagccaggctgtagaagtctgagctgaaaggaggtgaaggagaagaaagagggatgagtggcctcagggaggagggaagagagtagaggcccgcttacaggagcttctgtctctgcctgtgactcacagctgagtcagggacaagctggaggagggagtatggaagcaggtggcaggagaggtcccctggtgctcagagctcttctctaggctatgtatagactcattaggagactcaggactgtattcagttcttccatccaagcaagcccaggggagcttgggatttagtcctcctggcacttgtatctacagcttggggtgcagtagtacctcacatgggttgggaacctcacctcccttctcatgatcctcactctgcatgtggtgtaggggtgggcaccccagggtgagagggggctggcgctacatataaaaatctggttagatccgaagcagtctttgagaggagtggagtaactaacagacaccgctttggctcatctgctctccatccatttctaaatagatggataagccatcatccacatttatggagtcacaaaccagtcagatctttagattcccaatctataggcctttcctgctggatctgtgtttttgcaaaattgcctagtcataagaattacttgcctagggactggagagagatggcttaacagtcaaaaacactgctttagccgaggacccgagtttggttcccagcactctactaatgctcacagctgtctgtaacttcaattccagggacccactgatctcatagagcatctgggagcactaaactcacatggtacgcatatataccatcaaaaaactctcaggcacacaaaataaaaataaatacattgttaaaaactgaaaaagaaaggggctggagagatggctctgtgattacgtgtgctggctactcttccagaggacccacattcatttttcagcacatggagactcccagcaccagttcagacacacacacacacacacacacacacacacacacacacacacacacacacacacgaaggcaaaatacacacctaacaaagaaataaagcatttaaaaatacttggtaaaacaattacttgcctgggggactgggatgtggctcagttggtagagcacttgattagcatgcacaaagccctggttcaatccctagcacaataaactaggggtagtggcacatgcctgtgatcccaacatctgggagagtttcaagttcaaggtcatccttggctacatagtgagttcaaggtcaccctgggctgtatgatactctgccttaaagaaccaaacactgaaaataacaatgaaaaaacacaaggattactgactgccactgtcacaaatgctgttgcactgtaccttggagaatggatgggtggatctggagtaaggatttgtattctgaacatatccttttagaatgccttgtggtagatgcatttgggtggtgctatactggatcatacctctggtgcacccctatctgctggccaggaatattgtttgtgctgtggattatttcattccaatatcactgtgaggtccctctaacttccttaggtctggcactggtgtggcatccagcagtcccagctatgacactggagaatggctcaccagagtcaggctgaaaggaaacatttaaagggagggggttggaggacctccccccgggagacttcttgacatgttccaactcccagaatagtgatatgttgtgacaggctgagatcagacaacaggaattacagacaattttcttattcccttaccatcctgaataaaacttagctcatgaatagaaaaaaaaaaagccatcagagaaaatggcaaacgtaaatcatttttaaagggtaaaaattaaaagctttgctaacataactttcatgctaggaccaaaagtgggtggagaaaaaaatagtaaaatatatattacctattccaaaactgatttaattgcagccagaatcttatggaagtttagaagtgatgtatagagtacaggaatcacccatggaaattctaaggtcttagaaagcaaaaggttccctaccaggacctacctcctagtcacttgggattacctgtgaagctcaaaggccctagtggcatcaaaggtgagtaagaagaagccgagatgctttaagcaacagcgtgaggttggcatcaacggggcacatttgttcttcacagcaagcccagtgttttcccatcttacccaatgtggagctgggtctgaaagtgtgccaagtgatcacctattgccaaatagcttttagtctttagatggccttctgactgtccaggtcctaagcctacagtaatcacgggcccagcctctagtgtgttctcttcccaagcagatggatagtggagagagccctgactcaatattcactcacacatcattggtgaggagaagctaggaaggcaggcatttgccacttcatctatccacaggaggttccttgaagtctgccctgagaaggaggtgtctttgctggggaggatcttcagcatcagcatcaagctgtgaggggaaaggctttgacaaaagggttgccactttctgaattcttctcaaagaggaatttctaagccaagctacagattcatccaggctcagaattccatggctgtgggcaggagctgtcatcttcactatattttgagatacattttttttaggtagaactcgaggtccagatctagagggggataagggagatgagaaggataaagttgtggcagttgagctaaaagtcatgttcgagttttttggtgggtctgactggacaggggaaaatgtggtccgactccttttatctaaaaggttgggaaagatacccatagcttctctcttgccatgtttattaacaaagatgttagacactactccatgagaaatttccttgtgaaaataaaaaccatgccatcaaaagagtcgggtgcaaagacgcctacttcatgagaatcacctgcccagttgtttttgtgccttgtctgtgacatcaaaactgaaacatttatatcactgtcactcatggttttattttcctgtgtcatacatacaacgtgcatttgattgtaatgatttaaagtaaataaagcatttcatctacttttgtt

SEQ ID NO: 18 - DNA, 2861 bp, Mus musculus
gactagcaaatgttgtctttaataatattttcaaagtctcagttaaaagatttagaaaacaagtggatcagttacattacaaataggttccctagatttaaataaaaggaaattcaatatatttctaagtttttaaaaaataatcaaaatttctgttatttttctagctacatcataactccggtcctagttcataagtacttctgcacaaagcttggaagtgagaaatctgtgaccacatctttcttacattttttaggcaggggccagagttcaagctacagcccagtggacacaaaggctaagtccaccttccaaacttctggccttcacaaccacaaacacctgcaatcctttggtagggagggaaacaggtctacccaggccttaagtaggtctggtgagccttgggcagggcattacacagcaggagcatggtctaaaaggtaagtgaactgaaaccaaggtatatgtccttcaccttgactttgagccatttggagagcagaatgggcctcttctaaagcacggggttcatactggctctaaagaccccttttgggaccgggccagcagtagagaacatgctattagtagtggcttttttcccccttcctctcttggcccaacatagcctaaatcattgaagttcaccgcagtgattatataggacagagaaaaacatttgaacaagggagatagatgcacgaggaggccagctgcagacagcctgagttccgggaagcctgccttaggtagaacaaagacaattgtctccctattccaagaacagcatgtaggaagcctccctctctgtaagcaagttgggtttgagctggagccaattcctgctgagtaacacaaataccacctgtgagcatctacagctcacactggtcaggaccaaggctcccaggcagaagattctggaatatgcgatctcagcccttagcagcactcccttccaaccatttagaaaaccatggtgcctgcttttgttcctgcagataacaacaccatctcggaactcagctccctgcatgatgatgacagcaatttccgccagtcttaccaccagatgcggaataagcagttccctatgtctggagacctggaaagcaatcccgactactggtcaggtgtcatgggaggcaacagtgggaccaacagggggccagccttggagtataacaaagaggaccgtgagagcttcaggcacaggtgacggccatgagtgggaagggaccactgtgtatctgttcttctgtttctatagactatggaatatctcttacatatattacacccttgtgatactgtgtgtgagaagtaaccagttaagcctttttgaaatgagtgtcttgggccccgtaatgagacactctccatatgttttatcctagaacctttaaagaacccactatcttcaccaccctgatcatttgtcataagaatgataatcatgccaccatctcttgtaattaatccttatacttctaaagagcagctactgtttatgttcctattttaaggccaggaaatagaaagttccagatgctaaggaacttgcccagggtgataagtccaagcaacatttaataatctgtgtgacagcttgattcctgaatggcatgcttgtactcattatctgtccttggaggacagtaggtacccccatttcctttacctactgcagaggtctcaggcctcttgacttaataggcaacttggtccctgccccagagagagatacaatcctttctattttaccgattattcctggtctcctgggaccagagctgtgtgttgctgtttgctgtggttgtgagggtgggtgaagtaaaacatgtggctgtcacccaggggtctcaacacgataacaagctgatctgtgtgtttcagcactacacagatcacaaggtattttcagatacacaaccattctggtcttccacacaaactcaggagagagccaggattgctctggctgaactcgcagcacgaaaggtgccaaagttgatttatcctgctgggctgaggggtaagatacacctgggcccctgaaactccaggggcgcgctgcaaggtttccatgcagtaaccagtgaccatctgcccgcagccagcagcgctccaaatccgagatgctgtcgcggaagaactttgccacgggcgtgccggccgtgtcgatggacgagctggcagccttcgcagactcgtacggccagcggtcgcggcgcgccaatggcaacagccacgaggcgcgggcggggagccgcttcgagcgctcggagtcgcgggcccacggtgccttctaccaggacggctcgctggatgagtactacgggcgcggacgcagtcgcgagcccccgggagacggggagcgcggctggacctacagccccgcacccgcacgccgccggccgccggaggatgcgcctctgccgcgcctggtgagccggaccccgggcaccgcgcccaagtacgatcactcgtacctgagcagcgtgctggagcgccaggcgcggccggagagcagcagccgcgggggcagcctggagacgccgtccaagctgggcgcgcagctgggcccgcgcagcgcatcctactacgcctggtcgccgccagccacatacaaggctggggccagcgagggcgaagacgaggacgacgcggcggatgaggacgcgctgccaccctacagcgagctggagctgagccgcggagagctgagccggggcccgtcctaccgtgggcgtgacctgtccttccacagcaactcggagaagaggaggaaaaaggagcccgtcaagaaacccgtgaggactcacccccatgtctctggagctgggtccgggaat

SEQ ID NO: 19 - DNA, 2861 bp, Mus musculus; identical to SEQ ID NO: 18

SEQ ID NO: 20 - DNA, 2845 bp, Mus musculus
gactagtaaatgttgtctttaataatattttcaaagtctcagttaaaagatttagaaaacaagtggatcagttacattacaaataggttccctagatttaagtaaaaggaaattcaatatatttctaagtttttaaaaaataatcgaaatttctgttatttttctagctacatcataactctggtcctagttcataagtacttctgcacaaagctaggaagtgagaaatctgtgaccgcatctttcttacattttttaggcaggggccagagttcaagctacagcccagtggacacaaaggctaagtccaccttccaaacgtctggcctagccactcacaaccacaaacacctgcaatcctttgatagggagggaaacaggtctacccaggccttaagtaggtctggtgagccttgggcagggcattacacagcaggagcgtggtctaaaaggtaagtgaactgaaaccaaggtatatgtccttcaccttgactttgagccatttggagagcagaatgggcctcttctaaagcacggggttcatactggctctaaagacccccttttgggaccgggccagcagtagagaacatgctattagtagtggctttttttttccccttcctctcttggcccaacatagcctgaatcattgaagttcaccgcagtgattatataggacagagaaaaacatttgaacaagggagatccgggaagcctgccttaggtagaacaaagacaattgtctccctattccaagaacagcatgtaggaagcctccctctctgtaagcaagttgggtttgagatggagccaattcctgctgagtaacacaaataccacctgtgatcatctacagctcacactggtcaggaccatggctcccaggcagaagattctggaatatgcgatcatagcccttagcagcactcccttccaatcatttagaaaaccatggtgcctgcttttgttcctgcagataacaacaccatctcggaactcagctccctgcatgatgatgacagcaatttccgccagtcttaccaccagatgcggaataagcagttccctatgtctggagacctggaaagcaatcctgactactggtcaggtgtcatgggaggcaacagtgggaccaacagggggccagccttggagtataacaaagaggaccgtgagagcttcaggcacaggtgacggccatgagtgggaagggaccactgtgtatctgttcttctgtttctatagactatggaatatctcttacatatattacacccttgtgatactgtgtgtgagaagtaaccagttaagcctttttgaaatgagtgtcttgggtcccgtaatgagacactctctccatatgttttatcctagaacctttaaagaacccactatcttcaccaccctgatcatttgtcataagaatgatgatcatgccaccatctcttgtaattaatccttatacttctaaaaagcagctactgtttatgttcctattttaaggccaggaaatagaaagttccagatgctaaggaacttgcccagggtgataagtccaagcaacatttaataatctgtgtgacagctcgattcctgaatggcatgcctgcactcattatctgtccttggaggacagtaggtacctacccccccccccatttcctttacccactgcagaggtctcaggcctcttgacttaataggcaacttggtccctgccccggagagagatacaatcctttctatgttaacaattattcctggtctcctgggaccagagctgtgtgttgctgtttgctgtggttgtgagggtgggtgaagtaaaacatgtggctgtcacccaggggtctcaacacgataacaagctgatctgtgtgtttcagcactacacagatcacaaggtattttcagatacacaaccattctggtcttccacacaaactcaggagagagccaggattgctctggctgaactcgcagcacaaaaggtgccaaagttgattcatcctgctgggctgaggggtaagatacacctgggcccctgaaactccaggggcgcgctgcaaggtttccatgcaataaccagtgaccatctgcccgcagccagcagcgctccaaatctgagatgctgtcgcggaagaactttgccacgggcgtgccggccgtgtcgatggacgagctggcagccttcgcagactcgtacggccagcggtcgcggcgcgccaatggcaacagccacgaggcgcgggcggggagccgcttcgagcgctcggagtcgcgggcccacggtgccttctaccaggacggctcgctggatgagtactacgggcgcggacgcagtcgcgagccgccgggagacggggagcgtggctggacctacagccccgcacccgcacgccgccggccgccagaggatgcgcctctgccgcgcctggtgagccggaccccgggcaccgcgcccaagtacgatcactcgtacctgagcagcgtgctggagcgccaggcgcggccggagagcagcagccgcgggggcagcctggagacgccgtccaagctgggcgcgcagctgggcccgcgcagcgcatcctactacgcctggtcgccgccaaccacatacaaagctggggccagcgagggcgaagacgaggacgacgcggcggatgaggacgcgctgccaccctacagcgagctggagctgagccgcggagagctgagccggggcccgtcctaccgtgggcgtgacctgtccttccacagcaactcggagaagaggaggaaaaaggagcccgccaagaaacccgtgagggctcacccccatctctctggagctgggtccgggaat

SEQ ID NO: 21 - protein, 646 aa, Mus musculus
MDRVVLGWTAVFWLTAMVEGLQVTVPDKKKVAMLFQPTVLRCHFSTSSHQPAVVQWKFKSYCQDRMGESLGMSSPRAQALSKRNLEWDPYLDCLDSRRTVRVVASKQGSTVTLGDFYRGREITIVHDADLQIGKLMWGDSGLYYCIITTPDDLEGKNEDSVELLVLGRTGLLADLLPSFAVEIMPEWVFVGLVILGIFLFFVLVGICWCQCCPHSCCCYVRCPCCPDSCCCPQALYEAGKAAKAGYPPSVSGVPGPYSIPSVPLGGAPSSGMLMDKPHPPPLAPSDSTGGSHSVRKGYRIQADKERDSMKVLYYVEKELAQFDPARRMRGRYNNTISELSSLHDDDSNFRQSYHQMRNKQFPMSGDLESNPDYWSGVMGGNSGTNRGPALEYNKEDRESFRHSQQRSKSEMLSRKNFATGVPAVSMDELAAFADSYGQRSRRANGNSHEARAGSRFERSESRAHGAFYQDGSLDEYYGRGRSREPPGDGERGWTYSPAPARRRPPEDAPLPRLVSRTPGTAPKYDHSYLSSVLERQARPESSSRGGSLETPSKLGAQLGPRSASYYAWSPPTTYKAGASEGEDEDDAADEDALPPYSELELSRGELSRGPSYRGRDLSFHSNSEKRRKKEPAKKPGDFPTRMSLVV

SEQ ID NO: 22 - protein, 639 aa, Homo sapiens
MDRVLLRWISLFWLTAMVEGLQVTVPDKKKVAMLFQPTVLRCHFSTSSHQPAVVQWKFKSYCQDRMGESLGMSSTRAQSLSKRNLEWDPYLDCLDSRRTVRVVASKQGSTVTLGDFYRGREITIVHDADLQIGKLMWGDSGLYYCIITTPDDLEGKNEDSVELLVLGRTGLLADLLPSFAVEIMPEWVFVGLVLLGVFLFFVLVGICWCQCCPHSCCCYVRCPCCPDSCCCPQALYEAGKAAKAGYPPSVSGVPGPYSIPSVPLGGAPSSGMLMDKPHPPPLAPSDSTGGSHSVRKGYRIQADKERDSMKVLYYVEKELAQFDPARRMRGRYNNTISELSSLHEEDSNFRQSFHQMRSKQFPVSGDLESNPDYWSGVMGGSSGASRGPSAMEYNKEDRESFRHSQPRSKSEMLSRKNFATGVPAVSMDELAAFADSYGQRPRRADGNSHEARGGSRFERSESRAHSGFYQDDSLEEYYGQRSRSREPLTDADRGWAFSPARRRPAEDAHLPRLVSRTPGTAPKYDHSYLGSARERQARPEGASRGGSLETPSKRSAQLGPRSASYYAWSPPGTYKAGSSQDDQEDASDDALPPYSELELTRGPSYRGRDLPYHSNSEKKRKKEPAKKTNDFPTRMSLVV

SEQ ID NO: 23 - protein, 590 aa, Danio rerio
MFLLHAFWILFTLFSLQSCDGVQVVVKDEKKFAMLFSSIVLPCHYTTHSTQTAVVQWWYKSYCTDRTRDSFTFPESLGVHVSDLGASSHRDCSDNSRTVRIVASGQGASMTLAEHYKGRDISIINKADLHIGQLQWGDSGVYFCKVIISDDLEGKNEGQVELLVQGRTGVLDDILPEFDLEIMPEWAFVGVVVVGSILFLLLVGICWCQCCPHSCCCYVRCCCCPDTCCCPKHLYEAGKMAKSGQPPQITMYQPYYVPGVPVVPVVPPAASSIIEPKLPTVPPSVENNIAGTADNLSELSSLHDGDVDFRQTYRQVQRKALPPIIDHLDEPRLRTASIGHGLRPSHYQSDHSLDEHDNRWNCRSEHLPRKAFDSRGRTVSLDELEEFAMSYGPHGRRRGDIRGPQRDFEMAPRTRDHPTSYRNGPRYLREDDDSDWHRRGSPPSPPKRRDTADSERYVSRQRSYDDTYLNSLLERKARGHGERGGRVDDDSDTPSKGSSKKSSDCYQSRSPSNRPEEEDPLPPYSEREGERFRTEEPTGRERYRTADPAMRPFSYTRPPHGLSQTLQERREDRDKPRKLTTHLSRDSLIV

SEQ ID NO: 24 - protein, 573 aa, Mus musculus
MAPAASACAGAPGSHPATTIFVCLFLIIYCPDRASAIQVTVPDPYHVVILFQPVTLHCTYQMSNTLTAPIVIWKYKSFCRDRVADAFSPASVDNQLNAQLAAGNPGYNPYVECQDSVRTVRVVATKQGNAVTLGDYYQGRRITITGNADLTFEQTAWGDSGVYYCSVVSAQDLDGNNEAYAELIVLGRTSEAPELLPGFRAGPLEDWLFVVVVCLASLLFFLLLGICWCQCCPHTCCCYVRCPCCPDKCCCPEALYAAGKAATSGVPSIYAPSIYTHLSPAKTPPPPPAMIPMRPPYGYPGDFDRTSSVRSGYRIQANQQDDSMRVLYYMEKELANFDPSRPGPPNGRVERAMSEVTSLHEDDWRSRPSRAPALTPIRDEEWNRHSPRSPRTWEQEPLQEQPRGGWGSGRPRARSVDALDDINRPGSTESGRSSPPSSGRRGRAYAPPRSRSRDDLYDPDDPRDLPHSRDPHYYDDLRSRDPRADPRSRQRSHDPRDAGFRSRDPQYDGRLLEEALKKKGAGERRRVYREEEEEEEEGHYPPAPPPYSETDSQASRERRMKKNLALSRESLVV

SEQ ID NO: 25 - protein, 646 aa, Mus musculus; identical to SEQ ID NO: 21

SEQ ID NO: 26 - protein, 630 aa, Rattus norvegicus
MVEGLQVTVPDKKKVAMLFQPTVLRCHFSTSSHQPAVVQWKFKSYCQDRMGESLGMSSPRAQALSKRNLEWDPYLDCLDSRRTVRVVASKQGSTVTLGDFYRGREITIVHDADLQIGKLMWGDSGLYYCIITTPDDLEGKNEESVELLVLGRTGLLADLLPSFAVEIMPEWVFVGLVILGIFLFFVLVGICWCQCCPHSCCCYVRCPCCPDSCCCPQALYEAGKAAKAGYPPSVSGVPGPYSIPSVPLGGAPSSGMLTDKPHPPPLAPSDSTGGSHSVRKGYRIQADKERDSMKVLYYVEKELAQFDPARRMRGRYNNTISELSSLHDDDSNFRQSYHQMRNKQFPMSGDVESNPDYWSGVMGGNSGTNRGPALEYNKEDRESFRHSQPRSKSEMLSRKNFATGVPAVSMDELAAFADSYGQRSRRANGNSHEARAGSRFERSESRAHGAFYQDGSLDEYYGRGRSREPPGDGERGWTYSPAPARRRPPDDAALPRLVSRTPGTAPKYDHSYLSSVLERQARPESNSRGGSLETPSKLGAQLGPRSASYYAWSPPATYKAGASEGEDEDDAADEDALPPYSELELSRGELSRGPSYRGRDLSFHSNSEKRRKKEPAKKTGDFPTRMSLVV

SEQ ID NO: 27 - protein, 634 aa, Bos taurus
MAWVAVLWLTAMAEGLQVTVPDKKKVAMLFQPTVLHCRFSTSSHQPAVVQWKFKSYCQDRMGESLGMSSPRTQSLSKRNLEWDPYLDCLDSRRTVRVVASKQGSTVTLGDFYRGREITIVHDADLQIGKLMWGDSGLYYCIITTPDDLEGKNEDSVEVLVLGRTGLLADLLPSFAVEIMPEWVFVGLVILGVFLFFVLVGICWCQCCPHSCCCYIRCPCCPDSCCCPQALYEAGKAAKAGYPPSVSGVPGPYSIPSVPLGGAPSSGMLMDKPHPPPLAPSDSTGGSHSVRKGYRIQADKERDSMKVLYYVEKELAQFDPARRMRSRYNNTISELSSLHEEDSSLRQSYHQMRNKQFPVSGDLESNPDYWSGVMGGSSGASRGPSAMEYNKEDRESFRHSQQRSKSEMLSRKNFATGVPAVSMDELAAFADSYGPRSRRADGNKQDLRGGSRFERSEARAHGGLYQDGSLEEYYGPRSRSREPLTDADRGWSYSPPRRRPPDDAHLPRLVSRTPGTTPKYDHSFRGSGLERQVRPEGASRGGSLETPSKLSSQLGPLSASYYAWSPPATYEAGAPPDDEEDTPDDTLPPYSELELSRGPSYRGRDLPYHSNSEKKRKKETPAKKTDFPTRMSLVV

SEQ ID NO: 28 - protein, 586 aa, Canis familiaris
MLFQPTVLRCHFSTSSHQPAVVQWKFKSYCQDRMGESLGMASPRAQPLSKRNLEWDPYLDCLDSRRTVRVVASKQGSTVTLGDFYRGREITIVHDADLQIGKLMWGDSGLYYCIITTPDDLEGKNEDLAELLVLGRTGLLADLLPSFAVEIMPEWVFVGLVILGVFLFFVLVGICWCQCCPHSCCCYIRCPCCPDSCCCPQALYEAGKAAKAGYPPSVSGVPGPYSIPSVPLGGAPSSGMLMDKPHPPPLAPSDSTGGSHSVRKGYRIQADKERDSMKVLYYVEKELAQFDPARRMRGRYNNTISELSSLHEEDSNFRQAYHQMRSKQFPVSGDLESNPDYWSGVMGGSSGASRGPSAMEYNKEDRESFRYRMLSRKNFAAGVPAVSMDELAAFADSYGARSRRADGDSHEARGGGRFERPEARALGGFFQDGSPEGYYGRSRSREPLGDAGRAWAPSPPRRRPDDAPLPRLVSRTPGTAPKYEHAPRAGGLERQARPEGASRGGSLETPSRLSAQLGRRSASYYAWSPPATYKAAAPQDDDDDDDDDSADDALPPYSERELSRGPSYRGRDLPYHSNSEKKRKKE

SEQ ID NO: 29 - protein, 639 aa, Homo sapiens; identical to SEQ ID NO: 22

SEQ ID NO: 30 - protein, 639 aa, Pan troglodytes; identical to SEQ ID NO: 22

SEQ ID NO: 31 - protein, 592 aa, Macaca mulatta
MDRVLLRWISLFWLTAMVEGLQVTVPDKKKVAMLFQPTVLRCHFSTSSHQPAVVQWKFKSYCQDRMGESLGMSSTRAQSLSKRNLEWDPYLDCLDSRRTVRVVASKQGSTVTLGDFYRGREITIVHDADLQIGKLMWGDSGLYYCIITTPDDLEGKNEDSVELLVLGRTGLLADLLPSFAVEIMPEWVFVGLVLLGVFLFFVLVGICWCQCCPHSCCCYVRCPCCPESCCCPQALYEAGKAAKAGYPPSVSGVPGPYSIPSVPLGGAPSSGMLMDKPHPPPLAPSDSTGGSHSVRKGYRIQTDKERDSMKVLYYVEKELAQFDPARRMRGRYNNTISELSSLHEEDSNFRQSFRQMRSKQFPVSGDLESNPDYWSGVMGGSSGASRGPSAMEYNKEDRESFRHRILNISHLSRQGTLVITCVEDDSLEEYYGQRSRSREPLTDADRGWAFSPARRRPTEDAHLPRLVSRTPGTAPKYDHSYLGGARERQPRPEGASRGGSLETPSKRSAQLGPRSASYYAWSPPGTYKAGSSQDDQEDASDDALPPYSELELTRGPSYRGRDLPYHSNSEKRRKKEPAKKTNDFPTRMSLVV

SEQ ID NO: 32 - protein, 314 aa, Felix domestica
MADGLQVTVPDKKKVAMLFQPTVLRCHFSTSSHQPAVVQWKFKSYCQDRMGESLGMSSPRAQSLSKRNLEWDPYLDCLDSRRTVRVVASKQGSTVTLGDFYRGREITIVHDADLQIGKLMWGDSGLYYCIITTPDDLEGKNEDSAELLVLEWVFVGLVILGIFLFFVLVGICWCQCCPHSCCCYIRCPCCPDSCCCPQALYEAGKAAKAGYPPSVSGVPGPYSIPSVPLGGAPSSGMLMDKPHPPPLAPSDSTGGSHSVRKGYRIQADKERDSMKVLYYVEKELAQFDPARRMRGRCEHLLVLCDLRVMREFNA

SEQ ID NO: 33 - protein, 644 aa, Monodelphis domestica
MDRMSLGWIVLFWVTGVAEGLQVIVPEKTKKAMLFQPVVLSCRFSTSSQQPAVIQWKFKSYCQDRMKEALGMATAGAQPLSKRNLEWDPYLDCVDSRRTVRVVASKQGAVVTLGEFYRGRDITFGEGAELKIGKVMWGDSGLYYCIVTTPDDVEGKNEDSVELLVLGRTGWLAALLPSFAVKIMSEWVFVGLVILGVFLFFLLVGICWCQCCPHSCCCYVRCPCCPDSCCCPRALYEAGKAAKSGYPPSVSSVPGPYYIPSVPVGGVSSSAMLMDKPHPPPLASSDSIGGSQSVRKGYRIQADKERDSMKVLYYVEKELAQFDPARRMRERYNNTISELSSLHEDNGNFCQSYRQMRRKPLPSLGNIESDTDYWTGVMGNSGGSGHGPSSSNYNKEDRDSFRHSQQRCKSEMLSRKNFAMGMPAVSMDELAAFADSYSQRSHRGEGNSQEPRGGSRFERSESRAHGGLYHDGSLEEYYSKRSRSREPLTDSDRGWSYSPPRRRANEDKHLPRLVSRTPGVGQKYDHPYLSSVLERKSRGEGSSGGGSLETPSKRSSQPIQRSGSYYAWSPPSTYKAGSGQQPSPQAGEEDEMEDALPPYSELELTRGPSYRGRESLYHSNSEKKRKKDSLKKTNDFPTRMSLVV

SEQ ID NO: 34 - protein, 640 aa, Gallus gallus
MGGRLLGCVVLLWLSAVEGLQVTVPEKKKVAMLFQPALLRCHFSTSSTQPAVVQWRYKSYCQDRMGEALGMVTSGLQTMSKRNLDWDPYLDCVDSRRTVRVVASKQGSAVTIGDFYKERDVSIVHDADLQIGKLMWGDSGLYYCIIITPDDVEGKSEESVELLVLGRTGLLADLLPSFAVEIMPEWVFVGLVILGAFLFFLLVGICWCQCCPHSCCCYVRCPCCPESCCCPRALYVAGKAAKAGYPPVVSSIPGPYYIPSVPVAGVPSPAVLMDKSHPPPLAPSDTGGGNQNAVRKGYRIQTDKDRDSMKVLYYVEKELAQFDPARRMRERYNNTVSELSSLHEDDLNFRQPYRQARRKPLPPAEDLDGDAEYWAGVMGGGSTSRSQAISDYRDERDSFRHSQQRSKSEMLSRKSFSVGVPAVSMDELAAFAESYSQRARRADSQETRRFERSESRSGRGGGLTHQDSSMEEYYTKRSRGNREPLTDSDRGWSYSPPRRRAHEEKHLPRLVSRTPGGSQKYDHSYLSSVLERKSRSYDESGDPCETPSKLSSQPSQRGGGTYYAWSPPSTYKSDTSQQQQTPPPEQEEGEDTLPPYSERELSRGPSYRAREQAYLNASDKKRKKDPKKTNDFPTRMSLVV

SEQ ID NO: 35 - protein, 568 aa, Xenopus tropicalis
MAAGIVLGCIGLMCATGSMTYGIKVTMPEHKKVVMLFQSVLMRCQYATSSTQPVVVQWRYKSFCLDRMEEALGIGKVPGKGATGNQYLDCADGSRTVRTVASKQGSTVTLGDFYKGKDITIVNDADLQFGNMQWGDSGLYYCLVVTSDDLEGKNEDRVEILVLGQNGADQLVGAAADIRPEWAFVCLVILGTFLFFVMVGICWCQCCPHNCCCYVRCPCCPETCCCPRALYEAGKAAKVGYPPTVPTACPPYYISTIPVSQVPACRVMDKPHVPPLVQSDSLPGQNAVRKGYRIQADKERDSMKVLYYVEKELAQFDPARRMRERYSHTISELSSLHEDNTHFNHSYRQVRRKPLPPSCNADGDAEYWSGVVGSARPATYSKFREDRESFRSSLQRPTSEVLERKSFPMTIQAVSTDELAAFTDSYKQRPRRADSRGPGSAPRFERSETRGRSLYQDSSSDEYYGKRNHGRELFSDGERGWSFSPSRIRAAEDKHLPKRITRMGQSYDDAYLSRVLERKSRGLEDTTVTPSKLSLRQNSSRSYGRSPTFCVNDFEILTANPSGTFLSV

SEQ ID NO: 36 - protein, 628 aa, Danio rerio
MFLLHAFWILFTLFSVQSCDGVQVVVKDEKKFAMLFSSIVLPCHYTTHSTQTAVVQWWYKSYCTDRTRDSFTFPESLGVHVSDLGASSHRDCSDNSRTVRIVASGQGASMTLAEHYKGRDISIINKADLHIGQLQWGDSGVYFCKVIISDDLEGKNEGQVELLVQGRTGVLDDILPEFDLEIMPEWAFVGVVVVGSILFLLLVGICWCQCCPHSCCCYVRCCCCPDTCCCPKHLYEAGKMAKSGQPPQITMYQPYYVPGVPVVPVVPPAASSIIEPKLPTVPPSVENNIAGMRSGYRLQASQGQDAMKVVYYLERDLAQFHPTKGASHPSADNLSELSSLHDGDVDFRQTYRQVQRKALPPIIDHLDEPRLRTASIGHGLRPSHYQSDHSLDEHDNRWNCRSEHLPRKAFDSRGRTVSLDELEEFAMSYGPHGRRRGDIRGPQRDFEMAPRTRDHPTSYRNGPRYLREDDDSDWHRRGSPPSPPKRRDTADSERYVSRQRSYDDTYLNSLLERKARGHGERGGRVDDDSDTPSKGSSKKSSDCYQSRSPSNRPEEEDPLPPYSEREGERFRTEEPTGRERYRTADPAMRPFSYTRPPHGLSQTLQERREDRDKPRKLTTHLSRDSLIV

SEQ ID NO: 37 - protein, 601 aa, Homo sapiens
MALLAGGLSRGLGSHPAAAGRDAVVFVWLLLSTWCTAPARAIQVTVSNPYHVVILFQPVTLPCTYQMTSTPTQPIVIWKYKSFCRDRIADAFSPASVDNQLNAQLAAGNPGYNPYVECQDSVRTVRVVATKQGNAVTLGDYYQGRRITITGNADLTFDQTAWGDSGVYYCSVVSAQDLQGNNEAYAELIVLGRTSGVAELLPGFQAGPIEDWLFVVVVCLAAFLIFLLLGICWCQCCPHTCCCYVRCPCCPDKCCCPEALYAAGKAATSGVPSIYAPSTYAHLSPAKTPPPPAMIPMGPAYNGYPGGYPGDVDRSSSAGGQGSYVPLLRDTDSSVASEVRSGYRIQASQQDDSMRVLYYMEKELANFDPSRPGPPSGRVERAMSEVTSLHEDDWRSRPSRGPALTPIRDEEWGGHSPRSPRGWDQEPAREQAGGGWRARRPRARSVDALDDLTPPSTAESGSRSPTSNGGRSRAYMPPRSRSRDDLYDQDDSRDFPRSRDPHYDDFRSRERPPADPRSHHHRTRDPRDNGSRSGDLPYDGRLLEEAVRKKGSEERRRPHKEEEEEAYYPPAPPPYSETDSQASRERRLKKNLALSRESLVV

SEQ ID NO: 38 - protein, 591 aa, Pan troglodytes
MALLAGGLSRGLGSHPAAPGRDAVVFVWLLLSTWCTAPARAIQVTVSNPYHVVILFQPVTLPCTYQMTSTPTQPIVIWKYKSFCRDRIADAFSPASVDNQLNAQLAAGNPGYNPYVECQDSVRTVRVVATKQGNAVTLGDYYQGRRITITGNADLTFDQTAWGDSGVYYCSVVSAQDLQGNNEAYAELIVLGRTSGVAELLPGFQAGPMEDWLFVVVVCLAAFLIFLLLGICWCQCCPHTCCCYVRCPCCPDKCCCPEALYAAGKAATSGVPSIYAPSTYAHLSPAKTPPPPAMIPMGPAYNGYPGGYPGDVDRSSSAGGQGSYVPLLRDTDSSVASEVRSGYRIQASQQDDSMRVLYYMEKELANFDPSRPGPPNGRVERAMSEVTSLHEDDWRSRPSRGPALTPIRDEEWGGHSPRSPRGWDQEPAREQAGGGWRARRPRARSVDALDDLTPPSTAESGSRSPTSSGGRRSRAYMPPRSRSRDDLYDQDDSRDFPRSRDPHYDDLRSRERPPADPRSQHHRTRDPRDNGSRSGDLPYDGRLLQEAVRKKGSEERRRPHKEEEEEAYYPPAPPPYSETDSQASRERRLKK

SEQ ID NO: 39 - protein, 381 aa, Macaca mulatta
MALVAGGLCRGLGSHPAAPGRDAVVFVWLLLSTWFTAPARAIQVTVSDPYHVVILFQPVTLPCTYQMTSTPTQPIVIWKYKSFCRDRIADAFSPASVDNQLNAQLVAGNPGYNPYVECQDSVRTIRVVATKQGNAVTLGDYYQGRRITITGNADLTFDETAWGDSGVYYCSVVSAQDLEGNNEAYAELIVLGRTSGVAELLPGFQAGPMEDWLFVVVVCLAAFLVFLLLGICWCQCCPHTCCCYVRCPCCPDKCCCPEALYAAGKAATSGVPSIYAPSTYAHLSPAKTPPLPTVIPMGPAYNGYPGGYPGDLDRSSSAGGQGSYVPLLRDTDSSVASEVRSGYRIQASQQDDSMRVLYYMEKELANFDPSRPGPPNGRVER

SEQ ID NO: 40 - protein, 525 aa, Bos taurus
MAPLARPFSGGLESCPGTLSWGAVVFVWLFLSTSCTAPTSAIQVTVSDPYHVVILFQPVTLPCTYQLTTTPTAPIVIWKYKSFCRDRIADAFSPASVDNQLNAQLAAGNPGYNPYVECQDSARTVRVVATKQGNAVTLGDYYQGRRITITGSRTSGVAELLPGFQAGPMEDWLFVVVVCLAAFLVFLLLGICWCQCCPHTCCCYVRYAAGKAATSGVPSIYAPSTYAHLSPAKTPPPPAMIPMGPLYNGYSGDFDRNSSEIRSGYRIQANQQDDSMRVLYYMEKELANFDPSRPGPPNGRVERAMSEVTSLHEDDWRSRPSRGPALTPIRDEEWGHHSPRSSRRWEQEAPMERPGNSRGAGRPRARSVDALDDFTRPGSAESGRRSPPSSGRRGRAYAPPRSRSRDDLYDQDQDDSRHFPHSRDPHYDDFRSRDQPHGDPRARYQRSRDPRDDGSRSRDPPYDGRLLEEALRKKGPAERRPYREEEEEEAYYPPAPPPYSETDSQASRERRLKKNLALSRESLVV

SEQ ID NO: 41 - protein, 598 aa, Canis familiaris
MAPVARGLPGGVGPRPASRGWGAVVFGCLFLSTLCAAPASAIQVTVSDPYHVVILFQPVTLPCTYQLTTTPTAPIVIWKYKSFCRDRIADAFSPASADNQLNAQLAAGNPGYNPYVECQDSMRTVRVVATKQGNAVTLGDYYQGRRITITGNADLTFDQTGWGDSGVYYCSVVSAQDLQGNNEAYAELIVLGRTSGVAELLPGFQAGPMEDWLFVVVVCLAVFLVFLLLGICWCQCCPHTCCCYVRCPCCPEKCCCPEALYAAGKAATSGVPSIYAPSTYAHLSPAKTPPPPAMIPMGPLYNGYPGDFDRNSSVGGHSSQVPLLRDTDSSVTSEVRSGYRIQASQQDDSMRVLYYMEKELANFDPSRPGAPNGRVERAMSEVTSLHEDDWRSRPSRGPALTPIRDEEWDRHSPRSPRRWEQEPPTERPGSGWGAARPRARSVDALDDLTRPSSAESGRRSPPSRGRRGQAYGRPRSRSRDDLYDQDGPREFPHPRDPHYDDFRPRDRPHADPRSRNHRSRDSREDGSRSGDPQYDGRLLEEALRKKGPAERRRAYREEEEEEAYYPPAPPPYSETDSQASRERRLKKNLALSRESLIV

SEQ ID NO: 42 - protein, 594 aa, Mus musculus
MAPAASACAGAPGSHPATTIFVCLFLIIYCPDRASAIQVTVPDPYHVVILFQPVTLHCTYQMSNTLTAPIVIWKYKSFCRDRVADAFSPASVDNQLNAQLAAGNPGYNPYVECQDSVRTVRVVATKQGNAVTLGDYYQGRRITITGNADLTFEQTAWGDSGVYYCSVVSAQDLDGNNEAYAELIVLGRTSEAPELLPGFRAGPLEDWLFVVVVCLASLLFFLLLGICWCQCCPHTCCCYVRCPCCPDKCCCPEALYAAGKAATSGVPSIYAPSIYTHLSPAKTPPPPPAMIPMRPPYGYPGDFDRTSSVGGHSSQVPLLREVDGSVSSEVRSGYRIQANQQDDSMRVLYYMEKELANFDPSRPGPPNGRVERAMSEVTSLHEDDWRSRPSRAPALTPIRDEEWNRHSPRSPRTWEQEPLQEQPRGGWGSGRPRARSVDALDDINRPGSTESGRSSPPSSGRRGRAYAPPRSRSRDDLYDPDDPRDLPHSRDPHYYDDLRSRDPRADPRSRQRSHDPRDAGFRSRDPQYDGRLLEEALKKKGAGERRRVYREEEEEEEEGHYPPAPPPYSETDSQASRERRMKKNLALSRESLVV

SEQ ID NO: 43 - protein, 593 aa, Rattus norvegicus
MAPAAGACAGAPDSHPATVVFVCLFLIIFCPDPASAIQVTVSDPYHVVILFQPVTLPCTYQMSNTLTVPIVIWKYKSFCRDRIADAFSPASVDNQLNAQLAAGNPGYNPYVECQDSVRTVRVVATKQGNAVTLGDYYQGRRITITGNADLTFEQTAWGDSGVYYCSVVSAQDLDGNNEAYAELIVLGRTSEAPELLPGFRAGPLEDWLFVVVVCLASLLLFLLLGICWCQCCPHTCCCYVRCPCCPDKCCCPEALYAAGKAATSGVPSIYAPSIYTHLSPAKTPPPPPAMIPMGPPYGYPGDFDRHSSVGGHSSQVPLLRDVDGSVSSEVRSGYRIQANQQDDSMRVLYYMEKELANFDPSRPGPPNGRVERAMSEVTSLHEDDWRSRPSRAPALTPIRDEEWNRHSPQSPRTWEQEPLQEQPRGGWGSGRPRARSVDALDDINRPGSTESGRSSPPSSGRRGRAYAPPRSRSRDDLYDPDDPRDLPHSRDPHYYDDIRSRDPRADPRSRQRSRDPRDAGFRSRDPQYDGRLLEEALKKKGSGERRRVYREEEEEEEGQYPPAPPPYSETDSQASRERRLKKNLALSRESLVV

SEQ ID NO: 44 - protein, 596 aa, Monodelphis domestica
MAPAAPGPHGRTGAPLDPLGWNPRRRGALPLPLLLLLLALWCSAPVGCIQVTVSNPFQVVILFQPVTLPCSYQLSGVPTLPIVVWKYKSFCRNRITDAFSPASADSQLNAQLAAGNPGYNPYVECQDSVRTVRVVATKQGNAVTLGDFYQGRRITITGNADLTFDQTAWGDSGVYYCSVISAQDLQGNNEAYAELIVLGRTSGVAELLPDFQIGPMEDWLFVVVVGLAAFLVFLLLGICWCQCCPHTCCCYVRCPCCPEKCCCPEALYAAGKAATSGVPSIYAPSVFAPSTYAHLSPAKAPSPPPMIPLGPVYNDFDRQSSVGGHSSQVPLLRDTDSVRNSEVRSGYRIQANQQDDSMKVLYYMEKELANFDPSRPGLPNGRVERAMSEVTSLHEDDWRARPHRGPALTPIQDEDLDYHSRSPGGWGRERPHDRYGERPHDPYGDWGAGRPRARSVDALDDLARPSSVESGRTSPSERSRSKAYAPLRSRSRDDLYSRSGDPHYEDFRSRGRALDDSRRDPHENHRRSRDPEYDGRFLEEVMRKKGVGERRRPYREEEEEPYYPPAPPPYTETDSQASRERKLRKNLALSRESLVV

SEQ ID NO: 45 - protein, 613 aa, Danio rerio
MSLGVIFTLLLFPGVTTGINVICTYPRYVVIMFQPVTLRCDFTTTSTTPPLITWKYKSYCRDPIQAALNPSSADNAIAQSNPNYNPNIECADSARTVRIVASKQTAVTLGKEYQGRQISITNNADLSIVQTAWGDSGVYVCSAASAQDLSGNGECYTELIVLGRKSNTTDLLPGIDLLIMEDWLLVVLVVLGFLLLLLLIGICWCQCCPHTCCCYVRCPCCPERCCCPRALYEAGKMVKSGIPSQYAATAYAQSMYGQPAYGVGAAMPGIPMMPMQMGVGGPPSNGYGRDYDGASSIGQGSQVPLLQEHDAGGNRSGYRVQADQDGNPTRVLYYMEREVANLDPSRPGIAPVDGMSEVSSLHDGPESRNRGRARPPQLTTVYDDVDENMSTISSVSQHMRRDEPRRGADSRGRARSMENLDDISRGYRDRDDYPPARRDGGPRGGRRGSDDEWSSSGRGYDPVDDRRRRDYSPDNRPRRGDSFRGAGFQGRRTRSRDDLMDLVRDPGRGGRDEYDDSFLREAMEKKKLGEQQRGRSRERLDSESDRSDRYRGHHSGPPPLPLVPASGNPDRRGNHSNFPPPPPPYTEDTDSLPSSKKSNLKKNGAVSRESLVV

SEQ ID NO: 46 - protein, 546 aa, Homo sapiens
MAWPKLPAPWLLLCTWLPAGCLSLLVTVQHTERYVTLFASIILKCDYTTSAQLQDVVVTWRFKSFCKDPIFDYYSASYQAALSLGQDPSNDCNDNQREVRIVAQRRGQNEPVLGVDYRQRKITIQNRADLVINEVMWWDHGVYYCTIEAPGDTSGDPDKEVKLIVLHWLTVIFIILGALLLLLLIGVCWCQCCPQYCCCYIRCPCCPAHCCCPEEALARHRYMKQAQALGPQMMGKPLYWGADRSSQVSSYPMHPLLQRDLSLPSSLPQMPMTQTTNQPPIANGVLEYLEKELRNLNLAQPLPPDLKGRFGHPCSMLSSLGSEVVERRIIHLPPLIRDLSSSRRTSDSLHQQWLTPIPSRPWDLREGRSHHHYPDFHQELQDRGPKSWALERRELDPSWSGRHRSSRLNGSPIHWSDRDSLSDVPSSSEARWRPSHPPFRSRCQERPRRPSPRESTQRHGRRRRHRSYSPPLPSGLSSWSSEEDKERQPQSWRAHRRGSHSPHWPEEKPPSYRSLDITPGKNSRKKGSVERRSEKDSSHSGRSVVI

SEQ ID NO: 47 - protein, 546 aa, Pan troglodytes
MAWPKLPAPWLLLCTWLPAGCLSLLVTVQHTERYVTLFASIILKCDYTTSAQLQDVVVTWRFKSFCKDPIFDYYSASYQAALSLGQDPSNDCNDNQREVRIVAQRRGQNEPVLGVDYRQRKITIQNRADLVINEVMWWDHGVYYCTIEAPGDTSGDPDKEVKLIVLHWLTVIFIILGALLLLLLIGVCWCQCCPQYCCCYIRCPCCPAHCCCPEEALARHRYMKQAQALGPQMMEKPLYWGADRSSQVSSYPMHPLLQRDLSLRSSLPQMPMTQTTNQPPIANGVLEYLEKELRNLNLAQPLPPDLKGRFGHPCSMLSSLGSEVVERRIIHLPPLIRDLSSSRRTSDSLHQQWLTPIPSRPWDLREGRSHHHYPDFHQELQDRGPKSWALERRELDPSWSGRHRSSRLNGSPIHWSDRDSLSDVPSSSEARWRPSHPLFRSRCQERPRRPSPRESTQRDGRRRRHRSYSPPLPSGLSSWSSEEDKERQPQSWRAHRRGSHPPHWPEEIPPSYRSLDIIGGKNNKKKGSVERRSEKDSSHSGRSVVI

SEQ ID NO: 48 - protein, 546 aa, Pongo pygmaeus
MAWPKLPAPWLLLCTWLPAGCLSLLVTVQHTERYVTLFASIILKCDYTTSAQLQDVVVTWRFKSFCKDPIFDYYSASYQAALSLGQDPSNDCNDNQREVRIVAQRRGQNEPVLGVDYRQRKITIQNRADLVINEVMWWDHGVYYCTIEAPGDTSGDPDKEVKLIVLHWLTVIFIILGALLLLLLIGVCWCQCCPQYCCCYIRCPCCPARCCCPEEALARHRYMKQAQALGPQMMEKPLYWGADRSSQVSSYPMHPLLQRDLSLRSSLPQMPMTQTTNHPPIANGVLEYLEKELRNLNLAQPLPPDLKARFGHPCSMLSSLGSEVVERRFIHLPPLIRDLSSSRRTSDSLHQQWLTPIPSRPWDLREGRRQHHYPDFHQELQDRGPKSWALERRELDPSWSGRHRSSRLNGSPIHWSDRDSLSDVPSSIEARWQPSHPPFRSRCQERPRRPSPRESTQRHGRRRRHRSYSPPLPSGLSSWSSEEDKERQPQSWGAHRRRSHSPHWPEEKPPSYRSLDVTPGKNSRKKGSVERRSEKDSSHSGRSVVI

SEQ ID NO: 49 - protein, 537 aa, Mus musculus
MGCGLLAAGLLLFTWLPAGCLSLLVTVQHTERYVTLFASVTLKCDYTTSAQLQDVVVTWRFKSFCKDPIFDYFSASYQAALSLGQDPSNDCSDNQREVRIVAQRRGQSEPVLGVDYRQRKITIQNRADLVINEVMWWDHGVYYCTIEAPGDTSGDPDKEVKLIVLHWLTVIFIILGALLLLLLIGVCWCQCCPQYCCCYIRCPCCPTRCCCPEEALARHRYMKQVQALGPQMMEKPLYWGADRSSQVSSYAMNPLLQRDLSLQSSLPQMPMTQMAAHPPVANGVLEYLEKELRNLNPAQPLPADLRAKSGHPCSMLSSLGSAEVVERRVIHLPPLIRDPPSSRTSNPSHQQRLNAVSSRHCDLSERPRQRHHSDFLRELQDQGMRPWAPGRGELDPHWSGRHHRSRPSESSMPWSDWDSLSECPSSSEAPWPPRRPEPREGAQRRERRRHRSYSPPLPSGPSSWSSEEEKESLPRNWGAQRRHHHRRRRSQSPNWPEEKPPSYRSLDVTPGKNNRKKGNVERRLERESSHSGRSVVI

SEQ ID NO: 50 - protein, 538 aa, Rattus norvegicus
MGCGLLVAGLLLFTWLPAGCLSLLVTVQHTERYVTLFASVTLKCDYTTSAQLQDVVVTWRFKSFCKDPIFDYFSASYQAALSLGQDPSNDCSDNQREVRIVAQRRGQSEPVLGVDYRQRKITIQNRADLVINEVMWWDHGVYYCTIEAPGDTSGDPDKEVKLIVLHWLTVIFIILGALLLLLLIGVCWCQCCPQYCCCYIRCPCCPTHCCCPEEALARHRYMKQVQALGPQMMEKPLYWGADRSSQVSSYAMNPLLQRDLSLRSSLPQMPMTQMAAHPPVANGVLEYLEKELRNLNPAQPLPPDLRTKSGHPCSMLSSLGSAEVVERRVIHLPPLIRDPLPSRTSNSSHQQRLNPVPSRPRDPSEGRRQRNHSDFLRELQDRGMRPWAPGRGELDPHWSGRHHRSRPSESSMPWSDWDSLSECPSSSEAPWPSRRPEPREGSQRHGRRRHRSYSPPLPSGPSSWSSEEEKESLPRNWGAQRRHHRRSRRRSQSPNWLEEKPPSYRSLDVTPGKNNMKKGNVERRLERESSHSGRSVVI

SEQ ID NO: 51 - protein, 517 aa, Canis familiaris
MGPELPAPWLLLVAGLPAGCLSLLVTVQHTERYVTLFASIVLKCDYTTSAQLQDVVVTWRFKSFCKDPIFDYYSASYQAALSLGQDPSNDCNDSQREVRIVAQRRGQNEPVLGVDYRQRKITIQNRADLVINEVMWWDHGVYYCTIEAPGDTSGDPDKEVKLIVLHWLTVIFIILGALLLLLLIGVCWCQCCPQYCCCYIRCPCCPARCCCPEEALARHRYMKQAQALGPQMMEKPLYWGADRSSQVSSYPMNPLLQRDLSLRSSLPQMPMTQTAAAHPPVTNGVLEYLEKELRNLNPAQPLPPDLKTISGQACSMLSSLGSEVVERRIIHLPPLIRDLPPSWRTSSSSRQQWPAPGAPGPWGVSSDVHRELQGREPKRLRRGRHPCSRPHGSHAPWSDRDSLGDGPSSWEALGLGRGPRGDAQRPRRRRHRSYSPPSPSGLSSWSSEEEGEEGDRRPRGRGTPYSSQATTWATWAEEKPPSYRSLDVLPGRKGRRGGSVERRSERDSSHSGRSVVI

SEQ ID NO: 52 - protein, 545 aa, Xenopus laevis
MIPPRALLLISVWMVSGGRTLLVTVQDTQRYTMLFSSIILKCDYSTSAQIQDVAVTWRFKSFCKDPIFDYYSAAYQASLSLNQDPANDCNDNQREVRIVIQKRGQNEPVLGVDYRQRKITIQNKADLVISEVMWWDHGVYFCSVEAQGDTSGDPDKEVKLVVLHWLTVLFIILGALFLFLLIGICWCQCCPHCCCCYVRCPCCPTRCCCPEEALARHNYMKQMESMTPWMLDRPYYAGADRNSQHSSYQLNPLLQRDLSLQSSLPMPAPMSFSPPNNKVLDFLETEIKNLNTAQPLMSAPHYGGASHHPSMLSSLSEVGVREVDRRVIQLPPLVEHIVSSHRSSNSSHQRRNMGSWDPLDGERDRRRNRQLDDSLSNETNWRAQERQHSDRSSGHRRDPPNNRRPRRDVSPPRRYGDSYSDESANNDPRGRSNPHSDRARPTERRRSPERGDQGRRGSPDRYSRSQRHRRSYSPPHRRDSWSSEDETRNNQRGRGRRERSYEWPEEKPPSYKSLEICAGKAPTQRPGAVRQSDRASSRSGRSMVI

SEQ ID NO: 53 - protein, 540 aa, Gallus gallus
MARCGRCGQTLLLVWLLMACLPAGCLSLLVTVQDTERYTTLFASITLKCDYSTSAQLQDVVVTWRFKSFCKDPIFDYYSVSYQASLALGQDPSDDCNDVQRKVRIVIQKYGQNEPVLGVDYRQRKITIQNRADLVISEVMWWDHGVYYCTVEAPGDTSGDPDKEVKLIVLHWLTVLLIILGGLLLLLLIGICWCQCCPQHCCCHIRCVCCPTRCCCNEKVLERHRFMKRAQAFAPWMLPNMFYGGADRNSQLSSYQLNPLLQQDVSLQNSLPLVQPQARLSPNKGVLDYLESEIQNLNPSQPRPPSNQRQAVQPSLLSSLGSDIMQRGTNGLPPFTGHVSSSHGSSSSSRPQRTTRSLRTWGEDTAENRREDRRWPLPSSEDSRSSYSREPRDRQREDHPPRQRTGGYDGRSQYSRRDVSPTRQTERGKSSSSSCSFYSEEAKERSSHHRGRRQQPAVRREYQQHTRNSNNSRHRRHSYSPPSRRGSWSSSEEQIRLPATNRRRHNRSREWPEDKPPSYRSLEIIPDRDSKHREGAGPRS

SEQ ID NO: 54 - protein, 550 aa, Danio rerio
MKQKLRLKVLVLLLCVFPEEIFSIQVTVPETERHTMLFGSVTLRCDYSTSASQQDVLVTWRYKSFCLDPVLEYYSAAYQAALNMKQDPANDCPDSKRTVRIVIQKRGINEPVLGTEYRQRKISIKNSADLSMNEIMWWDNGMYFCSIDAPGDVVGDSDKEIRLIVYNWLTVLLIILGALLLIILFGVCCCQCCPQNCCCYVRCPCCPRTCCCPEKAVMRHKMMREAQKAMVPWFHGQPIYAPIASNASQANPLLYSGSFSEHSSKHNLPMAPMAIPPPQPVPQFVPSHGYHANGSMNGNVRANNQMLDFLENQVQGMDMAVPMLQPQHHYTGVPLQNHQPQYAAQQPHYASPPPQSIPQAVTFPARPPSMLSALDEMGVQGVERRVIQLPPILGRPKQSSRRTNDQRPRQSSQSSGSSNRNGVHRDPASSRRGNQRSYSDESDWDDRRGGRSSSGRRGESNRSRPRVRSKAELLEELEHATNNGNRSYSSPHRGSWSSDEEDSYRKGRRSQGKLSENPPAYSSIDILPGHSRRGEQLSDKSSRSGTSVVI

SEQ ID NO: 55 - protein, 646 aa, Mus musculus; identical to SEQ ID NO: 21

SEQ ID NO: 56 - protein, 594 aa, Mus musculus; identical to SEQ ID NO: 42

SEQ ID NO: 57 - protein, 537 aa, Mus musculus; identical to SEQ ID NO: 49

SEQ ID NO: 58 - protein, 87 aa, Mus musculus
MDRVVLGWTAVFWLTAMVEGLQVTVPDKKKVAMLFQPTVLRCHFSTSSHQPAVVQWKFKSYCQDRMGESLGMSSPRAQALSKRNLEW

SEQ ID NO: 59 - DNA, 21 bp, Artificial Sequence
  Description of Artificial Sequence Synthetic primer
accgctgtcttctggttaaca

SEQ ID NO: 60 - DNA, 25 bp, Artificial Sequence
  Description of Artificial Sequence Synthetic oligonucleotide
atgttgagtgtacttgagctggctc

SEQ ID NO: 61 - DNA, 25 bp, Artificial Sequence
  Description of Artificial Sequence Synthetic oligonucleotide
gaatgaaacacacttcctccagcat

SEQ ID NO: 62 - DNA, 28 bp, Artificial Sequence
  Description of Artificial Sequence Synthetic oligonucleotide
atgagcgtgtaacaaaaacatgatccag

SEQ ID NO: 63 - DNA, 25 bp, Artificial Sequence
  Description of Artificial Sequence Synthetic oligonucleotide
caactttgcactgtgccaaagaaag

SEQ ID NO: 64 - DNA, 24 bp, Artificial Sequence
  Description of Artificial Sequence Synthetic primer
tgcctatgcaaatgggagttggtg

SEQ ID NO: 65 - DNA, 24 bp, Artificial Sequence
  Description of Artificial Sequence Synthetic primer
ttggcaacctctcgctccatgtaa

SEQ ID NO: 66 - DNA, 24 bp, Artificial Sequence
  Description of Artificial Sequence Synthetic primer
caagggctccttcaagtacgcctg

SEQ ID NO: 67 - DNA, 21 bp, Artificial Sequence
  Description of Artificial Sequence Synthetic primer
ggaagaatggcatcaagggca

SEQ ID NO: 68 - DNA, 484 bp, Homo sapiens
agtaggaggcgctgcgcgggccgagctgcgcgctccgcttggatggcgtctccaggctgccaccgcggctggcgccctcgggccgcgcctggcgctcccgcgcgctgcccaggtacgagtggtcgtatttgggtgcggtgcctggcgtgcggctcaccagccgcggcaggtgtgcgtcctcggcgggtctgcggcgcgcggggctgaaggcccagccgcggtcagcatcggtcaggggctcgcggctgcggctgcgctgaccgtagtactcctccaaggagtcgtcctggtagaagccgctgtgcgcccgcgactccgagcgctcgaagcggctcccgccccgcgcctcgtgactgttgccgtctgcccggcggggccgctggccgtaggagtcagcgaaggccgccagctcgtccatggaaacggccggcacccccgtggcgaagttcttccgcgacagcatctccgacttggagcgcggctg

SEQ ID NO: 69 - protein, 10 aa, Homo sapiens
HQMRSKQFPV

SEQ ID NO: 70 - protein, 13 aa, Artificial Sequence
  Description of Artificial Sequence Synthetic peptide
IVHDADLQIGKLM

SEQ ID NO: 71 - protein, 10 aa, Artificial Sequence
  Description of Artificial Sequence Synthetic peptide
HQMRNKQFPM

SEQ ID NO: 72 - protein, 13 aa, Homo sapiens
IVHDADLQIGKLM

SEQ ID NO: 73 - DNA, 510 bp, Homo sapiens
gaaggcccagccgcggtcagcatcggtcaggggctcgcggctgcggctgcgctgaccgtagtactcctccaaggagtcgtcctggtagaagccgctgtgcgcccgcgactccgagcgctcgaagcggcatcccgccccgcgcctcgtgactgttgccgtctgcccggcggggccgctggccgtaggagtcagcgaaggccgccagctcgtccatggaaacggccggcacccccgtggcgaagttcttccgcgacagcatctccgacttggagcgcggctggctgcgggcagagaaggagggggtcagacggccggtccctccctggagctccagctccacttagtgctcatcttctcagcgcttttgcgttccattggaggagcatattcacactaaaaaaagaccactttctagattgaggacatgcgtcactctagcatctgaggatcccaccttcactttgtgagagcacagctctccttggaggcattttttattttttgaacatt

SEQ ID NO: 74 - DNA, 577 bp, Mus musculus
atcggtcaggggctcgcggctgcggctgcgctgaccgtagtactcctccaaggagtcgtcctggtagaagccgctgtgcgcccgcgactccgagcgctcgaagcggctcccgccccgcgcctcgtgactgttgccgtctgcccggcggggccgctggcctgtaggagtcagcgaaggccgccagctcgtccatggaaacggccggcacccccgtggcgaagttcttccgcgacagcatctccgacttggagcgcggctggctgcgggcagagaaggagggggtcagacggccggtccctccctggagctccagctccacttagtgctcatcttctcagcgcttttgcgttccattggaggagcatattcacactaaaaaaagaccactttctagattgaggacatgcgtcactctagcatctgaggatcccaccttcactttgtgagagcacaggagaagatccccgaactacggcgacgaggcctgcctgtggctcgcgttgctgatgccatcccttactgctccgcagactgggcgcttctgagggaggaagaaaaggagaaatacgcagaaatg

SEQ ID NO: 75 - DNA, 24 bp, Artificial Sequence
  Description of Artificial Sequence Synthetic primer
gcaaactaacccgcactaaactgg

SEQ ID NO: 76 - DNA, 24 bp, Artificial Sequence
  Description of Artificial Sequence Synthetic primer
agggactcaggaaaggtgaaggaa

SEQ ID NO: 77 - DNA, 25 bp, Artificial Sequence
  Description of Artificial Sequence Synthetic primer
cacggactttctctacatacttttg

SEQ ID NO: 78 - DNA, 22 bp, Artificial Sequence
  Description of Artificial Sequence Synthetic primer
ttcatccacatcatcgtacact

SEQ ID NO: 79 - DNA, 24 bp, Artificial Sequence
  Description of Artificial Sequence Synthetic primer
tttcactgcaaagttgtgatggcg

SEQ ID NO: 80 - DNA, 22 bp, Artificial Sequence
  Description of Artificial Sequence Synthetic primer
atgtcatccagcacacctgtcc

SEQ ID NO: 81 - DNA, 20 bp, Artificial Sequence
  Description of Artificial Sequence Synthetic primer
agccatgtacgtagccatcc

SEQ ID NO: 82 - DNA, 20 bp, Artificial Sequence
  Description of Artificial Sequence Synthetic primer
ctctcagctgtggtggtgaa

SEQ ID NO: 83 - DNA, 20 bp, Artificial Sequence
  Description of Artificial Sequence Synthetic primer
atctgctggtgccaatgctg

SEQ ID NO: 84 - DNA, 20 bp, Artificial Sequence
  Description of Artificial Sequence Synthetic primer
gccgtacgagtctgcgaagg

SEQ ID NO: 85 - DNA, 22 bp, Artificial Sequence
  Description of Artificial Sequence Synthetic primer
tgggccaacctgaagttgtggt

SEQ ID NO: 86 - DNA, 20 bp, Artificial Sequence
  Description of Artificial Sequence Synthetic primer
gcccttgggttttccaggct

SEQ ID NO: 87 - DNA, 25 bp, Artificial Sequence
  Description of Artificial Sequence Synthetic primer
ctgaatttgaccctgaaagaagagc

SEQ ID NO: 88 - DNA, 17 bp, Artificial Sequence
  Description of Artificial Sequence Synthetic primer
acttcccggtagagggc

SEQ ID NO: 89 - DNA, 25 bp, Artificial Sequence
  Description of Artificial Sequence Synthetic primer
aggtttaaccatgccaattatggac

SEQ ID NO: 90 - DNA, 21 bp, Artificial Sequence
  Description of Artificial Sequence Synthetic primer
ctctggggcttttcacaaact

SEQ ID NO: 91 - DNA, 18 bp, Artificial Sequence
  Description of Artificial Sequence Synthetic primer
tggagcttggtcgtgtag

SEQ ID NO: 92 - DNA, 18 bp, Artificial Sequence
  Description of Artificial Sequence Synthetic primer
ccacggtgccatcatcaa

SEQ ID NO: 93 - DNA, 20 bp, Artificial Sequence
  Description of Artificial Sequence Synthetic primer
aattctggagcagatgtggc

SEQ ID NO: 94 - DNA, 20 bp, Artificial Sequence
  Description of Artificial Sequence Synthetic primer
catggtcccaaactcgattc

SEQ ID NO: 95 - DNA, 22 bp, Artificial Sequence
  Description of Artificial Sequence Synthetic primer
atcgtcctgcgctacaagaccc

SEQ ID NO: 96 - DNA, 24 bp, Artificial Sequence
  Description of Artificial Sequence Synthetic primer
gggtcacagtctctgtcgtgttcc

SEQ ID NO: 97 - DNA, 16 bp, Artificial Sequence
  Description of Artificial Sequence Synthetic primer
gggagcgtgcgtcggt

SEQ ID NO: 98 - DNA, 25 bp, Artificial Sequence
  Description of Artificial Sequence Synthetic primer
aggactcggtagaagctatcctggc

SEQ ID NO: 99 - DNA, 20 bp, Artificial Sequence
  Description of Artificial Sequence Synthetic primer
gcagacacgctgctcatcgt

SEQ ID NO: 100 - DNA, 25 bp, Artificial Sequence
  Description of Artificial Sequence Synthetic primer
cgcgaacatggatttcatccgtacc

SEQ ID NO: 101 - DNA, 24 bp, Artificial Sequence
  Description of Artificial Sequence Synthetic primer
acgggaagtccatatttgaggccc

SEQ ID NO: 102 - DNA, 17 bp, Artificial Sequence
  Description of Artificial Sequence Synthetic primer
ggaggcgcacccgcttt

SEQ ID NO: 103 - DNA, 18 bp, Artificial Sequence
  Description of Artificial Sequence Synthetic primer
ggcgcacgaaggtgagcc

SEQ ID NO: 104 - DNA, 22 bp, Artificial Sequence
  Description of Artificial Sequence Synthetic primer
cctcgataccgcttgctagggt

SEQ ID NO: 105 - DNA, 23 bp, Artificial Sequence
  Description of Artificial Sequence Synthetic primer
accggattgcacatcatcgacca

SEQ ID NO: 106 - DNA, 18 bp, Artificial Sequence
  Description of Artificial Sequence Synthetic primer
ccccgctcgacgttcgga

SEQ ID NO: 107 - DNA, 19 bp, Artificial Sequence
  Description of Artificial Sequence Synthetic primer
cagtagccttgcccacggg

SEQ ID NO: 108 - DNA, 23 bp, Artificial Sequence
  Description of Artificial Sequence Synthetic primer
acctggtaagggcttgatgtcct

SEQ ID NO: 109 - DNA, 19 bp, Artificial Sequence
  Description of Artificial Sequence Synthetic primer
cttcgaggccattgcgccc

SEQ ID NO: 110 - DNA, 22 bp, Artificial Sequence
  Description of Artificial Sequence Synthetic primer
gggtcgcttatggtccttgccg

SEQ ID NO: 111 - DNA, 19 bp, Artificial Sequence
  Description of Artificial Sequence Synthetic primer
gcagcccaatcggactcta

SEQ ID NO: 112 - DNA, 20 bp, Artificial Sequence
  Description of Artificial Sequence Synthetic primer
acatcctggttggaaagtcg

SEQ ID NO: 113 - protein, 19 aa, Mus musculus
CLFLIIYCPDRASAIQVTV

SEQ ID NO: 114 - protein, 19 aa, Mus musculus
GWTAVFWLTAMVEGLQVTV

SEQ ID NO: 115 - protein, 10 aa, Mus musculus
LEDWLFVVVV

SEQ ID NO: 116 - protein, 10 aa, Mus musculus
MPEWVFVGLV

SEQ ID NO: 117 - protein, 36 aa, Mus musculus
CLASLLFFLLLGICWCQCCPHTCCCYVRCPCCPDKC

SEQ ID NO: 118 - protein, 36 aa, Mus musculus
ILGIFLFFVLVGICWCQCCPHSCCCYVRCPCCPDSC

SEQ ID NO: 119 - protein, 15 aa, Mus musculus
ERRRVYREEEEEEEE

SEQ ID NO: 120 - protein, 47 aa, Mus musculus
ESSSRGGSLETPSKLGAQLGPRSASYYAWSPPTTYKAGASEGEDEDD

SEQ ID NO: 121 - protein, 4 aa, Mus musculus
NPGY

SEQ ID NO: 122 - protein, 4 aa, Mus musculus
NPDY

SEQ ID NO: 123 - protein, 4 aa, Mus musculus
RSRS

SEQ ID NO: 124 - protein, 6 aa, Mus musculus
RSRASY

SEQ ID NO: 125 - protein, 6 aa, Mus musculus
RAGSRF

SEQ ID NO: 126 - protein, 4 aa, Artificial Sequence
  Description of Artificial Sequence Synthetic peptide
NNTI

SEQ ID NO: 127 - protein, 8 aa, Artificial Sequence
  Description of Artificial Sequence Synthetic peptide
SXLDXELX

SEQ ID NO: 128 - protein, 5 aa, Artificial Sequence
  Description of Artificial Sequence Synthetic peptide
DKERD

SEQ ID NO: 129 - protein, 88 aa, Mus musculus; identical to SEQ ID NO: 1

SEQ ID NO: 130 - DNA, 16 bp, Mus musculus
acacacacacacacac

SEQ ID NO: 131 - DNA, 37 bp, Mus musculus
agatgcacgaggaggccagctgcagacagcctgagtt

SEQ ID NO: 132 - DNA, 10 bp, Mus musculus
ctaccccccc

SEQ ID NO: 133 - protein, 21 aa, Mus musculus
VGGHSSQVPLLREVDGSVSSE

SEQ ID NO: 134 - protein, 135 aa, Mus musculus
GQSIMTVRTTHTEVEVHAGGTVELPCSYQLANDTQPPVISWLKGASPDRSTKVFKGNYNWQGEGLGFVESDSYKESFGDFLGRASVANLAAPTLRLTHVHPQDGGRYWCQVAQWSIRTEFGLDAKSVVLKVTGHT

SEQ ID NO: 135 - protein, 151 aa, Mus musculus
AMVEGLQVTVPDKKKVAMLFQPTVLRCHFSTSSHQPAVVQWKFKSYCQDRMGESLGMSSPRAQALSKRNLEWDPYLDCLDSRRTVRVVASKQGSTVTLGDFYRGREITIVHDADLQIGKLMWGDSGLYYCIITTPDDLEGKNEDSVELLVL

SEQ ID NO: 136 - protein, 6 aa, Artificial Sequence
  Description of Artificial Sequence Synthetic 6x His tag
HHHHHH

(19) United States
(12) Patent Application Publication
(10) Pub. No.: US 2013/0160150 A1
(43) Pub. Date: Jun. 20, 2013

(54) METHODS FOR IDENTIFYING COMPOUNDS THAT MODULATE LISCH-LIKE PROTEIN OR C1ORF32 PROTEIN ACTIVITY AND METHODS OF USE

(71) Applicants: Rudolph L. Leibel, New York, NY (US); Wendy K. Chung, Hackensack, NJ (US); Charles LeDuc, Hackensack, NJ (US); Stuart G. Fischer, New Rochelle, NY (US); Chouinard Roland, Fort Lee, NJ (US)

(72) Inventors: Rudolph L. Leibel, New York, NY (US); Marija Dokmanovic-Chouinard, New York, NY (US); Wendy K. Chung, Hackensack, NJ (US); Charles LeDuc, Hackensack, NJ (US); Stuart G. Fischer, New Rochelle, NY (US)

(73) Assignee: The Trustees of Columbia University in the City of New York, New York, NY (US)

(21) Appl. No.: 12/747,866

(22) PCT Filed: Dec. 12, 2008
(86) PCT No.: PCT/US08/86707

§ 371 Date: Nov. 2, 2011

Related U.S. Application Data
(60) Provisional application No. 61/013,194, filed on Dec. 12, 2007.
(60) Provisional application No. 61/047,667, filed on Apr. 24, 2008.

(51) Int. Cl. G01N 33/50

(52) U.S. Cl. CPC G01N 33/5008
USPC 800/3; 506/9; 530/350; 530/387.9; 536/23.5; 435/7.9; 435/6.12; 435/375; 424/93.7; 514/44 R; 514/6.9; 604/522

(57) ABSTRACT

The invention provides methods for reducing diabetes susceptibility in a subject and methods for increasing the expression of LL or CLORF32 in a subject. The invention further provides a method for identifying an agent which modulates expression of an Ll RNA or Clorf32 RNA comprising contacting a cell with an agent; determining expression of the Ll RNA or Clorf32 RNA in the presence and the absence of the agent; and comparing expression of the Ll RNA or Clorf32 RNA in the presence and the absence of the agent, wherein a change in the expression of the Ll RNA or Clorf32 RNA in the presence of the agent is indicative of an agent which modulates the level of expression of the RNA.

This application claims priority to U.S. Provisional Application No. 61/013,194 filed on Dec. 12, 2007 and U.S. Provisional Application No. 61/047,667 filed on Apr. 24, 2008, both of which are hereby incorporated by reference in their entireties.

This invention was made with government support under N.I.H. Grant Number DK66518. As such the United States government has certain rights in this invention.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

Throughout this application, various publications are referenced. The disclosures of these publications in their entireties are hereby incorporated by reference into this application in order to more fully describe the state of the art as known to those skilled therein as of the date of the invention described and claimed herein.

### BACKGROUND

Type 2 diabetes (T2DM) afflicts 246 million people worldwide, including 21 million in the United States. Another 54 million Americans have pre-diabetes. If the incidence of T2DM continues to increase at the present rate, one in three Americans, and one in two minorities born in 2000 will develop diabetes in their lifetime (Cowie C, MMWR 52: 833-837, 2003). In addition to the human cost, direct medical costs associated with diabetes in the United States currently exceed $132 billion a year and consume ˜10% of health care costs in industrialized nations (Saltiel A R Cell 104: 517-529, 2001). Diabetes is the leading cause of both end stage renal disease and blindness (in people aged 20-74 years), and its association with cardiovascular disease increases mortality rates two-fold.

Although intensive genetic analyses of human populations have confirmed contributory roles for some specific genes, these cannot account—even in the aggregate—for powerful genetic predisposition T2DM. The link between obesity and diabetes is the result of obesity-related insulin resistance stress on the insulin-producing cells of the pancreas. Genetic differences and differences in numbers of insulin producing beta cells can cause differential susceptibility among individuals to T2DM. Therefore, there is a need to identify relevant genes associated with susceptibility to diabetes. This invention addresses this need and provides treatment strategies for manipulating beta cells and treating T2DM.

### SUMMARY

The invention provides for a method for identifying an agent which modulates expression of a murine Ll RNA, the method comprising: contacting a murine cell with an agent, wherein the cell contains an L1 gene; determining expression of Ll RNA in the cell in the presence and absence of the agent; and comparing expression of Ll RNA in the cell in the presence and absence of the agent, wherein a change in the expression of the Ll RNA in the presence of the agent is indicative of an agent which modulates the level of expression of the RNA. The invention provides for a method for identifying an agent which modulates expression of a human C1orf32 RNA, the method comprising: contacting a human cell with an agent, wherein the cell contains a C1orf32 gene; determining expression of the C1orf32 RNA in the cell in the presence and the absence of the agent; and comparing expression of the C1orf32 RNA in the cell in the presence and the absence of the agent, wherein a change in the expression of the C1orf32 RNA in the presence of the agent is indicative of an agent which modulates the level of expression of the RNA. The invention also provides for a method for identifying an agent which modulates expression of an mRNA encoding a murine LL protein, or a fragment or an isoform thereof, the method comprising: contacting a cell with an agent; determining expression of the mRNA in the presence and the absence of the agent, and comparing the expression of the mRNA in the presence or the absence of the agent, wherein a change in the expression of the mRNA encoding LL protein in the presence of the agent is indicative of an agent which modulates the expression of the mRNA. The invention also provides for a method for identifying an agent which modulates expression level of an mRNA encoding the protein encoded by human C1ORF32 gene, the method comprising: contacting a cell expressing the C1ORF32 gene with an agent; determining expression levels of mRNA encoded by C1ORF32 in the presence and the absence of the agent; and comparing the expression level of the mRNA in the presence and the absence of the agent, wherein a change in the level of expression of the mRNA encoding C1ORF32 in the presence of the agent is indicative of an agent which modulates the expression level of the mRNA. The invention provides for a method for identifying an agent which modulates expression of murine Ll RNA, the method comprising: contacting a cell expressing L1 RNA with an agent; determining expression of an antisense RNA in the presence and the absence of the agent, wherein the antisense RNA comprises the sequence shown in SEQ ID NO: 18, 19 or 20; and comparing the expression of the antisense RNA in the presence and the absence of the agent, wherein a change in the expression of the antisense RNA is indicative of an agent which modulates the expression of the Ll RNA. The invention provides for a method for identifying an agent which modulates expression of C1orf32 RNA, the method comprising: contacting a cell expressing C1ORF32 RNA with an agent; determining expression of an antisense RNA in the presence and the absence of the agent, wherein the antisense RNA comprises the sequence shown in SEQ ID NO: 68, 73 or 74; and comparing the expression of the antisense RNA in the presence and the absence of the agent, wherein an a change in the expression of the antisense RNA is indicative of an agent which modulates the of expression of the C1orf32 RNA.

In one embodiment, the determining the expression comprises determining stability of RNA, determining level of RNA expression, determining level of expression of a type of C1ORF32 or LL RNA isoform or any combination thereof. The invention provides for a method for identifying an agent which modulates expression of an LL murine protein, the method comprising: contacting a cell expressing the LL protein with an agent; determining expression of the LL protein in the presence and the absence of the agent; and comparing the expression of the LL protein in the presence or the absence of the agent, wherein a change in the expression of the LL protein in the presence of the agent is indicative of an agent which modulates the expression of the LL protein.

The invention also provides for a method for identifying an agent which modulates expression of human C1ORF32 protein, the method comprising: contacting a cell expressing human C1ORF32, with an agent; determining expression of the human C1ORF32 protein in the presence and absence of the agent; and comparing the expression of the human C1ORF32 protein in the presence and absence of the agent, wherein a change in the expression of the C1ORF32 protein in the presence of the agent is indicative of an agent which modulates the expression of the human C1ORF32 protein. In one embodiment, the LL protein or the C1ORF32 protein comprises a label. In one embodiment, the label comprises a fluorescent label. In one embodiment, the fluorescent label comprises a Green, Yellow, Cyanne, Chemy, Fluorescent Protein or any variant thereof. In one embodiment, the change is an increase. In one embodiment, the change is a decrease. In one embodiment, the change is transient. In one embodiment, the change is in localization, stability, modification, processing, posttranslational modification, or any combination thereof.

In one embodiment, the Ll RNA or the C1orf32 RNA is endogenous. In one embodiment, the LL RNA or protein or the C1ORF3 RNA or protein is endogenous. In one embodiment, the cell is transfected with a nucleic acid comprising the nucleic acid of any of SEQ ID NO: 10-13, 15-20 or a nucleic acid which is at least 75% homologous to any of SEQ ID NO: 10-13, 15-20. In one embodiment, the cell comprises a fluorescently labeled C1ORF32. In one embodiment, the cell is transfected with a nucleic acid comprising the nucleic acid of C1orf32 cDNA sequence or genomic sequence, with regulatory elements or a nucleic acid which is at least 75% homologous to same. In one embodiment, the cell is derived from a diabetes-relevant tissue. In one embodiment, the tissue comprises liver, pancreatic islet, skeletal muscle, brain, adipose tissue, or combination thereof. In one embodiment, the cell comprises a pancreatic cell, a β-cell or an islet of Langerhans cell. In one embodiment, the cell comprises an insulin producing beta cell, a hepatocyte cell, or a hypothalamic cell. In one embodiment, the cell comprises a murine cell, a rat cell, or a human cell. In one embodiment, the method is performed in vivo or in vitro.

The invention provides for an isolated peptide consisting essentially of the amino acid sequence of SEQ ID NO: 6 or an isolated peptide which is at least 75% identical to SEQ ID NO: 6. The invention provides for an isolated peptide consisting essentially of the amino acid sequence of SEQ ID NO: 7 or an isolated peptide which is at least 75% identical to SEQ ID NO: 7. The invention provides for an isolated peptide consisting essentially of the amino acid sequence of SEQ ID NO: 8 or an isolated peptide which is at least 75% identical to SEQ ID NO: 8. The invention provides for an isolated peptide consisting essentially of the amino acid sequence of SEQ ID NO: 9 or an isolated peptide which is at least 75% identical to SEQ ID NO: 9. The invention provides for an isolated peptide consisting essentially of the amino acid sequence of SEQ ID NO: 70 or an isolated peptide which is at least 75% identical to SEQ ID NO: 70. The invention provides for an isolated peptide consisting essentially of the amino acid sequence of SEQ ID NO: 71 or an isolated peptide which is at least 75% identical to SEQ ID NO: 71. The invention provides for an isolated peptide consisting essentially of the amino acid sequence of SEQ ID NO: 72 or an isolated peptide which is at least 75% identical to SEQ ID NO: 72. The invention provides for an isolated peptide consisting essentially of the amino acid sequence of SEQ ID NO: 69 or an isolated peptide which is at least 75% identical to SEQ ID NO: 69. The invention provides for a mixture comprising at least two of any of these peptides.

The invention provides for an antibody which specifically binds to any of the peptides described herein. In one embodiment, the antibody is a polyclonal antibody. In one embodiment, the antibody is a monoclonal antibody. In one embodiment, the antibody is a soluble antibody fragment.

The invention provides for an isolated nucleic acid consisting essentially of SEQ ID NO: 18, 19 or 20 or an isolated nucleic acid which is at least 75% homologous to the nucleic acid of SEQ ID NO: 18, 19 or 20. The invention provides for an isolated nucleic acid consisting essentially of SEQ ID NO: 68, 73 or 74 or an isolated nucleic acid which is at least 75% homologous to the nucleic acid of SEQ ID NO: 68, 73, or 74. The invention provides for a composition comprising the nucleic acid described herein.

The invention provides for a method for detecting a predisposition to type 2 diabetes in a subject, the method comprising determining expression of C1orf32 RNA or C1ORF32 protein in a sample obtained from a subject, wherein decreased expression, compared to expression in a control sample from a subject known not to have type 2 diabetes, indicates that the subject is susceptible to type II diabetes. In one embodiment, determining comprises measuring expression level of C1orf32 RNA or C1ORF32 protein in the sample, or determining C1ORF32 protein localization or determining post-translational modification of C1ORF32 protein. In one embodiment, determining expression level of C1ORF32 protein comprises immunohistochemistry or Western blotting using an antibody which specifically binds to C1ORF32 protein. In one embodiment, the sample from the subject and the control sample are from a diabetes-relevant tissue or cell. In one embodiment, the diabetes-relevant tissue or cell comprises liver, pancreatic islet, skeletal muscle, brain, adipose tissue, adipose cell, or any combination thereof. In one embodiment, determining comprises quantifying RNA encoding the C1Orf32 polypeptide, a variant thereof, a fragment thereof, or any combination thereof.

The invention provides for a method for manipulating beta cell mass to treat a biological condition in a subject, comprising contacting a beta cell precursor with an agent which increases expression of C1orf32 mRNA or C1ORF32 protein, thereby manipulating beta cell mass in the subject. The invention provides for a method for manipulating beta cell mass to treat a biological condition in a subject, comprising contacting a beta cell precursor with a peptide or polypeptide of the invention, thereby manipulating beta cell mass in the subject.

The invention provides for a method for treating a biological condition associated with reduced beta cell mass in a subject, comprising administering to the subject an agent which increases expression of C1orf32 mRNA or C1ORF32, so as to increase beta cell mass in the subject thereby treating the biological condition. The invention provides for a method for treating a biological condition associated with reduced beta cell mass in a subject, comprising administering to the subject a peptide or polypeptide provided by the invention, so as to increase beta cell mass in the subject thereby treating the biological condition.

The invention provides for a method for treating a biological condition associated with reduced levels of C1orf32 mRNA or C1ORF32 in a subject, comprising administering an agent which increases expression of C1orf32 mRNA or C1ORF32, thereby treating the biological condition. In one embodiment, the biological condition is type II diabetes. In one embodiment, the expression of C1orf32 mRNA or C1ORF32 protein is increased in pancreas, in skeletal muscle, in adipose tissue, in brain hypothalamus, or any combination thereof. In one embodiment, the expression of C1orf32 mRNA or C1ORF32 protein is increased in beta cells. The invention provides for a method for treating a biological condition associated with reduced levels of C1orf32 mRNA or C1ORF32 in a subject, comprising administering a peptide or polypeptide of the invention, thereby treating the biological condition.

The invention provides for a method for increasing expression of C1orf32 RNA or C1ORF32 protein in a pancreatic cell, the method comprising contacting the cell with an agent which increases the levels of the C1orf32 RNA or C1ORF32 protein. In one embodiment, the pancreatic cell is a β-cell or an islet of Langerhans cell.

The invention provides for a method of modulating beta cell development, the method comprising contacting a pancreatic cell with an agent which increases the levels of C1orf32 mRNA or C1ORF32 protein. The invention provides for a method of modulating beta cell development, the method comprising contacting a pancreatic cell with a peptide or polypeptide of the invention.

The invention provides, a method for increasing beta cell mass, beta cell numbers or beta cell proliferation, the method comprising contacting a pancreatic cell with an agent which increases expression of C1orf32 mRNA or C1ORF32 protein. The invention provides a method for increasing beta cell mass, beta cell numbers or beta cell proliferation, the method comprising contacting a pancreatic cell with a peptide or polypeptide provided by the invention. In one embodiment, the method is performed in vivo. In one embodiment, the method is performed ex vivo.

The invention provides a method for treating a pre-diabetic or a diabetic subject, the method comprising administering to the subject a therapeutically effective amount of an agent which increases the expression of C1orf32 mRNA or C1ORF32 protein.

The invention provides a method for treating a pre-diabetic or a diabetic subject, the method comprising administering to the subject a therapeutically effective amount of a peptide or polypeptide provided by the invention. In one embodiment, the subject is suspected to have or has type2 diabetes (T2DM).

The invention provides a method for treating a subject suffering from a disease or disorder associated with defects in beta cell mass, beta cell proliferation or beta cell activity, the method comprising: isolating a pancreatic (beta cell) cell from a donor, introducing a nucleic acid which comprises a nucleic acid sequence encoding C1ORF32 polypeptide into the pancreatic cell; transferring the pancreatic cell of (b) in the subject, wherein the pancreatic cell grows, and differentiates into insulin producing beta cell.

In one embodiment, the donor is the subject. In one embodiment, optionally comprising a step of ex vivo expanding of the pancreatic cell of step (b). In one embodiment, the step of expanding is performed in the presence of growth factors. In one embodiment, the agent is a nucleic acid which comprises a nucleic acid sequence encoding a C1ORF32 protein, a C1ORF32 polypeptide, a C1ORF32 isoform, or a C1ORF32 functional fragment. In one embodiment, the agent is a C1ORF32 protein, a C1ORF32 polypeptide, a C1ORF32 isoform, or a C1ORF32 functional fragment.

In one aspect, the invention provides the identification of Lisch-like (Ll) as a gene involved in T2DM. Ll was identified quantitative trai loci (QTL) analysis of modifiers of T2DM in C57BL/DBA/2J F2/F3 Lep^{ob/ob} mice and gene cloning based in B6.DBA N14 congenic line phenotypes. Ll gene expression mediates susceptibility to T2DM by an effect on β cell development as well as other aspects of β cell/islet biology.

Ll gene encodes multiple, tissue-specific transcripts that are most highly expressed in brain, liver and islets. The functional consequences of hypomorphic (diabetes prone) DBA alleles of Ll in Lep^{ob/ob} mice are late embryonic and early postnatal reductions in β-cell mass due to diminished rates of β-cell replication, a recovery of β-cell mass by 2-3 months of age followed by mild glucose intolerance at >6 months of age.

In certain aspects, the invention provides that Ll, Ll homologues and Ll orthologues, regulate generation and survival of islet beta cells and control hepatic glucose homeostasis. The invention provides methods to measure protein biosynthesis, processing, sub-cellular localization, signaling properties and structure/function relationships to determine the effects of Ll in gain-of-function and loss-of-function experiments. In other aspects, the invention provides methods to determine the basis for the reduced expression of Ll in the diabetes-susceptible animals. In other aspects, the invention provides methods to determine the molecular and cell physiology of an animal, for example a mouse, in which the Ll gene has an induced mutation causing inactivation of the protein. In another aspect, the invention provides the human version of Ll gene, C1Orf32. C1ORF32, which is 90% identical to LL at the amino acid sequence level, is located in a region of the human genome that has been repeatedly linked to T2DM in genetic studies. In one embodiment, the invention provides methods to determine whether LL loss of function produces diabetes-susceptibility. In another embodiment, the invention provides methods to identify biological pathways critical to β cell development and survival in the context of insulin resistance and gluco-/lipotoxicity imposed by obesity.

In another aspect, the invention provides a method for manipulating beta cell mass to treat a biological condition in a subject, comprising contacting a beta cell precursor with a peptide having SEQ ID NO:1-9 or 69-72, or a C1ORF32 protein, a C1ORF32 polypeptide, a C1ORF32 isoform, or a C1ORF32 functional fragment, or any combination thereof, thereby manipulating beta cell mass in the subject. In another aspect, the invention provides a method for treating a biological condition associated with reduced beta cell mass in a subject, comprising administering to the subject a peptide having SEQ ID NO:1-9 or 69-72, or a C1ORF32 protein, a C1ORF32 polypeptide, a C1ORF32 isoform, or a C1ORF32 functional fragment, or any combination thereof, so as to increase beta cell mass in the subject thereby treating the biological condition. In one embodiment, the biological condition is type II diabetes, obesity, dyslipidemias, or any combination thereof.

In another aspect, the invention provides a method for treating a pre-diabetic or a diabetic subject, the method comprising administering to the subject a therapeutically effective amount of a peptide having SEQ ID NO:1-9 or 69-72, or a C1ORF32 protein, a C1ORF32 polypeptide, a C1ORF32 isoform, or a C1ORF32 functional fragment, or any combination thereof.

The invention provides a peptide having SEQ ID NO:1-9 or 69-72, or a C1ORF32 protein, a C1ORF32 polypeptide, a C1ORF32 isoform, or a C1ORF32 functional fragment for use in treating a pre-diabetic or a diabetic condition in a subject. The invention provides a peptide having SEQ ID NO:1-9 or 69-72, or a C1ORF32 protein, a C1ORF32 polypeptide, a C1ORF32 isoform, or a C1ORF32 functional fragment for use in treating a biological condition associated with reduced beta cell mass in a subject.

A peptide having SEQ ID NO:1-9 or 69-72, or a C1ORF32 protein, a C1ORF32 polypeptide, a C1ORF32 isoform, or a C1ORF32 functional fragment for use in manipulating beta cell mass to treat a biological condition in a subject.

### BRIEF DESCRIPTION OF THE FIGURES

FIG. 1 shows LOD scores and for chromosome 1 markers and a summary of terminal phenotypes. FIG. 1A shows LOD scores for markers along mouse chromosome 1 for fasting blood glucose (black) and pancreatic grade (blue) in F2 Lep^{ob}/Lep^{ob} B6/DBA mice. FIG. 1B shows a summary of terminal phenotypes by genotype at D1Mit110 at 169.9 Mb. Pancreatic grade is a subjective measure of number and size of islets and islet integrity with grading from 1 (many, large, intact isles) to 5 (few, small islets with little insulin signaling). P-value is for effect of the genotype.

FIG. 2 shows sub-congenic lines for genetic interval Chr 1 164-194 Mb. Above the map scale, markers in black type were used to genotype B6 and DBA alleles. D1mit110 is the peak of the F2/F3 QTL linkage map. Below map scale, RefSNP (rs) and D-markers in red type identify DBA sequence limits in respective congenic lines. Markers in blue type identify the closest, confirmed non-DBA (B6) sequence. Sequences in intervals between markers in red and blue type are DBA vs. B6 invariant. Gray bars are DBA-derived sequences. Yellow box corresponds to a 3.2 Mb interval, conserved between DBA and B6. The red box identifies the N-scan predicted gene, chr1.1224.1, subsequently identified as Lisch-like (Ll), extending centromerically from line 1jcdt. In the expanded view of L1, the B6 boundary (rs31968429) for lines 1jcdc, 1jcd, 1jcdt is 333 bp centromeric to exon 7; the DBA boundary, (rs33860076) is 2,700 bp telomeric to exon 7. 5330438103Rik is an anti-sense transcript. Marker positions are from the current mouse genome annotation (NCBI Build 36, February 2006).

FIG. 3 shows phenotypes of congenic animals.

FIG. 4 shows plasma glucose and insulin in 30 and 62 day old 1jc mice. FIG. 4A shows a scatter plot of plasma glucose and insulin in a scatter plot of 1jc male mice. FIG. 4B shows the Plasma Insulin/Glucose Ratio of 1jc Lep ob/ob mice. FIG. 4C shows plasma glucose and insulin levels 30 day and 62 day old mice.

FIG. 5 shows fasting glucose and glucose tolerance in congenic lines.

FIG. 5A shows blood glucose in Lep^{ob/ob} males congenic for the interval 1jcd fed regular mouse chow diet (9% fat) ad libitum. Determinations made were following a 4 h morning fast. From 4-13 animals per genotype group. Mean+/−SEM. * indicates p<0.05 (2-tailed t-test) for genotype effect. FIG. 5B shows blood glucose in Lep+/+ males congenic for the interval 1jcd fed high fat diet (60% of calories as fat) ad libitum for 13 wks, starting at 7 wks of age. N=8 BB; N=11 DD. Determinations were made following a 4 h morning fast. Mean+/−SEM. * indicates p<0.05 for genotype effect. FIG. 5C shows ipGTT in 60-day old Lepob/ob males congenic for the interval 1jcdc. N=7 BB; N=5 DD. Mean+/−SEM. * indicates p<0.05 for genotype effect. FIG. 5D shows ipGTT in 200-day old Lepob/ob males congenic for the interval 1jcdc. N=14 BB; N=8 DD. Mean+/−SEM. * indicates p<0.05 for genotype effect. FIG. 5E shows ipGTT in 14-wk old male Lep+/+ males congenic for the interval 1jc who had been fed the “Surwit” diet for 10 wks. N=6 BB; N=6 DD. Mean+/−SEM. * indicates p<0.05 for genotype effect.

FIG. 6 shows islet histology in 21-day old 1jcd male mice. 4 μm pancreatic sections from 21-day old Lepob/ob male B/B and D/D (1jcd) mice were insulin stained with anti-guinea pig IgG and visualized by light microscopy at 10× magnification. In D/D animals, islets were smaller and less numerous. By histomorphometry, the proportion of small islets (250-2000 μm2) in 21 day old Lepob/ob males was greater in D/D (1jc and 1jcd) mice (73%) than in B/B (60%); whereas the proportion of large islets (10,000-50,000 μm2) was lower (9% in D/D and 14% in B/B).

FIG. 7 shows relative β-cell area in male 1jcd lepob/ob mice. In 20, 60 and 150-day old males segregating for the 1jcd D/D sub-congenic interval, relative β-cell masses were approximately half those of B/B littermate controls at 60 and 150 days; B/D animals were intermediate at 150 days. N=10 for each of the 3 groups of animals. Mean+/−SEM. * indicates p<0.05 v. BB. These findings are consistent with in vivo data showing onset of elevated blood glucose at rest and during ipGTT by 60 days.

FIG. 8 shows β-cell replication rates in male 1jcd Lepob/ob mice. Rates of β-cell replication (Ki67) were determined in 1jcd congenic 1- and 21-day old Lepob/ob male mice. To estimate the proportion of dividing cells, the number of Ki67-positive β-cells was normalized to the total number of insulin-positive cells. Replication of β-cells in 1-day old D/D males was ˜⅓ that of B/B littermates (p=0.017). This difference was not present in 21-day old animals due to normally reduced β-cell replication by the time of weaning.

FIG. 9 shows genes and haplotypes in the minimal congenic interval. FIG. 9A. Haplotypes of diabetes-susceptible and resistant strains. Markers are from dbSNP/mouse. Blue bars (major allele); red bars (minor alleles). FIG. 9B. Genes. Gray bar corresponds to the minimal DBA “variable” interval from 168.1 Mb-169.9 Mb on Chr 1. Pink box between markers rs13476219 and rs222799 corresponds to a diabetes susceptibility interval defined by shared haplotypes among inbred strains. Genes in blue are from RefSeq; genes in black are predicted and locally confirmed.

FIG. 10 shows liver expression of lisch-like in 1jc males. The abundant L1 splice variants (iso1, iso2, iso4 and iso5) were collectively analyzed by qRT-PCR in B/B (N=3) and 1jc D/D (N=4) livers of 21, 60, 90 and 200-day old Lepob/ob male animals and shown as a ratio (×10-3) of isoform to β-actin expression. * indicates difference p<0.02. There is a trend towards persistence of this difference at 90 and 200 days, but a “recovery” of L1 expression in DD mice is congruent with their improved glucose homeostasis with age.

FIG. 11 shows the predicted structure of the L1 gene and an expanded view of 3 critical intervals.

FIG. 12 shows domain organization of LL proteins. Exon 1 includes the 5′ UTR and a sequence that encodes a cleavable signal peptide (SP). Exons 2-3 encode an immunoglobulin-like extra-cellular domain (Ig-1, Ig-2). At the carboxy end of exon 3 and the amino end of exon 4 are a cluster of potential di-leucine sorting signals. Exon 4 codes for a non-immunoglobulin-like extra-cellular domain (X). The amino half of exon 5 encodes a trans-membrane domain (Tm) and the carboxy half encodes an intra-cellular cysteine-rich domain (cys). Exons 6 and 8 code for proline-rich domains (pro 1 and pro 2). Exon 7 codes for a domain containing a tyrosine-dimer (tyr-tyr). Exon 9 codes for a long acidic domain and exon 10 codes for a domain that contains a PDZ-binding motif and the 3′ UTR. Mouse Ll isoforms: red bars signify deleted sequences compared to isoform 1. Human C1orf32 is NP_{—}955383 (SEQ ID NO:22). Zebrafish_{—}7.2 is similar to NP_{—}0010253630. The red arrow identifies the position and direction of a sequence used to generate a morpholino for Zebrafish studies. The predicted amino acid sequence of the full-length transcript was analyzed using the ELM server. Motifs shown as symbols in isoform 1 are identified at bottom followed by consensus amino acid sequence. Acidic and basic clusters, di-leucine cluster and alternating acid-base sequence) were identified by comparison to the mouse Lsr protein. The positions of the non-synonymous B6 to DBA substitutions of T572A and A632B are identified, respectively, by an encircled T and encircled A. The “STOP” sign marks the position of the exon 2 nonsense codon generated by ENU mutagenesis in a C3HeB/HₑJ (Ingenium) mouse. This mouse can be used for studies of the molecular physiology of Ll. In addition, several short binding motifs are distributed in a manner similar to those in Lsr. These include six potential SH3 ligands on exons 5 and 6, seven CK1 phosphorylation sites on exons 6-9, and twelve CK2 phosphorylation sites on exons 8 and 9. There are also three 14-3-3 mode 1 motifs, predicted at medium stringency on the Scansite server.

FIG. 13 shows Ll isoform frequency. The relative frequency of each isoform in B6 and DBA mice in liver, hypothalamus, and islets, was determined using isoform-specific primers. Amplification efficiency for primer pairs was >90%. There are differences between wild type B6 and DBA animals in the levels of expression of specific isoforms in organs. Note, for example the much higher levels of isoform 4 of Ll in B6 v. DBA liver, and of isoform 2 in hypothalamus.

FIG. 14 shows specificity of rabbit antibodies to intracellular and extracellular Ll domains. FLAG- and GFP-tagged full-length Ll cDNA was transiently transfected into human HEK293 cells and detected in whole cell lysates by Western blotting. The α-GFP and α-FLAG antibodies detected reporter-Ll fusion proteins of the predicted molecular weights, 98 kD and 72 kD (see arrows), respectively.

FIG. 15 shows immunohistochemical staining of Ll in pancreatic sections of 21-day old Lep^{ob/ob} B/B and D/D ijc males which showed a clear difference in LL protein levels in β cells. Triple staining with LL, insulin and DAPI showed that Ll was expressed specifically within β cells in B/B animals, and that Ll protein was low-to-undetectable in D/D islets, consistent with and more striking than the gene expression results.

FIG. 16 shows liver IHC of p28 1jc ob/ob males. The figure shows lower LL protein level in 28 day old D/D v. B/B mice. This is consistent with Ll gene expression levels in liver.

FIG. 17 shows morpholino knockdown of Lsr-like and Lisch-like at 48 hpf. Two dimensional ventral views (anterior towards top) of confocal stacks of 48 hpf embryos, uninjected or injected with 15 ng morpholino: control, Lsr-like sp1, and Lisch-like ATG. Gut-GFP transgene expression (green); insulin immunolabelling (red).

FIG. 18 shows analysis of constructs for the assessment of intracellular localization and trafficking of LL. FIG. 18A shows Full length C57BL/6 LL cDNA was cloned into the pEGFP-N3 vector. MIN6 (beta) cells were transfected and stained with monoclonal anti-GFP. This image (and B,C) show a punctate plasma membrane and cytoplasmic pattern, which can be consistent targeting to specialized plasma membrane compartments (caveolae, coated pits), lysosomes, and mitochondria. FIG. 18B shows MIN6 cells transfected with GFP-LL construct and co-stained with ICD LL rabbit antibody. FIG. 18C shows full length LL was cloned into CMV4A, containing the FLAG sequence. MIN6 cells were transfected and stained with monoclonal anti-flag. FIG. 18D. Knockdown. Three shRNA constructs were prepared with different 21-mer stem sequences designed to maximally reduce target message. The shRNA-containing plasmids and LL-GFP plasmids were co-transfected into HEK293 cells and the efficiency of knock down was measured. GFP intensity per cell was compared in samples transfected with GFP fusion LL vector with and without cotransfection with shRNA constructs. These data indicate that LL can be efficiently knocked-down using these constructs.

FIG. 19 shows positions LL amino acid sequence

(SEQ ID NO: 1)
MDRVVLGWTAVFWLTAMVEGLQVTVPDKKKVAMLFQPVTLRCHFSTSSHQ
PAVVQWKFKSYCQDRMGESLGMSSPRAQALSKRNLEWD

as well as computational analysis of ENU-induced mutations using SNAP, PolyPhen, SIFT, PAM250 matrix substitution weights and PROFacc algorithms. The LL sequences harboring ENU-induced mutations in the LL amino acid sequence are:

(SEQ ID NO: 2)
MDRVVLGWTAVFWLTAMVEGLQVTVPDKKKVAMLFQRVTLRCHFSTSSHQ
PAVVQWKFKSYCQDRMGESLGMSSPRAQALSKRNLEWD
(SEQ ID NO: 3)
MDRVVLGWTAVFWLTAMVEGLQVTVPDKKKVAMLFQPVTLRCHFSTSSLQ
PAVVQWKFKSYCQDRMGESLGMSSPRAQALSKRNLEWD
(SEQ ID NO: 4)
MDRVVLGWTAVFWLTAMVEGLQVTVPDKKKVAMLFQPVTLRCHFSTSSHQ
PAVVQWKFKSYCLDRMGESLGMSSPRAQALSKRNLEWD
(SEQ ID NO: 5)
MDRVVLGWTAVFWLTAMVEGLQVTVPDKKKVAMLFQPVTLRCHFSTSSHQ
PAVVQWKFKSYCQVRMGESLGMSSPRAQALSKRNLEWD
(SEQ ID NO: 58)
MDRVVLGWTAVFWLTAMVEGLQVTVPDKKKVAMLFQPVTLRCHFSTSSHQ
PAVVQWKFKSYCQDRMGESLGMSSPRAQALSKRNLEW

FIG. 20 shows genomic structure of the targeted L1 allele for conditional inactivation and activation. FIG. 20A shows conditional inactivation. FIG. 20B shows conditional activation. Exon 1 of the L1 gene (black rectangle), the PGKneo triple polyA cassette (white rectangle), loxP sites (black triangle) and FRT sites (white triangle) are depicted.

FIG. 21 shows the predicted structure of L1 gene with expanded views of critical regions. Lisch-like gene (middle) is the full-length, 10-exon, splice variant (iso1) and includes 872 bp upstream of the transcriptional start site. Predicted domains are below exons. Exon 1 includes the 5′ UTR (narrow orange bar) and cleavable signal peptide (SP). Exons 2-4 are extra-cellular, within which exons 2-3 code for an Ig-like domain. Exon 5 includes the TMD with a very cysteine-rich cluster in the carboxyl half; exons 6-10 code for a serine- and proline-rich intracellular domain; exon 10 also includes a long 3′ UTR. The red “Xs” identify exons deleted in isoforms 2-4. FIG. 21A. 5′ upstream interval (expanded view); Black bars correspond to BLAT displays vs. the reference B6 genome. DBA variants are below the DBA bar. Annotations are composites of displays from the UCSC Genome Browser on Mouse February 2006 Assembly. “Regulatory potential” compares frequencies of short alignment patterns between known regulatory elements and neutral DNA. “Conserved sequences”, from the track “vertebrate multiz alignment and conservation”, represents evolutionary conservation in vertebrates. Simple sequence motifs were located by the tandem repeat finder; the CpG island track, provided by the UCSC Genome Browser, generated using the unpublished cpglh program from Washington University (St. Louis) Genome Sequencing Center. FIG. 21B. Anti-sense interval corresponds to the sequences overlapping the Riken transcript 5339438103Rik. Cu_{—}42 is a 37 nt unique sequence insertion in DBA. The two non-synonymous sequence variants in exon 9 are shown. The marker rs33860076 is the centromeric end of the congenic interval. FIG. 21C. 3′ UTR interval; vertical black bars represent positions of 52 B6 vs. DBA nucleotide sequence variants.

FIG. 22 shows specificity of antibody to Ll intracellular domains. HEK293 cells were transiently transfected with cDNA for GFP fused to wild-type (wt) LL isoform 1 (GFP-LL), with a combined molecular weight=98.6 kDa, or with a cDNA coding for GFP fused to LL protein with a stop codon substituting for tryptophan at residue #87 (W87X). Cell lysates were divided and run on parallel NuPAGE 10% Bis-Tris gel with MagicMark XP Western Protein Standard (Invitrogen), Replica membranes were incubated with anti-GFP (1:5000) or anti-ICD (1:2000) antibodies in TBS-T with 5% milk (see Methods, Lisch-like Antibodies). Replica filters were stained with mouse monoclonal anti-beta-tubulin, clone AA2 (Millipore) to normalize loading.

FIG. 23 shows shows the sequences of the mouse peptides used to make antibodies to the LL protein. FIG. 23A shows the amino acid sequence of the Lisch-like α-intracellular domain antigen (amino acid #298-401) (SEQ ID NO: 6). FIG. 23B shows the amino acid sequence of the Lisch-like α-extracellular domain antigen (amino acid #22-186) (SEQ ID NO: 7). FIG. 23C shows the amino acid sequence of the human (C1orf32) cytoplasmic domain corresponding to amino acid 298-401 of Mouse Lisch-like (SEQ ID NO: 8). FIG. 23D shows the amino acid sequence of the human (C1orf32) intracellular domain corresponding to amino acid 22-186 of Mouse Lisch-like (SEQ ID NO: 9). FIG. 23E shows the Lisch-like β-intracellular domain antigen (amino acid #354-363) for the anti-intracellular-Lisch-like antibodies of the invention (SEQ ID NO: 71). FIG. 23F shows the Lisch-like β-extracellular domain antigen (amino acid #124-136) for the anti-extracellular-Lisch-like antibodies of the invention (SEQ ID NO: 70). FIG. 23G shows the amino acid sequence of the human (C1orf32) cytoplasmic domain corresponding to amino acid 354-363 of Mouse Lisch-like (SEQ ID NO: 69). FIG. 23H shows the amino acid sequence of the human (C1orf32) extracellular domain corresponding to amino acid 124-136 of Mouse Lisch-like (SEQ ID NO: 72).

FIG. 24 shows the location of variants in the 5′UTR of Lisch-like gene of DBA (SEQ ID NO: 10) and B6 (SEQ ID NO: 11) strain mice. Shown are the 854 nucleotides 5′ to the 1^{st} coding exon. The DBA sequence is numbered 1-854 above the B6 sequence, numbered 168090227-168091095 below. Positions of variants are highlighted yellow and bold. Above the position of each variant is the dbSNP (rs . . . ) or Columbia_SNP (cu_.) ID. The blue highlight of the genomic sequence identifies simple sequence. The green highlight corresponds to the position of the predicted CpG island; the yellow highlight in the DBA (upper) line, is the predicted upstream transcribed sequence.

FIG. 25 shows the location of variants in the coding exons of Lisch-like gene of B6 (SEQ ID NO: 12) and DBA (SEQ ID NO:13) strain mice. Shown is the 1941 nt coding sequence of the gene, with the B6 sequence on the lower line. The upper line shows the DBA variants in bold, with the dbSNP or Columbia_SNP ID adjacent. The ten coding exons are alternately highlighted yellow and blue. The amino acids coded by corresponding nucleotide variants are highlighted green, amino acids highlighted in gray are non-synonymous variants, where the DBA variant is to the right of the B6 variant (SEQ IB NO: 14).

FIG. 26 shows the location of variants in the 3′UTR of Lisch-like gene of DBA (SEQ ID NO: 15) and B6 (SEQ ID NO: 16) strain mice. Shown is 6052 nucleotides of the complete 3′UTR. DBA sequence shown in italics was not independently confirmed. Therefore, the 3′UTR variants, with the dbSNP IDs in red were identified only from public data.

FIG. 27 shows a summary of the DBA vs. B6 SNPs in the 5′UTR, Transcript, and the 3′UTR of the Lisch-like gene. Summarized are the variants in the Ll gene by position on the chr1 sequence map. For each position the, the dbSNP ID or Columbia_SNP ID is shown. “B6/DBA” shows the B6 nucleotides(s) and the DBA variant at the corresponding position. “AA B6/DBA” shows the B6 and DBA amino acid variants in single letter code. Non-synonymous variants are highlighted in red. The 5′UTR is highlighted in gray; translated exons are not highlighted and the 3′UTR is highlighted in yellow.

FIG. 28 shows the DBA Lisch-like gene 5′UTR, transcript and 3′UTR (SEQ ID NO:17). Shown are the DBA sequence of the 5′UTR, coding exons and 3′UTR of the Lisch-like gene. The positions corresponding to B6 variants are shown in uppercase and highlighted clear. The 5′UTR is highlighted green, and each exon is alternately highlighted in yellow and blue; the 3′UTR is highlighted in green.

FIG. 29 shows variant positions in the Lisch-like anti-sense Transcript in DBA and B6 mice (SEQ ID NO: 18). Shown is the genomic DBA sequence corresponding to the anti-sense transcript, 5330438103RiK. The sequences of the intron preceding exon 8 are highlighted green. Exon 8 is highlighted blue. The intron between exons 8 and 9 is highlighted green. Exon 9 is highlighted yellow. The intronic sequences telomeric to exon 9 and underlying the anti-sense transcript are shown in green.

FIG. 30 shows SNP variants and positions in the Lisch-like anti-sense Transcript in DBA (SEQ ID NO: 19) and B6 mice (SEQ ID NO: 20). Shown is a display generated by a BLAT analysis of the anti-sense transcript of the Ll gene in mouse strain DBA/2J on the reference c57BL/6j genomic sequence. Exons 8 and 9 are highlighted in blue. Annotation is otherwise the same as in FIGS. 24 to 26.

FIG. 31 shows a summary of the DBA vs. B6 SNPs in the Lisch-like anti-sense transcript sequence.

FIG. 32 shows ClustalW analysis of Lisch-like homologs and the LSR protein. ClustalW analysis was performed on the EMBL-EBI server using their default settings. Display was modified to emphasize exonic alignments. Positions of non-synonymous variants in exon 9 of Ll are identified by blue background. Non-homologous extension of mouse Lsr exon 6 (green background) is drawn beneath line. Abbreviations: B6, strain C57BL/6J; DBA, strain DBA/2J; ECD, extra-cellular domain; hpf, hours post-fertilization; Ig-like, immunoglobulin-like; ICD; intra-cellular domain; QTL, quantitative trait locus; TM, trans-membrane domain; T2DM, type 2 diabetes; UTR, untranslated region. Mm_Lisch-like (SEQ ID NO: 21); Hs_c1orf32 (SEQ ID NO: 22); Dr_Lisch-like (SEQ ID NO: 23); Mm_LSR (SEQ ID NO: 24). Also see Table 6.

FIG. 33 shows an alignment of comparative amino acid sequences for LL and related proteins. LL_Musmus (SEQ ID NO: 25); LL_Ratnor (SEQ ID NO: 26); LL_Bostau (SEQ ID NO: 27); LL_Canfam (SEQ ID NO: 28); LL_Homsap (SEQ ID NO: 29); LL_Pantro (SEQ ID NO: 30); LL_Macmul (SEQ ID NO: 31); LL_Feldom (SEQ ID NO: 32); LL_Mondom (SEQ ID NO: 33); LL_Galgal (SEQ ID NO: 34); LL_Xentro (SEQ ID NO:35); LL_Danrer (SEQ ID NO: 36); LSR_Homsap (SEQ ID NO: 37); LSR_Pantro (SEQ ID NO: 38); LSR_Macmul (SEQ ID NO: 39); LSR_Bostau (SEQ ID NO: 40); LSR_Canfam (SEQ ID NO: 41); LSR_Musmus (SEQ ID NO: 42); LSR_Ratnor (SEQ ID NO: 43); LSR_Mondom (SEQ ID NO: 44); LSR_Danrer (SEQ ID NO: 45); ILDR1_Homsap (SEQ ID NO: 46); ILDR1_Pantro (SEQ ID NO: 47); ILDR1_Ponpy (SEQ ID NO: 48); ILDR1_Musmus (SEQ ID NO: 49); ILDR1_Ratnor (SEQ ID NO: 50); ILDR1_Canfam (SEQ ID NO: 51); ILDR1_Xenla (SEQ ID NO: 52); ILDR1_Galgal (SEQ ID NO: 53); and ILDR1_Danrer (SEQ ID NO:54)

FIG. 34 shows exonic structure of the L1 gene and two homologues. The alignment can be used to orient antibody sequence. LL_Musmus (SEQ ID NO: 55); LSR_Musmus (SEQ ID NO: 56); ILDR1_Musmus (SEQ ID NO: 57).

FIG. 35 shows Fasting Glucose and Glucose Tolerance in Male Congenic Lines. (FIG. 35A) Blood glucose in Lep^{ob/ob} males congenic for the interval 1jcd fed regular mouse chow diet (9% fat) ad libitum. Determinations made following a 4-hour morning fast. D/D animals (in red; n=8); B/B (in blue; n=8). P-value at 60 days (0.002), 90 days (0.0009), 120 days (0.003). (FIG. 35B) Lep^{+/+} males congenic for the interval 1jcd fed high fat diet (60% of calories as fat) ad libitum for 13 weeks, starting at 7 weeks of age. Determinations made following a 4-hour morning fast. D/D animals (in red; n=11); B/B (in blue; n=8). P-values at 1 week (0.003), 2 weeks (0.004), 3 weeks (0.01), 5 weeks (0.01), 6 weeks (0.00008), 7 weeks (0.03), 8 weeks (0.001), 9 weeks (0.006), 11 weeks (0.09), 13 weeks (0.009). (FIG. 35C) Intraperitoneal glucose tolerance test (ipGTT) in 60-day old Lep^{ob/ob} males congenic for the interval 1jcdc. Mice were fasted overnight and 0.5 g/kg body weight of 50% dextrose was administered at time 0. D/D animals (in red; n=5); B/B (in blue; n=7). P-value at 120 minutes (0.05). (FIG. 35D) IpGTT in 200-day old Lep^{ob/ob} males congenic for the interval 1jcdc. Mice were fasted overnight and 0.5 g/kg body weight of 50% dextrose was administered at time 0. (FIG. 35E) 14-week old male Lep^{+/+} males congenic for the interval 1jc who had been fed the Surwit diet for 10 weeks were fasted overnight and 0.5 g/kg body weight of 50% dextrose was administered at time 0. Data points are mean+/−SEM. D/D (in red, n=6); B/B (in black; n=6). Asterisks denote significant difference (p<0.05).

FIG. 36 shows spliced and unspliced sequences of the human C1Orf32 Antisense RNA transcript. FIG. 36A shows the tequence of the unspliced human C1Orf32 Antisense RNA transcript (SEQ ID NO: 68). FIG. 36B shows DA322725, a spliced anti-sense transcript of human C1Orf32 corresponding to chr1:165156961-165228581 (SEQ ID NO: 73). FIG. 36C shows DA565656, a spliced anti-sense transcript of human C1Orf32 corresponding to chr1:165156982-165225636 (SEQ ID NO 74).

FIG. 37 shows hyperglycemic clamping in 100-day old 1jc males on Surwit Diet for 18 weeks. 1jc DD male mice fed a Surwit diet for 18 wks were clamped at a blood glucose level of 250 mg/dl for 1 h and serum insulin concentration was measured at 1 hr.

FIG. 38 shows glucose-stimulated insulin secretion in pancreatic islets in 28-day old 1jc Lep^{ob/ob} B/B and D/D males. All animals were 4 weeks old. Each genotype group consisted of 3 male animals. Negative control consisted of 3 4-week old diabetes-prone Lepr^{db/db} KsJ male animals that are hypo-responsive to glucose stimulation (Leiter E H (1989) The genetics of diabetes susceptibility in mice. FASEB J 3: 2231-2241), and positive control of 3 4-week old insulin resistant animals segregating for a diabetes-susceptibility QTL on Chr5@78cM, characterized by hyperglycemia and hyperinsulinemia. B/B and D/D show dose response, but no B/B vs. D/D difference at any concentration of glucose. Arginine (10 mM) response is shown in the same animals. Arginine control confirms that the β-cells of the B/B and D/D congenics are comparable with regard to insulin release to a non-glucose stimulus.

FIG. 39 shows tissue-specific expression analysis of genes in the “variable” interval. Data from table in Example 7 for hypothalamus, islets, liver and EDL-muscle are displayed graphically and in the table below the graph. 21-day old DD and BB Lep^{ob/ob} congenic animals were analyzed using Affymetrix #430A microarray.

FIG. 40 shows developmental expression of zebra fish Lisch-like and Lsr-like orthologs. Lisch-like RNA was hybridized in situ to whole-mount zebra fish embryos at) 48 hours post-fertilization (hpf), dorsal view with anterior towards the top, and 72 hpf, lateral view with anterior towards the top, ventral towards the right and yolk removed. Lsr-like RNA was hybridized at 48 hpf and 34 hpf. Ll panels show ventral views of embryos with yolks removed and anterior towards the top. Lsr-like panels show the same image captured in the focal plane of the anterior (ap) and posterior (pp) pancreatic buds, respectively. i, intestine; ph, pharynx; pn, pronephric ducts; l, liver; ap, anterior pancreatic bud; pp, posterior pancreatic bud; p, pancreas (after anterior and posterior bud fusion); b, brain; o, otic vesicle.

FIG. 41 shows phenotypes of mice segregating for the W87* allele of Lisch-like. FIG. 41A. Western analysis of Lisch-like in hypothalamus of 1jc and homozygous W87* mice. The Western immunoblot shows differences in Ll expression in hypothalami of B/B vs. 1jc D/D congenic males (left panel) and between wild-type C3HeB/FeJ and W87* C3HeB/FeJ males (right panel). The right panel immunoblot was incubated with rabbit anti-LL antiserum, prepared against a polypeptide corresponding to exons 7 and 8 of the ICD. The antiserum had been absorbed to fixed liver extracts from wild type mice in order to block non-specific proteins from interacting with the antibody. The LL transcript isomers are visible as a 65 and 70 kD doublet in the BB and C3HeB/FeJ wild-type lanes, but absent in the lanes of the 1jc-D/D congenic and C3HeB/FeJ W87* homozygous ENU mutants. FIG. 41B. Percent Replicating β-cells in 14-day old ENU-mutagenized mice. The percentage of Ki67-positive β-cells was used to determine the percentage of replicating β-cells in 14-day old C3HeB/FeJ ENU-mutagenized mice, who were either homozygous wild-type (+/+), heterozygous (+/−), or homozygous for the W87* LL amber mutation. ENU-W87* Ll −/− mice show reduced Ki67 staining vs. +/− and +/+ littermates. At 14 days there is 2-fold difference in the % of Ki67⁺ β-cells in +/+ (3.75%) vs. −/− (1.75%) ENU W87* mice; +/− were intermediate (2.5%). Non-overlapping images of longitudinal pancreatic sections (200 μm apart) were acquired and analyzed using ImageJ software version 1.37 (NIH) to count insulin-positive and Ki67⁺ cells. No differences in pancreatic weights of +/+ and −/−. FIG. 41C. Fasting glucose and glucose tolerance in W87* and wild-type mice. FIG. 41D. ipGTT on 50-day old Surwit-fed CH3.B6.N3F1 W87* males. Glucose intolerance is seen in C3H W87* mice. Mice were fasted overnight prior to dextrose injection (50% dextrose solution, 0.5 g/kg, ip). Capillary tail bleeds were performed at the specified time points to determine circulating glucose levels by glucometer (FreeStyle Flash, Abbott). Blood glucose concentrations that are marked with an asterisk are significantly different from each other (t-test; p<0.05; mean±SEM). Area under curve +/+v. −/− (p=0.0241).

FIG. 42 shows a GenTHREADER analysis of Lisch-like exons 2 and 3. FIG. 42A shows a sequence alignment. FIGS. 42B and C show a predicted ligand binding site in Lisch-like. See Example 22.

FIG. 43 shows a secondary structure reference sequence returned from the Robetta Structure Prediction Server after submission of the entire LL sequence. See Example 22.

### DETAILED DESCRIPTION

The issued patents, applications, and other publications that are cited herein are hereby incorporated by reference to the same extent as if each was specifically and individually indicated to be incorporated by reference.

All patent applications, published patent applications, issued and granted patents, texts, and literature references cited in this specification are hereby incorporated herein by reference in their entirety to more fully describe the state of the art to which the present disclosed subject matter pertains.

As various changes can be made in the methods and compositions described herein without departing from the scope and spirit of the disclosed subject matter as described, it is intended that all subject matter contained in this application and claims, shown in the accompanying drawings, or defined in the appended claims be interpreted as illustrative, and not in a limiting sense.

### DEFINITIONS

The singular forms “a,” “an,” and “the” include plural reference unless the context clearly dictates otherwise.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

As used herein the term “Ll RNA” includes any RNA, for example but not limited to unprocessed RNA, any mRNA of any splice variant (isoform), which encodes a full length Ll protein (LL), any fragment, any protein isoform, or any Ll protein variant thereof. The term Ll RNA also includes an antisense RNA to any Ll mRNA, including but not limited to an antisense RNA to a full length mRNA, any portion of the full length mRNA, or any splice variant.

As used herein the terms “LL” and “Ll” which are used interchangeably, include a full length LL protein, any LL protein fragment, LL isoform, or LL protein variant thereof.

As used herein the term “C1ORF32 RNA” includes but is not limited to unprocessed RNA, any mRNA of any splice variant (isoform), which encodes a full length C1ORF32 protein, any fragment, any protein isoform, or any C1ORF32 protein variant thereof. The term C1ORF32 RNA also includes an antisense RNA to any C1ORF32 mRNA, including but not limited to an antisense RNA to a full length mRNA, any portion of the full length mRNA, or any splice variant.

As used herein the terms “C1ORF32” and “C1Orf32”, which are used interchangabley, include a full length C1ORF32 protein, any C1ORF32 protein fragment, C1ORF32 isoform, or C1ORF32 protein variant thereof.

As used herein the term “variant” covers nucleotide or amino acid sequence variants which have about 95%, about 90%, about 85%, about 80%, about 85%, about 80%, about 75%, about 70%, or about 65% nucleotide identity, or about 95%, about 90%, about 85%, about 80%, about 85%, about 80%, about 75%, or about 70% amino acid identity, including but not limited to variants comprising conservative, or non-conservative substitutions, deletions, insertions, duplications, or any other modification. The term variant as used herein includes functional and non-functional variants, and variants with reduced or altered activity.

As used herein, the term “agent” include, but are not limited to, biological or chemical agents, such as peptides, peptidomimetics, amino acids, amino acid analogs, polynucleotides, polynucleotide analogs, nucleotides, nucleotide analogs, organic or inorganic compounds (i.e. including heteroorganic and organometallic compounds), and salts, esters, and other pharmaceutically acceptable forms of such compounds. Salts, esters, and other pharmaceutically acceptable forms of such compounds are also encompassed.

### T2M and Lisch-Like

The identification of susceptibility genes in humans is complicated by the polygenic nature of the phenotype (Cox et al, 1992, Diabetes 41:401-407). This is refected in convergent yet distinct metabolic processes producing identical phenotypes (phenocopies) in a background of gene/gene and gene/environment (e.g., obesity) interactions that characterize the disease. Clear genetic influences on the endophenotypes (intermediate phenotypes) of β cell mass/function and insulin resistance vary among ethnic groups (Pimenta et al, 1995, Jama 273:1855-1861; Gelding et al, 1995, Clin Endocrinol (Oxf) 42:255-264; Knowler et al, 1993, Care 16:216-227; Hanley et al, 2003, Diabetes 52:463-469). Although more than 20 genome scans in ethnic and racial groups have detected numerous diabetes-susceptibility intervals of modest statistical significance, many of these results have not been replicated in other populations. Despite some successes (e.g. PPARG, CAPN10, TCF7L2), the number of genes conveying diabetes can vary by race/environment (Permutt et al, 2005, J Clin Invest 115:1431-1439).

Like humans, mouse strains differ widely in susceptibility to diabetes when made obese. As described herein, the differential diabetes susceptibilities of the B6 and DBA strains segregating for the obesity mutation Lep^{ob} (Clee S M, Attie A D (2007) The genetic landscape of type 2 diabetes in mice. Endocr Rev 28: 48-83) were used to identify a diabetes susceptibility QTL in B6xDBA progeny and then used congenic lines derived from the implicated interval to clone a candidate gene accounting for the QTL. Similar strategies have been used to identify QTLs (and responsible genes) for other complex phenotypes in mice (Flint J, Valdar W, Shifman S, Mott R (2005) Strategies for mapping and cloning quantitative trait genes in rodents. Nat Rev Genet. 6: 271-286) such as type 1 diabetes (Todd J A (1999) From genome to aetiology in a multifactorial disease, type 1 diabetes. Bioessays 21: 164-174), diet-induced obesity (York B, Lei K, West D B (1996) Sensitivity to dietary obesity linked to a locus on chromosome 15 in a CAST/Ei×C57BL/6J F2 intercross. Mamm Genome 7: 677-681), tuberculosis susceptibility (Mitsos L M, Cardon L R, Fortin A, Ryan L, LaCourse R, et al. (2000) Genetic control of susceptibility to infection with Mycobacterium tuberculosis in mice. Genes Immun 1: 467-477), atherosclerosis (Welch C L, Bretschger S, Latib N, Bezouevski M, Guo Y, et al. (2001) Localization of atherosclerosis susceptibility loci to chromosomes 4 and 6 using the Ldlr knockout mouse model. Proc Natl Acad Sci U S A 98: 7946-7951), epilepsy (Legare M E, Bartlett F S, 2nd, Frankel W N (2000) A major effect QTL determined by multiple genes in epileptic EL mice. Genome Res 10: 42-48), schizophrenia (Joober R, Zarate J M, Rouleau G A, Skamene E, Boksa P (2002) Provisional mapping of quantitative trait loci modulating the acoustic startle response and prepulse inhibition of acoustic startle. Neuropsychopharmacology 27: 765-781) and, also, T2DM (Clee S M, Yandell B S, Schueler K M, Rabaglia M E, Richards O C, et al. (2006) Positional cloning of Sorcsl, a type 2 diabetes quantitative trait locus. Nat Genet. 38: 688-693; Goodarzi M O, Lehman D M, Taylor K D, Guo X, Cui J, et al. (2007) SORCS1: a novel human type 2 diabetes susceptibility gene suggested by the mouse. Diabetes 56: 1922-1929; Freeman H, Shimomura K, Horner E, Cox R D, Ashcroft F M (2006) Nicotinamide nucleotide transhydrogenase: a key role in insulin secretion. Cell Metab 3: 35-45; Freeman H C, Hugill A, Dear N T, Ashcroft F M, Cox R D (2006) Deletion of nicotinamide nucleotide transhydrogenase: a new quantitive trait locus accounting for glucose intolerance in C57BL/6J mice. Diabetes 55: 2153-2156).

In one aspect of this invention, these differential diabetes susceptibilities were exploited to map diabetes-susceptibility regions of the mouse genome using genetic crosses between a diabetes-susceptible (DBA) and a resistant strain (B6). In another aspect, the invention provides the identification of the genes responsible for the diabetes-related phenotypes of B6.DBA Lep^{ob/ob} F2 and F3 mice segregating for a QTL in the distal portion of Chr1. As described in the Examples of section herein, molecular genetic methods were used to identify Lisch-like (Ll), as a gene that accounts for diabetes susceptibility conveyed by the DBA interval in the intercross, and in B6.DBA N12-15 congenic progeny. The gene affects the early development and replication of beta cells and a reduced beta cell mass resulting in a predisposition to diabetes. In certain aspects, the invention provides methods to increase Ll activity to reverse these effects. The gene encodes multiple, tissue-specific transcripts in brain, liver and islets. The functional consequences of the hypomorphic DBA allele (diabetes-prone) in Lep^{ob/ob} mice appear to be late embryonic to early postnatal reductions in β-cell mass due to diminished rates of β-cell replication, some “catch-up” of β-cell mass by 2-3 months, followed by mild glucose intolerance at >6 months of age. These phenotypes are recapitulated in mice with an ENU-induced null allele of Ll.

Ll is a gene that produces multiple tissue-specific transcripts and is most highly expressed in brain, liver, and islets. Encoding a 10-exon 646 amino acid protein with significant homology to Lsr on Chr1qB1 and to Lldr1 on Chr16qB3, Ll spans 62.7 kb on Chr1qH2. The largest LL isoform is a predicted single-pass trans-membrane molecule with a signal sequence, an immunoglobulin-like extra-cellular domain and a serine/threonine rich intra-cellular domain that also contains a 14-3-3 binding domain and a terminal PDZ-binding motif (FIG. 32.).

The amounts of L1 transcripts are reduced 2-10 fold in these organs in mice segregating for DBA (v. B6) congenic intervals containing Ll. A recombination event between exons 8 and 9 of the 10 exon Ll gene, has allowed characterization of the phenotypes of lines segregating for the complete DBA allele of Ll versus B6.DBA lines containing only the distal portions (exons 9, 10 and 3′UTR) of the gene. The latter lines display phenotypes and organ-specific rates of Ll expression comparable to the line containing the entire DBA allele of L, implicating 3′ UTR-mediated effects on message stability as a potential primary mechanism for the DBA allele's affects on diabetes-related phenotypes. There is also a 2845 bp in-frame antisense transcript running centromeric from exon 9 of Ll. In one embodiment, this antisense sequence can be used to squelch message in DBA v. B6 alleles of Ll. In another embodiment, this antisense sequence can be used to protect message in DBA v. B6 alleles of Ll. (Lapidot and Pilpel 2006, EMBO Rep 7:1216-1222; Costa 2005, Gene 357:83-94.).

The amino acid sequence of Ll is highly homologous to the so-called “Lipolysis-stimulated receptor” (Lsr) (Yen et al, 1999, J Biol Chem 274:13390-13398). “Knockdown” of embryonic Zebrafish (D. rerio) paralogs of Ll and Lsr results in disruption of endodermal organization and the integrity of the single large pancreatic islet in these animals. The physiological role(s) of Lsr—an apparent plasma membrane receptor—are unclear. The molecule is expressed in different tissues, including brain and liver. Homozygosity for a null allele of Lsr is embryonic lethal at E12.5-15.5 and associated with hepatic hypoplasticity, whereas the heterozygotes appear normal (Mesli et al, 2004, Eur J Biochem 271:3103-3114). LSR binds to apoliproteins B/E in the presence of free fatty acids, and can assist in the clearance of triglyceride-rich lipoproteins (Yen et al, 1999, J Biol Chem 274:13390-13398; Yen et al, 1994, Biochemistry 33:1172-1180). While LSR and LL are structurally homologous and may have overlapping functions, they are distinct enough so that they may also have non-overlapping functions and that reagents designed to be specific to either protein would not be predicted to cross-react.

LSR protein domains are described in U.S. Pat. No. 7,291,709. The table below and description that follows show the sequence of several LSR domains compared to the corresponding aligned sequence in mouse LL. Start and end amino acid residues refer to SEQ ID NO:24 (mouse LSR) and SEQ ID NO:21 (mouse LL) (see FIG. 32).

| Domain in LSR | Amino acid sequence (LSR and LL) | |
|---|---|---|
| Potential fatty | LSR 23-41: | CLFLIIYCPDRASAIQVTV |
| acid binding | | ((SEQ ID NO: 113) |
| site | LL 7-25: | GWTAVFWLTAMVEGLQVTV |
| | | (SEQ ID NO: 114) |
| Transmembrane | LSR 204-213: | LEDWLFVVVV |
| domain | | (SEQ ID NO: 115) |
| | LL 184-193: | MPEWVFVGLV |
| | | (SEQ ID NO: 116) |
| Potential | LSR 214-249: | CLASLLFFLLLGICWCQCCPHTCCCYVRCPCCPDKC |
| cytokine | | (SEQ ID NO: 117) |
| receptor | LL 194-229: | ILGIFLFFVLVGICWCQCCPHSCCCYVRCPCCPDSC |
| site | | (SEQ ID NO: 118) |
| Potential | LSR 544-558: | ERR-------------------------------- |
| lipoprotein | | RVYREEEEEEEE |
| ligand | | (SEQ ID NO: 119) |
| binding | LL 540-586: | (SEQ ID NO: 120) |
| site | | ESSSRGGSLETPSKLGAQLGPRSASYYAWSPPTTYKAGASEGEDEDD |

There are other structural similarities between LSR and LL. For example, the NPGY sequence in LSR (104-107), referred to as a putative clathrin-binding sequence on LSR, is a phosphotyrosine binding ligand of the class NPXY, that is contained in β-amyloid precursor proteins. The sequence NPDY is found between residues 370-373 in LL. Additionally, the RSRS motif is within a proline-rich domain in LSR (470-473); a similar motif RSRASY (561-565 of LL) was identified by Motif Scan as a putative 14-3-3 Mode 1 binding motif. The LL sequence RAGSRF (451-456 of LL) was identified by the ELM Server as a potential 14-3-3 ligand.

LL may participate in a variety of processes. Like LSR, LL may be involved in the transport of fatty acids and/or cholesterol. LL is expressed in liver, islets and the hypothalamus, and, based upon developmental and physiological studies, has effects on beta cell development and, possibly, function. These effects could be conveyed directly on the beta cell, or could be secondary to changes in the liver and/or hypothalamus. The high specific expression of LL transcripts in the hypothalamus and the relatively high specific concentration of LL polypeptide in the hypothalamus are consistent with a role for LL in control of hepatic glucose homeostasis and/or beta cell function by autonomic efferents from the hypothalamus. These have not yet been directly tested.

Non-limiting examples include for islet cell ontogenesis, cellular lipid homeostasis, hepatic and muscle insulin responsiveness and islet 3 cell function and survival. Identification of such functions can be important for understanding aspects of the pathogenesis of T2DM. In certain aspects, the invention provides methods to characterize the molecular physiology of LL in mice.

The human ortholog of L1, C1ORF32, which is 90% identical to L1 at the amino acid level, maps to a region of Chr1q23 that has been repeatedly implicated in T2DM in seven ethnically diverse populations including Caucasians (Northern Europeans in Utah) (Elbein et al, 1999, Diabetes 48:1175-1182), Amish Family Study (Hsueh et al, 2003, Diabetes 52:550-557, St. Jean 2000, American Journal of Human Genetics 67), United Kingdom Warren 2 study (Wiltshire et al, 2001 Am J Hum Genet. 69:553-569), French families (Vionnet et al, 2000, Am J Hum Genet. 67:1470-1480), and Framingham Offspring study (Meigs et al, 2002, Diabetes 51:833-840), Pima Indians (Hanson et al, 1998, J Hum Genet. 63:1130-1138), and Chinese (Xiang, et al, 2004, Diabetes 53:228-234) with LOD scores as high as 4.3. There is evidence of association of alleles of C1Orf32 with T2D in several of these populations. The mouse congenic interval examined as described herein is in the middle of, and physically ˜10× smaller than, the 30 Mb human interval. Recent analysis of the broad interval ascertained in Utah identified two peaks, one of which, at D1S2762 (@163.6 Mb), is just 12 kb telomeric to the 5′ end of the C1ORF32 gene (Das S K, Elbein S C (2007) The search for type 2 diabetes susceptibility Loci: the chromosome 1q story. Curr Diab Rep 7: 154-164). The genes, and gene order, are generally conserved between mouse and human in the region syntenic to the congenic interval. The metabolic phenotypes documented in human subjects with T2DM linked to 1q23 resemble diabetic phenotypes observed in congenic mice segregating for the DBA interval in B6.DBA congenics examined here (McCarthy M, Shuldiner, A. R., Bogardus, C., Hanson, R. L., Elbein, S., (2004) Positional Cloning of a Type 2 Diabetes Susceptibility Gene on Chromosome 1q: A collaborative effort by the Chromosome 1q Diabetes Positional Cloning Consortium. 1-39), suggesting that the diabetes-susceptibility gene in congenic mice and human subjects may be the same gene, or among the genes, acting in the same genetic pathway(s). The syntenic interval in the GK rat also correlates with diabetes-susceptibility (Chung W K, Zheng M, Chua M, Kershaw E, Power-Kehoe L, et al. (1997) Genetic modifiers of Leprfa associated with variability in insulin production and susceptibility to NIDDM. Genomics 41: 332-344).

Data described herein identify two non-synonymous amino acid variants in LL of DD mice: T587A and A647V (both found in exon 9 in Ll). These positions correspond to Glycine-572 and Alanine-625 in human C1orf32, respectively. In certain aspects, the invention provides methods to determine whether these amino acid variants: (a) decrease protein stability and (b) change protein function in any way. To determine the effect of these amino acids changes, these mutation can be engineered in expression vectors for mammalian transfections, and functional characterization experiments as described herein can be carried out for the mutant Ll variants. The T587A mutation abolishes a potential phosphorylation site. Methods for inventigating the role of phosphorylation are well known to those skilled in the art.

Insight into the function(s) of the mouse Lisch-like protein may be gained from similarities in structure, expression, and cellular location with the human paralog, C1ORF32, and with genes encoding related trans-membrane receptors, Ildr1 (Hauge H, Patzke S, Delabie J, Aasheim H C (2004) Characterization of a novel immunoglobulin-like domain containing receptor. Biochem Biophys Res Commun 323: 970-978) and Lsr (Yen F T, Masson M, Clossais-Besnard N, Andre P, Grosset J M, et al. (1999) Molecular cloning of a lipolysis-stimulated remnant receptor expressed in the liver. J Biol Chem 274: 13390-13398). Splicing patterns of these genes generate isoforms, similar to those of Ll. Each gene's largest isoform includes an extra-cellular Ig-like domain, a single TMD, and a similar set of ICDs in related order. In one isoform of each protein, the TMD and cysteine-rich domains are absent. An evolutionary, regulatory relationship is suggested by the observation that the D-paralog and lldr1 are adjacent in the zebra fish genome (Zv6 assembly, UCSC Genome Browser). All three genes are abundantly expressed in the brain, liver and pancreas (and islets, where studied), and all are predicted to have 14-3-3 interacting domains (thus far experimentally verified for the human LSR) (Garcia-Ocana A, Takane K K, Syed M A, Philbrick W M, Vasavada R C, et al. (2000) Hepatocyte growth factor overexpression in the islet of transgenic mice increases beta cell proliferation, enhances islet mass, and induces mild hypoglycemia. J Biol Chem 275: 1226-1232). Although 14-3-3 interacting domains may be present on as many as 0.6% of human proteins, their occurrence on all of these Lisch-related proteins is notable, since among known 14-3-3-interacting proteins is phoshodiesterase-3B, which is implicated in diabetes and pancreatic β-cell physiology (Onuma H, Osawa H, Yamada K, Ogura T, Tanabe F, et al. (2002) Identification of the insulin-regulated interaction of phosphodiesterase 3B with 14-3-3 beta protein. Diabetes 51: 3362-3367; Xiang K, Wang Y, Zheng T, Jia W, Li J, et al. (2004) Genome-wide search for type 2 diabetes/impaired glucose homeostasis susceptibility genes in the Chinese: significant linkage to chromosome 6q21-q23 and chromosome 1q21-q24. Diabetes 53: 228-234; Pozuelo Rubio M, Geraghty K M, Wong B H, Wood N T, Campbell D G, et al. (2004) 14-3-3-affinity purification of over 200 human phosphoproteins reveals new links to regulation of cellular metabolism, proliferation and trafficking. Biochem J 379: 395-408), and others, such as the Cdc25 family members, important in regulating cell proliferation and survival (Meek S E, Lane W S, Piwnica-Worms H (2004) Comprehensive proteomic analysis of interphase and mitotic 14-3-3-binding proteins. J Biol Chem 279: 32046-32054; Hermeking H, Benzinger A (2006) 14-3-3 proteins in cell cycle regulation. Semin Cancer Biol 16: 183-192).

Screening Methods to Identify Agents which Modulate Expression of Ll or LL, C1Orf32 or C1ORF32.

In certain aspects the invention provides methods to identify agents which modulate expression of Ll or LL, C1Orf32 or C1ORF32, the method comprising determining expression in the absence of a candidate agent, contacting a cell with a candidate agent, determining expression in the presence of the candidate agent, and comparing the expression determined in the presence and the absence of the candidate agent. In certain aspects, the invention provides a method for identifying an agent which modulates expression of an Ll RNA comprising: (a) determining expression of an Ll RNA in a cell, (b) contacting the cell with an agent; and (c) determining expression of the Ll RNA in the presence of the agent, wherein a change in the expression of the Ll RNA in the presence of the agent, compared to the expression of the Ll RNA in the absence of the agent, is indicative of an agent which modulates the expression of the Ll RNA. In certain embodiments, the method comprises: (a) contacting a cell with an agent; (b) determining expression of the Ll RNA in the presence and the absence of the agent; and (c) comparing expression of the Ll RNA in the presence and the absence of the agent, wherein a change in the expression of the Ll RNA in the presence of the agent is indicative of an agent which modulates the level of expression of the RNA. In certain embodiments, the method measures expression of C1ORF32 RNA. In certain embodiments, the assay is carried out in a cell which is comprised in an animal. In a non-limiting example the animal is a mouse. In other embodiments, the assay is carried out in a cell which is comprised in a tissue culture and/or a cell line derived from tissues of a mouse, or a human subject. In certain aspects, the cell is comprised in a diabetes-relevant tissue. In other aspects, the cell is derived from any tissue or source which allows to determine modulation of expression of Ll or LL, C1Orf32 or C1ORF32. In non-limiting examples, the cell is a pancreatic cell, an isulin producing beta cell, or a hepatocyte, a hypothalamic or other brain cell, or any combination thereof.

In certain embodiments, the method is carried out in a cell which expresses endogenous Ll or LL, C1Orf32 or C1ORF32. In other embodiments, the method is carried out in a cell comprising an expression vector or a construct comprising nucleic acid which encodes Ll or LL, C1Orf32 or C1ORF32. The nucleic acid encoding Ll or LL, C1Orf32 or C1ORF32 can be a nucleic acid, for example encoding any splice variant, isoform, or a fragment, a genomic DNA, or any portion of the genomic DNA. In certain aspects, the expression vector is introduced by transfection into an autologous cell type. In other aspects, the expression vector is introduced by transfection into a non-autologous cell type. Methods to create expression vectors and constructs are well known in the art. Non-limiting examples of various expression vectors, cells, tissues, and cell lines are described herein. In certain embodiments, the cell can comprise any other suitable nucleic acid or an expression vectors comprising a nucleic acid which encodes such suitable nucleic acid. In non-limiting examples, such suitable nucleic acid can be a nucleic acid which encodes a L-l or LL-, C1Orf32- or C1ORF32-interacting, and/or regulatory partner.

In certain embodiments, determining comprises quantitative determination of the level of expression. In other embodiments, determining comprises quantitative determination of the stability or turnover of Ll or LL, C1Orf32 or C1ORF32. Methods for determining expression of a RNA or a protein, including quantitative and/or qualitative determinations, are described herein and well known in the art. In certain embodiments, the methods of the invention determine an increase in the expression. In other embodiments, the methods of the invention determine a decrease in the expression. The expression of a gene can be readily detected, e.g., by quantifying the protein and/or RNA encoded by the gene. Many methods standard in the art can be thus employed, including, but not limited to, immunoassays to detect and/or visualize protein expression, nonlimiting examples include western blot, immunoprecipitation followed by sodium dodecyl sulfate polyacrylamide gel electrophoresis (SDS-PAGE), immunocytochemistry, etc., and/or hybridization assays to detect gene expression by detecting and/or visualizing respectively RNA, including but not limited to mRNA encoding a gene (PCR, northern assays, dot blots, in situ hybridization, etc.). Such assays are routine and well known in the art (see, e.g., Ausubel et al, eds, 1994, Current Protocols in Molecular Biology, Vol. 1, John Wiley & Sons, Inc., New York, which is incorporated by reference herein in its entirety). Non-limiting exemplary assays are described herein.

In certain embodiments, the methods of the invention can determine changes in the expression, associated with changes in the localization, processing, trafficking, posttranslational modification, or any other cellular modification of Ll or LL, C1Orf32 or C1ORF32. Determining expression of Ll or LL, C1Orf32 or C1ORF32 can be carried out by any suitable method as described herein, or known in the art.

In certain embodiments, the step of contacting a cell with an agent is under conditions suitable for gene or protein expression. In certain embodiments, contacting step is in an aqueous solution comprising a buffer and a combination of salts. In certain embodiments, the aqueous solution approximates or mimics physiologic conditions.

In certain embodiments, once an agent has been identified to modulate expression, and optionally, the structure of the compound has been identified, the agent can be further tested for biological activity in additional assays and/or animal models for type 2 diabetes. In addition, a lead compound can be used to design analogs, and other structurally similar compounds.

In certain embodiments, the invention provides screening of libraries of agents, including combinatorial libraries, to identify an agent which modulate the expression. Libraries screened using the methods of the present invention can comprise a variety of types of compounds. Non-limiting examples of libraries that can be screened in accordance with the methods of the invention include, but are not limited to, peptoids; random biooligomers; diversomers such as hydantoins, benzodiazepines and dipeptides; vinylogous polypeptides; nonpeptidal peptidomimetics; oligocarbamates; peptidyl phosphonates; peptide nucleic acid libraries; antibody libraries; carbohydrate libraries; and small molecule libraries, for example but not limited to small organic molecules. In certain embodiments, the compounds in the libraries screened are nucleic acid or peptide molecules. In a non-limiting example, peptide molecules can exist in a phage display library. In other embodiments, the types of compounds include, but are not limited to, peptide analogs including peptides comprising non-naturally occurring amino acids, e.g., D-amino acids, phosphorous analogs of amino acids, such as .alpha.-amino phosphoric acids and .alpha.-amino phosphoric acids, or amino acids having non-peptide linkages, nucleic acid analogs such as phosphorothioates and PNAs, hormones, antigens, synthetic or naturally occurring drugs, opiates, dopamine, serotonin, catecholamines, thrombin, acetylcholine, prostaglandins, organic molecules, pheromones, adenosine, sucrose, glucose, lactose and galactose. Libraries of polypeptides or proteins can also be used in the assays of the invention.

In certain embodiments, the combinatorial libraries are small organic molecule libraries including, but not limited to, benzodiazepines, isoprenoids, beta carbalines, thiazolidinones, metathiazanones, pyrrolidines, morpholino compounds, small inhibitory RNAs short hairpin RNAs, and benzodiazepines. In another embodiment, the combinatorial libraries comprise peptoids; random bio-oligomers; benzodiazepines; diversomers such as hydantoins, benzodiazepines and dipeptides, vinylogous polypeptides; nonpeptidal peptidomirnetics; oligocarbamates; peptidyl phosphonates; peptide nucleic acid libraries; antibody libraries; or carbohydrate libraries. Combinatorial libraries are themselves commercially available from differente sources.

In a certain embodiments, the library is preselected so that the compounds of the library are more amenable for cellular uptake. For example, compounds are selected based on specific parameters such as, but not limited to, size, lipophilicity, hydrophilicity, and hydrogen bonding, which enhance the ability of compounds to enter into the cells. In other embodiments, the compounds are analyzed by three-dimensional or four-dimensional computer computation programs.

Methods to synthesize and screen combinatorial libraries are known in the art. In one embodiment, the combinatorial compound library can be synthesized in solution. In other embodiments the combinatorial libraries can be synthesized on solid support. For non-limitng examples of such methods see U.S. Pat. No. 5,866,341 to Spinella et al., U.S. Pat. No. 6,190,619 to Kilcoin et al., U.S. Pat. No. 6,194,612 to Boger et al.; Egner et al., 1995, J. Org. Chem. 60:2652; Anderson et al., 1995, J. Org. Chem. 60:2650; Fitch et al., 1994, J. Org. Chem. 59:7955; Look et al., 1994, J. Org. Chem. 49:7588; Metzger et al., 1993, Angew. Chem., Int. Ed. Engl. 32:894; Youngquist et al., 1994, Rapid Commun Mass Spect. 8:77; Chu et al., 1995, J. Am. Chem. Soc. 117:5419; Brummel et al., 1994, Science 264:399; and Stevanovic et al., 1993, Bioorg. Med. Chem. Lett. 3:431; Lam et al., 1997, Chem. Rev. 97:41-448; Ohlmeyer et al., 1993, Proc. Natl. Acad. Sci. USA 90:10922-10926 a Nefzi et al., 1997, Chem. Rev. 97:449-472; and references cited therein, all of which are hereby incorporated by reference in their entirety.

Agents that modulate expression, as identified by the methods described herein can be selected and characterized by methods known in the art. For example, if the library comprises arrays or microarrays of agents, wherein each agent has an address or identifier, the agent can be deconvoluted, e.g., by cross-referencing the positive sample to original compound list that was applied to the individual test assays. If the library is a peptide or nucleic acid library, the sequence of the compound can be determined by direct sequencing of the peptide or nucleic acid. Such methods are well known to one of skill in the art. A number of physico-chemical techniques can also be used for the de novo characterization of compounds that modulate expression as determined by the methods of the present invention. Examples of such techniques include, but are not limited to, mass spectrometry, NMR spectroscopy, X-ray crystallography and vibrational spectroscopy.

In certain aspects, the invention provides methods for identifying metabolic or environmental agents and/or stimuli (e.g., exposure to different concentrations of metabolites, nutrients, or the like, or of CO₂ and/or O₂, stress and different pHs,) that modulate untranslated region-dependent expression of a target gene utilizing the cell-based reporter gene assays described herein. In another embodiment, the environmental stimuli does not include a compound. In non-limiting examples, the metabolic agent is insulin, cAMP, glucose, free fatty acids, cholesterol or a combination thereof.

### Antibodies to Lisch-Like

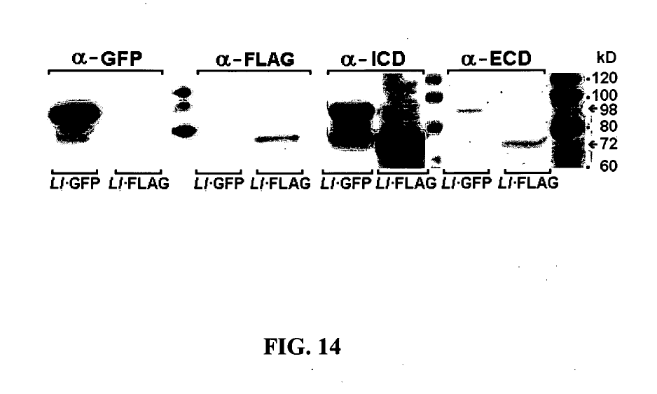

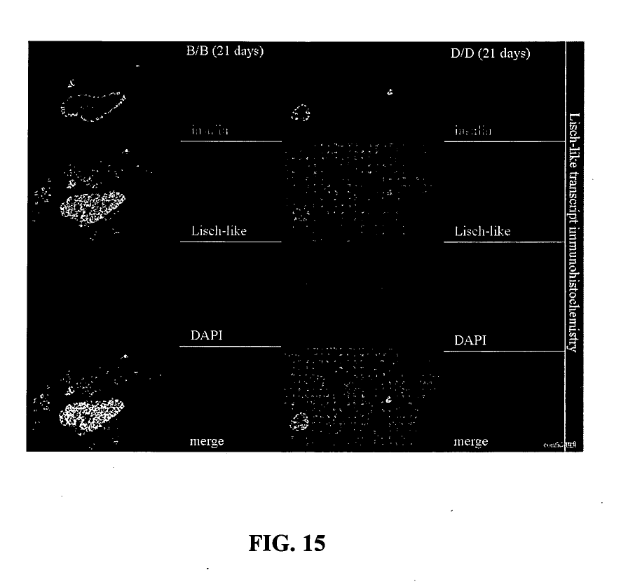

Using standard immunization protocols, polyclonal rabbit and guinea pig antibodies (Covance Research Products) were generated against the predicted extracellular domain (ECD) of LL spanning residues 22-186, and intracellular domain (ICD) spanning residues 298-401. α-ICD and α-ECD rabbit antibodies detected the appropriate fusion proteins, showing only minor cross-reactivity (FIG. 14). Another set of antibodies to smaller ECD and ICD epitopes (FIG. 23A and FIG. 23B) were generated to detect the localized expression pattern of Ll in pancreatic β cells in non-diabetic mice, as well as an undetectable LL protein level in diabetic D/D mice—that show reduced β cell replication and reduced islet mass—indicates that Ll can play a critical role in β cell development (FIG. 15).

In one aspect, the invention provides antibody that binds to the peptide which is from the extracellular domain (ECD) of LL spanning residues 22-186 (SEQ ID NO: 7), or a (poly)peptide which comprises the peptide of SEQ ID NO: 70. In another aspect of the invention provides antibody that binds to the peptide which is from the intracellular domain (ICD) of LL spanning residues 298-401 (SEQ ID NO: 6), or a (poly)peptide which comprises the peptide of SEQ ID NO: 71. In another aspect of the invention provides antibody that binds to the peptide which is from the extracellular domain (ECD) of C1ORFE32 spanning residues shown in SEQ ID NO: 9, or a (poly)peptide which comprises the peptide of SEQ ID NO: 72. In another aspect of the invention provides antibody that binds to the peptide which is from the intracellular domain (ICD) of C1ORF32 spanning residues shown in SEQ ID NO: 8, or a (poly)peptide which comprises the peptide of SEQ ID NO: 73.

In another aspect, the antibodies of the invention are isolated. The antibodies of the invention can be monoclonal or polyclonal. Methods for making polyclonal and monoclonal antibodies are well known in the art. Antibodies of the invention can be produced by methods known in the art in any suitable animal host such as but not limited to rabbit, goat, mouse, sheep. In one embodiment, the antibodies can be chimeric, i.e. a combination of sequences of more than one species. In another embodiment, the antibodies can be fully-human or humanized Abs. Humanized antibodies contain complementarity determining regions that are derived from non-human species immunoglobulin, while the rest of the antibody molecule is derived from human immunoglobulin. Fully-human or humanized antibodies avoid certain problems of antibodies that possess non-human regions which can trigger host immune response leading to rapid antibody clearance. In still another embodiment, antibodies of the invention can be produced by immunizing a non-human animal with an immunogenic composition comprising a polypeptide of the invention in the monomeric form. In other embodiments, dimeric or multimeric forms can be used. The immunogenic composition can also comprise other components that can increase the antigenicity of the inventive peptide. In one embodiment the non-human animal is a transgenic mouse model, for e.g., the HuMAb-Mouse™ or the Xenomouse®, which can produce human antibodies. Neutralizing antibodies against peptides of interest and the cells producing such antibodies can be identified and isolated by methods know in the art.

Making of monoclonal antibodies is well known in the art. In one embodiment, the monoclonal antibodies of the invention are made by harvesting spleen tissue from a rabbit which produces a polyclonal antibody. Harvested cells are fused with the immortalized myeloma cell line partner. After an initial period of growth of the fused cells, single antibody producing clones are isolated by cell purification, grown and analyzed separately using a binding assay (e.g., ELISA, or Western). Hybridomas can be selected based on the ability of their secreted antibody to bind to a peptide interest, including a polypeptide comprising SEQ ID NOs: 6-9 or 69-73. Variable regions can be cloned from the hybridomas by PCR and the sequence of the epitope binding region can be determined by sequencing methods known in the art.

The invention provides antibodies and antibody fragments of various isotypes. The recombined immunoglobulin (Ig) genes, for example the variable region genes, can be isolated from the deposited hybridomas, by methods known in the art, and cloned into an Ig recombination vector that codes for human Ig constant region genes of both heavy and light chains. The antibodies can be generated of any isotype such as IgG1, IgG2, IgG3, IgG4, IgD, IgE, IgM, IgA1, IgA2, or sIgA isotype. The invention provides isotypes found in non-human species as well such as but not limited to IgY in birds and sharks. Vectors encoding the constant regions of various isotypes are known and previously described. (See, for example, Preston et al. Production and characterization of a set of mouse-human chimeric immunoglobulin G (IgG) subclass and IgA monoclonal antibodies with identical variable regions specific for P. aeruginosa serogroup 06 lipopolysaccharide. Infect Immun 1998 September; 66(9):4137-42; Coloma et al. Novel vectors for the expression of antibody molecules using variable regions generated by polymerase chain reaction. J Immunol Methods. 1992 Jul. 31; 152(1):89-104; Guttieri et al. Cassette vectors for conversion of Fab fragments into full-length human IgG1 monoclonal antibodies by expression in stably transformed insect cells. Hybrid Hybridomics. 2003 June; 22(3):135-45; McLean et al. Human and murine immunoglobulin expression vector cassettes. Mol. Immunol. 2000 October; 37(14):837-45; Walls et al. Vectors for the expression of PCR-amplified immunoglobulin variable domains with human constant regions. Nucleic Acids Res. 1993 Jun. 25; 21(12):2921-9; Norderhaug et al. Versatile vectors for transient and stable expression of recombinant antibody molecules in mammalian cells. J Immunol Methods. 1997 Can 12; 204(1):77-87.)

The antibodies of the invention bind to a polypeptide having the sequence of any of SEQ ID NOs: 6-9 or 69-72, comprised in a longer polypeptide, in a specific manner. In one embodiment, the antibodies, or antibody fragments of the invention bind specifically to a peptide of SEQ ID NO: 6, 7, 8, or 9. In one embodiment, the antibodies, or antibody fragments of the invention bind specifically to a peptide of SEQ ID NO: 69, 70, 71, 72 or 73. For example, antibodies that bind specifically to a peptide that comprises a sequences shown in any of SEQ ID NOs: 6-9 or 69-73 will not bind to polypeptides which do not comprise the amino acid sequence of any of SEQ ID NO: 6-9 or 69-73 to the same extent and with the same affinity as they bind to a peptide that comprises a sequences shown in any of SEQ ID NOs: 6-9 or 69-73. In another embodiment, the antibody, or/and antibody fragments, of the invention can bind specifically to polypeptides which comprise any of SEQ ID NOs: 21-57, but this binding can occur with lesser affinity compared to the binding to a polypeptide that comprises a sequences shown in any of SEQ ID NOs: 6-9 or 69-73. Lesser affinity can include at least 10% less, 20% less, 30% less, 40% less, 50% less, 60% less, 70% less, 80% less, 90% less, or 95% less.

The present invention provides specific monoclonal antibodies, including but not limited to rabbit, mouse and human, which recognize a peptide of SEQ ID NO: 6, 7, 8 or 9, including a polypeptide comprising SEQ ID NO: 69, 70, 71, or 72. When used in vivo in humans, human monoclonal antibodies are far less likely to be immunogenic (as compared to antibodies from another species).

Variable region nucleic acids for the heavy and light chains of the antibodies can be cloned into an human Ig expression vector that contain any suitable constant region, for example (i.e., TCAE6) that contains the IgG1 (gamma 1) constant region coding sequences for the heavy chain and the lambda constant region for the light chains. (See, for example, Preston et al. Production and characterization of a set of mouse-human chimeric immunoglobulin G (IgG) subclass and IgA monoclonal antibodies with identical variable regions specific for P. aeruginosa serogroup O6 lipopolysaccharide. Infect Immun 1998 September; 66(9):4137-42.) The variable regions can be placed in any vector that encodes constant region coding sequences. For example, human Ig heavy-chain constant-region expression vectors containing genomic clones of the human IgG2, IgG3, IgG4 and IgA heavy-chain constant-region genes and lacking variable-region genes have been described in Coloma, et al. 1992 J. Immunol. Methods 152:89-104.) These expression vectors can then be transfected into cells (e.g., CHO DG44 cells), the cells are grown in vitro, and IgG1 are subsequently harvested from the supernatant. Resultant antibodies can be generated to posses human variable regions and human IgG1 and lambda constant regions. In other embodiments, the Fc portions of the antibodies of the invention can be replaced so as to produce IgM.

In other embodiments, the antibody of the invention also includes an antibody fragment. It is well-known in the art, only a portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford; and Pier G B, Lyczak J B, Wetzler L M, (eds) Immunology, Infection and Immunity (2004) 1.sup.st Ed. American Society for Microbiology Press, Washington D.C.). The pFc′ and Fc regions of the antibody, for example, are effectors of the complement cascade and can mediate binding to Fc receptors on phagocytic cells, but are not involved in antigen binding. An antibody from which the pFc′ region has been enzymatically cleaved, or which has been produced without the pFc′ region, e.g. an F(ab′)₂ fragment, retains both of the antigen binding sites of an intact antibody. An isolated F(ab′)₂ fragment is referred to as a bivalent monoclonal fragment because of its two antigen binding sites. Similarly, an antibody from which the Fc region has been enzymatically cleaved, or which has been produced without the Fc region, e.g. an Fab fragment, retains one of the antigen binding sites of an intact antibody molecule. Proceeding further, Fab fragments consist of a covalently bound antibody light chain and a portion of the antibody heavy chain denoted Fd (heavy chain variable region). The Fd fragments are the major determinant of antibody specificity (a single Fd fragment can be associated with up to ten different light chains without altering antibody specificity) and Fd fragments retain epitope-binding ability in isolation. An antibody fragment is a polypeptide which can be targeted to the nucleus. Methods to modify polypeptides for targeting to the nucleus are known in the art.

Additional methods of producing and using antibodies and antibody fragments comprising Fab, Fc, pFc′, F(ab′)₂ and Fv regions are well known in the art [Klein, Immunology (John Wiley, New York, N.Y., 1982); Clark, W. R. (1986) The Experimental Foundations of Modern Immunology (Wiley & Sons, Inc., New York); Roitt, I. (1991) Essential Immunology, 7th Ed., (Blackwell Scientific Publications, Oxford); and Pier G B, Lyczak J B, Wetzler L M, (eds). Immunology, Infection and Immunity (2004) 1.sup.st Ed. American Society for Microbiology Press, Washington D.C.].

Usually the CDR regions in humanized antibodies are substantially identical, and more usually, identical to the corresponding CDR regions of the donor antibody. However, in certain embodiments, it can be desirable to modify one or more CDR regions to modify the antigen binding specificity of the antibody and/or reduce the immunogenicity of the antibody. One or more residues of a CDR can be altered to modify binding to achieve a more favored on-rate of binding, a more favored off-rate of binding, or both, such that an idealized binding constant is achieved. Using this strategy, an antibody having high or ultra high binding affinity of can be achieved. Briefly, the donor CDR sequence is referred to as a base sequence from which one or more residues are then altered. Affinity maturation techniques can be used to alter the CDR region(s) followed by screening of the resultant binding molecules for the desired change in binding. The method can also be used to alter the donor CDR to be less immunogenic such that a potential chimeric antibody response is minimized or avoided. Accordingly, as CDR(s) are altered, changes in binding affinity as well as immunogenicity can be monitored and scored such that an antibody optimized for the best combined binding and low immunogenicity are achieved (see, e.g., U.S. Pat. No. 6,656,467 and U.S. Pat. Pub. Nos: US20020164326A1; US20040110226A1; US20060121042A1).

The antibodies of the invention can be used in a variety of applications including but not limited to (a) methods for diagnosing type 2 diabetes in a subject, wherein the antibody is used to determine different expression of C1ORF32 in a blood or other tissue sample from a subject compared to the expression of C1ORF32 in a control sample, (b) methods for screening agents, including but not limited to small molecule drugs, biological agents, in order to identify and monitor agents which can modulate the expression, production, localization, and/or stability of L1 or C1ORF32. Additionally, such antibodies could be used to affect the action or regulate the activity of the native peptide at surface of the cell, or to detect shed molecules in the circulation as a diagnostic.

In one aspect, the antibodies that specifically bind polypeptide of SEQ ID NO: 6-9 or 69-72 or a polypeptide which comprises the corresponding peptide, can be used in a screening method to evaluate agents designed to affect the levels of expression of LL and/or C1ORF32. Because the antibody can be used to quantitate protein levels and expression, protein localization, or protein modification of LL and/or C1ORF32. The effect, including the efficiency and/or potency, of the drug can be addressed by following its effect on the presence, or absence, or change, for example but not limited to change in levels of the LL and/or C1ORF32, which can be detected by the antibody of the invention.

The antibodies of the present invention, including fragments and derivatives thereof, can be labeled. It is, therefore, another aspect of the present invention to provide labeled antibodies that bind specifically to one or more of the polypeptides of the present invention, to one or more of the polypeptides encoded by the isolated nucleic acid molecules of the present invention, or the binding of which can be competitively inhibited by one or more of the polypeptides of the present invention or one or more of the polypeptides encoded by the isolated nucleic acid molecules of the present invention. The choice of label depends, in part, upon the desired use.

For example, when the antibodies of the present invention are used for immunohistochemical staining of tissue samples, the label can usefully be an enzyme that catalyzes production and local deposition of a detectable product. Enzymes useful as conjugates to antibodies to permit antibody detection are well known. Exemplary conjugataes are alkaline phosphatase, p-galactosidase, glucose oxidase, horseradish peroxidase (HRP), and urease. Exemplary substrates for production and deposition of visually detectable products are o-nitrophenyl-beta-D-galactopyranoside (ONPG); o-phenylenediamine dihydrochloride (OPD); p-nitrophenyl phosphate (NPP); p-nitrophenyl-beta-D-galactopryanoside (PNPG); 3′,3′-diaminobenzidine (DAB); 3-amino-9-ethylcarbazole (AEC); 4-chloro-1-naphthol (CN); 5-bromo-4-chloro-3-indolyl-phosphate (BCIP); ABTS®; BluoGal; iodonitrotetrazolium (INT); nitroblue tetrazolium chloride (NBT); phenazine methosulfate (PMS); phenolphthalein monophosphate (PMP); tetramethyl benzidine (TMB); tetranitroblue tetrazolium (TNBT); X-Gal; X-Gluc; and X-Glucoside.

Other substrates can be used to produce luminescent products for local deposition. For example, in the presence of hydrogen peroxide (H.sub.2O.sub.2), horseradish peroxidase (HRP) can catalyze the oxidation of cyclic diacylhydrazides, such as luminol Immediately following the oxidation, the luminol is in an excited state (intermediate reaction product), which decays to the ground state by emitting light. Strong enhancement of the light emission is produced by enhancers, such as phenolic compounds. Advantages include high sensitivity, high resolution, and rapid detection without radioactivity and requiring only small amounts of antibody. See, e.g., Thorpe et al., Methods Enzymol. 133: 331-53 (1986); Kricka et al., J. Immunoassay 17(1): 67-83 (1996); and Lundqvist et al., J. Biolumin. Chemiluimin. 10(6): 353-9 (1995). Kits for such enhanced chemiluminescent detection (ECL) are available commercially. The antibodies can also be labeled using colloidal gold.

As another example, when the antibodies of the present invention are used, e.g., for flow cytometric detection, for scanning laser cytometric detection, or for fluorescent immunoassay, they can usefully be labeled with fluorophores. There are a wide variety of fluorophore labels that can usefully be attached to the antibodies of the present invention. For flow cytometric applications, both for extracellular detection and for intracellular detection, common useful fluorophores can be fluorescein isothiocyanate (FITC), allophycocyanin (APC), R-phycoerythrin (PE), peridinin chlorophyll protein (PerCP), Texas Red, Cy3, CyS, fluorescence resonance energy tandem fluorophores such as PerCP-Cy5.5, PE-CyS, PE-Cy5.5, PE-Cy7, PE-Texas Red, and APC-Cy7.

Other fluorophores include, inter alia, Alexa Fluor® 350, Alexa Fluor® 488, Alexa Fluor® 532, Alexa Fluor® 546, Alexa Fluor® 568, Alexa Fluor® 594, Alexa Fluor® 647 (monoclonal antibody labeling kits available from Molecular Probes, Inc., Eugene, Oreg., USA), BODIPY dyes, such as BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY TR, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethylrhodamine, Texas Red (available from Molecular Probes, Inc., Eugene, Oreg., USA), and Cy2, Cy3, Cy3.5, Cy5, Cy5.5, Cy7, all of which are also useful for fluorescently labeling the antibodies of the present invention. For secondary detection using labeled avidin, streptavidin, captavidin or neutravidin, the antibodies of the present invention can usefully be labeled with biotin.

When the antibodies of the present invention are used, e.g., for western blotting applications, they can usefully be labeled with radioisotopes, such as .sup.33P, .sup.32P, .sup.35S, .sup.3H, and .sup.125I. As another example, when the antibodies of the present invention are used for radioimmunotherapy, the label can usefully be .sup.228Th, .sup.227Ac, .sup.225Ac, .sup.223Ra, .sup.213Bi, .sup.212Pb, .sup.212Bi, .sup.211At, .sup.203Pb, .sup.1940s, .sup.188Re, .sup.186Re, .sup.153Sm, .sup.149Tb, .sup.131I, .sup.125I, .sup.111In, .sup.105Rh, .sup.99 mTc, .sup.97Ru, .sup.90Y, .sup.90Sr, .sup.88Y, .sup.72Se, .sup.67Cu, or .sup.47Sc.

As another example, when the antibodies of the present invention are to be used for in vivo diagnostic use, they can be rendered detectable by conjugation to MRI contrast agents, such as gadolinium diethylenetriaminepentaacetic acid (DTPA), Lauffer et al., Radiology 207(2): 529-38 (1998), or by radioisotopic labeling.

The anti-bodies of the present invention, including fragments and derivatives thereof, can also be conjugated to toxins, in order to target the toxin's ablative action to cells that display and/or express the polypeptides of the present invention. The antibody in such immunotoxins is conjugated to Pseudomonas exotoxin A, diphtheria toxin, shiga toxin A, anthrax toxin lethal factor, or ricin. See Hall (ed), Immunotoxin Methods and Protocols (Methods in Molecular Biology, vol. 166), Humana Press (2000); and Frankel et al. (eds.), Clinical Applications of Immunotoxins, Springer-Verlag (1998).

The antibodies of the present invention can usefully be attached to a substrate, and it is, therefore, another aspect of the invention to provide antibodies that bind specifically to one or more of the polypeptides of the present invention, to one or more of the polypeptides encoded by the isolated nucleic acid molecules of the present invention, or the binding of which can be competitively inhibited by one or more of the polypeptides of the present invention or one or more of the polypeptides encoded by the isolated nucleic acid molecules of the present invention, attached to a substrate. Substrates can be porous or nonporous, planar or nonplanar. For example, the antibodies of the present invention can usefully be conjugated to filtration media, such as NHS-activated Sepharose or CNBr-activated Sepharose for purposes of immunoaffinity chromatography. For example, the antibodies of the present invention can usefully be attached to paramagnetic microspheres by, for example, biotin-streptavidin interaction. The microsphere can then be used for isolation of one or more cells that express or display the polypeptides of the present invention. As another example, the antibodies of the present invention can be attached to the surface of a microtiter plate for ELISA.

As noted herein, the antibodies of the present invention can be produced in prokaryotic and eukaryotic cells. It is, therefore, another aspect of the present invention to provide cells that express the antibodies of the present invention, including hybridoma cells, B6 cells, plasma cells, and host cells recombinantly modified to express the antibodies of the present invention.

In yet a further aspect, the present invention provides aptamers evolved to bind specifically to one or more of the LL proteins of the present invention or to polypeptides encoded by the nucleic acids of the invention.

In sum, one of skill in the art, provided with the teachings of this invention, has available a variety of methods which can be used to alter the biological properties of the antibodies of this invention including methods which can increase or decrease the stability or half-life, immunogenicity, toxicity, affinity or yield of a given antibody molecule, or to alter it in any other way that can render it more suitable for a particular application.

### Cellular Biology of Lisch-Like (Ll)

Embodiments and aspects described herein refer specifically to Ll, however, any of the described assays, techniques, reagents, experiments and so forth are equally applicable to determining and characterizing function and cellular biology of other LL homologues and orthologues, including but not limited to the human orthologue C1Orf32.

In certain aspects, the invention provides that LL promotes B6 cell growth, and can regulate peripheral metabolism through its effects on liver function. Both of these effects can be conveyed via the CNS/hypothalamus where LL is expressed. There are precedents for such effects on liver glucose metabolism and islet B6 cell function. LL function can be determined using assays of protein biosynthesis, processing, sub-cellular localization, signaling properties. Structure/function relationships are analyzed by way of gain- and loss-of-function experiments in appropriate cellular contexts.

In certain embodiments, the invention provides that highest levels of Ll expression are found in liver, brain, B6 cell/islet, and skeletal muscle. The metabolic properties of these organs are distinct, and make it difficult to identify an overarching function of the LL protein. Ki67 labeling studies indicate that B6 cell proliferation is reduced in the early post-natal period in DD (hypomorphic) congenics, indicating function for Ll in the regulation of β cell mass. Thus, LL modulates pancreatic B6 cell proliferation directly, or indirectly. LL cellular biological features can be determined by assays described herein and any other suitable method known in the art, in physiologically relevant cell types.

In certain aspects the invention provides antisera and antibodies against epitopes of predicted intra and extracellular domains that detect LL in immunoprecipitation, immunoblot and immunohistochemistry assays. These antibodies can be used to determine the cellular properties of the endogenous protein.

In other aspects the invention provides reagents to study the properties of Ll in gain-of-function experiments. Non-limiting examples of such reagents are FLAG epitope-tagged mammalian expression vectors. An LL-GFP fusion protein has been constructed and can be used to analyze sub-cellular localization. LL- and/or C1ORF32-fusion proteins to any other fluorescent protein variant, or any other protein reporter, or protein tag can also be generated. Also provided are mammalian expression vectors with N-terminal and C-terminal epitope tags and adenoviruses encoding WT Ll. Ll siRNA constructs have been tested and shown effective in HEK 293 cells. These probes can be engineered into adenoviral vectors for efficient gene knockdown in cultured cells and mice. siRNA-resistant rescue vectors can be generated in which synonymous nucleotide changes are introduced in the Ll cDNA to render it resistant to siRNA-mediated degradation. These constructs can be used to validate the specificity of the Ll siRNA. For most experiments described, mammalian expression vectors provide adequate expression levels, but to detect effects of LL on biological processes where high transfection and expression efficiency is needed, an adenovirus can be used.

### Expression Vectors, Host Cells and Recombinant Methods of Producing Polypeptides

Another aspect of the present invention provides vectors that comprise one or more of the isolated nucleic acid molecules of the present invention, and host cells in which such vectors have been introduced.

The vectors can be used, inter alia, for propagating the nucleic acid molecules of the present invention in host cells (cloning vectors), for shuttling the nucleic acid molecules of the present invention between host cells derived from disparate organisms (shuttle vectors), for inserting the nucleic acid molecules of the present invention into host cell chromosomes (insertion vectors), for expressing sense or antisense RNA transcripts of the nucleic acid molecules of the present invention in vitro or within a host cell, and for expressing polypeptides encoded by the nucleic acid molecules of the present invention, alone or as fusion proteins with heterologous polypeptides (expression vectors). Vectors are by now well known in the art, and are described, inter alia, in Jones et al. (eds.), Vectors: Cloning Applications Essential Techniques (Essential Techniques Series), John Wiley & Son Ltd. (1998); Jones et al. (eds.), Vectors: Expression Systems: Essential Techniques (Essential Techniques Series), John Wiley & Son Ltd. (1998); Gacesa et al., Vectors: Essential Data, John Wiley & Sons Ltd. (1995); Cid-Arregui (eds.), Viral Vectors: Basic Science and Gene Therapy, Eaton Publishing Co. (2000); Sambrook (2001), supra; Ausubel (1999), supra. Furthermore, a variety of vectors are available commercially. Use of existing vectors and modifications thereof are well within the skill in the art.

Nucleic acid sequences can be expressed by operatively linking them to an expression control sequence in an appropriate expression vector and employing that expression vector to transform an appropriate unicellular host. Expression control sequences are sequences that control the transcription, post-transcriptional events and translation of nucleic acid sequences. Such operative linking of a nucleic sequence of this invention to an expression control sequence, of course, includes, if not already part of the nucleic acid sequence, the provision of a translation initiation codon, ATG or GTG, in the correct reading frame upstream of the nucleic acid sequence.

A wide variety of host/expression vector combinations can be employed in expressing the nucleic acid sequences of this invention. Useful expression vectors, for example, can consist of segments of chromosomal, non-chromosomal and synthetic nucleic acid sequences.

In one embodiment, prokaryotic cells can be used with an appropriate vector. Prokaryotic host cells are often used for cloning and expression. In one embodiment, prokaryotic host cells include E. coli, Pseudomonas, Bacillus and Streptonzyces. In another embodiment, bacterial host cells are used to express the nucleic acid molecules and polypeptides of the invention. Useful expression vectors for bacterial hosts include bacterial plasmids, such as those from E. coli, Bacillus or Streptoinyces, including pBluescript, pGEX-2T, pUC vectors, col E1, pCR1, pBR322, pMB9 and their derivatives, wider host range plasmids, such as RP4, phage DNAs, e.g., the numerous derivatives of phage lambda, e.g., NM989, .lamda.GT10 and .lamda.GT11, and other phages, e.g., M13 and filamentous single stranded phage DNA. Where E. coli is used as host, selectable markers are, analogously, chosen for selectivity in gram negative bacteria: e.g., typical markers confer resistance to antibiotics, such as ampicillin, tetracycline, chloramphenicol, kanamycin, streptomycin and zeocin; auxotrophic markers can also be used.

In other embodiments, eukaryotic host cells, such as yeast, insect, mammalian or plant cells, can be used. Yeast cells, can be useful for eukaryotic genetic studies, due to the ease of targeting genetic changes by homologous recombination and the ability to easily complement genetic defects using recombinantly expressed proteins. Yeast cells are useful for identifying interacting protein components, e.g. through use of a two-hybrid system. In one embodiment, yeast cells are useful for protein expression. Vectors of the present invention for use in yeast can contain an origin of replication suitable for use in yeast and a selectable marker that is functional in yeast. Yeast vectors include Yeast Integrating plasmids (e.g., YIp5) and Yeast Replicating plasmids (the YRp and YEp series plasmids), Yeast Centromere plasmids (the YCp series plasmids), Yeast Artificial Chromosomes (YACs) which are based on yeast linear plasmids, denoted YLp, pGPD-2, 2 .mu.plasmids and derivatives thereof, and improved shuttle vectors such as those described in Gietz et al., Gene, 74: 527-34 (1988) (YIplac, YEplac and YCplac). Selectable markers in yeast vectors include a variety of auxotrophic markers, the most common of which are (in Saccharomyces cerevisiae) URA3, HIS3, LEU2, TRP1 and LYS2, which complement specific auxotrophic mutations, such as ura3-52, his3-D1, leu2-D1, trp1-D1 and lys2-201.

Insect cells can be chosen for high efficiency protein expression. Where the host cells are from Spodoptera frugiperda, e.g., Sf9 and Sf21 cell lines, and expresSF™ cells (Protein Sciences Corp., Meriden, Conn., USA), the vector replicative strategy can be based upon the baculovirus life cycle. Baculovirus transfer vectors can be used to replace the wild-type AcMNPV polyhedrin gene with a heterologous gene of interest. Sequences that flank the polyhedrin gene in the wild-type genome can be positioned 5′ and 3′ of the expression cassette on the transfer vectors. Following co-transfection with AcMNPV DNA, a homologous recombination event occurs between these sequences resulting in a recombinant virus carrying the gene of interest and the polyhedrin or p10 promoter. Selection can be based upon visual screening for lacZ fusion activity.

The host cells can also be mammalian cells, which can be useful for expression of proteins intended as pharmaceutical agents, and for screening of potential agonists and antagonists of a protein or a physiological pathway. Mammalian vectors intended for autonomous extrachromosomal replication can include a viral origin, such as the SV40 origin, the papillomavirus origin, or the EBV origin for long term episomal replication. Vectors intended for integration, and thus replication as part of the mammalian chromosome, can include an origin of replication functional in mammalian cells, such as the SV40 origin. Vectors based upon viruses, such as adenovirus, adeno-associated virus, vaccinia virus, and various mammalian retroviruses, can replicate according to the viral replicative strategy. Selectable markers for use in mammalian cells include, include but are not limited to, resistance to neomycin (G418), blasticidin, hygromycin and zeocin, and selection based upon the purine salvage pathway using HAT medium.

Expression in mammalian cells can be achieved using a variety of plasmids, including pSV2, pBC12BI, and p91023, as well as lytic virus vectors (e.g., vaccinia virus, adeno virus, and baculovirus), episomal virus vectors (e.g., bovine papillomavirus), and retroviral vectors (e.g., murine retroviruses). Useful vectors for insect cells include baculoviral vectors and pVL 941.

Plant cells can also be used for expression, with the vector replicon derived from a plant virus (e.g., cauliflower mosaic virus, CaMV; tobacco mosaic virus, TMV) and selectable markers chosen for suitability in plants.

It is known that codon usage of different host cells can be different. For example, a plant cell and a human cell can exhibit a difference in codon preference for encoding a particular amino acid. As a result, human mRNA can not be efficiently translated in a plant, bacteria or insect host cell. Therefore, another embodiment of this invention is directed to codon optimization. The codons of the nucleic acid molecules of the invention can be modified to resemble genes naturally contained within the host cell without altering the amino acid sequence encoded by the nucleic acid molecule.

Any of a wide variety of expression control sequences can be used in these vectors to express the nucleic acid molecules of this invention. Such useful expression control sequences include the expression control sequences associated with structural genes of the foregoing expression vectors. Expression control sequences that control transcription include, e.g., promoters, enhancers and transcription termination sites. Expression control sequences in eukaryotic cells that control post-transcriptional events include splice donor and acceptor sites and sequences that modify the half-life of the transcribed RNA, e.g., sequences that direct poly(A) addition or binding sites for RNA-binding proteins. Expression control sequences that control translation include ribosome binding sites, sequences which direct targeted expression of the polypeptide to or within cellular compartments, and sequences in the 5′ and 3′ untranslated regions that modify the rate or efficiency of translation.

Examples of useful expression control sequences for a prokaryote, e.g., E. coli, will include a promoter, often a phage promoter, such as phage lambda pL promoter, the trc promoter, a hybrid derived from the trp and lac promoters, the bacteriophage T7 promoter (in E. coli cells engineered to express the T7 polymerase), the TAC or TRC system, the major operator and promoter regions of phage lambda, the control regions of fd coat protein, and the araBAD operon. Prokaryotic expression vectors can further include transcription terminators, such as the aspA terminator, and elements that facilitate translation, such as a consensus ribosome binding site and translation termination codon, Schomer et al., Proc. Natl. Acad. Sci. USA 83: 8506-8510 (1986).

Expression control sequences for yeast cells can include a yeast promoter, such as the CYC1 promoter, the GAL1 promoter, the GAL10 promoter, ADH1 promoter, the promoters of the yeast .alpha.-mating system, or the GPD promoter, and can have elements that facilitate transcription termination, such as the transcription termination signals from the CYC1 or ADH1 gene.

Expression vectors useful for expressing proteins in mammalian cells will include a promoter active in mammalian cells. These promoters include, but are not limited to, those derived from mammalian viruses, such as the enhancer-promoter sequences from the immediate early gene of the human cytomegalovirus (CMV), the enhancer-promoter sequences from the Rous sarcoma virus long terminal repeat (RSV LTR), the enhancer-promoter from SV40 and the early and late promoters of adenovirus. Other expression control sequences include the promoter for 3-phosphoglycerate kinase or other glycolytic enzymes, the promoters of acid phosphatase. Other expression control sequences include those from the gene comprising the OSNA of interest. Often, expression is enhanced by incorporation of polyadenylation sites, such as the late SV40 polyadenylation site and the polyadenylation signal and transcription termination sequences from the bovine growth hormone (BGH) gene, and ribosome binding sites. Furthermore, vectors can include introns, such as intron II of rabbit .beta.-globin gene and the SV40 splice elements.

Nucleic acid vectors also include a selectable or amplifiable marker gene and means for amplifying the copy number of the gene of interest. Such marker genes are well known in the art. Nucleic acid vectors can also comprise stabilizing sequences (e.g., ori- or ARS-like sequences and telomere-like sequences), or can alternatively be designed to favor directed or non-directed integration into the host cell genome. In one embodiment, nucleic acid sequences of this invention are inserted in frame into an expression vector that allows a high level expression of an RNA which encodes a protein comprising the encoded nucleic acid sequence of interest. Nucleic acid cloning and sequencing methods are well known to those of skill in the art and are described in an assortment of laboratory manuals, including Sambrook (1989), supra, Sambrook (2000), supra; and Ausubel (1992), supra, Ausubel (1999), supra. Product information from manufacturers of biological, chemical and immunological reagents also provide useful information.

Expression vectors can be constitutive or inducible. Inducible vectors include naturally inducible promoters, such as the trc promoter, which is regulated by the lac operon, and the pL promoter, which is regulated by tryptophan, the MMTV-LTR promoter, which is inducible by dexamethasone, or can contain synthetic promoters and/or additional elements that confer inducible control on adjacent promoters. Examples of inducible synthetic promoters are the hybrid Plac/ara-1 promoter and the PLtetO-1 promoter. The PLtetO-1 promoter takes advantage of the high expression levels from the PL promoter of phage lambda, but replaces the lambda repressor sites with two copies of operator 2 of the Tn10 tetracycline resistance operon, causing this promoter to be tightly repressed by the Tet repressor protein and induced in response to tetracycline (Tc) and Tc derivatives such as anhydrotetracycline. Vectors can also be inducible because they contain hormone response elements, such as the glucocorticoid response element (GRE) and the estrogen response element (ERE), which can confer hormone inducibility where vectors are used for expression in cells having the respective hormone receptors. To reduce background levels of expression, elements responsive to ecdysone, an insect hormone, can be used instead, with coexpression of the ecdysone receptor.

In one embodiment of the invention, expression vectors can be designed to fuse the expressed polypeptide to small protein tags that facilitate purification and/or visualization. Such tags include a polyhistidine tag that facilitates purification of the fusion protein by immobilized metal affinity chromatography, for example using NiNTA resin (Qiagen Inc., Valencia, Calif., USA) or TALON™ resin (cobalt immobilized affinity chromatography medium, Clontech Labs, Palo Alto, Calif., USA). The fusion protein can include a chitin-binding tag and self-excising intein, permitting chitin-based purification with self-removal of the fused tag (IMPACT™ system, New England Biolabs, Inc., Beverley, Mass., USA). Alternatively, the fusion protein can include a calmodulin-binding peptide tag, permitting purification by calmodulin affinity resin (Stratagene, La Jolla, Calif., USA), or a specifically excisable fragment of the biotin carboxylase carrier protein, permitting purification of in vivo biotinylated protein using an avidin resin and subsequent tag removal (Promega, Madison, Wis., USA). As another useful alternative, the polypeptides of the present invention can be expressed as a fusion to glutathione-S-transferase, the affinity and specificity of binding to glutathione permitting purification using glutathione affinity resins, such as Glutathione-Superflow Resin (Clontech Laboratories, Palo Alto, Calif., USA), with subsequent elution with free glutathione. Other tags include, for example, the Xpress epitope, detectable by anti-Xpress antibody (Invitrogen, Carlsbad, Calif., USA), a myc tag, detectable by anti-myc tag antibody, the V5 epitope, detectable by anti-V5 antibody (Invitrogen, Carlsbad, Calif., USA), FLAG® epitope, detectable by anti-FLAG® antibody (Stratagene, La Jolla, Calif., USA), and the HA epitope, detectable by anti-HA antibody.

For secretion of expressed polypeptides, vectors can include appropriate sequences that encode secretion signals, such as leader peptides. For example, the pSecTag2 vectors (Invitrogen, Carlsbad, Calif., USA) are 5.2 kb mammalian expression vectors that carry the secretion signal from the V-J2-C region of the mouse Ig kappa-chain for efficient secretion of recombinant proteins from a variety of mammalian cell lines.

Expression vectors can also be designed to fuse proteins encoded by the heterologous nucleic acid insert to polypeptides that are larger than purification and/or identification tags. Useful protein fusions include those that permit display of the encoded protein on the surface of a phage or cell, fusions to intrinsically fluorescent proteins, such as those that have a green fluorescent protein (GFP)-like chromophore, fusions to the IgG Fc region, and fusions for use in two hybrid systems.

Vectors for phage display fuse the encoded polypeptide to, e.g., the gene III protein (pIII) or gene VIII protein (pVIII) for display on the surface of filamentous phage, such as M13. See Barbas et al., Phage Display: A Laboratory Manual, Cold Spring Harbor Laboratory Press (2001); Kay et al. (eds.), Phage Display of Peptides and Proteins: A Laboratory Manual, Academic Press, Inc., (1996); Abelson et al. (eds.), Combinatorial Chemistry (Methods in Enzymology, Vol. 267) Academic Press (1996). Vectors for yeast display, e.g. the pYD1 yeast display vector (Invitrogen, Carlsbad, Calif., USA), use the .alpha.-agglutinin yeast adhesion receptor to display recombinant protein on the surface of S. cerevisiae. Vectors for mammalian display, e.g., the pDisplay™ vector (Invitrogen, Carlsbad, Calif., USA), target recombinant proteins using an N-terminal cell surface targeting signal and a C-terminal transmembrane anchoring domain of platelet derived growth factor receptor.

A wide variety of vectors now exist that fuse proteins encoded by heterologous nucleic acids to the chromophore of the substrate-independent, intrinsically fluorescent green fluorescent protein from Aequorea Victoria (“GFP”) and its variants. The GFP-like chromophore can be selected from GFP-like chromophores found in naturally occurring proteins, such as A. Victoria GFP (GenBank accession number AAA2772 1), Renilla reniformis GFP, FP583 (GenBank accession no. AF168419) (DsRed), FP593 (AF27271 1), FP483 (AF168420), FP484 (AF168424), FP595 (AF246709), FP486 (AF168421), FP538 (AF168423), and FP506 (AF168422), and need include only so much of the native protein as is needed to retain the chromophore's intrinsic fluorescence. Methods for determining the minimal domain required for fluorescence are known in the art. See Li et al., J. Biol. Chem. 272: 28545-28549 (1997). Alternatively, the GFP-like chromophore can be selected from GFP-like chromophores modified from those found in nature. The methods for engineering such modified GFP-like chromophores and testing them for fluorescence activity, both alone and as part of protein fusions, are well known in the art. See Heim et al., Curr. Biol. 6: 178-182 (1996) and Palm et al., Methods Enzymol. 302: 378-394 (1999). A variety of such modified chromophores are now commercially available and can readily be used in the fusion proteins of the present invention. These include EGFP (“enhanced GFP”), EBFP (“enhanced blue fluorescent protein”), BFP2, EYFP (“enhanced yellow fluorescent protein”), ECFP (“enhanced cyan fluorescent protein”) or Citrine. EGFP (see, e.g, Cormack et al., Gene 173: 33-38 (1996); U.S. Pat. Nos. 6,090,919 and 5,804,387, the disclosures of which are incorporated herein by reference in their entireties) is found on a variety of vectors, both plasmid and viral, which are available commercially (Clontech Labs, Palo Alto, Calif., USA); EBFP is optimized for expression in mammalian cells whereas BFP2, which retains the original jellyfish codons, can be expressed in bacteria (see, e.g., Heim et al., Curr. Biol. 6: 178-182 (1996) and Cormack et al., Gene 173: 33-38 (1996)). Vectors containing these blue-shifted variants are available from Clontech Labs (Palo Alto, Calif., USA). Vectors containing EYFP, ECFP (see, e.g., Heim et al., Curr. Biol. 6: 178-182 (1996); Miyawaki et al., Nature 388: 882-887 (1997)) and Citrine (see, e.g., Heikal et al., Proc. Natl. Acad. Sci. USA 97: 11996-12001 (2000)) are also available from Clontech Labs. The GFP-like chromophore can also be drawn from other modified GFPs, including those described in U.S. Pat. Nos. 6,124,128; 6,096,865; 6,090,919; 6,066,476; 6,054,321; 6,027,881; 5,968,750; 5,874,304; 5,804,387; 5,777,079; 5,741,668; and 5,625,048, the disclosures of which are incorporated herein by reference in their entireties. See also Conn (ed.), Green Fluorescent Protein (Methods in Enzymology, Vol. 302), Academic Press, Inc. (1999); Yang, et al., J Biol Chem, 273: 8212-6 (1998); Bevis et al., Nature Biotechnology, 20:83-7 (2002). The GFP-like chromophore of each of these GFP variants can usefully be included in the fusion proteins of the present invention.

### Polypeptides, Including Fragments Mutant Proteins, Homologous Proteins, Allelic Variants, Analogs and Derivatives

Another aspect of the invention relates to polypeptides encoded by the nucleic acid molecules described herein. In one embodiment, the polypeptide is an LL polypeptide. A polypeptide as defined herein can be produced recombinantly, as discussed supra, can be isolated from a cell that naturally expresses the protein, or can be chemically synthesized following the teachings of the specification and using methods well known to those having ordinary skill in the art.

Polypeptides of the present invention can also comprise a part or fragment of a LL. In one embodiment, the fragment is derived from a polypeptide having an amino acid sequence selected from the group consisting of SEQ ID NO: 1-9, 14, 21-54 or 58. Polypeptides of the present invention comprising a part or fragment of an entire LL protein can or can not be LL proteins. A polypeptide that is not an LL protein, whether it is a fragment, analog, mutant protein, homologous protein or derivative, is nevertheless useful, especially for immunizing animals to prepare anti-LL protein antibodies. In one embodiment, the part or fragment is an LL protein. Methods of determining whether a polypeptide of the present invention is a LL protein are described herein.

Polypeptides of the present invention comprising fragments of at least 8 contiguous amino acids, often at least 15 contiguous amino acids, are useful as immunogens for raising antibodies that recognize polypeptides of the present invention. See, e.g., Lerner, Nature 299: 592-596 (1982); Shinnick et al., Annu. Rev. Microbiol. 37: 425-46 (1983); Sutcliffe et al., Science 219: 660-6 (1983). As further described in the references cited herein, 8-mers, conjugated to a carrier, such as a protein, prove immunogenic and are capable of eliciting antibody for the conjugated peptide; accordingly, fragments of at least 8 amino acids of the polypeptides of the present invention have utility as immunogens.

Polypeptides comprising fragments of at least 8, 9, 10 or 12 contiguous amino acids are also useful as competitive inhibitors of binding of the entire polypeptide, or a portion thereof, to antibodies (as in epitope mapping), and to natural binding partners, such as subunits in a multimeric complex or to receptors or ligands of the subject protein; this competitive inhibition permits identification and separation of molecules that bind specifically to the polypeptide of interest. See U.S. Pat. Nos. 5,539,084 and 5,783,674, incorporated herein by reference in their entireties.

The polypeptides of the present invention thus can be at least 6 amino acids in length, at least 8 amino acids in length, at least 9 amino acids in length, at least 10 amino acids in length, at least 12 amino acids in length, at least 15 amino acids in length, at least 20 amino acids in length, at least 25 amino acids in length, at least 30 amino acids in length, at least 35 amino acids in length, at least 50 amino acids in length, at least 75 amino acids in length, at least 100 amino acids in length, or at least 150 amino acids in length. Polypeptides of the present invention can also be larger and comprise a full-length LL protein and/or an epitope tag and/or a fusion protein.

One having ordinary skill in the art can produce fragments by truncating the nucleic acid molecule, encoding the polypeptide and then expressing it recombinantly. Alternatively, one can produce a fragment by chemically synthesizing a portion of the full-length polypeptide. One can also produce a fragment by enzymatically cleaving a recombinant polypeptide or an isolated naturally occurring polypeptide. Methods of producing polypeptide fragments are well known in the art. See, e.g., Sambrook (1989), supra; Sambrook (2001), supra; Ausubel (1992), supra; and Ausubel (1999), supra. In one embodiment, a polypeptide comprising only a fragment can be produced by chemical or enzymatic cleavage of a LL polypeptide.

Polypeptides of the present invention are also inclusive of mutants, fusion proteins, homologous proteins and allelic variants.

A mutant protein can have the same or different properties compared to a naturally occurring polypeptide and comprises at least one amino acid insertion, duplication, deletion, rearrangement or substitution compared to the amino acid sequence of a native polypeptide. Small deletions and insertions can often be found that do not alter the function of a protein. The mutant protein can be a polypeptide that comprises at least one amino acid insertion, duplication, deletion, rearrangement or substitution compared to the amino acid sequence of SEQ ID NO: 1-9, 14, 21-54 or 58. Accordingly, in one embodiment, the mutant protein is one that exhibits at least 60% sequence identity, at least 70%, or at least 80% sequence identity, at least 85% sequence identity, at least 90% sequence identity, at least 95% sequence identity, at least 97%, sequence identity at least 985, sequence identity at least 99% or sequence identity at least 99.5% to an LL protein.

A mutant protein can be produced by isolation from a naturally occurring mutant cell, tissue or organism. A mutant protein can be produced by isolation from a cell, tissue or organism that has been experimentally mutagenized. Alternatively, a mutant protein can be produced by chemical manipulation of a polypeptide, such as by altering the amino acid residue to another amino acid residue using synthetic or semi-synthetic chemical techniques. In one embodiment, a mutant protein is produced from a host cell comprising a mutated nucleic acid molecule compared to the naturally occurring nucleic acid molecule. For instance, one can produce a mutant protein of a polypeptide by introducing one or more mutations into a nucleic acid molecule of the invention and then expressing it recombinantly. These mutations can be targeted, in which encoded amino acids are altered, or can be untargeted, in which random encoded amino acids within the polypeptide are altered. Mutant proteins with random amino acid alterations can be screened for a biological activity or property. Multiple random mutations can be introduced into the gene by methods well known to the art, e.g., by error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis and site-specific mutagenesis. Methods of producing mutant proteins with targeted or random amino acid alterations are well known in the art. See, e.g., Sambrook (1989), supra; Sambrook (2001), supra; Ausubel (1992), supra; and Ausubel (1999), as well as U.S. Pat. No. 5,223,408, which is herein incorporated by reference in its entirety.

The invention also contemplates polypeptides that are homologous to a polypeptide of the invention. By homologous polypeptide it is means one that exhibits significant sequence identity to an LL protein. By significant sequence identity it is meant that the homologous polypeptide exhibits at least exhibits at least 60% sequence identity, at least 70%, or at least 80% sequence identity, at least 85% sequence identity, at least 90% sequence identity, at least 95% sequence identity, at least 97%, sequence identity at least 985, sequence identity at least 99% or sequence identity at least 99.5% to an LL protein. In one embodiment, the amino acid substitutions of the homologous polypeptide are conservative amino acid substitutions.

Homologous polypeptides of the present invention can be naturally occurring and derived from another species, especially one derived from another primate, such as chimpanzee, gorilla, rhesus macaque, or baboon, wherein the homologous polypeptide comprises an amino acid sequence that exhibits significant sequence identity to a polypepetide of the invention. The homologous polypeptide can also be a naturally occurring polypeptide from a human, when the LL protein is a member of a family of polypeptides. The homologous polypeptide can also be a naturally occurring polypeptide derived from a non-primate, mammalian species, including without limitation, domesticated species, e.g., dog, cat, mouse, rat, rabbit, guinea pig, hamster, cow, horse, goat or pig. The homologous polypeptide can also be a naturally occurring polypeptide derived from a non-mammalian species, such as birds or reptiles. The naturally occurring homologous protein can be isolated directly from humans or other species. Alternatively, the nucleic acid molecule encoding the naturally occurring homologous polypeptide can be isolated and used to express the homologous polypeptide recombinantly. The homologous polypeptide can also be one that is experimentally produced by random mutation of a nucleic acid molecule and subsequent expression of the nucleic acid molecule. Alternatively, the homologous polypeptide can be one that is experimentally produced by directed mutation of one or more codons to alter the encoded amino acid of an LL protein.

Relatedness of proteins can also be characterized using a second functional test, the ability of a first protein competitively to inhibit the binding of a second protein to an antibody. It is, therefore, another aspect of the present invention to provide isolated polpeptide not only identical in sequence to those described herein, but also to provide isolated polypeptide (“cross-reactive proteins”) that can competitively inhibit the binding of antibodies to all or to a portion of various of the isolated polypeptides of the present invention. Such competitive inhibition can readily be determined using immunoassays well known in the art.

As discussed herein, single nucleotide polymorphisms (SNPs) occur frequently in eukaryotic genomes, and the sequence determined from one individual of a species can differ from other allelic forms present within the population. Thus, polypeptides of the present invention are also inclusive of those encoded by an allelic variant of a nucleic acid molecule encoding an LL protein.

Polypeptides of the present invention are also inclusive of derivative polypeptides encoded by a nucleic acid molecule according to the invention. Also inclusive are derivative polypeptides having an amino acid sequence selected from the group consisting of an LL protein or a polypeptide of SEQ ID NO: 1-9, 14, 21-54 or 58 and which has been acetylated, carboxylated, phosphorylated, glycosylated, ubiquitinated or other post-translational modifications. In another embodiment, the derivative has been labeled with, e.g., radioactive isotopes such as .sup.125I, .sup.32P, .sup.35S, and .sup.3H. In another embodiment, the derivative has been labeled with fluorophores, chemiluminescent agents, enzymes, and antiligands that can serve as specific binding pair members for a labeled ligand.

Polypeptide modifications are well known to those of skill and have been described in detail in the scientific literature. Several common modifications, such as glycosylation, lipid attachment, sulfation, gamma-carboxylation of glutamic acid residues, hydroxylation and ADP-ribosylation, for instance, are described in most basic texts, such as, for instance Creighton, Protein Structure and Molecular Properties, 2nd ed., W.H. Freeman and Company (1993). Many detailed reviews are available on this subject, such as, for example, those provided by Wold, in Johnson (ed.), Posttranslational Covalent Modification of Proteins, pgs. 1-12, Academic Press (1983); Seifter et al., Meth. Enzymol. 182: 626-646 (1990) and Rattan et al., Ann. N.Y. Acad. Sci. 663: 48-62 (1992).

One can determine whether a polypeptide of the invention will be post-translationally modified by analyzing the sequence of the polypeptide to determine if there are peptide motifs indicative of sites for post-translational modification. There are a number of computer programs that permit prediction of post-translational modifications. See, e.g., expasy with the extension .org of the world wide web (accessed Nov. 11, 2002), which includes P SORT, for prediction of protein sorting signals and localization sites, SignalP, for prediction of signal peptide cleavage sites, MITOPROT and Predotar, for prediction of mitochondrial targeting sequences, NetOGlyc, for prediction of type O-glycosylation sites in mammalian proteins, big-PI Predictor and DGPI, for prediction of prenylation-anchor and cleavage sites, and NetPhos, for prediction of Ser, Thr and Tyr phosphorylation sites in eukaryotic proteins. Other computer programs, such as those included in GCG, also can be used to determine post-translational modification peptide motifs.

Examples of types of post-translational modifications include, but are not limited to: (Z)-dehydrobutyrine; 1-chondroitin sulfate-L-aspartic acid ester; 1′-glycosyl-L-tryptophan; 1′-phospho-L-histidine; 1-thioglycine; 2′-(S-L-cysteinyl)-L-histidine; 2′-[3-carboxamido(trimethylammonio)propyl]-L-histidine; 2′-alpha-mannosyl-L-tryptophan; 2-methyl-L-glutamine; 2-oxobutanoic acid; 2-pyrrolidone carboxylic acid; 3′-(1′-L-histidyl)-L-tyrosine; 3′-(8alpha-FAD)-L-histidine; 3′-(S-L-cysteinyl)-L-tyrosine; 3′,3″,5′-triiodo-L-thyronine; 3′-4′-phospho-L-tyrosine; 3-hydroxy-L-proline; 3′-methyl-L-histidine; 3-methyl-L-lanthionine; 3′-phospho-L-histidine; 4′-(L-tryptophan)-L-tryptophyl quinone; 42 N-cysteinyl-glycosylphosphatidylinositolethanolamine; 43-(T-L-histidyl)-L-tyrosine; 4-hydroxy-L-arginine; 4-hydroxy-L-lysine; 4-hydroxy-L-proline; 5′-(N-6-L-lysine)-L-topaquinone; 5-hydroxy-L-lysine; 5-methyl-L-arginine; alpha-1-microglobulin-Ig alpha complex chromophore; bis-L-cysteinyl bis-L-histidino diiron disulfide; bis-L-cysteinyl-L-N3′-histidino-L-serinyl tetrairon′ tetrasulfide; chondroitin sulfate D-glucuronyl-D-galactosyl-D-galactosyl-D-xylosyl-L-serine; //D-alanine; D-allo-isoleucine; D-asparagine; dehydroalanine; dehydrotyrosine; dermatan 4-sulfate D-glucuronyl-D-galactosyl-D-galactosyl-D-xylosyl-L-serine; D-glucuronyl-N-glycine; dipyrrolylmethanemethyl-L-cysteine; D-leucine; D-methionine; D-phenylalanine; D-serine; D-tryptophan; glycine amide; glycine oxazolecarboxylic acid; glycine thiazolecarboxylic acid; heme P450-bis-L-cysteine-L-tyrosine; heme-bis-L-cysteine; hemediol-L-aspartyl ester-L-glutamyl ester; hemediol-L-aspartyl ester-L-glutamyl ester-L-methionine sulfonium; heme-L-cysteine; heme-L-histidine; heparan sulfate D-glucuronyl-D-galactosyl-D-galactosyl-D-xylosyl-L-serine; heme P450-bis-L-cysteine-L-lysine; hexakis-L-cysteinyl hexairon hexasulfide; keratan sulfate D-glucuronyl-D-galactosyl-D-galactosyl-D-xylosyl-L-threonine; L oxoalanine-lactic acid; L phenyllactic acid; 1′-(8alpha-FAD)-L-histidine; L-2′,4′,5′-topaquinone; L-3′,4′-dihydroxyphenylalanine; L-3′,4′,5′-trihydroxyphenylalanine; L-4′-bromophenylalanine; L-6′-bromotryptophan; L-alanine amide; L-alanyl imidazolinone glycine; L-allysine; L-arginine amide; L-asparagine amide; L-aspartic 4-phosphoric anhydride; L-aspartic acid 1-amide; L-beta-methylthioaspartic acid; L-bromohistidine; L-citrulline; L-cysteine amide; L-cysteine glutathione disulfide; L-cysteine methyl disulfide; L-cysteine methyl ester; L-cysteine oxazolecarboxylic acid; L-cysteine oxazolinecarboxylic acid; L-cysteine persulfide; L-cysteine sulfenic acid; L-cysteine sulfinic acid; L-cysteine thiazolecarboxylic acid; L-cysteinyl homocitryl molybdenum-heptairon-nonasulfide; L-cysteinyl imidazolinone glycine; L-cysteinyl molybdopterin; L-cysteinyl molybdopterin guanine dinucleotide; L-cystine; L-erythro-beta-hydroxyasparagine; L-erythro-beta-hydroxyaspartic acid; L-gamma-carboxyglutarnic acid; L-glutamic acid 1-amide; L-glutamic acid 5-methyl ester; L-glutamine amide; L-glutamyl 5-glycerylphosphorylethanolamine; L-histidine amide; L-isoglutamyl-polyglutamic acid; L-isoglutamyl-polyglycine; L-isoleucine amide; L-lanthionine; L-leucine amide; L-lysine amide; L-lysine thiazolecarboxylic acid; L-lysinoalanine; L-methionine amide; L-methionine sulfone; L-phenyalanine thiazolecarboxylic acid; L-phenylalanine amide; L-proline amide; L-selenocysteine; L-selenocysteinyl molybdopterin guanine dinucleotide; L-serine amide; L-serine thiazolecarboxylic acid; L-seryl imidazolinone glycine; L-T-bromophenylalanine; L-T-bromophenylalanine; L-threonine amide; L-thyroxine; L-tryptophan amide; L-tryptophyl quinone; L-tyrosine amide; L-valine amide; meso-lanthionine; N-(L-glutamyl)-L-tyrosine; N-(L-isoaspartyl)-glycine; N-(L-isoaspartyl)-L-cysteine; N,N,N-trimethyl-L-alanine; N,N-dimethyl-L-proline; N2-acetyl-L-lysine; N2-succinyl-L-tryptophan; N4-(ADP-ribosyl)-L-asparagine; N4-glycosyl-L-asparagine; N4-hydroxymethyl-L-asparagine; N4-methyl-L-asparagine; N5-methyl-L-glutamine; N6-1-carboxyethyl-L-lysine; N6-(4-amino hydroxybutyl)-L-lysine; N6-(L-isoglutamyl)-L-lysine; N6-(phospho-5′-adenosine)-L-lysine; N6-(phospho-5′-guanosine)-L-lysine; N6,N6,N6-trimethyl-L-lysine; N6,N6-dimethyl-L-lysine; N6-acetyl-L-lysine; N6-biotinyl-L-lysine; N6-carboxy-L-lysine; N6-formyl-L-lysine; N6-glycyl-L-lysine; N6-lipoyl-L-lysine; N6-methyl-L-lysine; N6-methyl-N6-poly(N-methyl-propylamine)-L-lysine; N6-mureinyl-L-lysine; N6-myristoyl-L-lysine; N6-palmitoyl-L-lysine; N6-pyridoxal phosphate-L-lysine; N6-pyruvic acid 2-iminyl-L-lysine; N6-retinal-L-lysine; N-acetylglycine; N-acetyl-L-glutamine; N-acetyl-L-alanine; N-acetyl-L-aspartic acid; N-acetyl-L-cysteine; N-acetyl-L-glutamic acid; N-acetyl-L-isoleucine; N-acetyl-L-methionine; N-acetyl-L-proline; N-acetyl-L-serine; N-acetyl-L-threonine; N-acetyl-L-tyrosine; N-acetyl-L-valine; N-alanyl-glycosylphosphatidylinositolethanolamine; N-asparaginyl-glycosylphosphatidylinositolethanolamine; N-aspartyl-glycosylphosphatidylinositolethanolamine; N-formylglycine; N-formyl-L-methionine; N-glycyl-glycosylphosphatidylinositolethanolamine; N-L-glutamyl-poly-L-glutamic acid; N-methylglycine; N-methyl-L-alanine; N-methyl-L-methionine; N-methyl-L-phenylalanine; N-myristoyl-glycine; N-palmitoyl-L-cysteine; N-pyruvic acid 2-iminyl-L-cysteine; N-pyruvic acid 2-iminyl-L-valine; N-seryl-glycosylphosphatidylinositolethanolamine; N-seryl-glycosyOSPhingolipidinositolethanolamine; O-(ADP-ribosyl)-L-serine; O-(phospho-5′-adenosine)-L-threonine; O-(phospho-5′-DNA)-L-serine; O-(phospho-5′-DNA)-L-threonine; O-(phospho-5′ rRNA)-L-serine; O-(phosphoribosyl dephospho-coenzyme A)-L-serine; O-(sn-1-glycerophosphoryl)-L-serine; O4′-(8alpha-FAD)-L-tyrosine; O4′-(phospho-5′-adenosine)-L-tyrosine; O4′-(phospho-5′-DNA)-L-tyrosine; O4′-(phospho-5′-RNA)-L-tyrosine; O4′-(phospho-5′-uridine)-L-tyrosine; O4-glycosyl-L-hydroxyproline; O4′-glycosyl-L-tyrosine; O4′-sulfo-L-tyrosine; O5-glycosyl-L-hydroxylysine; O-glycosyl-L-serine; O-glycosyl-L-threonine; omega-N-(ADP-ribosyl)-L-arginine; omega-N-omega-N′-dimethyl-L-arginine; omega-N-methyl-L-arginine; omega-N-omega-N-dimethyl-L-arginine; omega-N-phospho-L-arginine; O′ octanoyl-L-serine; O-palmitoyl-L-serine; O-palmitoyl-L-threonine; O-phospho-L-serine; O-phospho-L-threonine; O-phosphopantetheine-L-serine; phycoerythrobilin-bis-L-cysteine; phycourobilin-bis-L-cysteine; pyrroloquinoline quinone; pyruvic acid; S hydroxycinnamyl-L-cysteine; S-(2-aminovinyl)methyl-D-cysteine; S-(2-aminovinyl)-D-cysteine; S-(6-FW-L-cysteine; S-(8alpha-FAD)-L-cysteine; S-(ADP-ribosyl)-L-cysteine; S-(L-isoglutamyl)-L-cysteine; S-12-hydroxyfarnesyl-L-cysteine; S-acetyl-L-cysteine; S-diacylglycerol-L-cysteine; S-diphytanylglycerot diether-L-cysteine; S-farnesyl-L-cysteine; S-geranylgeranyl-L-cysteine; S-glycosyl-L-cysteine; S-glycyl-L-cysteine; S-methyl-L-cysteine; S-nitrosyl-L-cysteine; S-palmitoyl-L-cysteine; S-phospho-L-cysteine; S-phycobiliviolin-L-cysteine; S-phycocyanobilin-L-cysteine; S-phycoerythrobilin-L-cysteine; S-phytochromobilin-L-cysteine; S-selenyl-L-cysteine; S-sulfo-L-cysteine; tetrakis-L-cysteinyl diiron disulfide; tetrakis-L-cysteinyl iron; tetrakis-L-cysteinyl tetrairon tetrasulfide; trans-2,3-cis 4-dihydroxy-L-proline; tris-L-cysteinyl triiron tetrasulfide; tris-L-cysteinyl triiron trisulfide; tris-L-cysteinyl-L-aspartato tetrairon tetrasulfide; tris-L-cysteinyl-L-cysteine persulfido-bis-L-glutamato-L-histidino tetrairon disulfide trioxide; tris-L-cysteinyl-L-N3′-histidino tetrairon tetrasulfide; tris-L-cysteinyl-L-NM'-histidino tetrairon tetrasulfide; and tris-L-cysteinyl-L-serinyl tetrairon tetrasulfide.

Additional examples of post translational modifications can be found in web sites such as the Delta Mass database based on Krishna, R. G. and F. Wold (1998). Posttranslational Modifications. Proteins—Analysis and Design. R. H. Angeletti. San Diego, Academic Press. 1: 121-206.; Methods in Enzymology, 193, J. A. McClosky (ed) (1990), pages 647-660; Methods in Protein Sequence Analysis edited by Kazutomo Imahori and Fumio Sakiyama, Plenum Press, (1993) “Post-translational modifications of proteins” R. G. Krishna and F. Wold pages 167-172; “GlycoSuiteDB: a new curated relational database of glycoprotein glycan structures and their biological sources” Cooper et al. Nucleic Acids Res. 29; 332-335 (2001) “O-GLYCBASE version 4.0: a revised database of O-glycosylated proteins” Gupta et al. Nucleic Acids Research, 27: 370-372 (1999); and “PhosphoBase, a database of phosphorylation sites: release 2.0.”, Kreegipuu et al. Nucleic Acids Res 27(1):237-239 (1999) see also, WO 02/211 39A2, the disclosure of which is incorporated herein by reference in its entirety.

Disease states are often accompanied by alterations in the post-translational modifications of proteins. Thus, in another embodiment, the invention provides polypeptides from diseased cells or tissues that have altered post-translational modifications compared to the post-translational modifications of polypeptides from normal cells or tissues. A number of altered post-translational modifications are known. One common alteration is a change in phosphorylation state, wherein the polypeptide from the diseased cell or tissue is hyperphosphorylated or hypophosphorylated compared to the polypeptide from a normal tissue, or wherein the polypeptide is phosphorylated on different residues than the polypeptide from a normal cell. Another common alteration is a change in glycosylation state, wherein the polypeptide from the diseased cell or tissue has more or less glycosylation than the polypeptide from a normal tissue, and/or wherein the polypeptide from the diseased cell or tissue has a different type of glycosylation than the polypeptide from a non-diseased cell or tissue.

Another post-translational modification that can be altered in diseased cells is prenylation. Prenylation is the covalent attachment of a hydrophobic prenyl group (farnesyl or geranylgeranyl) to a polypeptide. Prenylation is required for localizing a protein to a cell membrane and is often required for polypeptide function. For instance, the Ras superfamily of GTPase signalling proteins must be prenylated for function in a cell. See, e.g., Prendergast et al., Semin. Cancer Biol. 10: 443-452 (2000) and Khwaja et al., Lancet 355: 741-744 (2000).

Other post-translation modifications that can be altered in diseased cells include, without limitation, polypeptide methylation, acetylation, arginylation or racemization of amino acid residues. In these cases, the polypeptide from the diseased cell can exhibit increased or decreased amounts of the post-translational modification compared to the corresponding polypeptides from non-diseased cells.

Other polypeptide alterations in diseased cells include abnormal polypeptide cleavage of proteins and aberrant protein-protein interactions. Abnormal polypeptide cleavage can be cleavage of a polypeptide in a diseased cell that does not usually occur in a normal cell, or a lack of cleavage in a diseased cell, wherein the polypeptide is cleaved in a normal cell. Aberrant protein-protein interactions can be covalent cross-linking or non-covalent binding between proteins that do not normally bind to each other. Alternatively, in a diseased cell, a protein can fail to bind to another protein to which it is bound in a non-diseased cell. Alterations in cleavage or in protein-protein interactions can be due to over- or underproduction of a polypeptide in a diseased cell compared to that in a normal cell, or can be due to alterations in post-translational modifications of one or more proteins in the diseased cell. See, e.g., Henschen-Edman, Ann. N.Y. Acad. Sci. 936: 580-593 (2001).

Alterations in polypeptide post-translational modifications, as well as changes in polypeptide cleavage and protein-protein interactions, can be determined by any method known in the art. For instance, alterations in phosphorylation can be determined by using anti-phosphoserine, anti-phosphothreonine or anti-phosphotyrosine antibodies or by amino acid analysis. Glycosylation alterations can be determined using antibodies specific for different sugar residues, by carbohydrate sequencing, or by alterations in the size of the glycoprotein, which can be determined by, e.g., SDS polyacrylamide gel electrophoresis (PAGE). Other alterations of post-translational modifications, such as prenylation, racemization, methylation, acetylation and arginylation, can be determined by chemical analysis, protein sequencing, amino acid analysis, or by using antibodies that bind a post-translational modification. Changes in protein-protein interactions and in polypeptide cleavage can be analyzed by any method known in the art including, without limitation, non-denaturing PAGE (for non-covalent protein-protein interactions), SDS PAGE (for covalent protein-protein interactions and protein cleavage), chemical cleavage, protein sequencing or immunoassays.

In another embodiment, the invention provides polypeptides that have been post-translationally modified. In one embodiment, polypeptides can be modified enzymatically or chemically, by addition or removal of a post-translational modification. For example, a polypeptide can be glycosylated or deglycosylated enzymatically. Similarly, polypeptides can be phosphorylated using a purified kinase, such as a MAP kinase (e.g, p38, ERK, or JNK) or a tyrosine kinase (e.g., Src or erbB2). A polypeptide can also be modified through synthetic chemistry. Alternatively, one can isolate the polypeptide of interest from a cell or tissue that expresses the polypeptide with the desired post-translational modification. In another embodiment, a nucleic acid molecule encoding the polypeptide of interest is introduced into a host cell that is capable of post-translationally modifying the encoded polypeptide in the desired fashion. If the polypeptide does not contain a motif for a desired post-translational modification, one can alter the post-translational modification by mutating the nucleic acid sequence of a nucleic acid molecule encoding the polypeptide so that it contains a site for the desired post-translational modification Amino acid sequences that can be post-translationally modified are known in the art. See, e.g., the programs described herein on the Expasy website. The nucleic acid molecule can also be introduced into a host cell that is capable of post-translationally modifying the encoded polypeptide. Similarly, one can delete sites that are post-translationally modified by mutating the nucleic acid sequence so that the encoded polypeptide does not contain the post-translational modification motif, or by introducing the native nucleic acid molecule into a host cell that is not capable of post-translationally modifying the encoded polypeptide.

Polypeptides are not always entirely linear. For instance, polypeptides can be branched as a result of ubiquitination, and they can be circular, with or without branching, as a result of posttranslation events, including natural processing event and events brought about by human manipulation which do not occur naturally. Circular, branched and branched circular polypeptides can be synthesized by non-translation natural process and by entirely synthetic methods, as well. Modifications can occur anywhere in a polypeptide, including the peptide backbone, the amino acid side-chains and the amino or carboxyl termini. In fact, blockage of the amino or carboxyl group in a polypeptide, or both, by a covalent modification, is common in naturally occurring and synthetic polypeptides and such modifications can be present in polypeptides of the present invention, as well. For instance, the amino terminal residue of polypeptides made in E. coli, prior to proteolytic processing, almost invariably will be N-formylmethionine.

Useful post-synthetic (and post-translational) modifications include conjugation to detectable labels, such as fluorophores. A wide variety of amine-reactive and thiol-reactive fluorophore derivatives have been synthesized that react under nondenaturing conditions with N-terminal amino groups and epsilon amino groups of lysine residues, on the one hand, and with free thiol groups of cysteine residues, on the other.

Kits are available commercially that permit conjugation of proteins to a variety of amine-reactive or thiol-reactive fluorophores: Molecular Probes, Inc. (Eugene, Oreg., USA), e.g., offers kits for conjugating proteins to Alexa Fluor 350, Alexa Fluor 430, Fluorescein-EX, Alexa Fluor 488, Oregon Green 488, Alexa Fluor 532, Alexa Fluor 546, Alexa Fluor 546, Alexa Fluor 568, Alexa Fluor 594, and Texas Red-X A wide variety of other amine-reactive and thiol-reactive fluorophores are available commercially (Molecular Probes, Inc., Eugene, Oreg., USA), including Alexa Fluor® 350, Alexa Fluor® 488, Alexa Fluor® 532, Alexa Fluor® 546, Alexa Fluor® 568, Alexa Fluor® 594, Alexa Fluor® 647 (monoclonal antibody labeling kits available from Molecular Probes, Inc., Eugene, Oreg., USA), BODIPY dyes, such as BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY TR, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethylrhodamine, Texas Red (available from Molecular Probes, Inc., Eugene, Oreg., USA).

The polypeptides of the present invention can also be conjugated to fluorophores, other proteins, and other macromolecules, using bifunctional linking reagents. Common homobifunctional reagents include, e.g., APG, AEDP, BASED, BMB, BMDB, BMH, BMOE, BM[PEO]3, BM[PEO]4, BS3, BSOCOES, DFDNB, DMA, DMP, DMS, DPDPB, DSG, DSP (Lomant's Reagent), DSS, DST, DTBP, DTME, DTSSP, EGS, HBVS, Sulfo-BSOCOES, Sulfo-DST, Sulfo-EGS (available from Pierce, Rockford, Ill., USA); common heterobifunctional cross-linkers include ABH, AMAS, ANB-NOS, APDP, ASBA, BMPA, BMPH, BMPS, EDC, EMCA, EMCH, EMCS, KMUA, KMUH, GMBS, LC-SMCC, LC-SPDP, MBS, M2C2H, MPBH, MSA, NHS-ASA, PDPH, PMPI, SADP, SAED, SAND, SANPAH, SASD, SATP, SBAP, SFAD, SIA, SLAB, SMCC, SMPB, SMPH, SMPT, SPDP, Sulfo-EMCS, Sulfo-GMBS, Sulfo-HSAB, Sulfo-KMUS, Sulfo-LC-SPDP, Sulfo-MBS, Sulfo-NHS-LC-ASA, Sulfo-SADP, Sulfo-SANPAH, Sulfo-SIAB, Sulfo-SMCC, Sulfo-SMPB, Sulfo-LC-SMPT, SVSB, TFCS (available Pierce, Rockford, Ill., USA).

Polypeptides of the present invention, including full length polypeptides, fragments and fusion proteins, can be conjugated, using such cross-linking reagents, to fluorophores that are not amine- or thiol-reactive. Other labels that usefully can be conjugated to polypeptides of the present invention include radioactive labels, echosonographic contrast reagents, and MRI contrast agents.

Polypeptides of the present invention, including full length polypeptide, fragments and fusion proteins, can also usefully be conjugated using cross-linking agents to carrier proteins, such as KLH, bovine thyroglobulin, and even bovine serum albumin (BSA), to increase immunogenicity for raising anti-LL protein antibodies.

Polypeptides of the present invention, including full length polypeptide, fragments and fusion proteins, can also usefully be conjugated to polyethylene glycol (PEG); PEGylation increases the serum half life of proteins administered intravenously for replacement therapy. Delgado et al., Crit. Rev. Ther. Drug Carrier Syst. 9(3-4): 249-304 (1992); Scott et al., Curr. Pharm. Des. 4(6): 423-38 (1998); DeSantis et al., Curr. Opin. Biotechnol. 10(4): 324-30 (1999). PEG monomers can be attached to the protein directly or through a linker, with PEGylation using PEG monomers activated with tresyl chloride (2,2,2-trifluoroethanesulphonyl chloride) permitting direct attachment under mild conditions.

Polypeptides of the present invention are also inclusive of analogs of a polypeptide encoded by a nucleic acid molecule according to the invention. In one embodiment, this polypeptide is an LL protein. In another embodiment the analog polypeptide comprises one or more substitutions of non-natural amino acids or non-native inter-residue bonds compared to the naturally occurring polypeptide. In one embodiment, the analog is structurally similar to an LL protein, but one or more peptide linkages is replaced by a linkage selected from the group consisting of —CH.sub.2NH—, —CH.sub.2S—, —CH.sub.2-CH.sub.2-, —CH.dbd.CH—(cis and trans), —COCH.sub.2-, —CH(OH)CH.sub.2- and —CH.sub.2SO—. In another embodiment, the analog comprises substitution of one or more amino acids of a LL protein with a D-amino acid of the same type or other non-natural amino acid in order to generate more stable peptides. D-amino acids can readily be incorporated during chemical peptide synthesis: peptides assembled from D-amino acids are more resistant to proteolytic attack; incorporation of D-amino acids can also be used to confer specific three-dimensional conformations on the peptide. Other amino acid analogues that can be added during chemical synthesis include ornithine, norleucine, phosphorylated amino acids (for example, phosphoserine, phosphothreonine, phosphotyrosine), L-malonyltyrosine, a non-hydrolyzable analog of phosphotyrosine (see, e.g., Kole et al., Biocheem. Biophlys. Res. Com. 209: 817-821 (1995)), and various halogenated phenylalanine derivatives.

Non-natural amino acids can be incorporated during solid phase chemical synthesis or by recombinant techniques. Solid phase chemical synthesis of peptides is well established in the art. Procedures are described, inter alia, in Chan et al. (eds.), Fmoc Solid Phase Peptide Synthesis: A Practical Approach (Practical Approach Series), Oxford Univ. Press (March 2000); Jones, Amino Acid and Peptide Synthesis (Oxford Chemistry Primers, No 7), Oxford Univ. Press (1992); and Bodanszky, Principles of Peptide Synthesis (Springer Laboratory), Springer Verlag (1993).

Amino acid analogues having detectable labels are also usefully incorporated during synthesis to provide derivatives and analogs. Biotin, for example can be added using biotinoyl-(9-fluorenylmethoxycarbonyl)-L-lysine (FMOC biocytin) (Molecular Probes, Eugene, Oreg., USA). Biotin can also be added enzymatically by incorporation into a fusion protein of a E. coli BirA substrate peptide. The FMOC and tBOC derivatives of dabcyl-L-lysine (Molecular Probes, Inc., Eugene, Oreg., USA) can be used to incorporate the dabcyl chromophore at selected sites in the peptide sequence during synthesis. The aminonaphthalene derivative EDANS, the most common fluorophore for pairing with the dabcyl quencher in fluorescence resonance energy transfer (FRET) systems, can be introduced during automated synthesis of peptides by using EDANS-FMOC-L-glutamic acid or the corresponding tBOC derivative (both from Molecular Probes, Inc., Eugene, Oreg., USA). Tetramethylrhodamine fluorophores can be incorporated during automated FMOC synthesis of peptides using (FMOC)-TMR-L-lysine (Molecular Probes, Inc. Eugene, Oreg., USA).

Other useful amino acid analogues that can be incorporated during chemical synthesis include aspartic acid, glutamic acid, lysine, and tyrosine analogues having allyl side-chain protection (Applied Biosystems, Inc., Foster City, Calif., USA); the allyl side chain permits synthesis of cyclic, branched-chain, sulfonated, glycosylated, and phosphorylated peptides.

A large number of other FMOC-protected non-natural amino acid analogues capable of incorporation during chemical synthesis are available commercially, including, e.g., Fmoc-2-aminobicyclo[2.2.1]heptane-2-carboxylic acid, Fmoc-3-endo-aminobicyclo[2.2.1]heptane-2-endo-carboxylic acid, Fmoc-3-exo-aminobicyclo[2.2.1]heptane-2-exo-carboxylic acid, Fmoc-3-endo-amino-bicyclo[2.2.1]hept-5-ene-2-endo-carboxylic acid, Fmoc-3-exo-amino-bicyclo[2.2.1]hept-5-ene-2-exo-carboxylic acid, Fmoc-cis-2-amino-1-cyclohexanecarboxylic acid, Fmoc-trans-2-amino-1-cyclohexanecarboxylic acid, Fmoc-1-amino-1-cyclopentanecarboxylic acid, Fmoc-cis-2-amino-1-cyclopentanecarboxylic acid, Fmoc-1-amino-1-cyclopropanecarboxylic acid, Fmoc-D-2-amino-4-(ethylthio)butyric acid, Fmoc-L-2-amino-4-(ethylthio)butyric acid, Fmoc-L-buthionine, Fmoc-5-methyl-L-Cysteine, Fmoc-2-aminobenzoic acid (anthranillic acid), Fmoc-3-aminobenzoic acid, Fmoc-4-aminobenzoic acid, Fmoc-2-aminobenzophenone-2′-carboxylic acid, Fmoc-N-(4-aminobenzoyl)-.beta.-alanine, Fmoc-2-amino-4,5-dimethoxybenzoic acid, Fmoc-4-aminohippuric acid, Fmoc-2-amino-3-hydroxybenzoic acid, Fmoc-2-amino-5-hydroxybenzoic acid, Fmoc-3-amino-4-hydroxybenzoic acid, Fmoc4-amino-3-hydroxybenzoic acid, Fmoc-4-amino-2-hydroxybenzoic acid, Fmoc-5-amino-2-hydroxybenzoic acid, Fmoc-2-amino-3-methoxybenzoic acid, Fmoc4-amino-3-methoxybenzoic acid, Fmoc-2-amino-3-methylbenzoic acid, Fmoc-2-amino-5-methylbenzoic acid, Fmoc-2-amino-6-methylbenzoic acid, Fmoc-3-amino-2-methylbenzoic acid, Fmoc-3-amino-4-methylbenzoic acid, Fmoc-4-amino-3-methylbenzoic acid, Fmoc-3-amino-2-naphtoic acid, Fmoc-D,L-3-amino-3-phenylpropionic acid, Fmoc-L-Methyldopa, Fmoc-2-amino-4,6-dimethyl-3-pyridinecarboxylic acid, Fmoc-D,L-amino-2-thiophenacetic acid, Fmoc-4-(carboxymethyl)piperazine, Fmoc-4-carboxypiperazine, Fmoc-4-(carboxymethyl)homopiperazine, Fmoc-4-phenyl-4-piperidinecarboxylic acid, Fmoc-L-1,2,3,4-tetrahydronorharman-3-carboxylic acid, Fmoc-L-thiazolidine-4-carboxylic acid, available from—The Peptide Laboratory (Richmond, Calif., USA).

Non-natural residues can also be added biosynthetically by engineering a suppressor tRNA by chemical aminoacylation with the desired unnatural amino acid. Conventional site-directed mutagenesis is used to introduce the chosen stop codon UAG at the site of interest in the protein gene. When the acylated suppressor tRNA and the mutant gene are combined in an in vitro transcription/translation system, the unnatural amino acid is incorporated in response to the UAG codon to give a protein containing that amino acid at the specified position. Liu et al., Proc. Natl. Acad. Sci. USA 96(9): 4780-5 (1999); Wang et al., Science 292(5516): 498-500 (2001).

### Fusion Proteins

Another aspect of the present invention relates to the fusion of a polypeptide of the present invention to heterologous polypeptides. In one embodiment, the polypeptide of the present invention is an LL protein or is a mutant protein, homologous polypeptide, analog or derivative thereof.

The fusion proteins of the present invention will include at least one fragment of a polypeptide of the present invention, which fragment is at least 6 amino acids in length, at least 8 amino acids in length, at least 9 amino acids in length, at least 10 amino acids in length, at least 12 amino acids in length, at least 15 amino acids in length, at least 20 amino acids in length, at least 25 amino acids in length, at least 30 amino acids in length, at least 35 amino acids in length, at least 50 amino acids in length, at least 75 amino acids in length, at least 100 amino acids in length, or at least 150 amino acids in length. Fusions proteins that include the entirety of a polypeptide of the present invention are also useful.

The heterologous polypeptide included within the fusion protein of the present invention is at least 6 amino acids in length, often at least 8 amino acids in length, and can be at least 15, 20, or 25 amino acids in length. Fusions that include larger polypeptides, such as the IgG Fc region, and even entire proteins (such as GFP chromophore-containing proteins) can be useful.

Heterologous polypeptides to be included in the fusion proteins of the present invention can usefully include those designed to facilitate purification and/or visualization of recombinantly-expressed proteins. See, e.g., Ausubel, Chapter 16, (1992), supra. Although purification tags can also be incorporated into fusions that are chemically synthesized, chemical synthesis can also provides sufficient purity. Such tags can retain their utility even when the protein is produced by chemical synthesis, and when so included render the fusion proteins of the present invention useful as directly detectable markers of the presence of a polypeptide of the invention.

Heterologous polypeptides to be included in the fusion proteins of the present invention can usefully include those that facilitate secretion of recombinantly expressed proteins into the periplasmic space or extracellular milieu for prokaryotic hosts or into the culture medium for eukaryotic cells through incorporation of secretion signals and/or leader sequences. For example, a His.sup.6 tagged protein can be purified on a Ni affinity column and a GST fusion protein can be purified on a glutathione affinity column. Similarly, a fusion protein comprising the Fc domain of IgG can be purified on a Protein A or Protein G column and a fusion protein comprising an epitope tag such as myc can be purified using an immunoaffinity column containing an anti-c-myc antibody. The epitope tag can be separated from the protein encoded by the essential gene by an enzymatic cleavage site that can be cleaved after purification. See also the discussion of nucleic acid molecules encoding fusion proteins that can be expressed on the surface of a cell.

Other useful fusion proteins of the present invention include those that permit use of the polypeptide of the present invention as bait in a yeast two-hybrid system. See Bartel et al. (eds.), The Yeast Two-Hybrid System, Oxford University Press (1997); Zhu et al., Yeast Hybrid Technologies, Eaton Publishing (2000); Fields et al., Trends Genet. 10(8): 286-92 (1994); Mendelsohn et al, Curr. Opin. Biotechnol. 5(5): 482-6 (1994) Luban et al., Curr. Opin. Biotechnol. 6(1): 59-64 (1995); Allen et al., Trends Biochem. Sci. 20(12): 511-6 (1995); Drees, Curr. Opin. Cliem. Biol. 3(1): 64-70 (1999); Topcu et al, Pharm. Res. 17(9): 1049-55 (2000); Fashena et al., Gene 250(1-2): 1-14 (2000); Colas et al., Nature 380, 548-550 (1996); Norman, T. et al., Science 285, 591-595 (1999); Fabbrizio et al., Oncogene 18, 4357-4363 (1999); Xu et al., Proc Natl Acad Sci USA. 94, 12473-12478 (1997); Yang, et al., Nuc. Acids Res. 23, 1152-1156 (1995); Kolonin et al., Proc Natl Acad Sci USA 95, 14266-14271 (1998); Cohen et al., Proc Natl Acad Sci U S A 95, 14272-14277 (1998); Uetz, et al. Nature 403, 623-627 (2000); Ito, et al., Proc Natl Acad Sci USA 98, 4569-4574 (2001). Such fusion can be made to E. coli LexA or yeast GAL4 DNA binding domains. Related bait plasmids are available that express the bait fused to a nuclear localization signal.

Other useful fusion proteins include those that permit display of the encoded polypeptide on the surface of a phage or cell, fusions to intrinsically fluorescent proteins, such as green fluorescent protein (GFP), and fusions to the IgG Fc region, as described herein.

The polypeptides of the present invention can also usefully be fused to protein toxins, such as Pseudomonas exotoxin A, diphtheria toxin, shiga toxin A, anthrax toxin lethal factor, ricin, in order to effect ablation of cells that bind or take up the proteins of the present invention.

Fusion partners include, inter alia, myc, hemagglutinin (HA), GST, immunoglobulins, p-galactosidase, biotin trpE, protein A, .beta.-lactamase, .alpha.-amylase, maltose binding protein, alcohol dehydrogenase, polyhistidine (for example, six histidine at the amino and/or carboxyl terminus of the polypeptide), lacZ, green fluorescent protein (GFP), yeast a mating factor, GALA transcription activation or DNA binding domain, luciferase, and serum proteins such as ovalbumin, albumin and the constant domain of IgG. See, e.g., Ausubel (1992), supra and Ausubel (1999), supra. Fusion proteins can also contain sites for specific enzymatic cleavage, such as a site that is recognized by enzymes such as Factor XIII, trypsin, pepsin, or any other enzyme known in the art. Fusion proteins can be made by recombinant nucleic acid methods or chemically synthesized using techniques well known in the art (e.g., a Merrifield synthesis), or produced by chemical cross-linking.

Another advantage of fusion proteins is that the epitope tag can be used to bind the fusion protein to a plate or column through an affinity linkage for screening binding proteins or other molecules that bind to the LL protein.

The polypeptides of the present invention can readily be used as specific immunogens to raise antibodies that specifically recognize polypeptides of the present invention including LL proteins and their allelic variants and homologues. The antibodies can be used to specifically to assay for the polypeptides of the present invention with the use of several techniques, for example ELISA, immunohistochemistry, laser scanning cytometry, flow cytometry, immunoprecipitation, immunoblotting and for detection of LL proteins or for use as specific agonists or antagonists of LL proteins.

One can determine whether polypeptides of the present invention including LL proteins, mutant proteins, homologous proteins or allelic variants or fusion proteins of the present invention are functional by methods known in the art. For instance, residues that are tolerant of change while retaining function can be identified by altering the polypeptide at known residues using methods known in the art, such as alanine scanning mutagenesis, Cunningham et al., Science 244(4908): 1081-5 (1989); transposon linker scanning mutagenesis, Chen et al., Gene 263(1-2): 39-48 (2001); combinations of homolog- and alanine-scanning mutagenesis, Jin et al., J. Mol. Biol. 226(3): 851-65 (1992); combinatorial alanine scanning, Weiss et al., Proc. Natl. Acad. Sci. USA 97(16): 8950-4 (2000), followed by functional assay. Transposon linker scanning kits are available commercially (New England Biolabs, Beverly, Mass., USA, catalog. no. E7-1025; EZ::TN™ In-Frame Linker Insertion Kit, catalogue no. EZIO4KN, (Epicentre Technologies Corporation, Madison, Wis., USA).

Purification of the polypeptides or fusion proteins of the present invention is well known and within the skill of one having ordinary skill in the art. See, e.g., Scopes, Protein Purification, 2d ed. (1987). Purification of recombinantly expressed polypeptides is described herein. Purification of chemically-synthesized peptides can readily be effected, e.g., by HPLC.

Accordingly, it is an aspect of the present invention to provide the isolated polypeptides or fusion proteins of the present invention in pure or substantially pure form in the presence of absence of a stabilizing agent. Stabilizing agents include both proteinaceous and non-proteinaceous material and are well known in the art. Stabilizing agents, such as albumin and polyethylene glycol (PEG) are known and are commercially available.

Although high levels of purity can be useful when the isolated polypeptide or fusion protein of the present invention are used as therapeutic agents, such as in vaccines and replacement therapy, the isolated polypeptides of the present invention are also useful at lower purity. For example, partially purified polypeptides of the present invention can be used as immununogens to raise antibodies in laboratory animals. The purified and substantially purified polypeptides of the present invention are in compositions that lack detectable ampholytes, acrylamide monomers, bis-acrylamide monomers, and polyacrylamide.

The polypeptides or fusion proteins of the present invention can usefully be attached to a substrate. The substrate can be porous or solid, planar or non-planar; the bond can be covalent or noncovalent. For example, the peptides of the invention can be stabilized by covalent linkage to albumin. See, U.S. Pat. No. 5,876,969, the contents of which are hereby incorporated in its entirety.

For example, the polypeptides or fusion proteins of the present invention can usefully be bound to a porous substrate or a membrane such as nitrocellulose, polyvinylidene fluoride (PVDF), or cationically derivatized, hydrophilic PVDF. When bound the polypeptides or fusion proteins of the present invention can be used to detect and quantify antibodies, e.g. in serum, that bind specifically to the immobilized polypeptide or fusion protein of the present invention.

As another example, the polypeptides or fusion proteins of the present invention can usefully be bound to a substantially nonporous substrate, such as plastic, to detect and quantify antibodies, e.g. in serum, that bind specifically to the immobilized protein of the present invention. Such plastics include polymethylacrylic, polyethylene, polypropylene, polyacrylate, polymethylmethacrylate, polyvinylchloride, polytetrafluoroethylene, polystyrene, polycarbonate, polyacetal, polysulfone, celluloseacetate, cellulosenitrate, nitrocellulose, or mixtures thereof; when the assay is performed in a standard microtiter dish, the plastic can be polystyrene.

The polypeptides and fusion proteins of the present invention can also be attached to a substrate suitable for use as a surface enhanced laser desorption ionization source; so attached, the polypeptide or fusion protein of the present invention is useful for binding and then detecting secondary proteins that bind with sufficient affinity or avidity to the surface-bound polypeptide or fusion protein to indicate biologic interaction there between. The polypeptides or fusion proteins of the present invention can also be attached to a substrate suitable for use in surface plasmon resonance detection; so attached, the polypeptide or fusion protein of the present invention is useful for binding and then detecting secondary proteins that bind with sufficient affinity or avidity to the surface-bound polypeptide or fusion protein to indicate biological interaction there between.

### Alternative Transcripts

In another aspect, the present invention provides splice variants of genes and proteins encoded thereby. The identification of a splice variant which encodes an amino acid sequence with a region can be targeted for the generation of reagents for use in detection and/or treatment of diabetes. The amino acid sequence can lead to a unique protein structure, protein subcellular localization, biochemical processing or function of the splice variant. This information can be used to directly or indirectly facilitate the generation of additional or therapeutics or diagnostics. The nucleotide sequence in this splice variant can be used as a nucleic acid probe for the diagnosis and/or treatment of diabetes.

Specifically, the newly identified sequences can enable the production of antibodies or compounds directed against the region for use as a therapeutic or diagnostic. Alternatively, the newly identified sequences can alter the biochemical or biological properties of the encoded protein in such a way as to enable the generation of improved or different therapeutics targeting this protein.

### Tissues, Cells, Cell Lines: Protein Synthesis, Processing, Degradation

Ll is expressed as several variably spliced isoforms with specificity by strain and organ. In certain aspects, the invention provides a full-length cDNA cloned in a mammalian expression vector, adding C-terminal and/or N-terminal tags—as noted—to facilitate detection following transfection. In certain embodiments, transient transfection assays can be carried out in β-TC3 insulinoma cells and SV40-transformed hepatocytes (Rother, 1998, J Biol Chem 273:17491-17497) followed by immunoprecipitation with anti-HA antiserum and immunoblot with anti-L1 antiserum. These cell lines have been chosen because they maintain at least some physiologic properties of β cells and hepatocytes. Moreover, they are well characterized, easy to maintain, and handle transfecting/transducing them with a variety of expression and viral vectors. These lines were successfully transfected with full length Ll constructs. Using these lines, experiments can be performed in the presence and absence of cycloheximide to block protein synthesis and visualize on the blots the molecular weight of the expressed products, how rapidly they are degraded, and whether they differ in different cell types. Transient transfection assays can be used for this type of experiment because they are easier and prevent clonal artifact. Although transfection efficiency is irrelevant in this context, this technique can be optimized in these cell types. Using modified lipofection reagents, 30-40% efficiency can be achieved in SV40 hepatocytes. Using the Amaxa system, up to 80-90% of β-TC3 cells can be transfected.

In alternative embodiments of the methods of the present invention, different insulinoma cells, such as Ins1, MIN-6 or HIT can be transfected. In other embodiments, screening methods of the invention, or basic studies of (cell) biology of LL or C1ORF32 can be carried out in HEK293 or 3T3 cells. The former cells have the advantage of being easily transfectable but—HEK293 being a human kidney-derived cell line—Ll processing can or can not reflect that in murine Ll target tissues. To circumvent this problem, murine 3T3 cells, or any other suitable cell type can be used.

### Sub-Cellular Localization

Ll is predicted to encode a single membrane-spanning domain, with a large extracellular domain and a C-terminal intracellular domain. Data in Min6 cells transfected with Ll-GFP reveal a plasma membrane and punctate cytoplasmic pattern, which can be consistent with targeting to specialized plasma membrane compartments (caveolae, coated pits), lysosomes, and mitochondria. This question will be addressed using immunofluorescence in cells expressing LL-GFP. These experiments will use the same cell types described herein, and confocal microscopy, to detect LL localization. In certain embodiments, cycloheximide can be used to determine whether LL localization changes as a function of protein turnover. Time-lapse microscopy will be used to visualize protein fate in the presence of cycloheximide. The GFP tag is located at the C-terminus of LL. Thus, if LL is cleaved during its intracellular journey, this construct will only allow detection of the C-terminal domain. To circumvent this potential problem, immunocytochemistry will be performed with HA antiserum in cells transfected with Ll constructs bearing a double tag N-terminal (HA) and C-terminal (FLAG-tag). In one embodiment, LL can be processed as a single peptide with a stable sub-cellular localization. In this case, the L1-GFP construct and the double-tag construct will yield overlapping patterns of sub-cellular localization. In another embodiment, LL can be processed into different peptides, each with a distinct sub-cellular localization in a manner that may be similar to Tubby (Santagata et al, 2001, Science 292:2041-2050; Boggon et al, 1999, Science 286:2119-2125) and SREBP1C proteins, which are proteolytically cleaved to activate their transcriptional functions can be considered (Horton et al, 2002, J Clin Invest 109:1125-1131). In this case, the subcellular localization of the HA-tagged and FLAG-tagged constructs will differ, and only the FLAG-tagged construct will overlap with L1-GFP—appropriate cellular markers can be used to identify cellular compartments to which LL localizes; LL sub-cellular localization, as a single peptide, or as multiple processed products, changes in response to various cues—the effect of various hormonal and metabolic treatments on this process can be examined. In non-limiting examples, in B6 cells, the effects of glucose and cAMP can be determined, while in liver the effect of insulin and cAMP can be determined. In both cell types, the effects of FFA and lipoproteins can be determined. As a control for these experiments, Foxo-GFP, which undergoes rapid sub-cellular re-localization in response to these various agents, can be used. Actual experimental details (dose response, time course, etc) will be patterned according to prior experience in this area (Nakae et al, 2001, J Clin Invest 108:1359-1367; Nakae et al, 2000, Embo J 19:989-996).

### Phosphorylation

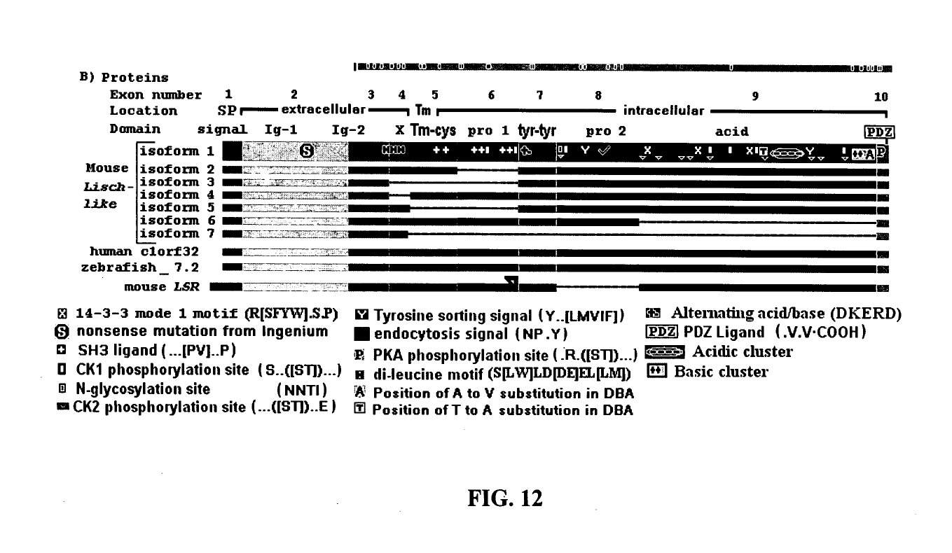

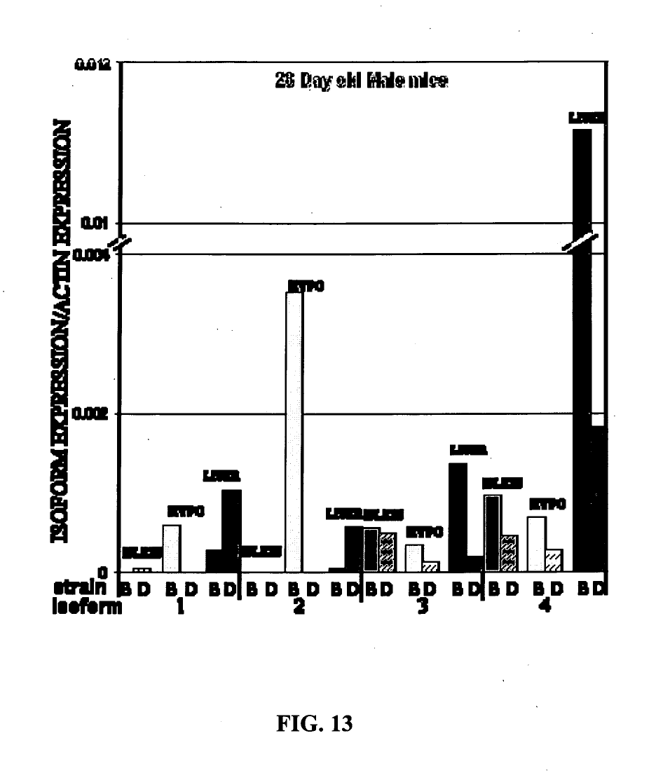

Many proteins with metabolic functions are modified via phosphorylation by tyrosine and serine/threonine kinases. As indicated, the putative intracellular domain of LL contains several putative sites for Ser/Thr kinases. Using 32P-orthophosphate labeling of intact cells, it can be determined whether LL is phosphorylated in vivo and whether changes in the cell's metabolic status affect LL phosphorylation. The initial experiments will be carried out by in vivo labeling followed by immunoprecipitation and autoradiography. If required, phospho-peptide maps will be employed (Accili et al, 1991, J Biol Chem 266:434-439) and mass spectrometry to identify individual phosphorylation sites. If LL phosphorylation changes with the cell's hormonal/nutritional status, further experiments will be conducted to identify phosphorylation sites on LL and relevant kinases. There are a number of potential Ser/Thr phosphorylation sites in the intracellular domain of LL (FIG. 12). Of special interest are four PKA sites (at amino acid residue 307, 352, 399, 403), an Akt site at position 618, and a CDK site at position 550. Given that PKA and Akt are activated in response to glucagon and insulin signaling, respectively, it will be of interest to determine whether these agents affect LL phosphorylation. If so, these sites will be mutated to probe their involvement in LL phosphorylation and function. Similarly, it will be important to test LL phosphorylation as function of cell cycle progression, given preliminary data that in dd mice (with low LL levels) replication of B6 cells is decreased. If there are changes in LL phosphorylation as function of cell cycle progression, the CDK phosphorylation site can be mutated to determine whether LL function is affected. One of the two non-conservative nucleotide substitutions identified in DD mice abolishes a potential Ck1 site (T572A). Thus, the phosphorylation state of the WT vs T572A mutant LL will be compared to determine whether (a) the site is phosphorylated and (b) its mutation into a non-phosphorylatable amino acid changes localization, signaling or bioeffects of L1. Candidate phosphorylation sites described herein will be replaced by non-phosphorylatable amino acids (alanine) to generate phosphorylation-deficient mutants, or by charged amino acids (aspartic or glutamic acid) to mimic the phosphorylated state and generate “constitutively phosphorylated” mutants

### Readout Assays of Ll Gain-of-Function

In certain aspects, the basic cell biology of Ll can be characterized. In other aspects, transgenic and knockout mice can be generated and characterized by methods and techniques as described herein, and also known in the art.

In certain aspects, the invention provides that Ll function is related to decrease in B6 cell mass, which is secondary to reduced proliferation. In other aspects, the invention provides that LL has a role to bind lipids—based upon close sequence homology to LSR (lipolysis-stimulated receptor). To further characterize these, β-TC3 cells (very low in endogenous L1) will be transfected with WT (B6-derived) HA-L1, and B6 cell proliferation will be measured. Gain of Ll function can result in increased B6 cell proliferation. To carry out these experiments it can be necessary to achieve high transfection frequency to measure an effect in an unselected cell population. In non-limiting examples, transfection efficiency can be monitored using tagged constructs, or/and carrying out immunocytochemistry (for HA-tagged constructs) or fluorescence (for GFP-tagged constructs) with Ki67 or BrdU immunocytochemistry to co-localize transfected Ll with in actively replicating cells. Ll-expressing cells will stain positive for Ki67 or BrdU enable measurement of replication rates using pulse-chase experiments. Because β-TC3 cells express very low levels of endogenous Ll, transfection of recombinant Ll can result in a gain-of-function that may not be apparent in other B6 cell lines expressing higher levels of L1 where pathways may active due to endogenous Ll. Tet-dependent β-TC3 clones exist in which addition of tetracycline to the medium results in rapid cell cycle arrest (Efrat et al, 1998, Proc Natl Acad Sci USA 85:9037-9041). Thus, if the replication rates of β-TC3 are unaffected by Ll in regular culture conditions, the ability of Ll over-expression to promote cell cycle progression in Tet-arrested β-TC3 cells can be studied.

To examine the mechanism of Ll-induced changes in cellular proliferation, markers of cell cycle progression, including Foxo1/3, p27kip, p21 and pRb will be analyzed (Okamoto et al, 2006, J Clin Invest 116:775-782; Buteau et al, 2006, Diabetes 55:1190-1196; Kitamura et al, 2005, Cell Metab 2:153-163; Kitamura et al, 2002, J Clin Invest 110:1839-1847). LL can also affect proliferation by reducing apoptosis. Rate of apoptosis can be determined in cultured β cells, and in vivo. In certain aspects, the invention provides that DD mice, have reduced B6 cell proliferation in the early post-natal stage. A physiologic remodeling of β-cell mass occurs in rodents at this stage (Scaglia et al, 1997, Endocrinology 138:1736-1741), due to a wave of apoptosis. LL can be involved in this process. Apoptosis markers such as Fas1, Caspase-3, -8, Bax and Bim will be examined.

In addition to cell replication, insulin secretion assays in response to glucose and other secretagogues, as well as mitochondrial function experiments to measure mitochondrial integrity will be performed (Buteau et al, 2006, Diabetes 55:1190-1196). Because insulin secretion and β cell proliferation are linked (Okamoto et al, 2006, J Clin Invest 116:775-782), LL can affect primarily secretion, which secondarily impairs β cell proliferation. The expression of markers of terminally differentiated B6 cells, such as MafA, a transcription factor expressed at low levels in B6-TC3 cells, which makes them an ideal system to study MafA induction (Kitamura 2005) will be determined Foxo1-3, Pdx1, Nkx2.2 and Hnf4 will be measured. LL can beneficially affect stimulus/secretion coupling in the 13 cell, and thus upregulate expression of relevant transcription factors.

### Signaling Pathways Activated by LL and Protein/Protein Interactions

In certain aspects, the invention provides that LL function affects signaling pathways in insulinoma cells. Following Ll over-expression activation of candidate pathways, including but not limited to PI 3-kinase/Akt, mTOR/S6k, AMPK/Acc, cAMP/PKA pathways will be measured (Buteau et al, 2006, Diabetes 55:1190-1196; Kitamura et al, 2005, Cell Metab 2:153-163). These assays can be carried out in an unselected population of cells after transient transfection. In other embodiments, similar experiments can be carried in cells transduced with Ll adenovirus (Kitamura et al, 2005, Cell Metab 2:153-163).

### Loss-of-Function Experiments

In other aspects, the invention provides methods to determine the effect of Ll reduction or ablation on the aforementioned parameters and characteristics in islet cells. Because B6-TC3 cells express low endogenous Ll levels and are not suitable for this purpose, these experiments will be carried out in MIN-6 cells. To carry out these experiments, high-efficiency transfection with the Amaxa system, or siRNA adenovirus will be used (Matsumoto et al, 2006, J Clin Invest 116:2464-2472). As control, transfections of mutant siRNA or siRNA-resistant Ll will be used. In certain aspects, the invention provides that gain of Ll function increases cellular proliferation and loss of Ll function decreases it. In certain embodiments, the invention provides methods to determine Ll function in primary cultures of mouse islets transduced with adenoviral constructs (Kitamura et al, 2005, Cell Metab 2:153-163).

### LL Functions in the Hepatocyte

In liver, the outcome of functional experiments is more complex. Proliferation of hepatocytes, while important in many pathophysiologic conditions, is not considered a predisposing factor in diabetes/insulin resistance. Thus, the actions of LL in hepatocytes must be deduced from other assays. The phenotypes of the ENU Ll-null mice (and a transgenic or conditional knockout mouse) will guide experimental approach to LL function in hepatocytes. In certain aspects, the invention provides methods to carry out gain-of-function experiments in hepatocytes to study Ll's cell biological properties: localization, processing, signaling properties. These experiments will employ SV40-transformed hepatocytes, a cell type that retains many of the properties of terminally differentiated hepatocytes (Rother et al, 1998, J Biol Chem 273:17491-17497; Kim et al, 2001, Endocrinology 142:3354-3360; Park et al, 1999, Biochemistry 38:7517-7523). Processing, turnover, localization and phosphorylation can be examined as described herein and by any other suitable method known in the art. Among the signaling pathways that can be studied following Ll over-expression are: cAMP and insulin signaling, as well as adiponectin, lipids (FFA) and bile acids-activated signaling. Candidate effectors of LL signaling and/or, Srebp1c□include PI 3-kinase, mTOR/S6 kinase, AMP kinase, Ppar induction. The biological responses that can be measured include glucose production, glycogen synthesis, TG content and synthesis, ApoB and LDL/VLDL secretion (Han et al, 2006, Cell Metab 3:257-266; Matsumoto et al, 2006, J Clin Invest 116:2464-2472). The liver, in which there are large differences in B6 v. DBA expression of Ll, affects B6 cells by a metabolic, e.g. lipoprotein, or endocrine pathway, hepatokine production, or by agents in these pathways. Liver-mediated effects on B6 cell development/function can be examined by co-culture of congenic line or knockout hepatocytes with suitable B6 cell line, expression arrays, and analysis of isolated liver proteins by 2-D gel and mass spectrometry.

### Ll Alternatively Spliced Isoforms

Ll is expressed as several different transcripts (FIG. 12). Notably, the abundance and assortment of transcripts varies from cell type to cell type, and by strain. Complete transcripts from 7 isoforms were isolated. However, isoforms 5,6,7 were only isolated in trace quantities from cDNA libraries (FIG. 13). Isoform 1 contains the ten exons intact, while the others have missing or truncated exons. Complete transcripts for isoforms 1-4 were isolated and partial transcripts in trace quantities were isolated from pooled DBA cDNA libraries for isoforms 5-7.

Evaluating the full spectrum of the functions of these various isoforms can be carried out by methods as described herein and by any suitable methods know in the art (Liu et al, 1998, Mamm Genome 9:780-781; Chua et al, 1997, Genomics 45:264-270). One determination includes whether these spliced isoforms are translated. A protein isoform expression survey using western blot analysis will be carried out. If different molecular species are observed, tissue expression and mRNA variants will be monitored. Some of these isoforms have reduced stability, and that alternative message splicing provides a mechanism to indirectly regulate LL levels by altering its post-transcriptional or translational degradation. Certain isoforms are secreted and can be detected in the circulation, acting as a decoy receptor for a putative LL ligand. This will easily become apparent from western blot surveys of various tissues/cell types and incubation media in different conditions, as described herein. To address the issue of secreted isoforms, serum protein will also be included in the tissue survey. The turnover rates of the most prominent splice variants will be investigated using pulse-chase experiments with cycloheximide, and survey their intracellular localization by immunocytochemistry.

The putative transmembrane structure of LL shows that LL can be a cell surface receptor. This is supported by the presence of several Ig repeats in the putative extracellular domain, a defining feature of cell adhesion molecules and various cell surface receptors. Methods of identifying ligands for cell surface receptors are well known in the art and can be readily used to identify a ligand for LL or LL homologs.

### Molecular Basis of Decreased Ll Expression in DD Congenic Mice

In certain aspects, the invention provides that the DBA allele decreases Ll expression levels through a cis-acting DNA element(s). The mechanism can be explained by: (a) reduced gene transcription; (b) decreased mRNA stability, and/or (c) increased protein degradation; these are not mutually exclusive. In other aspects, the invention provides that the DBA allele of Ll results in reduced protein levels in hepatocytes, B6 cells and the brain. Understanding the relevant mechanism(s) will help to elucidate the molecular physiology of LL.

The Ll gene encodes large, alternatively spliced transcripts. Coding (exon 9) and non-coding (mainly 3′ UTR) sequence changes can be evaluated in the DDA vs. BBA strains as candidate mutations causing alterations of mRNA levels. Because the extent of the decrease in mRNA levels is different from tissue to tissue (Table 4), tissue-specific factors can contribute to the process. Because the largest differences in mRNA levels were found in the liver, cis-acting vibrations in Ll can be examined in this tissue. The results described herein show that the region downstream of exon 8 is implicated in conveying diabetes susceptibility. Because this is a region of sequence overlap within Ll in the congenic lines described herein such analysis can be used to determine whether the 5′UTR is cis-acting region that can contribute variation to differences in gene expression among the congenic lines. For examples, regulatory DNA elements acting upstream of the transcription start site may interact with elements downstream of exon 8 to decrease mRNA transcription/stability. These experiments can determine whether the low levels of Ll transcripts seen in liver are due to decreased transcription. mRNA stability and decay can be also analyzed.

### Changes in Gene Transcription

The promoter regions of Ll in DD and BB mice are extremely well conserved. Although, there are no nucleotide substitution S detected in the 10 kb upstream of the transcription start site, cis-acting elements controlling Ll expression have not been mapped may reside outside the sequenced regions. In one embodiment, in vivo run-on studies using livers of DD vs DB mice can be performed to determine if the two alleles are transcribed at different rates. Because the mRNA levels in liver differ >10-fold between the two strains (Table 4), one can detect a difference, if indeed mRNA transcription is responsible for the molecular phenotype. Methods known in the art can be used to address these questions (McKeon et al, 1997, Biochem Biophys Res Commun 240:701-706; McKeon et al, 1990, Mol Endocrinol 4:647-656). In another embodiment, primary hepatocytes from the two strains can be prepared and run-on experiments can be performed in this culture system, which is more amenable to hormonal/metabolic control (i.e., it can be determined if the process is critically dependent on various hormone/metabolic cues). Comparison of a strain that segregates for DBA alleles only in exons 8-10+3′ UTR (e.g. 1 jcdt) to one in which the entire Ll gene is DBA (1jc) can allow apportioning effects via the 5′ promoter region.

### In Vivo Analysis of Ll Function in Mice

In certain aspects, the invention provides that loss or reduction of Ll function predisposes to diabetes in mice, of a susceptible genetic background by impairing β cell proliferation and hepatic metabolism. In other aspect, the invention provides that loss or reduction of C1Orf32 function predisposes human subject to diabetes.

In certain aspects, the invention provides that loss-of-function conveyed by the DBA allele of Ll is the cause of diabetes susceptibility in DD mice. Thus, conference of diabetes susceptibility can be achieved by introducing loss of Ll function in diabetes-susceptible strains.

ENU mutagenesis provides a powerful tool to introduce mutations in the mouse genome. In certain embodiments, the invention provides an ENU-mutagenized mouse (C3HeB/FeJ) segregating for a W87* (stop) mutation in L1. The ENU amber mutation in exon 2 of Ll can produce a completely inactive allele. Because, the mutation is on a C3HeB/FeJ background, a C57BL/6J conditional knockout of Ll can be made with or without a knockout vicinal genes. In other embodiments, the invention provides methods to characterize LL knockout mice by a number of metabolic abnormalities related to diabetes. In certain embodiments, characterization can be made by measuring the β cell response, hepatic glucose, or lipid metabolism.

ENU-mutagenized mice, as well as knockout strains which can be generated as described herein and by methods known in the art, can be characterized at various developmental stages using several parameters. Exemplary parameters are somatic growth curves, body composition, plasma glucose and insulin levels in fasted and fed states, lipid profile (triglycerides, cholesterol, FFAs), glucose tolerance tests, insulin release tests, pyruvate challenge, glucose clamps, functional, histological and immunohistochemical characterization of pancreatic islets as indicated below. Assays and techniques to carry out these characterizations are described herein and known in the art.

Non-limiting methods include calorimetry and euglycemic hyperinsulinemic clamp studies. Euglycemic hyperinsulinemic clamp studies—euglycemic clamps will be perfomed in conscious, unrestrained, catheterized mice as previously described (Okamoto et al, 2005, J Clin Invest 115:1314-1322). A solution of glucose (10%) will be infused at a variable rate as required to maintain euglycemia (7 mM). Mice will receive a constant infusion of HPLC-purified [3-³H] and insulin (18mU/kg body wt/min). Thereafter, plasma will be collected to determine glucose levels at times 10, 20, 30, 40, 50, 60, 70, 80, and 90 min, as well as the specific activities of [3-³H] glucose and tritiated water at times 30, 40, 50, 60, 70, 80, and 90 min. Steady-state conditions can be achieved for both plasma glucose concentration and specific activity by 30 minutes in these studies. [U-¹⁴C] lactate (5 μCi bolus/0.25 μCi/min) will be infused during the last 10 min of the study. β-cell “phenotyping”. Numerous assays have been described herein and are known in the art to evaluate β-cell function in mouse models of diabetes. Ki67 immunoreactivity will be used to assess B6 cell proliferation. Detection of apoptosis can be carried out using immunohistochemistry with caspase-3. Because apoptosis occurs at specific developmental stages, time course analysis can be performed in 1 to 4 week-old mice. Islets can be isolated from mice by in vivo collagenase perfusion, and insulin release under different experimental conditions can be determined. If mutations result in developmental abnormalities, embryonic analysis can be performed by delivering embryos at various gestational stages by Caesarian section. The analysis can comprise identification of the pancreatic buds, dissection, histological or morphometric analysis of islet number, size and composition. Electron microscopy can be performed as described (Cinti et al, 1998, Diabetologia 41:171-177).

In certain embodiments, the −/− ENU mice, can be characterized by stressing the β cells using low dose streptozotocin, dexamethasone, dietary manipulations, etc.

### Targeted Mutations

Targeted mutations in animals can be generated with ENU mice segregating on the basis of a stop codon in exon 2.

### Conventional Knock-Out

A gene targeting vector, as described herein, can be designed to carry out a conventional gene inactivation experiment. The vector can be used for both ubiquitous and conditional inactivation of Ll. For conventional gene knockout, the sequence flanked by loxP sites can be excised in vitro, using transfections of ES cells carrying the gene-targeted allele (Bruning et al, 1998, Mol Cell 2:559-569), or by intercrossing mice carrying a floxed allele with “deleter” cre transgenics, leading to removal of the lox-flanked sequence in germ cells (Okamoto et al, 2004, J Clin Invest 114:214-223; Bruning et al, 1998, Mol Cell 2:559-569; Han et al, 2006, Cell Metab 3:257-266; Xuan et al, 2002, J Clin Invest 110:1011-1019; Okamoto et al, 2005, J Clin Invest 115:1314-1322).

### Conditional Knock-Out

Cre-loxP technology known in the art can be used to introduce mutations in an organ or in a developmental stage-specific fashion. As described herein, Ll ablation in 13 cells can affect their ability to proliferate, thus modulating diabetes susceptibility in vivo. Conditional Ll knockouts can be generated at various developmental stages during endocrine pancreas differentiation using crosses of mice homozygous for a floxed Ll allele with Neurogenin 3-cre, Pdx-cre and Insulin-cre transgenic mice. Each cre transgenic can cause Ll inactivation at a different stage in pancreas development, and can thus provide insight into the developmental role of Ll in this process.

### Pdx-Cre Knock-Out

In certain embodiments, Pdx-Cre can be used to inactivate Ll in pancreatic progenitors, prior to the differentiation of the endocrine, exocrine and ductal lineages. If Ll plays a role in the determination of the pancreatic lineages, ablation of Ll driven by this Cre mice can result in widespread alterations of exocrine and endocrine cell number, characteristics, as well as islet number, size, distribution.

### Neurogenin 3-Cre Knock-Out

In other embodiments, Neurogenin 3-Cre mice can be generated to direct ablation of Ll in the endocrine progenitor cell in the pancreas and entero-endocrine system, after the endocrine/exocrine split has occurred, but prior to final specification of individual islet cell types. If LL plays a role in endocrine cell differentiation, the effects of its ablation can be determined in non-β cell types (α, δ, ε, PP). This can also drive inactivation of LL in entero-endocrine cells and result in inactivation of Ll in incretin-producing cells (K and L cells in the gut). Because incretin production is observed in diabetes, incretin response can be characterized in Neurogenin3-Cre/L1 knockouts (Buteau et al, 2006, Diabetes 55:1190-1196).

### Insulin-Cre Knock-Out

In other embodiments, Insulin-cre can inactivate Ll in terminally differentiated B6 cells. As such, the phenotype of these mice cann reflect the function of Ll in daily maintenance of the phenotype/function of B6 cells. This phenotype can resemble aspects of the diabetes susceptibility seen in DD mice. In certain embodiments, stress on the B6 cell can be imposed using standard approaches such as low-dose streptozotocin, high-dose dexamethasone, high-fat, high-sucrose diet, and partial pancreatectomy.

### Conditional Knock-Out in Liver

In other embodiments, Albumin-cre and α1-antitrypsin/cre mice can be used to generate L1 knock out in the liver. Albumin-cre and α1-antitrypsin/cre mice have been used to ablate genes in hepatocytes, with the α1-antitrypsin/cre line being being useful for earlier-onset ablation during fetal development, and the albumin-cre mice being useful for post-natal knockout (Postic et al, 2000, Genesis 26:149-150). Analyses of the knockout can be performed by protein- and mRNA-based expression assays.

The characterization of any of the knock out mice described herein, can include hepatic metabolism, hepatic glucose production (GTTs, hyperinsulinemic/euglycemic clamps, gene expression, pyruvate challenge tests) and lipid metabolism (Tota1 and Hd1 cholesterol, hepatic TG content, gene expression, ApoB levels and secretion using Triton inhibition of lipoprotein clearance; VLDL and LDL measurements by FPLC and ultracentrifugation will help identify variations in lipoprotein composition). The role of altered lipid metabolism in LL function can be examined the liver conditional Ll knockout mice.

### Ttr-Cre Knock-Out

In certain aspects, the invention provides unique liver/β-cell combination of expression driven by the transthyretin promoter to probe the role of the B6 cell/liver axis in metabolic control (Okamoto et al, 2004, J Clin Invest 114:214-223; Okamoto et al, 2006, J Clin Invest 116:775-782; Okamoto et al, 2005, J Clin Invest 115:1314-1322; Nakae et al, 2002, Nat Genet. 32:245-253). Because Ll is prominently expressed in liver and B6 cells, it can be useful to the generate of a double knockout driven by Ttr-cre to studying role the role of Ll in these tissues.

### Genetic and Environmental Interactions of the Ll Mutation

In addition to analyzing Ll mutant mice according to genetic background, the invention provides methods to determine the contribution of Ll loss-of-function to other forms of insulin-resistant diabetes. In certain aspects, dietary manipulations such as high fat and “Surwit” high fat-high sucrose diets can be used to examine the contribution of Ll to the environmental determinants of diabetes. The genetic component can be assessed by crossing Ll knockouts with Insulin Receptor heterozygous knockouts as a model of insulin resistance (Kido et al, 2000, J Clin Invest 105:199-205), or Irs2 knockouts (Kitamura et al, 2002, J Clin Invest 110:1839-1847), as a model of β-cell failure (Accili 2004, Diabetes 53:1633-1642).

### Metabolic Characterization

Metabolic characterization can be carried out for β cells, hepatocytes and other cell, tissue or organ of interest. Non-limiting examples of such tissues or organs are muscle, brain or the gut.

Conditional activation of Ll

Phenotypical analysis of mice carrying the ENU amber mutation can yield preliminary insights into the developmental phenotypes of Ll-deficient animals. Such Ll -nullizygous mice can be tailored to develop normally and show increased susceptibility to diabetes at early post-natal stages. Ll function can then be restored to alleviate or cure the disease. For example, if C57BL/6 Ll-deficient mice are viable and develop diabetes postnatally, tissue-specific reactivation of Ll expression can be used to rescue the phenotype. In certain embodiments, the invention provides a conditional re-activatable Ll allele generated by inserting a loxP-flanked STOP cassette consisting of an artificial splice acceptor site and a neomycin selection marker cassette into the first intron of the Ll gene (FIG. 20). In this approach the presence of the STOP cassette in intron 1 can cause splicing to this artificial exon and termination of transcription by the triple SV40 polyA signal to efficiently prevent expression of the Ll allele in the absence of cre (Hingorani et al, 2003, Cancer Cell 4:437-450; Ventura et al, 2007, Nature 445:661-665). Ll function can then be restored in a tissue-specific manner employing the cre lines used for conditional inactivation of the gene. In other aspects, the invention provides animals carrying one or more re-activatable alleles described herein.

The following examples illustrate the present invention, and are set forth to aid in the understanding of the invention, and should not be construed to limit in any way the scope of the invention as defined in the claims which follow thereafter.

### EXAMPLES

### Example 1

### Genetic Map of Diabetes QTL and Related Congenic Lines

A QTL for diabetes-related phenotypes was identified in obese F2 and F3 progeny of an intercross between diabetes-resistant (C57BL/6J) and diabetes-susceptible (DBA/2J) mice segregating for Lep^{ob}. Phenotypes including fasting blood glucose, HbA1c and islet histology mapped with LOD >8 around D1Mit110 on distal Chr 1@169.6 Mb (details in Methods, Mapping T2D-related Phenotypes). By producing congenic and sub-congenic B6.DBA lines also segregating for Lep^{ob}, the interval was refined to 5.0 Mb between rs31968429 at 168.1 Mb and rs31547961 at 173.1 Mb (FIG. 2) where all four congenic lines overlap for DBA (FIG. 2; details in Methods: B6.DBA Congenic Lines: Creation and Fine Mapping).

The search was further restricted (FIG. 2) by identifying a haplotype block (Wade C M, Kulbokas E J, 3rd, Kirby A W, Zody M C, Mullikin J C, et al. (2002) The mosaic structure of variation in the laboratory mouse genome. Nature 420: 574-578) conserved between B6 and DBA that extends 3.2 Mb from D1mit370 at 169.9 Mb to rs31547961 at 173.1 Mb. Only eleven unvalidated B6 vs. DBA SNPs in this interval are listed in the Mouse SNP database; however, among fragments we could amplify containing nine of these putative SNPs, no sequence variants were dectected. Moreover, no coding sequence/expression difference was found between B6 and DBA among all genes and transcripts in the “conserved” interval by computation, direct sequencing, and quantitative mRNA expression analysis. Thus, it is unlikely that the variant(s) in the genetically-defined interval with peak at 169.6 Mb mediating differential diabetes susceptibility between these two strains is within the “conserved region.” The 3 kb interval between rs31968429 and rs33860076 at the centromeric end of subcongenic line 1jcdt was sequences and no variants between the two strains were detected. Therefore, experiments were focused on the 1.8 Mb B6 vs. DBA “variable” interval, between rs33860076@168.1 Mb and D1mit370@169.9 Mb.

### Example 2

### Metabolic and Anatomic Phenotypes of Congenic Lines

T2DM can be a result of (1) ineffective glucose disposal and increased hepatic glucose production due to peripheral insulin resistance, and (2) relative hypoinsulinemia (DeFronzo et al. 1992, Diabetes Care 15:318-368). Obesity increases peripheral insulin resistance, by a combination of adipocyte-secreted proteins (Mora and Pessin, J. E. 2002. Diabetes Metab Res Rev 18:345-356), effects of free fatty acids (Boden, G., and Shulman, G. I. 2002. Eur J Clin Invest 32 Suppl 3:14-23) and other aspects of insulin signaling in liver and skeletal muscle (Kahn et al. 2006, Nature 444:840-846). Peripheral hyporesponsiveness to insulin increases metabolic demands on the β cell. Many obese individuals are insulin-resistant, but do not become overtly diabetic provided that increased demand for insulin is effectively met (Haffner, S. M. 2006, Obesity (Silver Spring) 14 Suppl 3:121 S-127S; Hossain et al, 2007, N Engl J Med 356:213-215). However, if beta cell mass or function is insufficient to meet this requirement, overt hyperglycemia and T2DM ensue (DeFronzo et al. 1992, Diabetes Care 15:318-368). In autopsy series of subjects with T2DM, total beta cell mass is decreased (Kloppel, et al. 1985. Sury Synth Pathol Res 4:110-125.10, 11). Primary reductions of beta cell mass predisposed to diabetes in some rodent models (Miralles et al, 2001 Diabetes 50 Suppl 1:S84-88; Leiter et al, 1989 Faseb J 3:2231-2241; Zucker et al, 1972, Endocrinology 90:1320-133012-14) and in some forms of MODY (maturity onset diabetes of youth) (Frayling et al, 2001 Diabetes 50 Suppl 1:S94-100). Such reductions can predispose to some instances of T2DM.

Susceptibility to T2DM is strongly inherited as evidenced by the >80% concordance rates in monozygotic twins (Barnett et al, 1981, Diabetologia 20:87-93; Lo et al, 1991, Diabetes Metab Rev 7:223-238; Kahn et al, 1996, Annu Rev Med 47:509-531; Medici et al, 1999, Diabetologia 42:146-150.), familial aggregation, and ethnic predispositions (June et al, 1999, Adv Drug Deliv Rev 35:157-177). Heritability of subphenotypes related to T2DM, for example, insulin resistance and β cell function is even higher (Permutt et al, 2005, J Clin Invest 115:1431-1439). Environmental factors also clearly play an important role in T2DM (Florez et al, 2003, Annu Rev Genomics Hum Genet. 4:257-291). Several genes for relatively rare monogenic forms of diabetes such as MODY, syndromic (Wolfram syndrome), lipoatrophic, and mitochondrial-inherited diabetes have been identified (Saltiel, 2001, Cell 104:517-529; Khanim et al, 2001, Hum Mutat 17:357-367). However, the underlying genetic basis for the more common and genetically complex T2DM, accounting for >95% of patients, has remained elusive.

In the neonatal rodent, remodeling of β cells occurs as a result of simultaneous activation of both apoptosis and β cell replication (Bonner-Weir, 2000, Endocrinology 141:1926-1929). Between 4 and 24 weeks, postnatally, β cell mass is estimated to increase 10 fold, related in part to increased body mass (Bonner-Weir, 2000, Endocrinology 141:1926-1929). Compensation for β cell stress/loss in adult rodents is primarily by β cell hypertrophy and β cell proliferation (Dor et al, 2004, Nature 429:41-46). In rats, β cell proliferation rates decline from −20% per day in pups, to −10% per day at 6-8 weeks, and to −2% shortly thereafter (Finegood et al, 1995, Diabetes 44:249-256). However, even this low rate of turnover apparently does not persist in adulthood. Using continuous long term BrdU labeling in C57x129Sv and BALB/C one year-old mice were shown to have extremely low replacement rates (˜ 1/1400 mature β cells/day) (Teta et al, 2005 Diabetes 54:2557-2567). These results show that β cell mass established in the first 6-8 weeks of life can be critical to the ability to meet subsequent stresses on β cell function imposed by obesity, hyperglycemia, etc. Based on this formulation, transient interruptions can result in permanent effects on cell mass or function or both (Hales and Barker 2001, Br Med Bull 60:5-20). Hypoactivity of the candidate T2D modifier gene (Lisch-like) can mediate such effects on establishment of initial β cell mass, and/or later responses of cell hypertrophy/replication by β cell-autonomous effects or in response to an exogenous ligand for this putative receptor.

To identify genes mediating differential susceptibility to diabetes in the context of obesity, C57BL/6J (resistant) and DBA/2J (susceptible). These are inbred strains that are discordant for type 2 diabetes when made obese (Coleman et al, 1973, 9:287-293). In obese F2 and F3 progeny of a B6/DBA cross segregating for Lep^{ob}, a quantitative trait locus (QTL) for T2DM associated with fasting blood glucose, glycosylated hemoglobin, and islet histology in 120-150 day old male mice was mapped to a region of Chr1. The peak statistical significance was at D1Mit 110 at 169.6 Mb from the centromere (p<10⁻⁸) (FIG. 2, Table 1 and Table 2).

In over 400 Lep^{ob/ob} F2 progeny of a C57BL/6J×DBA2J intercross, a DBA-related quantitative trait locus (QTL) was mapped to distal Chr1@169.6 Mb, centered about D1Mit110, for diabetes traits that included blood glucose, HbA1c and pancreatic islet histology. The interval was refined to 1.8 Mb in a series of B6.DBA congenic/subcongenic lines (to N15) also segregating for Lep^{ob}. The phenotypes of B6.DBA congenic mice included reduced beta cell replication rates at 1 day of age, reduced beta cell mass by 60 days, and mild hypoinsulinemic hyperglycemia up to 150 days of age.

The genetic interval on Chr1 to 0.5 cM (D1mit401@87.8 cM to D1mit370@88.3 cM) was refined by producing congenic and sub-congenic B6.DBA lines, and identifying diabetes endophenotypes that segregate as qualitative rather than quantitative traits. B6.DBA congenic mice were generated by intercrossing Lep^{ob}/Lep⁺ C57BL/6J and DBA/2J mice from Jackson Laboratory to generate F1 progeny, followed by backcrossing to the recurrent C57BL/6J strain using a speed congenic approach in subsequent generations (Visscher 1999, Genet Res 74:81-85). At the eighth backcross, a genomic scan was performed in all breeders using polymorphic markers at 20 cM intervals. In the mouse line that was continued, all non-contiguous markers outside the interval were homozygous B6. Over the next two generations, there were two recombination events, one that eliminated the telomeric DBA interval (line 1jc) and one that preserved approximately half of the originally defined interval (line 1jcd). The 1jcd mouse was bred repeatedly to B6 mice, giving rise, by meiotic recombination, to 2 additional subcongenic lines (1jcdt and 1 jcdc). Preservation of the phenotypes in the original B6.DBA and DBA.B6 F2/F3 progeny was assessed by longitudinal and end-point measurements of fasting glucose, insulin, glycosylated hemoglobin and islet morphology. At N12, ob/+ mice B6/DBA (B/D) for the congenic interval were intercrossed to produce N12F1 progeny. Obese progeny were used for fine mapping and phenotyping experiments. Ob/+ animals D/D for the congenic interval were recurrently intercrossed or crossed to B6 ob/+ animals to generate ob/ob animals with D/D and B/D genotypes for the Chr1 interval, respectively.

A schematic representation of the B6.DBA sub-congenic lines for the Chr1 interval segregating diabetes-related phenotypes is shown in FIG. 2. These lines display phenotypes of hypoinsulinemic hyperglycemia in association with histologic evidence of a relative reduction in β cell mass in the first 21 days of life due to reduced β cell proliferation. Phenotypes were more prominent in male animals. These phenotypes, by line, are described herein.

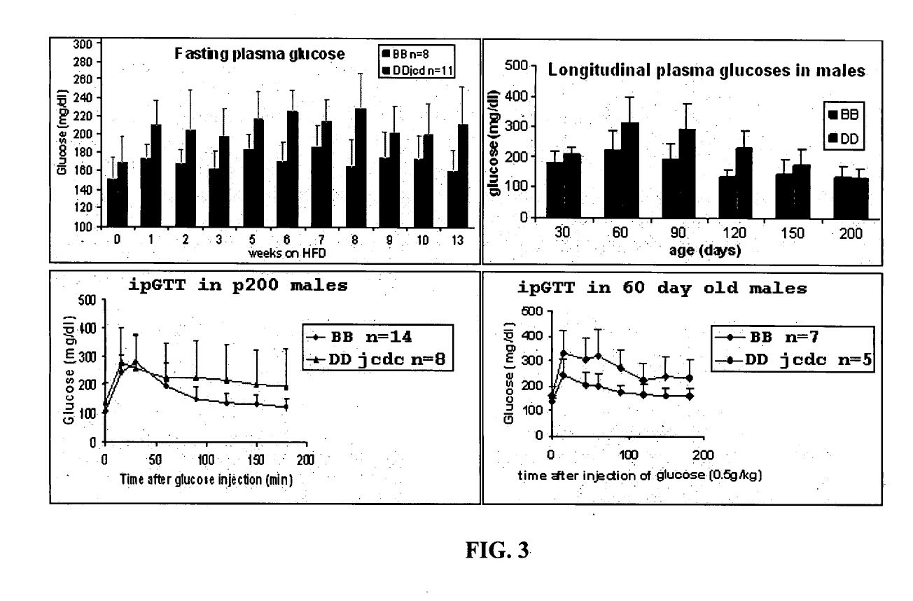

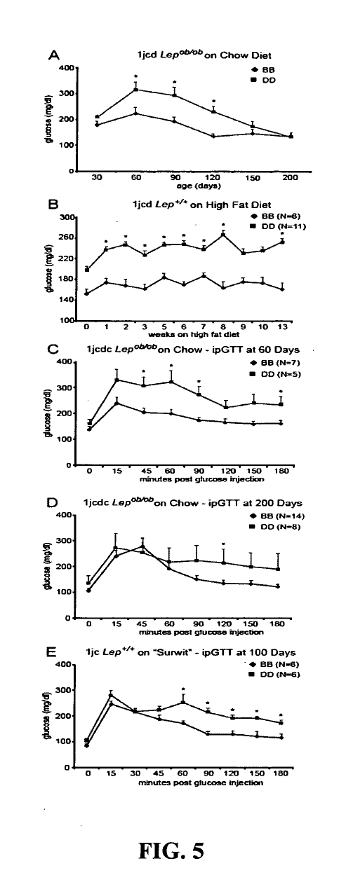

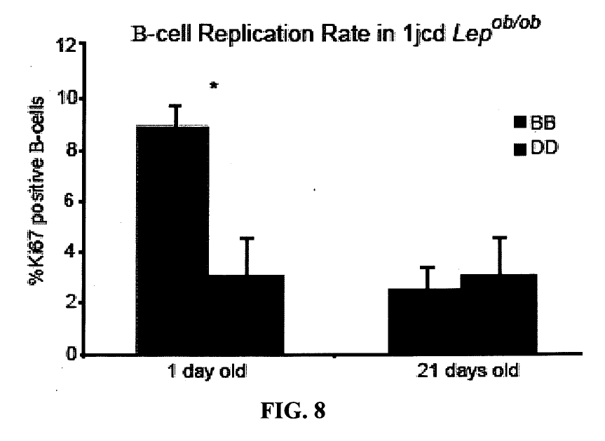

The congenic/subcongenic lines shown in FIG. 2 displayed phenotypes of hypoinsulinemic hyperglycemia in association with histological evidence of a relative reduction in beta-cell mass in the first 21-28 days of life due to reduced beta-cell proliferation (see FIGS. 7-8). Phenotypes were generally more salient in male animals. Genotype in the congenic interval (B6 or DBA) per se did not affect body weight or composition in the congenic lines as described herein. Elevations in fasting plasma glucose were observed by 4 weeks of age in Lep^{ob/ob} males on a standard (9% fat) chow diet who were D/D (D/D=DBA/DBA) for the congenic interval designated 1jcd in FIG. 2; these concentrations were higher up to 120 days. After 120 days, there were no significant differences in fasting glucose between D/D and B/B (B/B=B6/B6) mice (FIGS. 3, 5A and 35A). The decline in pre-prandial blood glucose levels in Lep^{ob/ob} males between 90 and 200 days is probably attributable to a slight expansion of β-cell mass in response to transient insulin resistance occurring as a normal consequence of sexual maturation (˜60 days of age) (Leiter E H (1989) The genetics of diabetes susceptibility in mice. FASEB J 3: 2231-2241; Leiter E H, Chapman H D, Coleman D L (1989) The influence of genetic background on the expression of mutations at the diabetes locus in the mouse. V. Interaction between the db gene and hepatic sex steroid sulfotransferases correlates with gender-dependent susceptibility to hyperglycemia. Endocrinology 124: 912-922). To examine diabetes susceptibility in D/D animals that were obese independent of leptin deficiency, lean (Lep^{+/+}) 1jcd males were fed a high-fat diet (60% kcal from fat) for 13 weeks, starting at 7 weeks of age. These D/D (Lep^{+/+}) mice became more hyperglycemic than B/B mice (FIGS. 5B and 35B), showing a persistence of this difference—similar to the animals in 2A—up to age ˜140 days when the study ended. Intraperitoneal glucose tolerance testing (ipGTT) was used to delineate acute differences in glucose handling between the D/D and B/B animals. Lep^{ob/ob} Ijcdc D/D males were less glucose tolerant than B/B by intraperitoneal glucose tolerance testing (ipGTT) at 60 days (FIGS. 5D and 35C), but were not significantly different from B/B by 200 days (FIG. 35D). 100-day old Lep^{+/+} 1jc D/D males who had been fed the Surwit (high fat, high sucrose) diet for 10 weeks were also more glucose intolerant than littermate B/B males (FIGS. 5E and 35E), indicating, again, that the Lep^{ob} was not necessary for the occurrence of the diabetes-related phenotype.

TABLE 1
Results from Analysis of Variance of terminal phenotypes by genotype at
D1Mit110 in 404 F2 Lep^{ob} / Lep^{ob} B6.DBA and DBA.B6 mice at 120-150 days of age.
Pancreatic grade is a subjective measure of number and size of islets and islet integrity
with grading from 1 (many, large, intact islets) to 5 (few, small islets with little insulin staining).

| | Female | | | Male | | | |
|---|---|---|---|---|---|---|---|
| | B/B | B/D | D/D | B/B | B/D | D/D | P |
| Weight (g) | 61.1 | 63.5 | 57.5 | 60.3 | 56.4 | 54.0 | 0.001 |
| HbA1c (%) | 8.7 | 9.6 | 12.2 | 11.6 | 14.1 | 14.3 | 0.00001 |
| [Glucose] (mg/dl) | 565 | 645 | 773 | 646 | 770 | 784 | 0.00001 |
| [Insulin] (μU/ml) | 258.3 | 272.2 | 178.5 | 235.1 | 83.1 | 148.1 | 0.01 |
| Pancreatic Grade | 2.4 | 2.7 | 3.7 | 2.7 | 3.5 | 3.6 | 0.00001 |
| Pancreatic [insulin] | 64674 | 35963 | 7696 | 15890 | 9445 | 8384 | 0.00001 |
| (μU/mg protein) | | | | | | | |
| Pancreatic | 0.8 | 0.54 | 0.12 | 0.25 | 0.17 | 0.13 | 0.00001 |
| [insulin]/[glucagon] | | | | | | | |
| No. islets | 18.0 | 17.6 | 11.5 | 13.6 | 9.8 | 10.9 | 0.005 |
| No. hyperplastic islets | 3.0 | 2.3 | 0.9 | 1.7 | 0.5 | 0.6 | 0.0001 |
| Average islet size (mm²) | 0.0248 | 0.0208 | 0.0171 | 0.022 | 0.0166 | 0.0171 | 0.00001 |
| Islet area (mm²) | 0.53 | 0.42 | 0.22 | 0.31 | 0.17 | 0.2 | 0.0001 |
| Islet area/total area (%) | 2.091 | 1.603 | 0.864 | 1.3 | 0.668 | 0.787 | 0.00001 |

Elevations in fasting plasma glucose were observed in ob/ob Ijcd D/D males by 4 weeks of age, and increased progressively to 90 days (FIG. 3). After 120 days, differences in fasting glucose between D/D and B/B mice were less pronounced (FIG. 3; upper right). To examine diabetes susceptibility in D/D animals that were obese independent of leptin deficiency, lean (Lep^{+/+}) 1jcd males were fed a high-fat diet (60% kcal from fat) for 13 weeks, starting at 7 weeks of age. Starting at 1 week after the high-fat diet treatment, and persisting throughout the 13-week study, D/D (Lep^{+/+}) mice were more hyperglycemic than B/B mice (FIG. 3; upper left). Lep^{ob/ob} Ijcd D/D males were glucose intolerant at 60 days (FIG. 3; lower right), but were not significantly different from B/B by 200 days (lower left). The hyperglycemia observed in D/D male mice was due to hypoinsulinemia, which is evident as early as 4 weeks in 1jc and 1jcd D/D animals. Genotype in the congenic interval (B or D) did not affect body weight of composition.

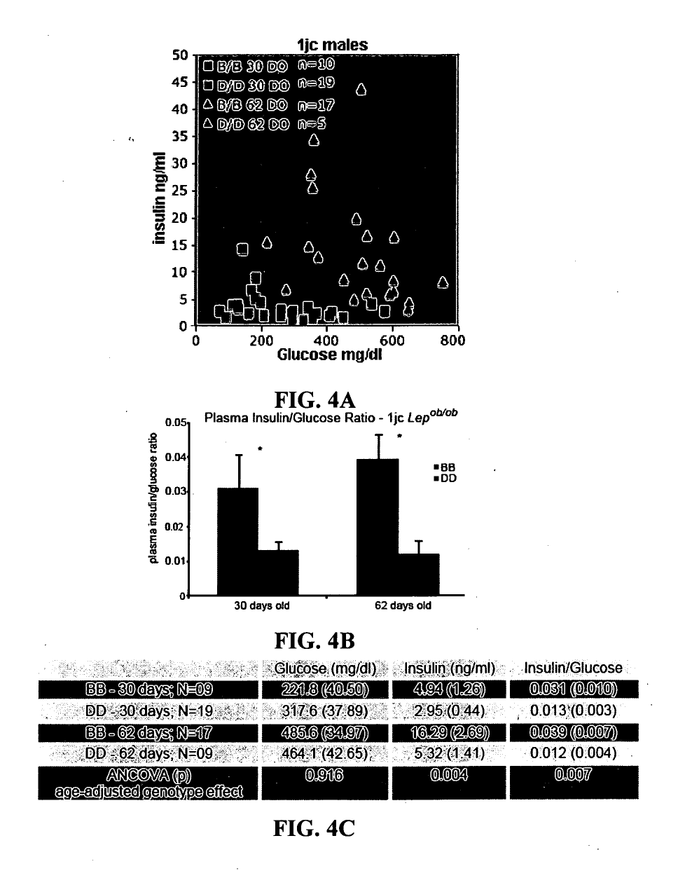

The hyperglycemia observed in D/D male mice was due to relative hypoinsulinemia, evident as early as 4 weeks in 1jc Lepob/ob D/D animals fed a chow diet (FIG. 4B). At mean ages of both 30 and 62 days of age, the D/D mice displayed lower age-adjusted plasma insulin concentrations per mg blood glucose than did the B/B animals (p=0.0003). This difference was due to age-adjusted genotype effects on plasma insulin: lower in D/D (p=0.0004) not higher blood glucose in D/D (p=0.916). Consistent with these ratios, D/D Lep^{+/+} males showed a 40% decrease in insulin secretion when clamped at a blood glucose level of 250 mg/dl for an hour (FIG. 37). No difference in insulin sensitivity was detected by euglycemic—hyperinsulinemic clamping.

The hyperglycemia observed in D/D male mice was due to relative hypoinsulinemia, evident as early as 4 weeks in 1jc and 1jcd Lep^{+/+}D/D animals fed the “Surwit” diet (FIG. 4A, FIG. 4B, and FIG. 4C). The D/D mice displayed lower plasma insulin concentrations per mg blood glucose that the B/B animals.

By 4 weeks of age, fasting plasma glucose was elevated in Lepob/ob males who were D/D (DBA/DBA) for the congenic interval 1jcd and fed standard (9% fat) chow; glucose concentrations were higher up to 120 days. After 120 days, there were no significant differences in fasting glucose between D/D (DBA/DBA) and B/B (B6/B6) mice (FIG. 5A). To examine diabetes susceptibility in D/D animals that were obese independent of leptin deficiency, lean (Lep+/+) 1jcd males were fed a high-fat diet (60% kcal from fat) for 13 weeks, starting at 7 weeks of age. These mice became more hyperglycemic than B/B mice (FIG. 5B), showing a persistence of this difference—similar to the animals in FIG. 5A—up to age ˜140 days when the study ended.

Intraperitoneal glucose tolerance testing (ipGTT), which was used to delineate differences in acute glucose handling between the D/D and B/B animals, showed that at 60 days Lepob/ob 1jcdc D/D males were less glucose tolerant than B/B (FIG. 5C), but by 200 days, strain differences were insignificant (FIG. 5D). 100-day old Lep+/+ 1jc D/D males fed the Surwit (high fat, high sucrose) diet for 10 weeks were also more glucose intolerant than littermate B/B males (FIG. 5E), indicating, again, that Lepob was not necessary for the occurrence of the diabetes-related phenotype.

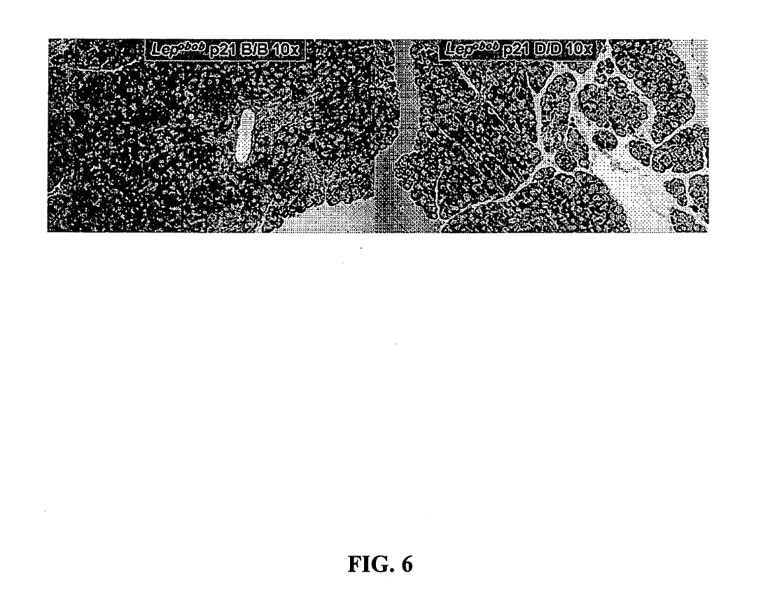

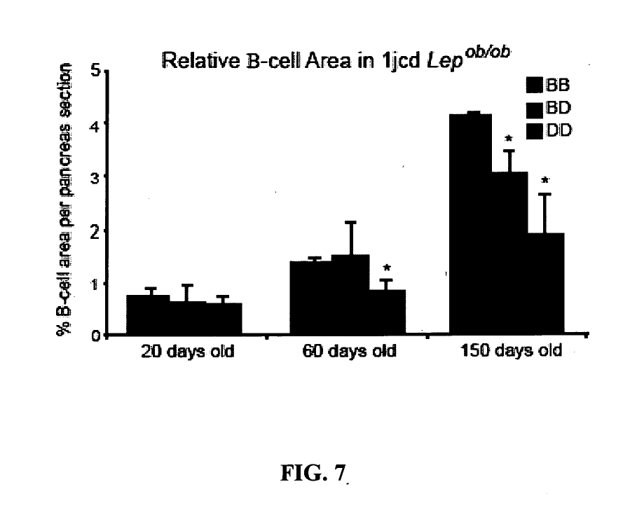

Consistent with their hypoinsulinemic hyperglycemia, 21 day old DD 1jcd males have smaller islets than their B/B counterparts (FIG. 6). Islets isolated from 28 day old D/D 1jcd males responded to graded glucose concentrations (2.8 mM-16.8 mM) or arginine 15 (10 mM) by secreting comparable amounts of insulin to age- and sex-matched B/B littermates. Thus, a qualitative cell-autonomous β-cell defect in insulin secretion is unlikely to be the primary functional defect in D/D animals, since islets isolated from 28-day old 1jcd D/D males responded to graded glucose concentrations (2.8 mM -16.8 mM) or 10 mM arginine by secreting amounts of insulin comparable to age- and sex-matched B/B littermates (FIG. 38). Also consistent with insulin/glucose ratios and hyperglycemic clamping results, isolated islets from 60 day old 1jc Lep^{ob/ob} males fed normal chow and 100-day old 1jc Lep +/+ on the Surwit diet showed reduced insulin secretion at 2.8 mM and 5.6 mM [glucose] in D/D vs. B/B littermates. For reasons indicated below, the early glucose intolerance of D/D mice is probably due, in part, to a deficiency of β-cell mass.

Islets of 60 day old D/D males released less insulin in response to graded concentrations of glucose (2.8 mM-16.7 mM), compared to B/B littermates, indicating the presence of a β cell defect that can be primary and/or reflect effects of higher in vivo ambient glucose or other hepatic effects in the D/D animals (Prentki, M., and Nolan, C. J. 2006. J Clin Invest 116:1802-1812.). There is an age-related decrease in the relative proportion of total pancreatic area occupied by β cells (Kido et al, 2000. J Clin Invest 105:199-205) in males segregating for the Ijcd D/D sub-congenic interval from 20- to 150 days of age, when β cell masses are half those of BB littermate controls, and BD animals have β cell masses that are ⅔ those of BB littermate controls. The fractional area of the pancreas accounted for by β-cells in Lepob/ob males segregating for the 1jcd D/D sub-congenic interval was examined in 20, 60 and 150 day-old mice. There was a trend to reduced β-cell area in DD by 60 days. By 150 days of age, β-cell mass of the 1jcd D/D sub-congenics was about half that of B/B littermate controls, and B/D animals had β-cell masses that were about two-thirds of B/B littermate controls (FIG. 7). These findings are consistent with in vivo data showing onset of elevated blood glucose and decreasing ipGTT (FIG. 5C) in D/D animals at ˜60 days of age, and persistence of hyperglycemia for a period thereafter, and lower circulating insulin concentrations (relative to glucose) in 1jc D/D sub-congenics at 60 days (FIG. 4B). The lower relative β-cell mass in D/D animals reflects fewer numbers of β-cells, rather than smaller sized β-cells. There were no differences in pancreatic weight between D/D and B/B male animals. These findings are consistent with in vivo data showing onset of elevated blood glucose and decreasing IPGTT (FIG. 3) in D/D animals at ˜60 days of age, and progressive hyperglycemia thereafter. The decrease in relative β cell mass in D/D animals is due to decreased numbers of individual β cells, rather than β cell size.

To assess the basis for the difference in β cell mass by 60 days, rates of β cell replication and apoptosis were measured. Pancreatic sections in Ijcd congenic 1- and 21-day old Lep^{ob}Lep^{ob} male mice were co-stained with insulin antibodies and Ki67, a nuclear marker of proliferation expressed during all stages of the cell cycle except G0 (Stanton et al, Am J Surg 186:486-492). The number of Ki67 positive β cells was normalized to the total number of insulin positive cells to estimate the proportion of dividing cells.

Each group consisted of 4 B/B and 4 D/D 1-day old mice and 4 B/B, and 8 D/D 21 day old mice. β cell replication in 1 day old D/D males was ˜⅓ that of B/B littermates (FIG. 8). This difference was not present in 21 day old animals due to normally reduced β cell replication by the time of weaning (Bonner-Weir, S. 2000, Endocrinology 141:1926-1929; Bonner-Weir, S. 2000, Trends Endocrinol Metab 11:375-378; Bonner-Weir, S. 2001, Diabetes 50 Suppl 1:S20-24). In lean (non ob/ob), 1 day old D/D males, the percentage of Ki67 positive β cells was ˜50% that of BB littermates (FIG. 7), indicating that this effect was not dependent on the absence of leptin (Covey et al. 2006, Cell Metab 4:291-302).

The proportion of small islets (250-2000 μm2) in 21 day old Lepob/ob males was greater in D/D Mc and 1jcd) mice (73%) than in B/B (60%); whereas the proportion of large islets (10,000-50,000 μm2) was lower (9% in D/D and 14% in B/B). This finding is consistent with the β-cell replication studies in P1 mice (FIG. 8), and recently reported evidence that β-cells are derived from replication of pre-existing β-cells.

At 13 days of age, when β cell apoptosis is active in mice (Scaglia et al. 1997, Endocrinology 138:1736-1741)—no significant differences between B/B and D/D islets in β cell apoptosis were detected using a TUNEL assay and caspase-3 staining. Thus, the lower number of β cells in D/D mice is a result of lower rates of proliferation of β cells in the perinatal period.

The strain-dependent susceptibility to T2DM in the context of monogenic obesity was apparent in the phenotypic differences between the original instances of what have since been identified as Leptin (Lep) and Leptin receptor (Lepr) mutations in mice (Coleman, 1982, 31:1-6). The original obese (Lep^{ob}) mutation arose in Stock V, but was transferred to the C57BL/6J background on which the mutation demonstrated obesity, transient hyperglycemia in puberty, and insulin resistance, but no sustained hyperglycemia (Leiter, 1989, Faseb J 3:2231-2241; Leibel et al, 1997, J Biol Chem 272:31937-31940). In contrast, mice homozygous for diabetes (Lepr^{db}) on the C57BL/KsJ strain background are as obese as Lep^{ob/ob} mice, but develop relative insulinopenia, profound T2DM, and die prematurely of their diabetes ((Leiter, 1989, Faseb J 3:2231-2241). A recent review by Clee and Attie (Clee and Attie 2006. The Genetic Landscape of Type 2 Diabetes in Mice. Endocr Rev.) provides a description of the effects of strain backgrounds on diabetes susceptibility in mice. During work on the cloning of the Lep (Friedman et al, 1991, Genomics 11:1054-1062) and Lepr (Chua et al, 1996, Science 271:994-996) genes, it was noted that there are differences in the occurrence of T2DM in Lep^{ob/ob} F2 progeny of B6/DBA intercrosses. The differential diabetes susceptibilities of the C57BL/6J and DBA/2J strains segregating for Lep^{ob} (Clee and Attie, 2006. The Genetic Landscape of Type 2 Diabetes in Mice. Endocr Rev) were exploited to identify diabetes susceptibility QTLs in B6xDBA progeny. Similar strategies have been used to identify QTLs (and responsible genes) for other complex phenotypes in mice (Flint, 2005, Nat Rev Genet. 6:271-286) such as type 1 diabetes (Todd 1999, Bioessays 21:164-174), diet-induced obesity (York et al, 1996, Mamm Genome 7:677-681), tuberculosis susceptibility (Mitsos et al, 2000, Genes Immun 1:467-477), atherosclerosis (Welch et al, 2001, Proc Natl Acad Sci USA 98:7946-7951), epilepsy (Legare et al, 2000, Genome Res 10:42-48), schizophrenia (Joober et al, 2002, Neuropsychopharmacology 27:765-781) and, most recently, T2DM (Clee et al, 2006, Nat Genet 38:688-693; Freeman et al, 2006, Cell Metab 3:35-45; Freeman et al, 2006, Diabetes 55:2153-2156). T2DM QTL's have been identified in rats (Chung et al, 1997, Genomics 41:332-344; Gauguier et al, 1996, Nat Genet 12:38-43).

### Example 3

### Islet Morphology and β-Cell Replication and Apoptosis

There was an age-related decrease in the fractional area of the pancreas accounted for by β-cells (Kido et al, 2000, J Clin Invest 105: 199-205) in males segregating for the Ijcd D/D sub-congenic interval from 20- to 150 days of age. By 150 days of age, β-cell mass of the D/D sub-congenics was approximately ½ that of B/B littermate controls, and B/D animals had β-cell masses that were approximately ⅔ those of B/B littermate controls (FIG. 7). These findings are consistent with in vivo data showing onset of elevated blood glucose and decreasing ipGTT (FIGS. 5A and 35) in D/D animals at ˜60 days of age, and persistence of hyperglycemia for a period thereafter, and reduced circulating insulin concentrations (relative to glucose) in D/D 1jc at 60 days (FIG. 4A, FIG. 4B and FIG. 4C). The decrease in relative β-cell mass in D/D animals was due to decreased numbers of individual β-cells, rather than decreased β-cell size. There were no differences in pancreatic weight between D/D and B/B male animals.

To assess the basis for the difference in β-cell mass by 60 days, rates of β-cell replication and apoptosis were measured. Pancreatic sections in Ijcd congenic 1- and 21-day old Lep^{ob/ob} male mice were co-stained with insulin antibodies and Ki67, a nuclear marker of proliferation expressed during all stages of the cell cycle except G0 (Stanton et al, 2003, Am J Surg 186: 486-492). The number of Ki67 positive β-cells was normalized to the total number of insulin positive cells to estimate the proportion of dividing β-cells. Each group consisted of 4 B/B and 4 D/D 1-day old mice and 4 B/B, and 8 D/D 21-day old mice. β-cell replication in 1-day old D/D males was ˜⅓ that of B/B littermates (FIG. 8). This difference was not present in 21-day old animals as a result of normally reduced β-cell replication by the time of weaning (Bonner-Weir 2000, Endocrinol Metab 11: 375-378; Bonner-Weir 2000, Endocrinology 141: 1926-1929; Bonner-Weir 2001, Diabetes 50 Suppl 1: S20-24).

In lean (non Lep^{ob/ob}) 1-day old D/D males, the percentage of Ki67 positive β-cells was ˜50% that of B/B littermates, indicating that this effect was not dependent on the absence of leptin (Covey et al, 2006, Cell Metab 4: 291-302). Compared to Lep^{ob/ob} B/B animals, Lep^{ob/ob} Ijc and Ijcd D/D males also had a significantly greater number of very small islets (250-2000 mm²) (72-73% D/D vs. 60% B/B), and fewer medium-sized and large-sized islets. The proportion of small islets (250-2000 mm²) in 21 day old Lep^{ob/ob} males was greater in D/D Mc and 1jcd) mice (73%) than in B/B (60%); whereas the proportion of large islets (10,000-50,000 μm²) was lower (9% in D/D and 14% in B/B). This finding is consistent with the β-cell replication studies in P1 mice (FIG. 8), and recently reported evidence that new β-cells are derived from replication of pre-existing β-cells (Dor Y, Brown J, Martinez O I, Melton D A (2004) Adult pancreatic beta-cells are formed by self-duplication rather than stem-cell differentiation. Nature 429: 41-46).

At 13 days of age, when β-cell apoptosis is active in mice (Scaglia et al, 1997, Endocrinology 138: 1736-1741), no significant differences were detected between B/B and D/D islets in β-cell apoptosis using a TUNEL assay and caspase-3 staining. Thus, the smaller number of β-cells in D/D mice is primarily a result of lower rates of proliferation of β-cells in the perinatal period.

### Example 4

### Defining the Minimum Genetic Interval for Diabetes-Susceptibility

The four congenic lines overlap for DBA in the 5.0 Mb interval corresponding to line 1jcdt (between rs31968429 at 168.1 Mb and rs31547961 at 173.1 Mb). The candidate-gene interval was further narrowed by identifying a haplotype block (Wade et al, 2002, Nature 420: 574-578) conserved between B6 and DBA that extends 3.2 Mb from D1mit370 at 169.9 Mb to rs31547961 at 173.1 Mb. Only eleven potential B6 vs. DBA SNPs in this interval are listed in the Mouse SNP database and, since all are “called” from only one sequence trace for the DBA or B6 variant, their validity is suspect. Among fragments containing nine of these putative SNPs that were amplified, no sequence variants were detected. Moreover, no coding sequence/expression difference was found between B6 and DBA among genes and transcripts in the “conserved” interval by computation, direct sequencing, and quantitative mRNA expression analysis. Thus, the variant(s) in the genetically-defined interval with peak at 169.6 Mb controlling differential diabetes susceptibility is not between these two strains is within the “conserved region.” The 3 kb between rs31968429 and rs33860076 at the centromeric end of 1jcdt were also sequenced and no sequence variants were detected between the two strains. Therefore, efforts were focused on the 1.8 Mb B6 vs. DBA “variable” interval, between rs33860076 (Cen) and D1mit370 (Tel) (FIG. 2).

### Example 5

### Genes in the Minimal DBA Interval Conveying Diabetes Susceptibility

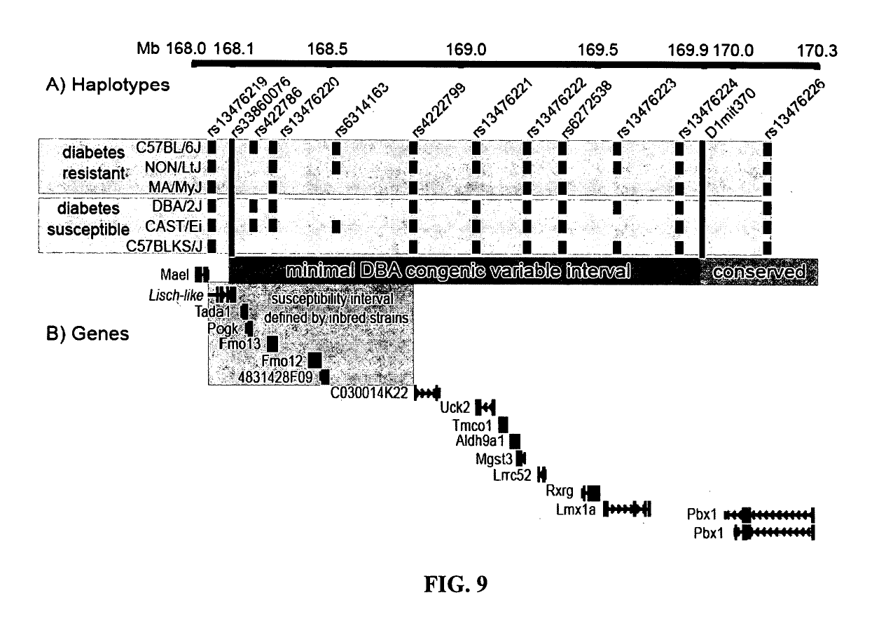

The region between 169.9-170.3 Mb in DBA v B6 is invariant (FIG. 2), does not contain the gene(s) of interest. The “variable” interval from 168.1-169.9 Mb contains 14 genes (FIG. 9) flanked by the genes Mael and Pbx1. Eleven genes are listed in RefSeq, and three predicted genes (chr1.1224.1 and FMOs 12 and 13), were confirmed in this study by rtPCR amplification of full-length transcripts from cDNA libraries. All 11 RefSeq genes, and three predicted genes (chr1.1224.1 and FMOs 12 and 13) were confirmed by rtPCR amplification of full-length transcripts from cDNA libraries. By identifying shared haplotypes in strains susceptible or resistant to diabetes (Clee et al, 2006, Nat Genet. 38: 688-693), the interval was further refined to 920 kb from rs4222799 to rs13476219 that includes just seven of these genes (FIG. 9B). Nonetheless, transcripts in the variable region were analyzed.

Within the “variable” interval, shared haplotypes in strains susceptible or resistant to diabetes (Friedman et al. 1991, Genomics 11:1054-1062) identify a 920 kb sub-interval that includes just seven genes and five amino acid substitutions within the congenic interval. Nonetheless transcripts in the entire “variable” region were analyzed. The prime candidate chr1.1224.1 (“Lisch-like”) is in the haplotype-reduced sub-interval.

### Example 6

### Computational Analysis of the “Variable” Interval 168.1-169.9 Mb

To identify genes in the minimal DBA interval, 277 genes and transcripts, computationally predicted by GenScan, TwinScan, FGeneSH, Otto, or SGP2, were screened and deposited into a structured query language database and manually curated. 50 single-exon transcripts were not further analyzed—these genes can be pseudogenes (Wang et al, 2003, Nat Rev Genet. 4:741-749)—which did not belong to a transcript cluster and were not homologous to transcripts in the syntenic human interval. 16 ribosomal gene transcripts unique to this interval, that were not specifically amplified due to genomic redundancy were not further analyzed. Of the remaining 211 predicted transcripts, 63 that did not amplify in RNA/cDNA pools from multiple organs/ages of B6 and DBA mice were rejected, and 148 were confirmed (see Methods: Testing for Predicted Transcripts in cDNA Pools). Using BLASTn, these 148 transcripts were clustered into 18 groups, corresponding to 5 predicted genes that were validated by amplification in cDNA pools and 13 known genes (Table 2). Subsequent refinement of the interval reduced the list in Table 2 to the 14 genes shown in FIG. 9. A map of the “variable” interval shows 14 genes, flanked by Mael and Pbx1 (FIG. 9). By identifying shared haplotypes (FIG. 9A) in strains susceptible or resistant to diabetes, the interval was refined to 677 kb from rs33860076 at the centromeric boundary of the minimal DBA congenic boundary to rs13476221 to include just six of these genes (FIG. 9B). Nonetheless, transcripts in the entire “variable” region were analyzed.

Analysis of Genes in the Variable Interval.

The genetic variation accounting for differential diabetes-susceptibility in mice segregating B/B vs. D/D in the congenic intervals can be due to (1) coding sequence variant(s) that alter the amino acid sequence of a protein (or proteins) and/or (2) regulatory variants, including anti-sense transcripts that affect expression and stability, and 3′ untranslated region (UTR) variants; and/or (3) splicing variants.

Non-Synonomous Sequence Variants.

Computational methods were used to identify non-synonymous B6/DBA single nucleotide DNA sequence variants within the 168.1-169.9 Mb interval. Genomic sequence for B6 and DBA strains were collected from databases at NCBI and Celera (Lindblad-Toh et al, 2001, Genesis 31:137-141), and any sequence gaps were filled using bi-directional sequencing to achieve 100% coverage of coding sequences in both strains. Coding sequence variants were validated by bi-directionally re-sequencing gene fragments encompassing each variant in both B6 and DBA strains. Consequently, the following non-synonymous single nucleotide variants were found: one in each of three FMO-like (flavin mono-oxygenase) genes, and two variants in chr1.1224.1 (FIG. 9 and Table 2). The latter gene, was designated “Lisch-like” (L1) because of its sequence similarity to a gene in mouse and rat, formerly known as Lisch7, but now known as Lsr (lipolysis stimulated receptor).

Computational analysis of LL and the three FMO-like proteins using SNAP (Bromberg Y, Rost B (2007) SNAP: predict effect of non-synonymous polymorphisms on function. Nucleic Acids Res 35: 3823-3835), PolyPhen (Ramensky V, Bork P, Sunyaev S (2002) Human non-synonymous SNPs: server and survey. Nucleic Acids Res 30: 3894-3900), SIFT (Ng P C, Henikoff S (2003) SIFT: Predicting amino acid changes that affect protein function. Nucleic Acids Res 31: 3812-3814), PAM250 matrix substitution weights (Dayhoff M (1978) Atlas of Protein Sequence and Structure. In: Dayhoff M, editor. National Biochemical Research Foundation. Washington, D.C. pp. 353-358) and PROFacc (Rost B, Sander C (1994) Combining evolutionary information and neural networks to predict protein secondary structure. Proteins 19: 55-72) predicted that all of the amino acid substitutions were benign with respect to function. The SNAP scores obtained for our variant alleles, −1 (FMO13, K282E), −2 (FMO12, V239I), −3 (LL, A647V), and −6 (LL, T587A; FMO9, Q5R), indicate that there is a ˜60%, ˜69%, ˜79%, and ˜90% respective chance of the non-synonymous variants being neutral. Similarly, PolyPhen classified all variations as “benign” and SIFT scores were well above 0.05 (neutral). PAM weights of 0 and above suggest interchangeability of the respective amino acids throughout evolution. The % differences were low, suggesting that the DBA and B6 variants are equally likely to occur in related sequences (see Methods: Computational Methods for Evaluating Effects of nsSNPs).

There are two non-synonymous SNPs in Ll within the region of overlap among the congenic lines, in exon 9. However, their effects on protein function are predicted to be minor and it is unlikely that they determine the differences in either transcript abundance or protein level seen in the congenics. Variants in other intervals are more likely relevant.

In the 5′ UTR, all but one of the eight variants are in simple repeats, where they are likely less significant. The interval underlying the anti-sense transcript contains 45 D/B variants, including a long, unique insertion. A regulatory role for the Ll anti-sense transcript is suggested by the similar location of anti-sense transcripts at the 3′ ends of the human C1ORF32 (human ortholog of Ll) gene (e.g., DA322725 from hippocampus), the human LSR gene (DA320945, also from hippocampus), the human ILDR1 gene (AW851103), and the mouse Lsr gene (BY747866). Moreover, comparative inter-species transcriptomic analysis has identified the 3′ regions of transcripts as important in anti-sense regulation, and conserved overlap between species may be evidence of function (Numata K, Okada Y, Saito R, Kiyosawa H, Kanai A, et al. (2007) Comparative analysis of cis-encoded antisense RNAs in eukaryotes. Gene 392: 134-141). For a recent review of anti-sense regulatory mechanisms, see (Lapidot M, Pilpel Y (2006) Genome-wide natural antisense transcription: coupling its regulation to its different regulatory mechanisms. EMBO Rep 7: 1216-1222).

### Example 7

### Identification of Chr1.1224.1 (Ll) as Primary Candidate Gene

For each gene and confirmed transcript, expression in tissues and organs key to diabetes (pancreatic islets, liver, skeletal muscle, adipose tissue and hypothalamus/brain) were quantified in 28-day old Lep^{ob/ob} male D/D and B/B 1jc animals. 28 day-old mice were chosen because D/D animals at this age are not yet diabetic. Thus, transcriptional differences represent primary effects, as opposed to changes induced by metabolic derangements due to overt T2DM. Real-time qPCR results for the genes within the variable region of the interval are summarized in Table 5 in the column designated “transcript ratio.”

Affymetrix microarrays were used to quantify those transcripts in the minimum congenic interval that had been validated by PCR-amplification (see Methods: Testing for Predicted Transcripts in cDNA Pools). Hypothalamus, islets, liver, soleus and EDL skeletal muscle from DD and BB Lep^{ob/ob} congenic animals were examined (see Methods: Microarray Gene Expression Analysis). These arrays did not contain elements for all of the 14 genes we confirmed in the interval: missing from the array were the 3 FMO genes. Therefore, real-time qPCR was also used to quantify expression of each gene and confirmed transcript in tissues and organs central to diabetes (pancreatic islets, liver, skeletal muscle, adipose tissue and hypothalamus) in 90-day old male Lep^{ob/ob} 1jc D/D and B/B animals (see Methods: real time qPCR). Results of the microarray and qPCR experiments are shown in the table below and summarized in FIG. 39.

BB/DD Transcript Ratios of Genes in the Variable Interval 168.1-169.9 Mb.

| | BB/DD Transcript ratio | | | | | | | | | |
|---|---|---|---|---|---|---|---|---|---|---|
| | Liver | | Brain | | Islets | | EDL | Soleus | | |
| Confirmed | μ- | | μ- | | μ- | | μ- | μ- | Muscle | Adipose |
| Genes | array | qPCR | array | qPCR | array | qPCR | array | array | qPCR | qPCR |
| chr1.1224.1 | 2.5 | 40 | 1.6 | 2.1 | 2.9 | 2.9 | 2.6 | 4.1 | 2.9 | 2.7 |
| (Lisch-like) | 9 × 10⁻⁴ | | 5 × 10⁻⁷ | | 2 × 10⁻³ | | 4 × 10⁻⁴ | 7 × 10⁻⁴ | | |
| Lisch-like | 0.5 | | 0.3 | | NE | | NE | NE | | |
| antisense | 6 × 10⁻³ | | 3 × 10⁻⁹ | | | | | | | |
| Tada1l | 1 | 1.4 | 1.4 | 0.9 | 0.9 | 1.05 | 1.1 | 0.9 | 0.5 | 1.1 |
| | NS | | 1 × 10⁻⁸ | | NS | | NS | NS | | |
| Pogk | .9 | 1.3 | .8 | 0.73 | 1.0 | 1.2 | 1.0 | 0.9 | 0.7 | 1.05 |
| | NS | | 1 × 10⁻³ | | NS | | NS | NS | | |
| FMO13 | Not on array- “inclusive-only” | | | | | | | | | |
| FMO12 | Not on array- “inclusive-only” | | | | | | | | | |
| FMO9 | Not on array- “inclusive-only” | | | | | | | | | |
| C030014K22 | NE | 1.1 | NE | 1.9 | NE | NE | NE | NE | 1.4 | 1.1 |
| Uck2 | NE | 1.4 | 0.6 | 0.8 | 1.4 | 1.2 | 1.0 | 1.3 | 0.7 | 0.8 |
| | | | NS | | 2 × 10−2 | | NS | NS | | |
| Tmco1 | 0.8 | 1.5 | 0.9 | 0.8 | 0.7 | 1.0 | 0.8 | 0.9 | 0.7 | 1.5 |
| | 2 × 10⁻⁵ | | 3 × 10⁻⁴ | | 2 × 10⁻⁴ | | 2 × 10⁻² | 3 × 10⁻² | | |
| Aldh9a1 | 1.4 | 1.3 | 1.5 | 1.4 | 1.9 | 1.5 | 1.6 | 1.7 | 1.3 | 1.6 |
| | 1 × 10⁻⁶ | | 3 × 10⁻⁷ | | 4 × 10⁻⁵ | | 3 × 10⁻⁴ | 3 × 10⁻² | | |
| Mgst3 | 0.4 | 1.0 | 0.6 | 0.6 | 1.3 | 0.8 | 0.7 | 0.8 | 0.6 | 0.7 |
| | 1 × 10⁻⁹ | | 2 × 10⁻⁸ | | NS | | 7 × 10⁻⁶ | NS | | |
| Lrrc52 | NE | | NE | | NE | | NE | NE | | |
| Rxrg | NE | 0.7 | NE | 1.6 | NE | 1.0 | 1.2 | NE | 0.9 | 1.0 |
| | | | | | | | NS | | | |
| Lmx1a | NE | nd | NE | 2.0 | NE | 0.9 | NE | NE | 1.0 | 1.7 |

Tissue-specific cDNAs of genes in the “variable interval” (see FIG. 9) from 10 21-day old DD and BB Lep^{ob/ob} congenic animals were analyzed using Affymetrix #430A microarray (μ-array), as described in Methods (see “Microarray Gene Expression Analysis”). Samples for qPCR analysis (see “Real-time qPCR” in Methods), were prepared from 5 BB and 5 DD 90-day old Lep^{ob/ob} 1jc males on 2 occasions. RefSeq genes are in bold type; predicted transcripts, locally-confirmed, are in regular type. “Inclusive only” transcripts were detected only in a cDNA pool that included whole embryos, 1-day old pups, and other tissues, but not in the cDNA pool prepared from diabetes-relevant organs. Probes for these genes were neither on the array nor analyzed by qPCR. Primer-pairs used for this analysis amplify transcripts of Ll isoforms 1, 2, 4, and 5 (see FIG. 21 and associated text), which, collectively, comprise >90% of the total number of transcripts of all Ll isoforms. NE; not expressed. NS; not significant p<0.05). In microarray analysis, the ratio, is the average B/B signal divided by the average D/D signal in the organ; in qPCR, ratios represent transcript copies. Number on lower line of microarray cells is p-value, 2-sided t-test, comparing the set of 10 BB mice in the specific organ to the set of 10 DD mice in the same organ.

Several genes within the region, including Lmx1a (German et al, 2004, Genomics 24: 403-404), and Rxrg (Hsieh et al, 2006, Hum Mol Genet. 15: 2701-2708), constitute candidates for susceptibility to T2DM; however, no nsSNPs were identified in these genes, and no multi-organ differences in expression levels were appreciated between B/B and D/D animals.

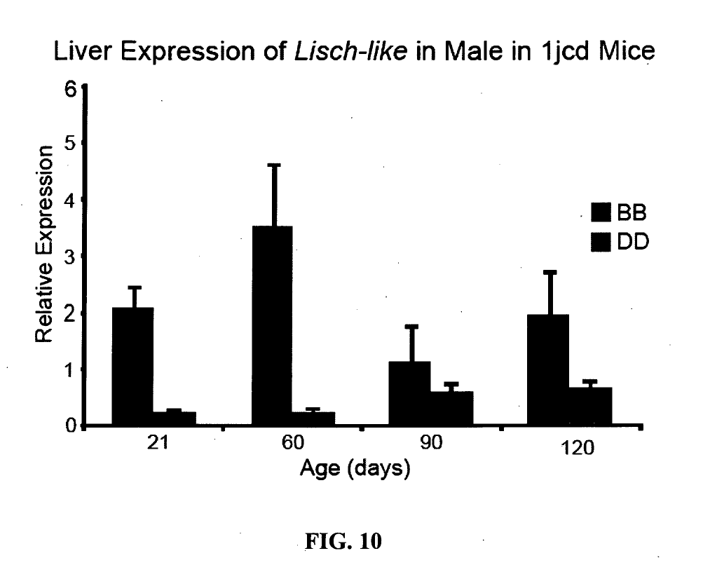

The most prominent differences in expression were observed for chr1.1224.1 (Ll) which was two to four-fold lower in 21-day old Lep^{ob/ob} D/D mice than in B/B mice in the diabetes-relevant tissues/organs by microarray analysis and up to twenty-fold lower by qPCR (FIG. 39). (Also shown herein is that Ll protein in hypothalamus is reduced in 1jc D/D vs. B/B; see FIG. 41A). The difference in Ll gene expression in liver persists with age (FIG. 10) as does the difference in glucose tolerance in response to overt glucose challenge see FIG. 5D). Whether the differences in hepatic Ll expression are related to differences in glucose homeostasis are unknown at this point; LL may influence hepatic gluconeogensis, or the hepatic differences could simply mirror parallel and more physiological relevant changes in β-cells. which was significantly lower in diabetes-relevant tissues/organs studied (liver, pancreatic islets, skeletal muscle, brain and adipose tissue) in 28-day old Lep^{ob/ob} D/D (vs. B/B) mice (Table 5). Chr1.1224.1 mRNA was lower in the livers of the 1jc subcongenic line Lep^{ob/ob} D/D vs. B/B males at the ages studied (21 days, 60 days, 90 days, 120 days) (FIG. 10).

The Chr1.1224.1 gene is within the minimum DBA interval (crossing the centromeric boundary of lines 1jcdc, 1jcd and 1jcdt), showed expression differences consistent with a role in diabetes-susceptibility, and has amino acid sequence variants between DBA and B6. It thus qualified as a candidate diabetes-susceptibility gene. Using primer-pairs flanking the first and last predicted exons, transcripts including coding sequences for chr1.1224.1 were amplified from B6 and DBA cDNA libraries from a wide range of tissue types. The gene was designated “Lisch-like” (Ll) because of its sequence similarity to a gene in mouse and rat, formerly known as Lisch7, but now known as Lsr (lipolysis stimulated receptor protein). The rat Lsr gene product is a predicted membrane-bound protein that has a high affinity for chylomicrons and very low density lipoproteins, is primarily expressed in the liver, and is “activated” by free fatty acids (Yen et al, 1999, J Biol Chem 274: 13390-13398).

With regard to Ll, the subcongenic lines investigated have the important characteristic that three of the lines (1jcd , 1jcdt and 1jcdc) contain DBA DNA only 3′ of exon 7, while line Ijc is DBA for the entire gene and actually extends (DBA) another 3 Mb 5′ of Ll. One inference is that coding and/or non-coding DBA v. B6 variant(s) in the region of DBA overlap among the congenic lines accounts for the phenotypic differences between the DBA congenic lines and animals segregating for B6 alleles in this region. In the region of overlap that includes the DBA v. B6 variable region (FIG. 9), Ll is the gene showing anticipated differences in coding sequence, gene expression and protein levels by IHC. These findings strongly support the role of Ll alleles in conveying the phenotypic differences seen between the various DD and BB congenic lines. The phenotypes of the Ll W87* C3H mice also support the finding regarding the candidacy of Ll based upon the B.D congenics. Other candidate genes in the interval (e.g., Pbx1 and Rxrg) were eliminated by examination of transcript and protein levels.

Computational analysis of LL and the three FMO-like proteins using SNAP and PROFacc (Rost and Sander1994, Proteins 19: 55-72) predicted that the amino acid substitutions were benign with respect to function. The SNAP scores attained for the variant alleles described herein, −1 (FMO13, K282E), −2 (FMO12, V239I), −3 LL, A647V), and −6 (LL, T587A; FMO9, Q5R), indicate that there is a ˜60%, ˜69%, ˜79%, and ˜90% respective chance of the non-synonymous variants being neutral. Similarly PolyPhen classified the variations as “benign” and SIFT scores were well above 0.05 (neutral). PAM weights of 0 and above indicate interchangeability of the given amino acids throughout evolution. The percentage differences are low showing that the DBA and B6 variants can occur in related sequences.

Computational analysis of amino acid substitutions in 4 genes using SNAP (Bromberg, Y. a. R., B. 2006. SNAP: prediction of functional effects of non-synonymous polymorphisms), PolyPhen (Ramensky et al. 2002, Nucleic Acids Res 30:3894-3900), SIFT (Ng and Henikoff 2003, Nucleic Acids Res 31:3812-3814), PAM250 matrix substitution weights (Dayhoff, M. 1978. Atlas of Protein Sequence and Structure. Washington, D.C.: National Biochemical Research Foundation. 6 pp) and PROFacc (Rost and Sander, 1994, Proteins 19:55-72), indicated that these amino acid changes were detrained to not affect function. Nonetheless, these variants can be tested for functional effects.

Several genes within the region, including Lmx1a (German et al, 1994, Genomics 24:403-404), and Rxrg (Hsieh et al, 1996, Hum Mol Genet. 15:2701-2708), constitute candidates for susceptibility to T2DM; however, no nsSNPs were identified in these genes and no multi-organ differences in expression levels (or protein expression as determined by western) were appreciated between B/B and D/D animals (Table 2).

The most prominent differences in expression were observed in chr1.1224.1 which was two to ten fold lower in diabetes-relevant tissues/organs studied (liver, pancreatic islets, skeletal muscle, brain and adipose tissue) in 28 day old Lep^{ob/ob} D/D (v. B/B) mice. Chr1.1224.1 mRNA was down-regulated in the livers of the 1jc subcongenic line Lep^{ob/ob} D/D v. B/B males at the ages studied (21 days, 60 days, 90 days, 200 days) (FIG. 10). Moreover, L1 expression was significantly lower in the livers of Lepob/ob 1jc D/D vs. B/B males at 21 and 60 days of age, with a tendency to recover by 90-200 days, in conjunction with improvements in glucose homeostasis (FIG. 10). L1 transcript were also detected in e7, e11, e15, and e17 whole mouse embryos and in testis, kidney, heart, lung, uterus, eye, thymus and spleen. For the anti-sense interval between intron 9 and intron 7 (see below and FIGS. 2 and 21), higher expression levels were found in liver and hypothalamus of D/D v. B/B animals. This difference is consistent with a possible suppressive role for the D/D anti-sense transcript (see below). The Aldh9a gene, known to be highly expressed in human embryonic brain and involved in glycolysis and fatty acid metabolism, showed qualitative changes comparable to those seen in L1. The mapping experiment that identified the interval of mouse Chr1 containing statistical signals related to T2D phenotypes, would be expected to enrich for regions in which several genes might contribute to the phenotypes. It is possible that Aldh9a is such a gene. Ll showed the most quantitative differences between D/D and B/B animals. In 21-day old 1jcd males, the D/D animals showed a 3-6 fold greater expression of the anti-sense transcript in islets, brain and liver than B/B (probe ID, Affymetrix MOE430-2 microarrays).

For each gene and confirmed transcript, expression in tissues and organs key to diabetes (pancreatic islets, liver, skeletal muscle, adipose tissue and hypothalamus/brain) were quantified in 28 day old Lep^{ob/ob} male D/D and B/B 1jc animals using real-time qPCR. 28 day-old mice were chosen because D/D animals at this age are not yet diabetic. Thus, transcriptional differences can represent primary effects, rather than changes induced by metabolic derangements due to overt T2DM. Real-time qPCR results for the genes within the variable region of the interval are summarized in Table 2.

TABLE 2
Summary of variants in the 168.1-169.9 Mb interval. “Genes” include known and confirmed
predicted transcripts. Amino acid changes were confirmed by bidirectional sequencing in both strains.
Transcript ratios determined by qRTPCR analysis, using a Roche LifeCycler 2.0, normalized to actin,
in the 1jc congenic line. Tissue-specific cDNA pools were prepared from five animals of each genotype
(BB, DD) ob/ob on 2 occasions. Transcript ratios were reproducible across the pools.

| | | Amino acid | | |
|---|---|---|---|---|
| | | changes | | Transcript ratio |
| Gene | Type | B6 > DBA | name/gene family/annotation | BB/DD > 2 |
| chr1.1224.1 | predicted | T572A | Lisch-like | Liver >10x |
| | | A632V | (lipolysis-stimulated remnant | Adipose 2x |
| | | | receptor-related) | Brain 2x |
| | | | | Islets 2x |
| | | | | Muscle 2x |
| Tadal1 | known | none | SPT3-associated factor 42 | Same |
| Pogk | known | none | pogo transposable element with | Same |
| | | | KRAB domain | |
| LOC226601 | predicted | K282E | flavin-containing monooxygenase | * |
| (FMO13) | | | family; FMO-like | |
| 4831428F09 | known | Q5R | flavin-containing monooxygenase | * |
| | | | family; FMO-like | |
| LOC226604 | predicted | V239I | flavin-containing monooxygenase | * |
| (FMO12) | | | family; FMO-like | |
| C030014K22Rik | known | none | unknown | Same |
| Uck2 | known | none | uridine monophosphate kinase | Same |
| Tmco1 | known | none | membrane protein of unk. function | Same |
| Aldh9a1 | known | none | Aldehyde dehydrogenase 9, subfamily A1 | Same |
| Mgst3 | known | none | microsomal glutathione-S-transferase 3 | Same |
| Lrrc52 | predicted | none | Leucine-rich repeat (LRR) protein of | Same |
| | | | unk. function | |
| Rxrg | known | none | retinoid X receptor, gamma | Brain 2x |
| Lmx1a | known | none | LIM homeobox transcription factor 1, α | Brain 0.5x |

Asterisk (*)indicates failure to detect the transcript in a cDNA pool of diabetes-relevant tissues and organs from 4 week-old male mice (pancreatic islets, liver, skeletal muscle, brain and adipose tissue). These asterisked transcripts were only present in a cDNA pool consisting of large intestine, small intestine, eyes, skin, tongue, spinal cord, kidney, testes/ovaries, E7 fetuses, E20 fetuses, p1 pups, (“same” indicates no detectable difference in expression BB v. DD in pancreatic islets, brain, liver and adipose tissue, brain of 28-day old mice).

### Example 8

### “Lisch-Like” (Ll) Gene Structure and Splice Variants

Complete Gene Sequence.

To identify 3′ and 5′ untranslated regions (UTRs) flanking the isolated transcripts of Lisch-like (Ll), each transcript was mapped onto the UCSC Mouse Genome Browser and included contiguous 5′ and 3′ ESTs. Sequences in the predicted extensions can be confirmed by RACE extension and PCR amplification of cDNA libraries using intron-spanning primers from exon 2 (for 5′ analysis) and exon 9 (for 3′ analysis).

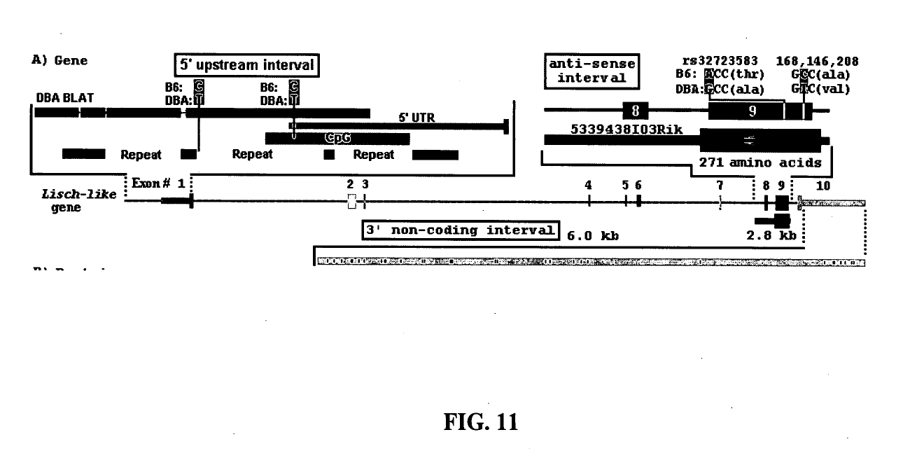

The Ll gene spans 62,714 bp on mouse Chr. 1, from 168,090,795-168,153,508 (FIG. 11). The full-length, 10-exon transcript, isoform 1 (iso1), is 8,279 nucleotides. It comprises a 301 nt 5′ non-coding sequence, a 1941 nt coding sequence (including stop codon), encoding a 646 amino acid polypeptide, and a 6,037 nt 3′ UTR. The 5′ upstream interval includes a CpG island that can overlap the 5′UTR of exon 1. By sequencing this interval, one B6 v DBA sequence variant (C to T) was discovered within the CpG island, and not in a simple repeat. A second, upstream (C to T) variant, is telomeric to a repeat. Further upstream, 3 single base variants surround a simple sequence interval deleted in DBA. Two other short DBA deletions are also in simple repeats. These variants are not identified in the public database. The predicted protein includes a cleavable, signal peptide (SP; exon 1) an extra-cellular domain (ECD; exons 2-4), a trans-membrane domain (TMD; the amino-half of exon 5) and a large intra-cellular domain (ICD; from the cysteine-rich, carboxy-half of exon 5-exon 10). Exons 2 and 3 of the ECD are immunoglobulin-like (Ig-like) V-type domain. Exon 6 is proline-rich and the ICD is overall serine/threonine-rich.

Isoforms.

Complete transcripts for 7 isoforms of Ll were isolated by PCR amplification of cDNAs using primer-pairs flanking the first and last predicted exons (see Methods: Cloning and Sequencing of Lisch-like Isoforms). Four major isoforms shown in FIG. 21 and 3 minor isoforms were identified. Exons 5 and 6 are absent in iso5; exon 9 is absent in iso6; and exons 5-9 are absent in iso7.

5′ Upstream Interval.

The 5′ upstream interval shown (FIG. 21A) includes 569 nt upstream of the predicted first transcribed base of the 5′ UTR. A CpG island is predicted to overlap the 5′ UTR. By sequencing this interval in DBA BAC 95f9 (MM_DBA library, Clemson University Genomics Institute), 8 DBA vs. B6 nucleotide variants were discovered that are not in the public database. Of these, only variant cu_{—}7a, (a C to T substitution within a CpG island) is outside a repeat element.

Anti-Sense Interval.

A 2,845 nt anti-sense transcript (FIG. 21B) of Ll, from adult male pituitary gland (5330438I03Rik; red bar in FIG. 2), starts 42 bp telemetric of exon 9, crosses exons 9 and 8, and terminates in the intron between exons 7 and 8. The centromeric end of the anti-sense transcript is just 506 bp from rs33860076 at the centromeric end of the minimum DBA congenic interval in lines Ijcd, Ijcdt and lcdc. An open reading frame (ORF) encodes a polypeptide of 271 amino acids, with no identifiable domain, and homologous only to the translated anti-sense strand of Ll in other species. The interval contains 45 DBA vs. B6 variants, five of which, underlying exon 9, are listed in dbSNP. One newly discovered variant in the intron preceding exon 8, is an insertion in DBA of a 37 nt unique sequence that is homologous to sequences in the intervals of three unrelated mouse genes. As noted in FIG. 39, the DBA transcript is expressed 2-3 fold higher than B6 in hypothalamus and liver. The regulatory potential for the Ll anti-sense transcript is supported by the observation that the human C1ORF32 gene and the mouse Lsr gene each contain an anti-sense transcript spanning an overlapping interval at their 3′ end. In the Ll sense transcript, the interval corresponding to exon 9 contains five B6 v DBA SNPS (four from dbSNP and one identified in this study). Two of these SNPs generate non-synonymous amino acid substitutions (T572A; A632V). That these sequence variants fall within the anti-sense interval, show that the transcript can regulate Ll gene expression in a way that is affected by B6 v DBA strain-specific sequence differences (for recent reviews of anti-sense regulatory mechanisms see Lapidot and Pilpel, 2006, EMBO Rep 7:1216-1222. Comparative inter-species transcriptomic analysis identifies the 3′ regions of transcripts as important in anti-sense regulation, and conserved overlap between species (see below) can be evidence of function (Numata et al, 2006. Comparative analysis of cis-encoded antisense RNAs in eukaryotes. Gene). As further evidence of this, in 21 day-old 1 jcd males, the DD animals showed a 3-6 fold greater expression of the anti-sense transcript in islets, brain and liver than BB (Affymetrix MOE430-2 microarrays). This effect is correlated with reciprocal decreases in the levels of sense transcript in the same organs.

3′ UTR.

The long (6 kb) 3′UTR of the Ll transcript contains 33 B/D sequence variants that can be involved in regulating expression differences between B6 and DBA. It is estimated that the stability of 35% of yeast transcripts are regulated by motifs in the 3′ UTR (Shalgi et al, 2005, Genome Biol 6:R86) and regulatory motifs, at a similar density, have been identified in the 3′ UTRs of several mammals, including mice (Xie et al, 2005, Nature 434:338-345). 52 B/D sequence variants were identified in the long (6 kb) 3′UTR of the Ll transcript (FIGS. 21C and 27). Of these, 32 were found in the Mouse Build 36.1 SNP database and 20 were identified by the sequencing described herein. Some of these SNPs can be involved in regulating expression differences between B6 and DBA, as it is estimated that the stability of 35% of yeast transcripts are regulated by motifs in the 3′UTR (Shalgi et al, 2005, Genome Biol 6: R86) Regulatory motifs, at a similar density, have been identified in the 3′UTRs of several mammals, including mice (Xie et al, 2005, Nature 434: 338-345).

### Example 9

### Splice Variants of Ll and Target Abundance

Complete transcripts for 7 isoforms of Ll from liver, brain (Clontech, panel 636747; BALB/c mice) and islets (extracted by us) were isolated by PCR amplification of cDNAs using primer-pairs flanking the first and last predicted exons. In addition to the 4 major forms shown in FIG. 21, 3 minor forms were identified. Exons 5 and 6 are absent in form 5; exon 9 is absent in form 6; and exons 5-9 are absent in form 7. The exonic organization and domain structure of the mouse Ll protein is nearly identical to that of the human C1ORF32 protein at 1q24.1 (chr.1 165,154,620-165,211,185; NCBI Build 36.1), which is the product of a gene highly expressed in the developing human retina and brain (Schulz (2003) Towards a Comprehensive Description of the Human Retinal Transcriptome: Identification and Characterization of Differentially Expressed Genes [PhD dissertation]: University of Wurzberg. 5 p.), and also similar to a zebra fish (Danio rerio) gene on chromosome 9@31.6 Mb. These genes are also structurally similar to the mouse Lsr gene, except that Lsr has a short extension to exon 6, and no equivalent to exon 8. Ll and Lsr also have similar splicing patterns with the mouse Ildr1 (Ig-like domain receptor 1) gene (Hauge, H.; Patzke, S.; Delabie, J.; Aasheim, H.-C. Characterization of a novel immunoglobulin-like domain containing receptor. Biochem. Biophys. Res. Commun. 323: 970-978, 2004.)

The relative abundance of the major isoforms, by strain and organ, are shown in FIG. 13. There are striking differences between wild type B6 and DBA animals in the levels of expression of specific isoforms in organs, for example, there is much higher -levels of isoform 4 of Ll in B6 vs. DBA liver, and of isoform 2 in hypothalamus.

As noted herein, Ll expression was detected in mouse in organs relevant to diabetes pathogenesis (islets, hypothalamus, liver, muscle, WAT). Ll was detected also in testis, kidney, heart, lung, uterus, eye, thymus and spleen. By qPCR Ll was detected in e7, e11, e15, and e17 whole mouse embryos from a commercially available cDNA library (Clontech).

Insight into function of the mouse Lisch-like protein can derive from similarities in structure, expression, and cellular location with the human paralog, C1ORF32, and with genes encoding related trans-membrane receptors, Ildr1 (Ig-like domain receptor) (Hauge et al, 2004, Biochem Biophys Res Commun 323: 970-978) and Lsr (lipolysis-stimulated receptor) (Yen et al, 1999, J Biol Chem 274: 13390-13398). Splicing patterns of these genes generate isoforms, similar to those of Ll. Each gene's largest isoform includes an extra-cellular Ig-like domain, a single TM domain, and a similar set of ICDs in related order. In one isoform of each protein, the TM and cysteine-rich domains are absent. An evolutionary, regulatory relationship is indicated by the observation that the Ll-paralog and lldr1 are adjacent in the zebra fish genome (Zv6 assembly, UCSC Genome Browser). The three genes are abundantly expressed in the brain, liver and pancreas (and islets, where studied), and are predicted to have 14-3-3 interacting domains (thus far experimentally verified for the human LSR) (Jin et al, 2004, Curr Biol 14: 1436-1450). Although 14-3-3 interacting domains can be present on as many as 0.6% of human proteins, their occurrence on these Lisch-related proteins is notable, since among known 14-3-3-interacting proteins is phoshodiesterase-3B, which is relevant to diabetes and pancreatic β-cell physiology (Onuma et al, 2002, Diabetes 51: 3362-3367; Xiang et al, 2004, Diabetes 53: 228-234; Pozuelo Rubio et al, 2004, Biochem J 379: 395-408), and others, such as the Cdc25 family members, important in regulating cell proliferation and survival (Meek et al, 2004, J Biol Chem 279: 32046-32054; Hermeking et al, 2006, Semin Cancer Biol 16: 183-192).

The human ortholog of Ll, C1ORF32, which is 90% identical to Ll at the amino acid level, maps to a region of Chr1q23-24 that has been repeatedly implicated in T2DM in seven ethnically diverse populations including Caucasians (Northern Europeans in Utah) (Elbein et al, 1999, Diabetes 48: 1175-1182), Amish Family Study (Hsueh et al, 2003, Diabetes 52: 550-557), United Kingdom Warren 2 study (Wiltshire et al 2001, Am J Hum Genet. 69: 553-569), French families (Vionnet et al, 2002, Am J Hum Genet. 67: 1470-1480), and Framingham Offspring study (Meigs et al, 2002, Diabetes 51: 833-840), Pima Indians (Hanson et al, 1998, Am J Hum Genet. 63: 1130-1138), and Chinese with LOD scores as high as 4.3.

The mouse congenic interval examined here is in the middle of, and physically ˜10× smaller than, the 30 Mb human interval. The genes, and gene order, are conserved between mouse and human in the region syntenic to the congenic interval. The metabolic phenotypes documented in human subjects with T2DM linked to 1q23 closely resemble diabetic phenotypes observed in congenic mice segregating for the DBA interval in B6.DBA congenics examined here (McCarthy et al 2004, Diabetes Positional Cloning Consotrium), indicating that the diabetes-susceptibility gene in congenic mice and human subjects can be the same gene, or among the genes, acting in the same genetic pathway(s). The syntenic interval in the GK rat also correlates with diabetes-susceptibility (Chung et al, 1997, Genomics 41:332-344).

The chr1.1224.1 gene, which is within the minimum DBA interval (crossing the centromeric boundary of lines 1jcdc, 1 jcd and 1 jcdt), shows expression differences consistent with a role in diabetes-susceptibility, and has amino acid sequence variants between DBA and B6. It thus meets the criteria of a candidate diabetes-susceptibility gene. Using primer-pairs flanking the first and last predicted exons, transcripts including coding sequences for chr1.1224.1 were isolated from B6 and DBA cDNA libraries from a wide range of tissue types. The Lsr gene can provide useful insights into Ll structure/function. The rat Lsr gene product is a predicted membrane-bound protein that has a high affinity for chylomicrons and very low density lipoproteins, is primarily expressed in the liver, and is “activated” by free fatty acids (Yen et al. 1999, J Biol Chem 274:13390-13398).

The human ortholog of Ll, C1ORF32, was resequenced and identified 8 polymorphisms (4 promoter, 2 intronic, 1 coding, and 1 3′UTR). The polymorphism in the 3′UTR was associated with diabetes in 405 African Americans (p<0.03) but not 384 Caucasians. 15 single nucleotide polymorphisms in and around C1orf32 were studied in diabetic cases and controls in eight populations (384 African-Americans, 2814 Caucasians, 288 Chinese, and 1132 Pima Indians) (Zeggini et al, 2006, Diabetes 55:2541-2548). Nine of the 15 SNPs showed association in one or more of the populations. Notably, seven of the SNPs showed association in the Utah Caucasian population. Additionally, RS231267 showed association in 3 populations: Utah Caucasians, UK Caucasians, and African Americans. Therefore, one or more of these variants in C1orf32 can play a role in T2DM in humans. In this study, C1ORF32 was resequenced in 35 families with 3 or more generations of Maturity Onset Diabetes of Youth (MODY) that are mutation-negative for the known genetic causes of MODY (HNF1a, HNF4a GCK, NEUROD1, IPF1, and HNF1B). In these families, two intronic (IVS2 nt+7A>G and IVS3 nt-4C>T) and one synonymous coding variant (Ser208Ser) were identified. For each of these three variants, an unaffected individual was found to carry the polymorphism thereby excluding these variants as etiologies for MODY. Studies of LL are underway in the cohorts of T2D just reported by Froguel and collaborators (Sladek et al. 2007, A genome-wide association study identifies novel risk loci for type 2 diabetes. Nature). A “signal” from the LL region has been detected in these individuals.

Human Association Studies in region of C1orf32 (human ortholog of Lisch-Like)

The human syntenic interval corresponding to the location of Ll is on 1q23, a major diabetes-susceptibility interval identified in linkage analysis in multiple populations including Pima Indians, Utah Mormons, Old Order Amish, French Caucasians, Han Chinese, Mexican Americans, and UK, Shanghai and Hong Kong Chinese populations. The mouse congenic interval examined is in the middle of, and physically ˜10× smaller than, the 30 Mb human interval. The genes, and gene order, are conserved between mouse and human in the region syntenic to the congenic interval. The metabolic phenotypes documented in human subjects with T2DM linked to 1q23 closely resemble diabetic phenotypes observed in congenic mice segregating for the DBA interval in B6.DBA congenics examined, suggesting that the diabetes-susceptibility gene in congenic mice and human subjects may be the same gene, or among the genes, acting in the same genetic pathway(s). The syntenic interval in the GK rat also correlates with diabetes-susceptibility. rs1543315, within 65 kb of C1orf32, is significantly associated (p<0.0001) with diabetes in a study of 4 groups of Caucasian subjects. rs6695609, within 21 kb of C1Orf32, is significantly associated (p<0.0002) with diabetes in a genome wide association study (GWAS) of 3 groups of Caucasians Froguel and associates have demonstrated association of rs2075982, 2.5 kb 5′ to exon 2 of the C1orf32 gene with obesity in a GWAS of 600 obese and 2000 lean Caucasian children (p<0.002).

The human ortholog of Ll, C1orf32, was resequenced and 8 polymorphisms were identified (4 promoter, 2 intronic, 1 coding, and 1 3′UTR). The polymorphism in the 3′UTR was associated with diabetes in 405 African Americans (p<0.03) but not 384 Caucasians. In collaboration with the 1q consortrium, 15 single nucleotide polymorphisms were studied in and around C1orf32 in diabetic cases and controls in eight populations (384 African-Americans, 2814 Caucasians, 288 Chinese, and 1132 Pima Indians). Nine of the 15 SNPs showed association in one or more of the populations. Seven of the SNPs showed association in the Utah Caucasian population. Additionally, rs231267 3 Mb from C1Orf32 showed association in 3 populations: Utah Caucasians, UK Caucasians, and African Americans. Therefore, one or more of these variants in C1orf32 may play a role in T2DM in humans.

Antisense Transcripts in Exon 9.

DD mice have reduced Ll transcript levels, associated with decreased B6 cell proliferation during early post-natal development. An anti-sense transcript of 2.8 kb has been detected in mouse Ll and Lsr (AK154275), and in human pituitary. The transcript is neither homologous to any known protein, nor preserved in these species. However, mouse Lsr contains an anti-sense transcript (AK154275) that spans a similar interval. Because anti-sense transcripts can affect stability and degradation of corresponding sense transcripts (Coudert et al, 2005, Nucleic Acids Res 33:5208-5218; Werner and Berdal, 2005, Physiol Genomics 23:125-131; Blin-Wakkach et al, 2001, Proc Natl Acad Sci USA 98:7336-7341), the Ll antisense can be responsible for regulation of Ll levels. In certain aspects, the invention provides methods to quantify antisense transcripts in DD vs. BB mice. Higher levels of the antisense transcript in DD mice can be a cause of reduced Ll mRNA. In one embodiment, expression of the LL antisense transcript can be used to reduce LL mRNA levels. In another embodiment, a nucleic acid molecule having a sequence complementary to a region of the LL antisense transcript can be used to increase LL mRNA levels.

Ll antisense RNA (SEQ ID NO: 19 or 20) can be expressed in MIN-6 and SV40 hepatocytes, and measure whether it affects LL levels. These results can provide an interesting disease mechanism for T2DM. The focus of the investigations will then shift to identifying causes of increased anti-sense transcripts in dd mice.

3′ UTR Variants are Implicated by the Congenic Lines.

Several nucleotide substitutions are present in the 3′ UTR, a region with known function to regulate mRNA stability and degradation. A relevant example derived from the diabetes field is the identification of 3′UTR variants in the PPP3R gene in diabetic Pima Indians (Xia et al, 1998, Diabetes 47:1519-1524). To evaluate whether 3′ UTR is implicated in Ll regulation, reporter plasmids bearing 6 kb of DD and BB 3′UTR, and spanning the 33 identified nucleotide changes will be constructed. The reporter constructs will be cloned between the stop codon and the first polyadenylation site of the rabbit beta globin gene (Xia et al, 1998, Diabetes 47:1519-1524). The vector drives 13-gal expression, which can be readily assessed by colorimetric methods, as described herein and known in the art. Control constructs will contain the sequences in reverse orientation. SV40-transformed mouse hepatocytes or primary hepatocytes will be transiently transfected, and then treated with Actinomycin DBA to inhibit transcription, and measure the disappearance of the reporter activity and mRNA over a chase period of 24-48 hrs. The prediction is that, if the 3′UTR of the DBA allele contains destabilizing mutations, the half-life of the DBA reporter 13-gal and transcript will be shorter than the control cells. In addition to liver cells, similar experiments can be performed in insulinoma cells.

The two non-synonymous coding SNPs in Ll are in exon 9, within the region of overlap among the congenic lines. These variants can account for the differences in transcript abundance and protein levels seen in the congenics. However, these variants seem do not account for the differences in transcript abundance and protein levels seen in the congenics. Also, relevant intronic and/or 3′ UTR variants are present. A 3′UTR polymorphism between two putative mRNA destabilizing motifs in PPPIR3 (muscle-specific glycogen-targeting regulatory PP1 subunit) has been genetically (Xia, et al, 1998, Diabetes 47:1519-1524) and functionally (Xia et al, 1999, Mol Genet Metab 68:48-55) related to T2D.

Based upon a QTL analysis of modifiers of T2DM in Lep^{ob/ob} mice, Lisch-like (Ll) was identified that as a mediator of susceptibility to T2DM by effect on β-cell development, and other aspects of β-cell/islet biology. On the C57BL/6J strain background, the presence of the DBA congenic interval(s) produced relatively mild glucose intolerance that seemed to improve after 150-200 days of age. Phenotypes can be more or less severe on other strain backgrounds.

With regard to Ll, the subcongenic lines investigated have the important characteristic that three of the lines (1jcd , 1jcdt and 1jcdc) contain DBA DNA only 3′ of exon 7, while line Ijc is DBA for the entire gene and extends DBA for another 3 Mb 5′ of Ll. One reasonable inference is that coding and/or non-coding DBA vs. B6 variant(s) in the region of DBA overlap among the congenic lines accounts for the phenotypic differences between the DBA congenic lines and animals segregating for B6 alleles in this region. In the region of overlap that includes the DBA vs. B6 “variable region” (FIG. 9), Ll is the gene showing anticipated differences in coding sequence, gene expression, and protein levels by IHC.

The two non-synonymous coding SNPs in Ll are in exon 9, within the region of overlap among the congenic lines. However, for reasons described herein, these variants do not account for the differences in transcript abundance and protein levels seen in the congenics and the relevant intronic and/or 3′UTR variants are present. A 3′UTR polymorphism between two putative mRNA destabilizing motifs in PPPIR3 (muscle-specific glycogen-targeting regulatory PP1 subunit) has been genetically (Xia et al, 1998, Diabetes 47: 1519-1524) and functionally (Xia et al, 1999, Mol Genet Metab 68: 48-55) related to T2DM. Variants in the 3′UTR can also affect regulation by microRNAs (miRNAs). The 3′UTR is the target of mammalian microRNAs (miRNAs) (Grimson et al, 2007, Mol. Cell. 2007 Jul. 6; 27(1):91-105.) and their relevance to diabetes is underscored by the finding that a mouse islet-specific microRNA, miR-375, affects insulin secretion (Poy et al, 2004, Nature. 2004 Nov. 11; 432(7014):226-30).

The physiological role of Ll is unknown. Based upon the apparent effects of DBA alleles on β-cell production rates in 1-day old animals—reduced in D/D (but recovered by 21 days), the periods of relatively mild hyperglycemia (60-120 days), and reduced proportions of β-cells to islet area by 150 days (FIG. 7), Ll can influence early β-cell differentiation/turnover in a manner that predisposes obese animals to later failure of β-cells by effects on mass and function (Prentki et al, 2006, J Clin Invest 116: 1802-1812; Stanger et al, 2007, Nature 445: 886-891). That these phenotypes are recapitulated in W87* L1 C3H mice is supporting evidence. In the neonatal rodent, remodeling of β-cells occurs as a result of simultaneous activation of both apoptosis and β-cell replication (Bonner-Weir 2000, Trends Endocrinol Metab 11: 375-378). Between 4 and 24 weeks, postnatally, β-cell mass is estimated to increase 10 fold, related in part to increased body mass. Compensation for β-cell stress/loss in adult rodents is primarily by β-cell hypertrophy and β-cell proliferation (Dor et al, 2004, Nature 429: 41-46). In rats, β-cell proliferation rates decline from ˜20% per day in pups, to ˜10% per day at 6-8 weeks, and to ˜2% shortly thereafter (Finegood et al, 1995, Diabetes 44: 249-256). However, even this low rate of turnover apparently does not persist in adulthood. Using continuous long term BrdU labeling in C57x129Sv and BALB/C one year-old mice, Teta et al. (Teta et al, 2005, Diabetes 54: 2557-2567) have reported extremely low replacement rates (˜ 1/1400 mature β-cells/day). Consistent with this finding, Stanger et al recently showed that pancreas mass in the mouse is irreversibly constrained by the size of a progenitor pool in the embryonic pancreatic bud (Stanger et al, 2007, Nature 445: 886-891). These data indicate that β-cell mass established in the first 6-8 weeks of life can be critical to the ability to meet subsequent stresses on β-cell function imposed by e.g. obesity, hyperglycemia, dyslipidemia. The molecular regulation of these processes is incompletely understood, but even transient interruptions can, based upon this formulation, result in permanent effects on cell mass, or function, or both (Hales et al, 2001, Br Med Bull 60: 5-20). Hypoactivity of the candidate T2D modifier gene (Ll) reported here can mediate such effects on establishment of initial β-cell mass, and/or later responses of cell hypertrophy/replication by β-cell-autonomous effects or in response to an exogenous ligand for this putative receptor.

Observations that expression levels of Ll are most strikingly affected in liver, the effects of the zebra fish knockdowns on general endodermal development, and structure/function considerations raised by the homologous LSR molecule (Yen et al, 1999, J Biol Chem 274: 13390-13398), are consistent with the mechanism(s) by which Ll conveys effects on cell mass/function can relate, in part, to consequences of putative effects on hepatic development/function. IGF1 (Leahy et al, 1990, Endocrinology 126: 1593-1598) and hepatic growth factor (Garcia-Ocana et al, 2000, J Biol Chem 275: 1226-1232) are examples of such β-cell “hepatokines” affecting beta cell function.

Accession Numbers.

The Genbank accession numbers for the M. musculus genes described herein are as follows: Lisch-like (XM_{—}001473525); Lsr (NM_{—}017405); Ildr1 (NM_{—}134109); Tadall (NM_{—}030245); Pogk (NM_{—}175170); FMO13 (XM_{—}136366); FMO9 (NM_{—}172844) FMO12 (XM_{—}136368); CO30014K22Rik (NM_{—}175461); Uck2 (NM_{—}030724); Tmcol (NM_{—}001039483); Aldh9a1 (NM_{—}019993); Mgst3 (NM_{—}025569); Lrrc52 (NM_{—}00103382); Rxrg (NM_{—}009107); Lmx1a (NM_{—}033652); Pbx1 (NM_{—}008783); H. sapiens C1ORF32 (NM_{—}199351); LSR (NM_{—}015925); ILDR1 (NM_{—}175924); D. rerio Ll paralog (NM_{—}001030192.1); Lsr paralog (NM_{—}001025472.1); R. rattus Lsr (NM_{—}032616)

The Genbank accession numbers for protein sequences used in this paper are as follows: M. musculus Lisch-like (amino acid residues 150-795 XP_{—}001473575); (Lsr) (NP_{—}059101); Ildr1 (NP_{—}598870); H. sapiens C1orf32 (NP_{—}955383); LSR (NP_{—}057009); ILDR1 (NP_{—}787120); D. rerio (Lisch-like paralog) (NP_{—}001025363); Lsr paralog (NP_{—}001020643); R. rattus Lsr (NP_{—}116005)

### Example 10

### Lisch-Like Immunohistochemistry

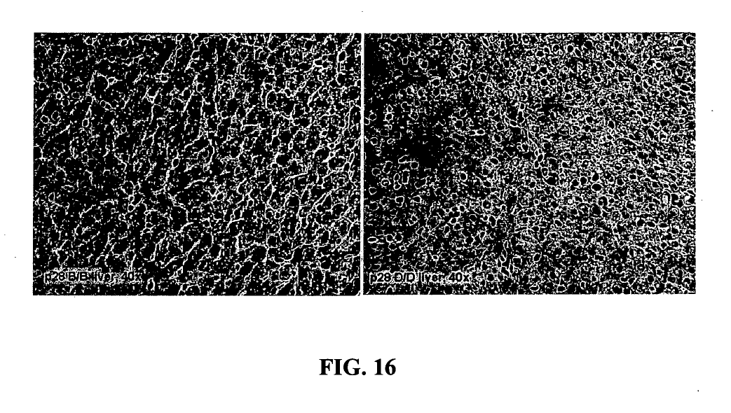

Antibodies to the intracellular domain of LL (see Methods), used for immunohistochemical (IHC) staining of Ll protein in pancreatic sections of 21-day old Lep^{ob/ob} B/B and D/D 1jc males, show clear reduction in LL protein levels in β-cells (FIG. 15) and hepatocytes (FIG. 16) of D/D animals, consistent with the gene expression results. The localized expression pattern of Ll in pancreatic β-cells in non-diabetic mice, in conjunction with the low level of LL staining in D/D mice (that show reduced β-cell replication and reduced islet mass) indicate that Ll can play a role in β-cell development.

### Example 11

### W87 Stop Mutatio of Ll in C3HeB/FeJ Mice

To examine phenotypes of mice segregating for a null allele for Ll, a repository of ENU-generated (N-ethyl-N-nitrosourea) mutant sperm DNAs from 18,000 C3HeB/FeJ G1 males was screened for mutations in Lisch-like (Augustin M, Sedlmeier R, Peters T, Huffstadt U, Kochmann E, et al. (2005) Efficient and fast targeted production of murine models based on ENU mutagenesis. Mamm Genome 16: 405-413). A G/A substitution was detected that encodes an amber stop mutation at threonine-87 [W87*] and also creates an EcoN1 cleavage site, which was used to genotype for the mutation. By in vitro fertilization, W87* heterozygotes were generated on the C3HeB/FeJ background, and these animals were bred to generate progeny that were homozygous wild-type (+/+), homozygous mutant (−/−) or heterozygous (+/−) for the W87* mutation. Progeny were born at the anticipated Mendelian ratios, and the −/− animals did not appear grossly compromised.

To verify that the W87* homozygous mutant was hypomorphic for LL protein, we compared a Western blot of hypothalamic extracts prepared from C3HeBFeJ wild-type (+/+) and mutant (−/−) mice, with a second blot of hypothalamic extracts prepared from B/B and 1jc-D/D congenic mice. Both sets of filters were probed with a polyclonal rabbit antibody generated to a conjugated polypeptide, corresponding to exons 7 and 8 of isoform 1, in the predicted ICD of LL. LL protein was greatly reduced in the brains of D/D vs B/B congenics and in the ENU-treated W87* homozygotes vs. the wild-type animals (FIG. 41A).

By 14 days of age reductions in β-cell replication rates are detectable that are similar to those seen in the DD congenic lines (5B). There is a >2-fold difference in the proportion of Ki67-positive β-cells in 14-day old wild-type (3.75%) vs. homozygous W87* mice (1.75%), with heterozygotes intermediate (2.5%) (FIG. 41B). Insulin concentrations in Ll W87* homozygotes are reduced by the time of sexual maturation (FIG. 41C) and, consistent with this difference, at 50 days of age, homozygous W87* males show an increased glucose AUC during iPGTT (FIG. 41D). A significant decrease in β-cell mass is also detected in W87* homozygotes (1.05%±0.117, n=3, p=0.0113) v. +/+ littermates (2.74±0.364; n=3) at 150 days of age.

These phenotypes were detected despite the segregation of the mutation on a different background strain (C3HeB/FeJ) than the congenics (C57BL/6J), and in the absence of co-segregation of the Lep^{ob}. These data support the candidacy of Ll as the gene accounting for the diabetes-related phenotypes of the DD congenic lines.

### Example 12

### Cross-Species Comparisons of Ll Sequence and Transcript Abundance

From the Ensemb1 database, zebra fish orthologs of Ll and Lsr were identified. The clustalW pair-wise similarity scores for the predicted protein coded for by the zebra fish gene zgc:114089 (Lsr ortholog) is 42 vs, the mouse LSR protein, and 29 vs. the mouse LL protein. The similarity scores for the predicted protein coded for by the zebra fish gene zgc:110016 (Lisch-like ortholog) are 36 vs. LL and 28 vs. LSR. ClustalW analysis was performed (FIG. 32) between the mouse LL-iso1 protein and three related proteins: 1) the human C1ORF32 protein at 1q24.1 (chr.1 165,154,620-165,211,185; NCBI Build 36.1), which is the product of a gene highly expressed in the developing human retina and brain (Schulz H (2003) Towards a Comprehensive Description of the Human Retinal Transcriptome: Identification and Characterization of Differentially Expressed Genes [PhD dissertation]: University of Wurzberg. 5 p.); 2) the predicted protein sequence for the zebra fish Lisch-like ortholog, zgc:110016 located on zebra fish chromosome 9@31.6 Mb; and 3) the mouse LSR protein, transcribed from a gene on chromosome 7@30.7 Mb. Pair-wise similarity scores for the intact proteins and major domains are shown in the legend. The human homolog is similar throughout, but diverges slightly in the putative intracellular domain (ICD). The zebra fish Lisch-like ortholog and mouse LSR proteins are most alike in the TMD, less so in the Ig-like domain, and most dissimilar in the ICD. The Lsr protein has a short extension to exon 6, and no exon 8 equivalent. Ll and Lsr also have splicing patterns similar to the mouse Ildr1 (Ig-like domain receptor 1) gene (Hauge H, Patzke S, Delabie J, Aasheim H C (2004) Characterization of a novel immunoglobulin-like domain containing receptor. Biochem Biophys Res Commun 323: 970-978), and the proteins they encode all belong to the Lisch7 family (IPRO08664).

ClustalW analysis was performed between the mouse LL protein (isoform 1; 646 amino acids), and each of three related proteins: human C1orf32, zebrafish (Dr.7.2) and the mouse (Mm) Lsr. Table 3 shows pairwise similarity scores for the intact proteins and for each major domain with ClustalW analysis. ClustalW analysis was performed on the EMBL-EBI server using default settings. Mouse LSR sequence is NP_{—}059101; mouse Ll is identical to the N-scan predicted sequence chr1.1224.1; human C1orf32 sequence is NP_{—}955383; zebrafish Ll sequence is NP_{—}001025363 (RefSeq NM_{—}001030192.1).

TABLE 3
ClustalW analysis of Lisch-like homologs and the LSR protein

| Protein | residues | intact | Ig-like | Tm | ICD |
|---|---|---|---|---|---|
| Hs.C1orf32 | 639 | 90 | 98 | 98 | 87 |
| Dr.7.2 | 629 | 36 | 51 | 70 | 26 |
| Mm. Lsr | 594 | 34 | 47 | 70 | 25 |

Ll expression was detected in mouse in organs relevant to diabetes pathogenesis (islets, hypothalamus, liver, muscle, WAT), and in testis, kidney, heart, lung, uterus, eye, thymus and spleen. By qPCR Ll was detected in e7, e11, e15, and e17 whole mouse embryos.

TABLE 4
Localization of L1 expression
Summary of L1 expression in tissues of humans
(adults) and Mice (~12 wks of age)

| | Normalized values of L1 expression | |
|---|---|---|
| Tissues | L1/Actin ratios ×10*3 | |
| Brain | 430 | (human) |
| | 45.5 | (mouse) |
| Hypothalamus | 58.6 | (mouse) |
| White adipose tissue | 47.6 | (human) |
| | 1.9 | (mouse) |
| Pancreas | 12.0 | (human) |
| | 4.02 | (mouse) |
| Islets | 75.6 | (human) |
| | 3.9 | (mouse) |
| Skeletal muscle | 9.9 | (human) |
| | .48 | (mouse) |
| Liver | 3.8 | (human) |
| | 13.8 | (mouse) |
| 11DO embryo | 4.24 | (mouse) |

### Example 13

### Knockdown of Ll and Lsr Paralogs in Zebra Fish

To assess the function of Ll in islet/β-cell ontogenesis, the expression pattern and the effects of morpholino-mediated knockdown in the zebra fish embryo were examined. Morpholinos are modified anti-sense oligonucleotides that produce a strong hypomorphic “knockdown” phenotype (Draper 2001, Genesis 30: 154-156) by inhibiting proper splicing of the pre-RNA transcript (Draper 2001, Genesis 30: 154-156) or by ATG-blocking of translation (Nasevicius and Ekker 2000, Nat Genet. 26: 216-220). Morpholino knockdown has been used to demonstrate a role for the endocrine hormones GnRH, GHRH and PACAP during development (Kim et al, 2006, Mol Endocrinol 20: 194-203; Field et al, 2003a, Dev Biol 261: 197-208; Sherwood et al, 2005, Gen Comp Endocrinol 142: 74-80; McGonnell and Fowkes 2006, J Endocrinol 189: 425-439). Many of the molecular mechanisms regulating pancreas development appear to be conserved among zebra fish and other vertebrates (Gnugge et al, 2004, Methods Cell Biol 76: 531-551), and the single zebra fish islet provides an excellent model of vertebrate development.

Zebra fish paralogs of two Lisch-related proteins were identified. NM_{—}001030192.1 on Chr 9 at 31.6 Mb, is homologous to Ll/C1ORF32 (Ll paralog). NM_{—}001025472.1 on Chr 15 at 39.0 Mb, is homologous to Lsr (Lsr-like paralog). Using whole mount in situ hybridization (FIG. 40) Lisch-like ortholog zgc:110016 was expressed in the brain and otocyst by 48 hours post fertilization (hpf), and by 72 hpf expression was evident in the intestine. The Lsr ortholog zgc:114089, located on Chr 15 at 39.0 Mb, was expressed in pancreas at 48 and 72 hpf, (similar to our postnatal observations in mouse with Ll), intestine, liver, pharynx, pronehphros and otocyst for 48 hpf, and, at 34 hpf, in both pancreatic buds. Since the anterior bud gives rise to exocrine tissue, pancreatic duct, and a small number of endocrine cells, while the posterior bud gives rise only to endocrine tissue (Field 2003, Dev Biol 261: 197-208) Lsr-like expression throughout this stage is consistent with a role in the ontogeny of pancreatic endocrine tissue.

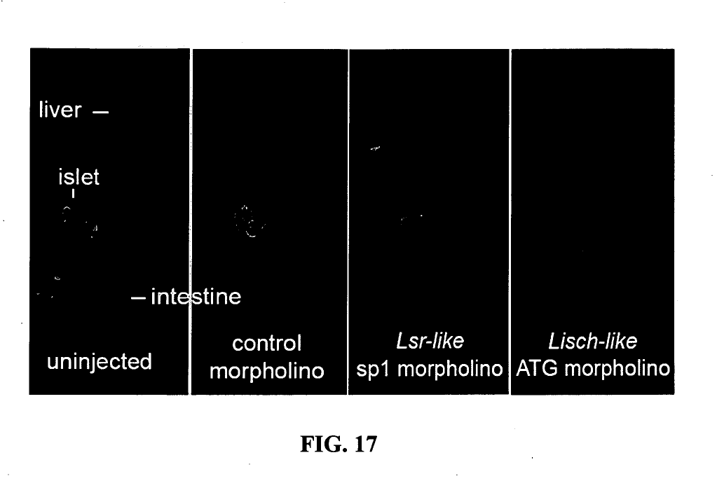

The close structural similarities among Lisch-related genes (Table 6) (FIG. 32) indicated that functional data on both zebra fish geness can be physiologically relevant and, therefore, the involvement in islet development of both paralogs was studied. To study the function of zebrafish Lisch-like in development, in separate experiments, morpholinos for both genes were injected into embryos homozygous for the gut-GFP transgene to fluorescently visualize developing endodermal organ (FIG. 17) (Field et al, 2003, Dev Biol 253:279-290).

β-cell development was assessed with an anti-insulin antibody at 48 hpf or by insulin in situ hybridization at 24 hpf. To assess morpholino specificity, the effects of two separate, non-overlapping morpholinos were analyzed for each gene. Both morpholinos for each ortholog independently produced similar phenotypes, providing evidence that the effects (described below) were the result of specific gene knockdown and not due to nonspecific morpholino-related effects.

FIG. 11 shows that both Lsr-like and Ll morpholinos injected at 15 ng/embryo produced general developmental delay in the endodermal organs, evidenced by a smaller liver, a smaller, straighter intestine, and a smaller pancreas that does not extend as much as in wild-type. The Lsr-like morpholinos disrupt β-cells more severely (note ectopic insulin-positive cells in the cephalad region of the pancreas) than do the Ll morpholinos (note the milder local dispersion of insulin-positive cells); 48/72 and 25/144 embryos injected with morpholinos targeting Lsr-like and Ll, respectively, displayed a scattered β-cell phenotype. These effects were rarely observed in uninjected sibling embryos (0/25) or embryos injected with a control morpholino (1/35). Lower doses of Lsr-like and Ll morpholinos (˜7-10 ng) resulted in a lower frequency of β-cell scattering and higher doses (˜20-25 ng) resulted in embryonic toxicity, which is common with high doses of morpholinos. The efficacy of the splice-blocking Lsr-like and Ll morpholinos was assessed via RT-PCR and all were found to strongly and specifically inhibit proper splicing of their respective target transcripts at the 15 ng dose. In combination, the expression analyses and morpholino knockdown studies provide support for a role of Lisch gene family members in endodermal development, and suggest specific effects on the embryonic β-cell.

RT-PCR showed that both morpholinos strongly and specifically inhibit proper splicing of the transcript. At 34 hpf, Lsr-like is expressed in islet, liver (similar to postnatal observations in mouse with Ll) and in both pancreatic buds. The anterior bud gives rise to exocrine tissue, pancreatic duct, and a small amount of endocrine cells, while the posterior bud gives rise only to endocrine tissue (Field, H. A. e. a. 2003a, Dev Biol 261:197-208). Lsr-like expression throughout this stage is consistent with its role in pancreatic endocrine tissue development. Expression patterns viewed using whole mount in situ hybridization the Lisch-like paralog, is highly expressed in the brain and eye by 48 hpf and by 72 hpf, expression is evident in the intestine. At 48 hpf, both Lsr-like and Lisch-like morpholinos produced general developmental delay, evidenced by a smaller liver and smaller, straighter intestine, and a smaller pancreas that does not extend as much as in wild-type. The Lsr morpholino disrupts β-cells more severely (note ectopic insulin-positive cells cephalad of the pancreas) than does the Ll morpholino (note the milder local dispersion of insulin-positive cells). These effects were not observed in uninjected sibling embryos or embryos injected with a control morpholino. The relevance of such studies to mammalian pancreas development has been shown earlier for Ptf1a (Zecchin et al, 2004, Dev Biol 268: 174-184; Lin et al, 2004, Dev Biol 274: 491-503) and for Pdx1 (Yee et al, 2001, Genesis 30: 137-140).

Levels of LL expression in different cell lines. Analysis by qPCR by methods as described herein. Primers (exons 5-9) detected isoforms of LL. LL is present in rat INS1 cells.

| | | | SH - | | | | |
|---|---|---|---|---|---|---|---|
| | HEPG2 | HEK 293 | fibroblasts | GT7-HT | 3T3L1 | MIN6 | BTC3 |
| Cell Lines | (human) | (human) | (mouse) | (mouse) | (mouse) | (mouse) | (mouse) |
| Ll/Actin x | 125 | 6.0 | .00304 | 3.03 | .0061 | 3.01 | .117 |
| 10³ qPCR | | | | | | | |

### Example 14

### Constructs for Assessment of Intracellular Trafficking of LL

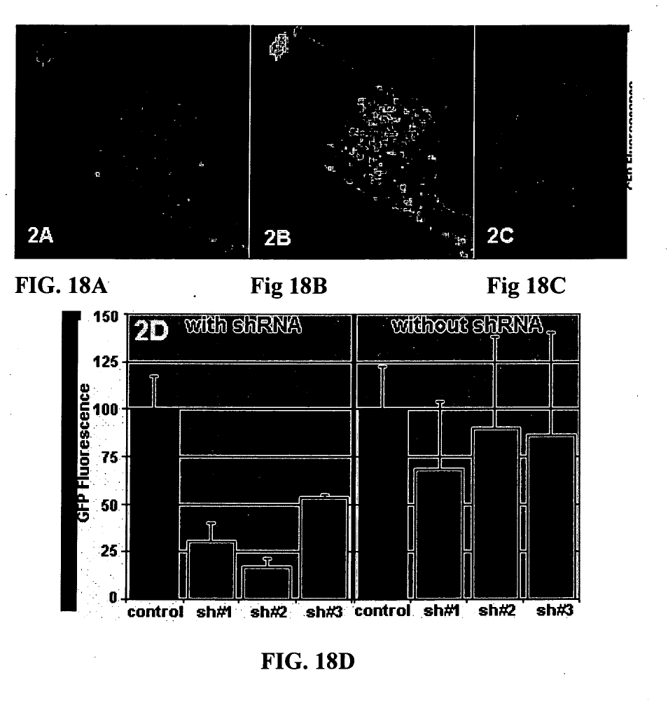

Full length C57BL/6 LL cDNA was cloned into the pEGFP-N3 vector and used to transfect MIN6 cells Immunohitochmical staining with monoclonal anti-GFP, reveals a punctate plasma membrane and cytoplasmic pattern, which can be consistent with targeting to specialized plasma membrane compartments (caveolae, coated pits), lysosomes, and mitochondria (FIG. 18 A). MIN6 cells transfected with GFP-LL construct and co-stained with ICD LL rabbit antibody (FIG. 18B). Full length LL was cloned into CMV4A, containing the FLAG sequence. MIN6 cells were transfected and stained with monoclonal anti-flag (FIG. 18 C). Three shRNA constructs (Moffat et al, 2006, Cell 124:1283-1298) were prepared with different 21-mer stem sequences designed to maximally reduce target message (Khvorova et al, 2003, Cell 115:209-216; Schwarz et al, 2003, Cell 115:199-208). The shRNA-containing plasmids and LL-GFP plasmids were co-transfected into HEK293 cells and the efficiency of knock down was measured as previously described (Antinozzi et al, 2006, Proc Natl Acad Sci USA 103:3698-3703). GFP intensity per cell was compared in samples transfected with GFP fusion LL vector with and without cotransfection with shRNA constructs (FIG. 18D). These data indicate that LL can be efficiently knocked-down using these constructs. siRNA for in exon 1 target sequence ACCGCTGTCTTCTGGTTAACA (SEQ ID NO: 59) is synthesized and can be tested.

### Example 15

### ENU-Mutagenized Mice

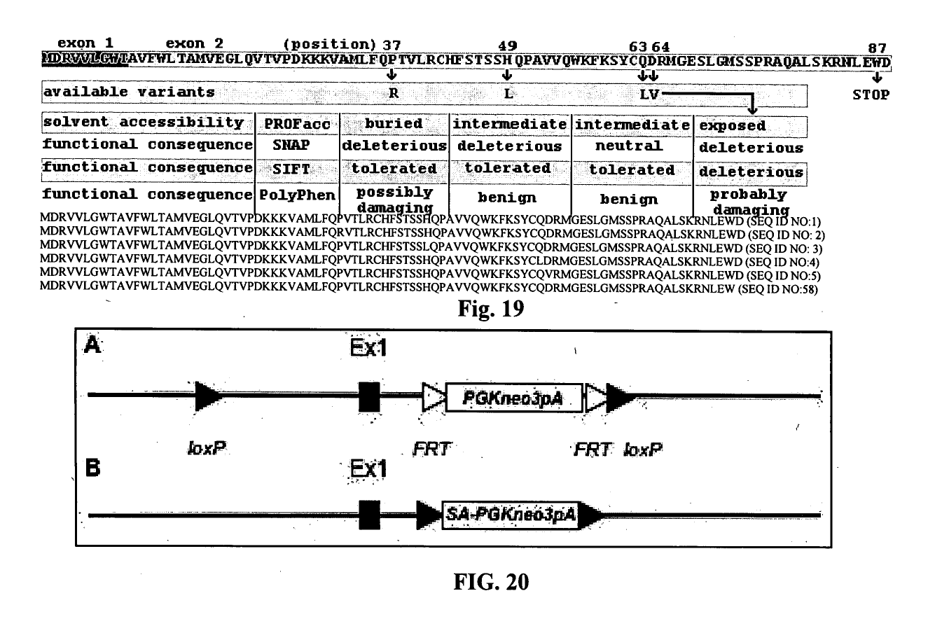

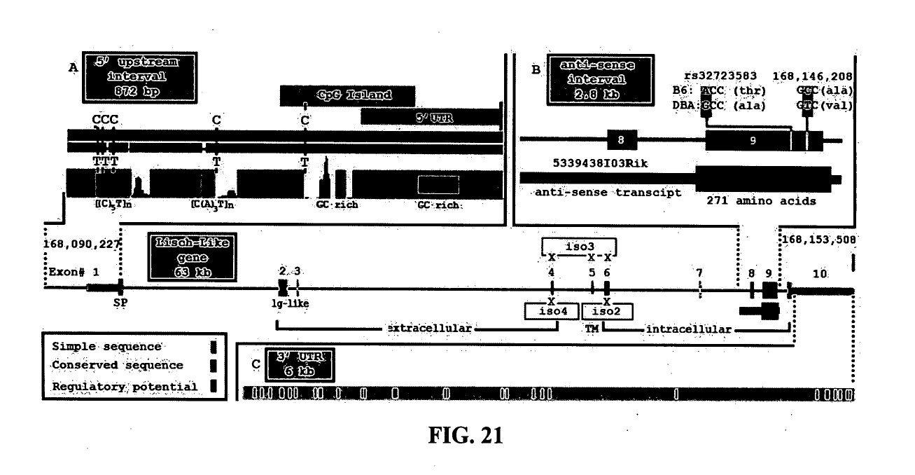

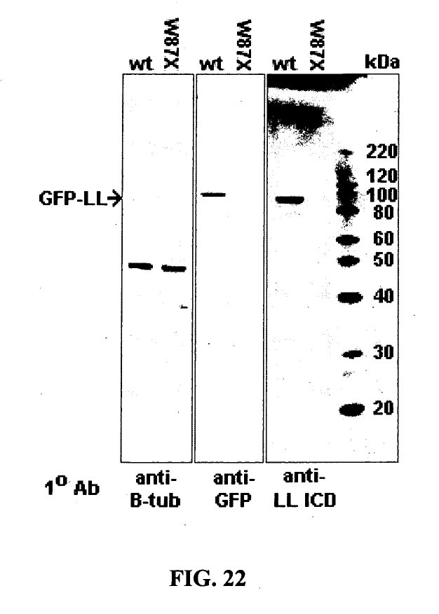

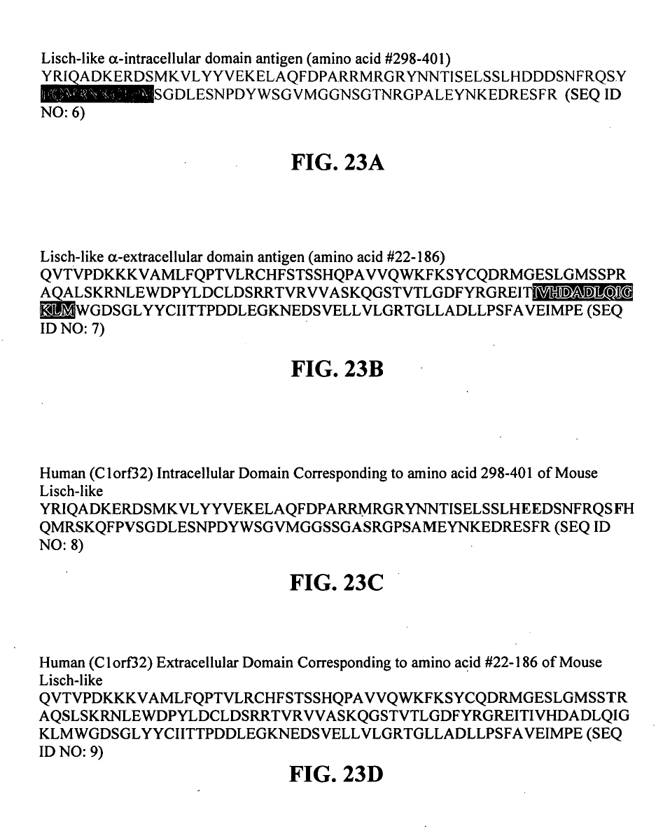

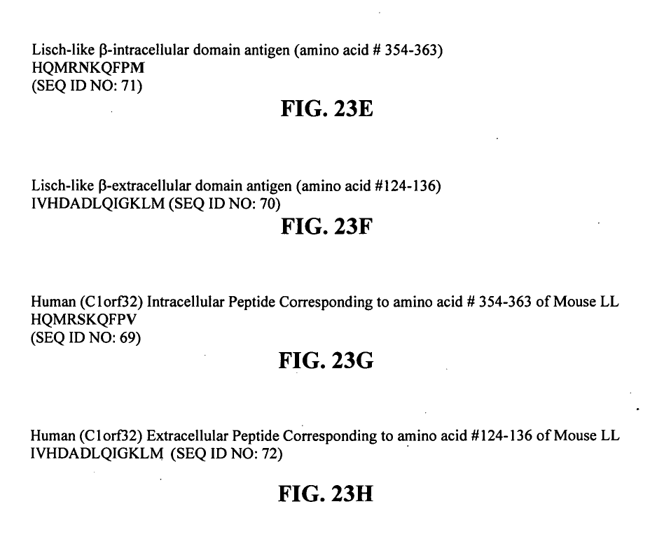

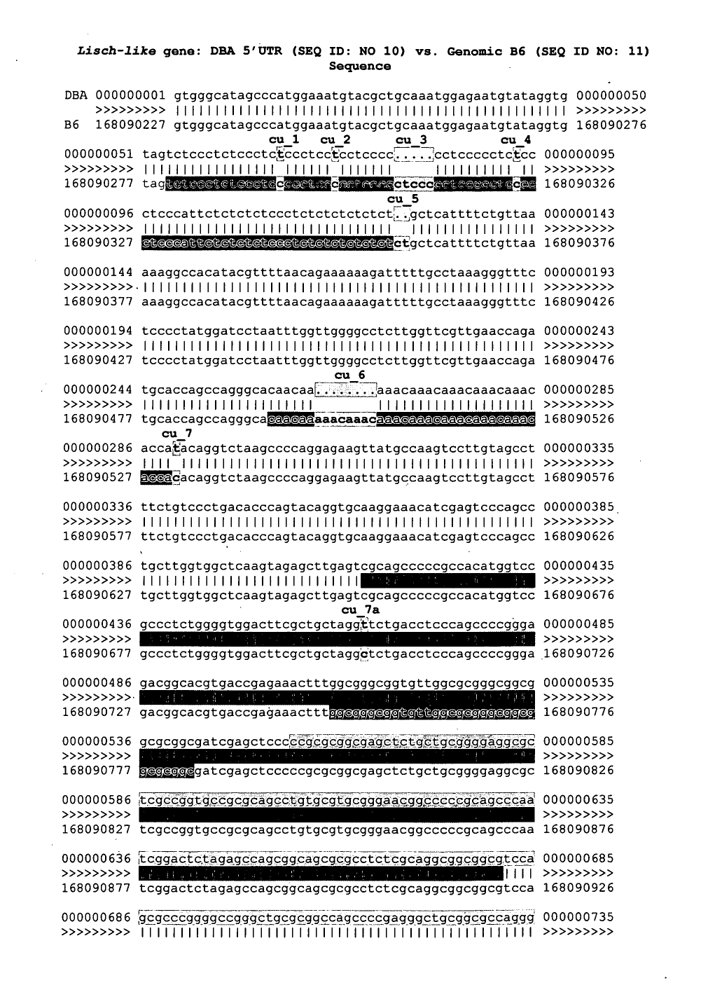

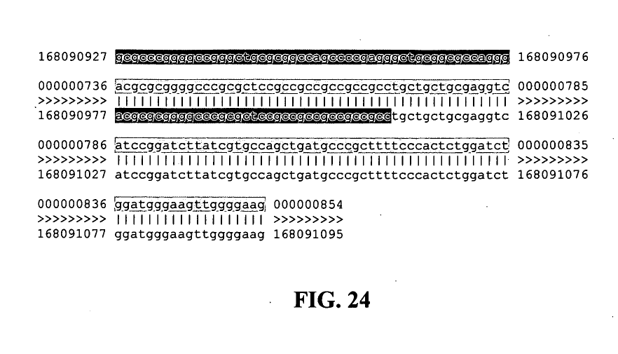

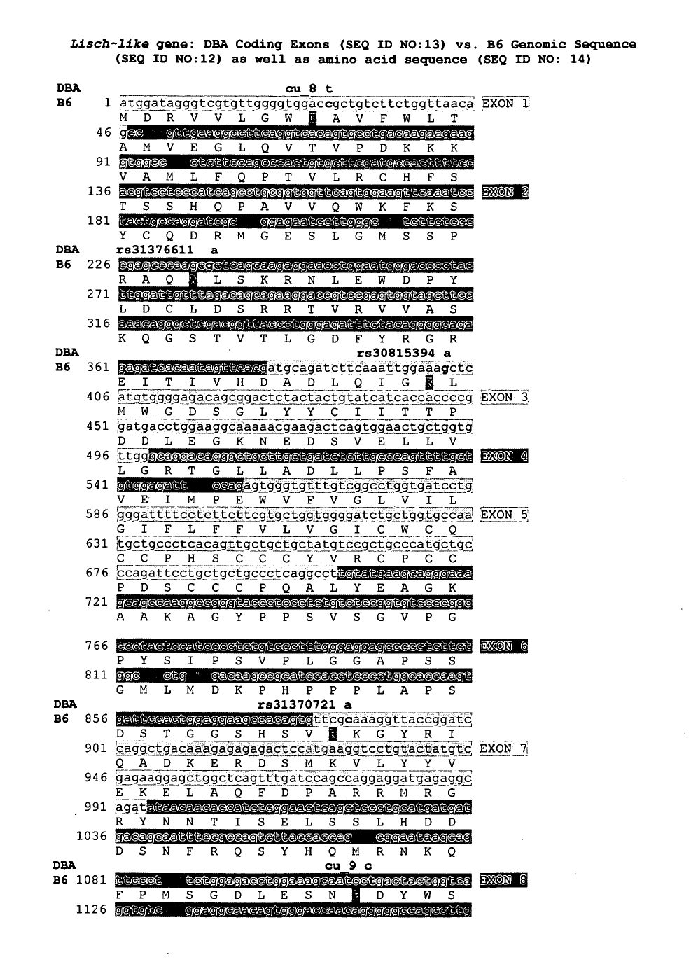

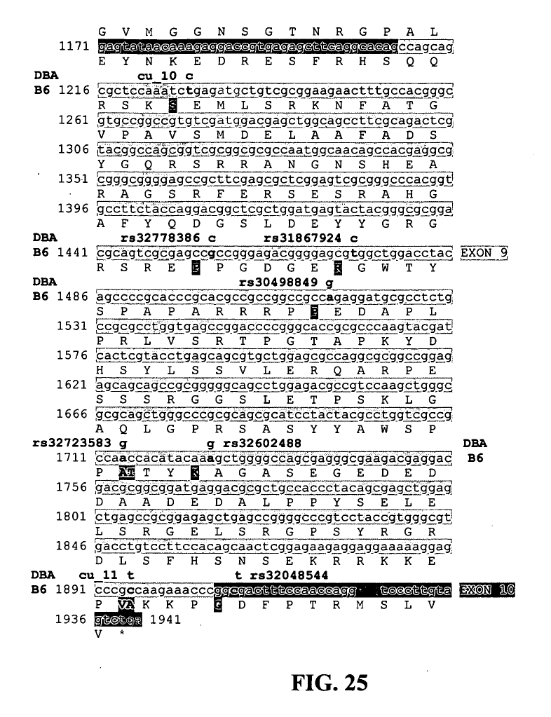

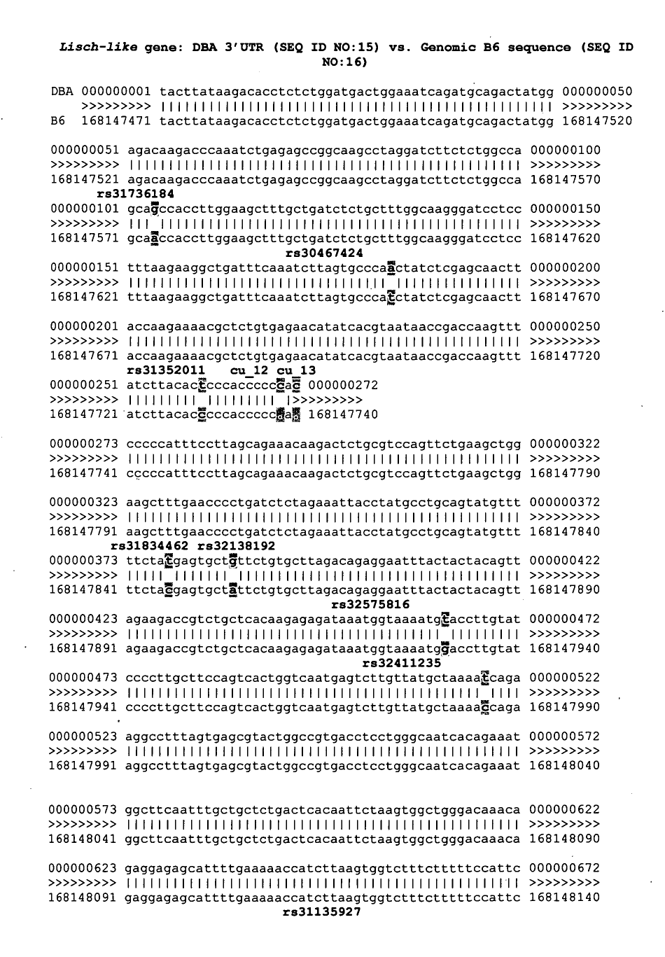

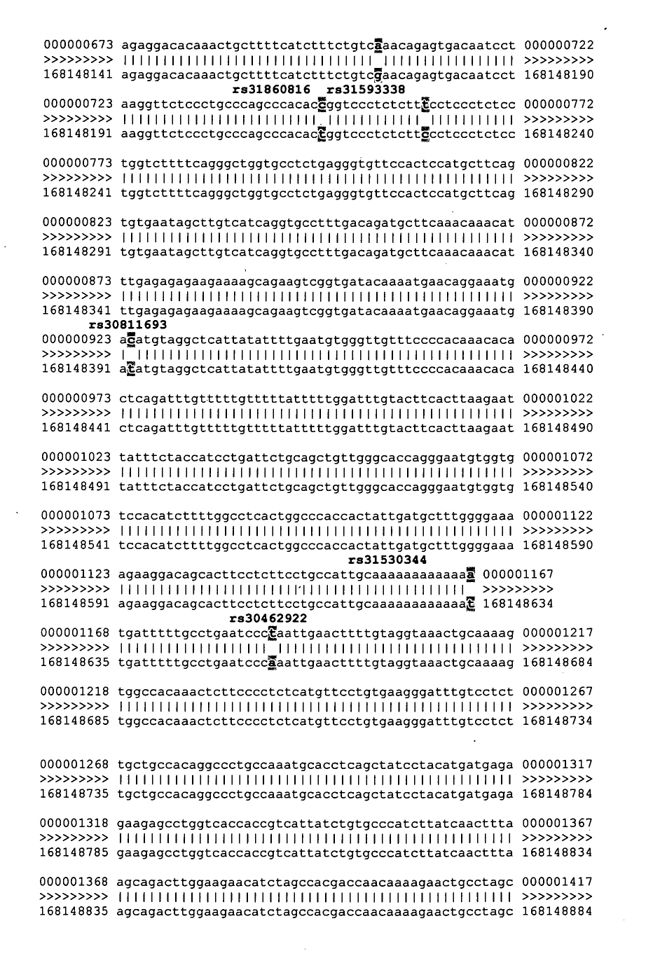

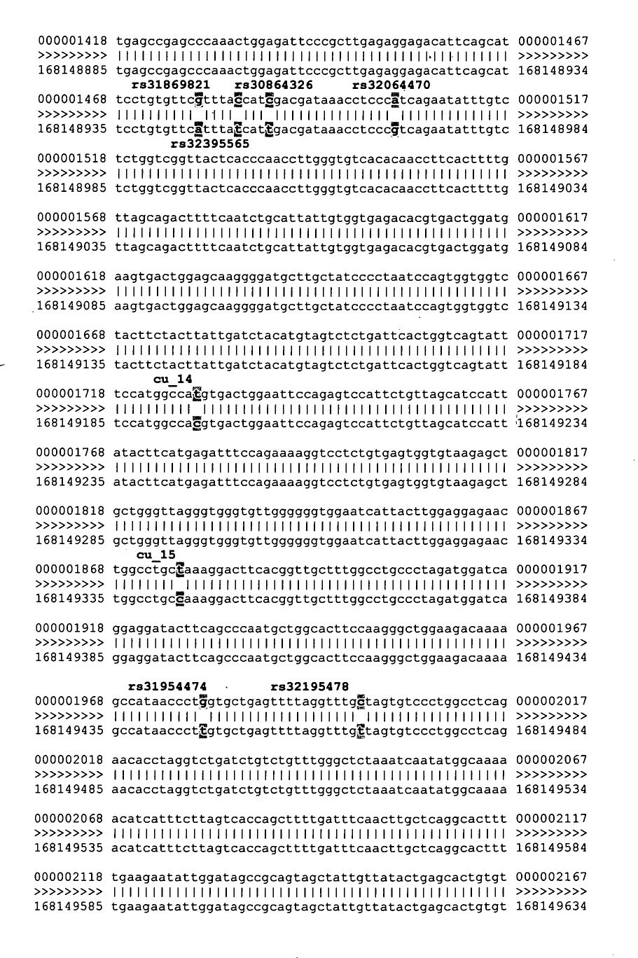

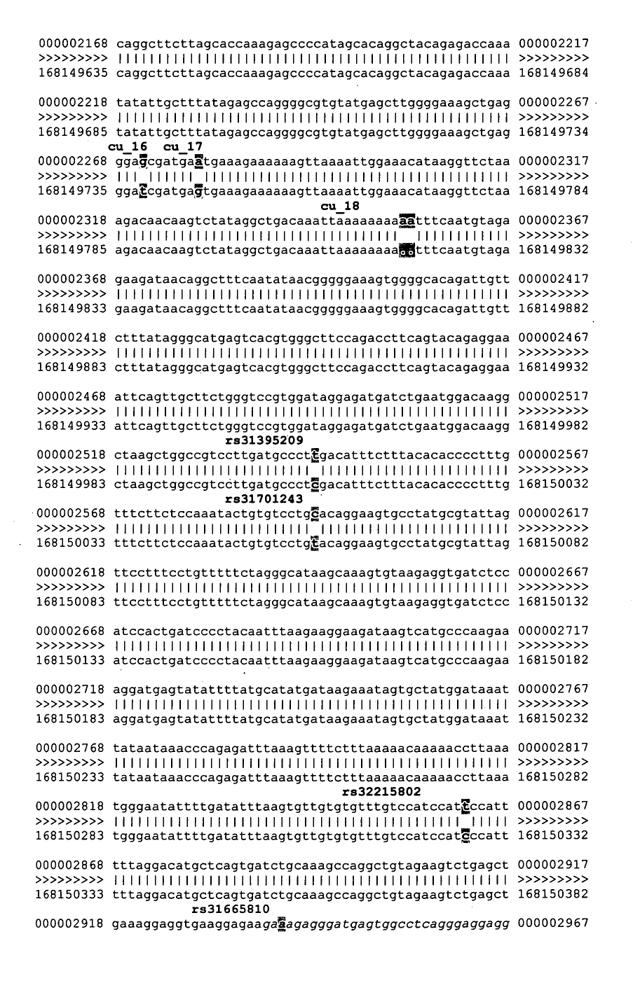

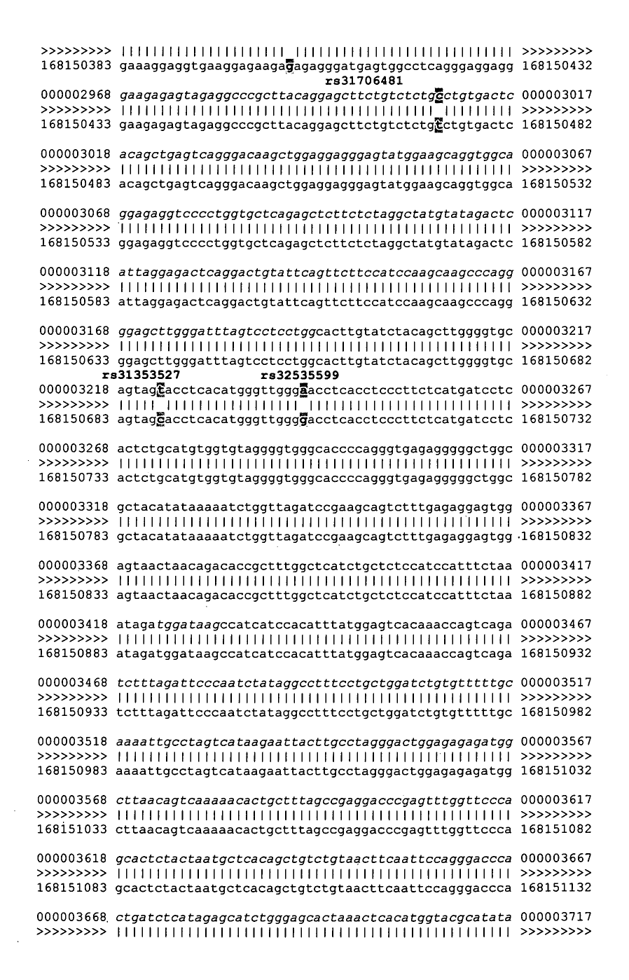

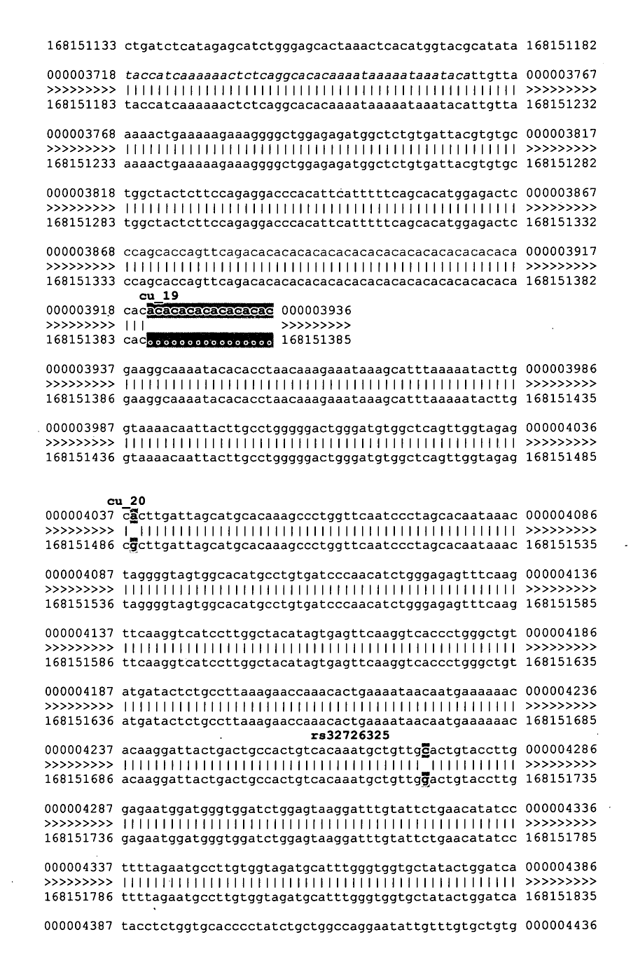

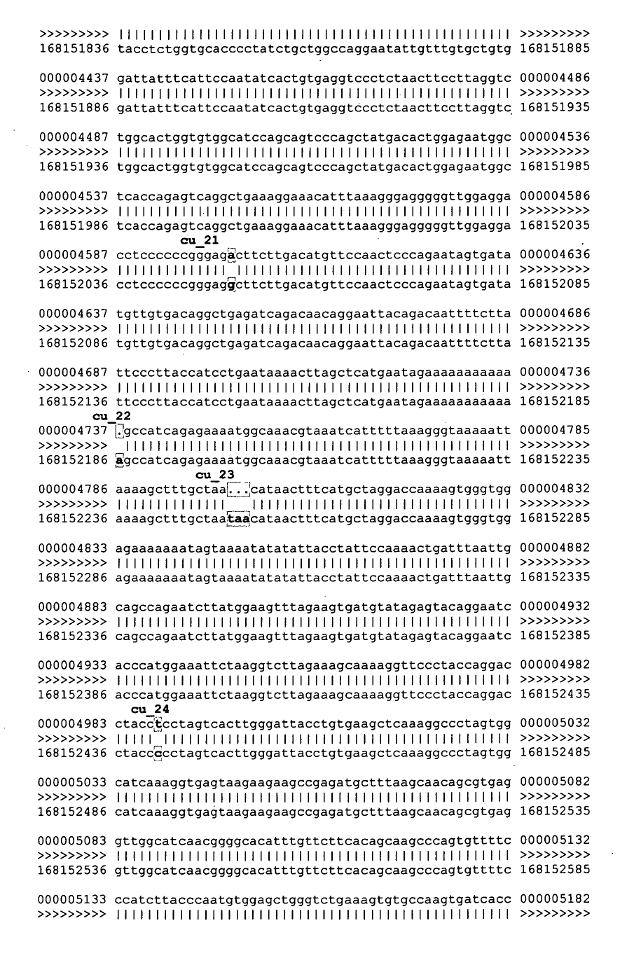

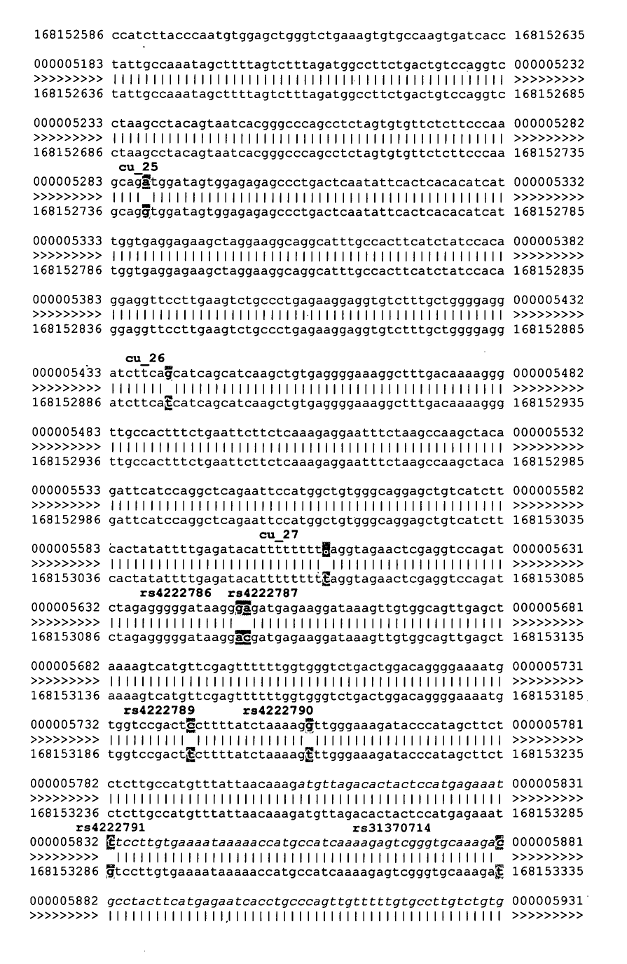

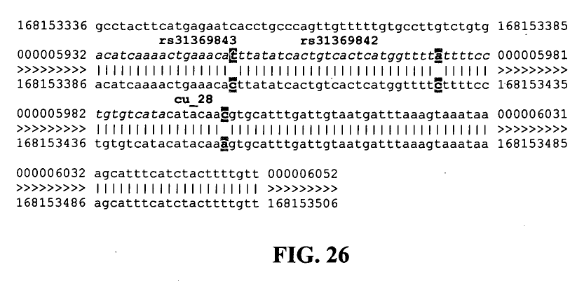

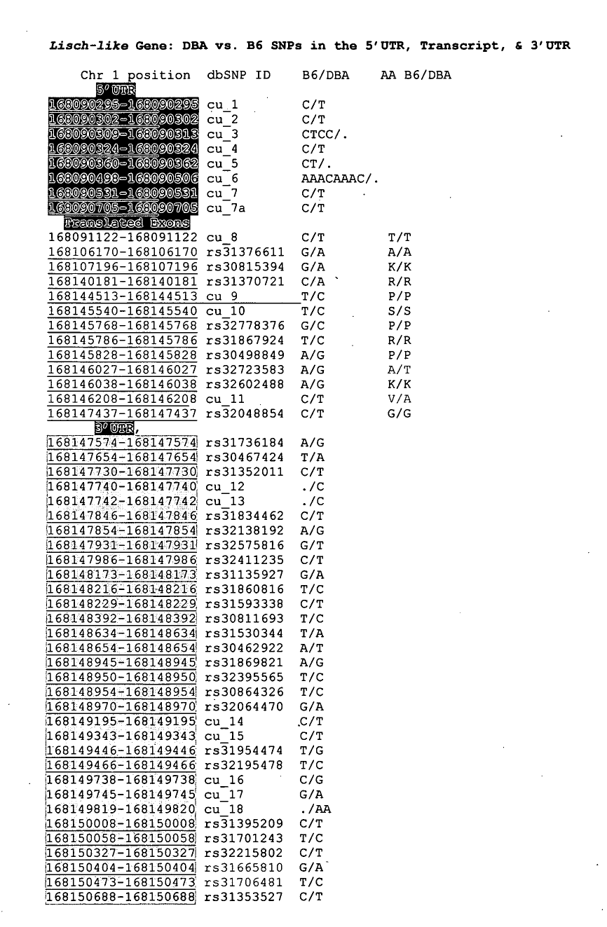

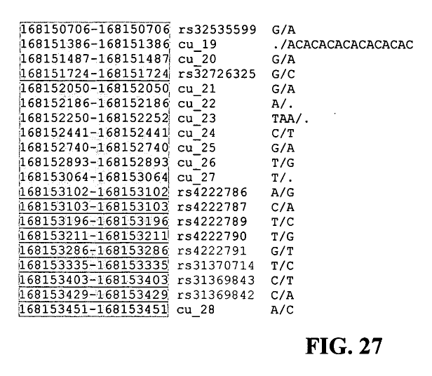

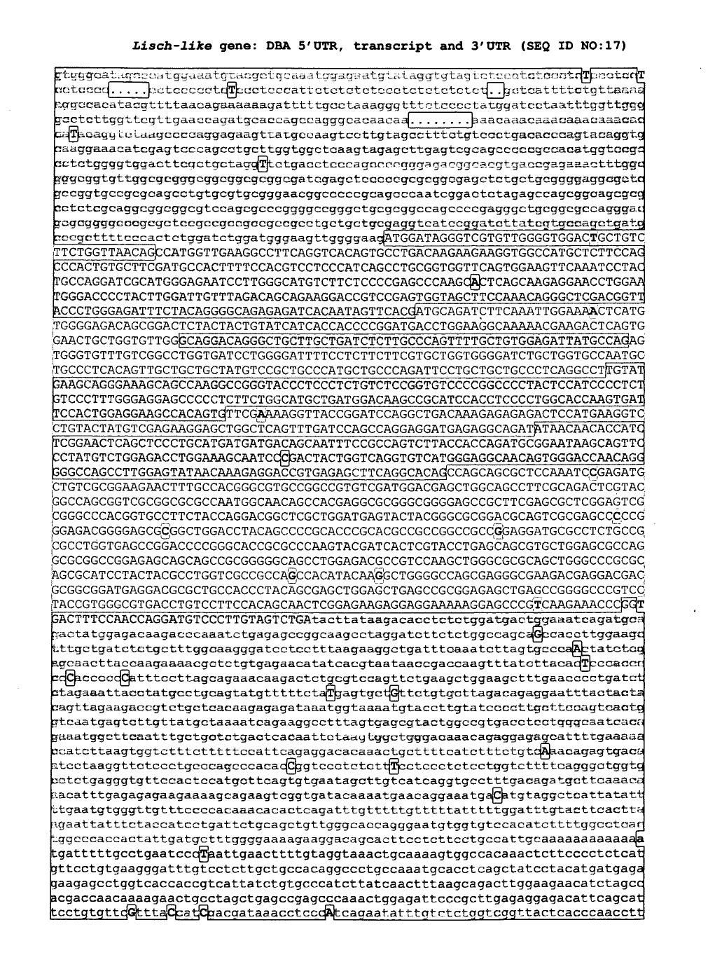

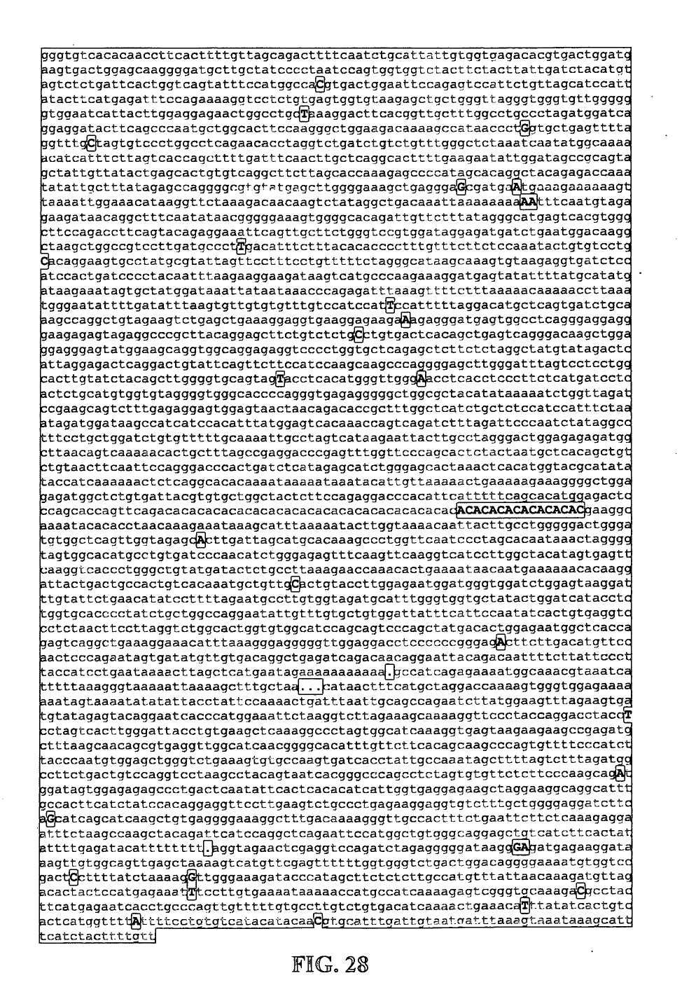

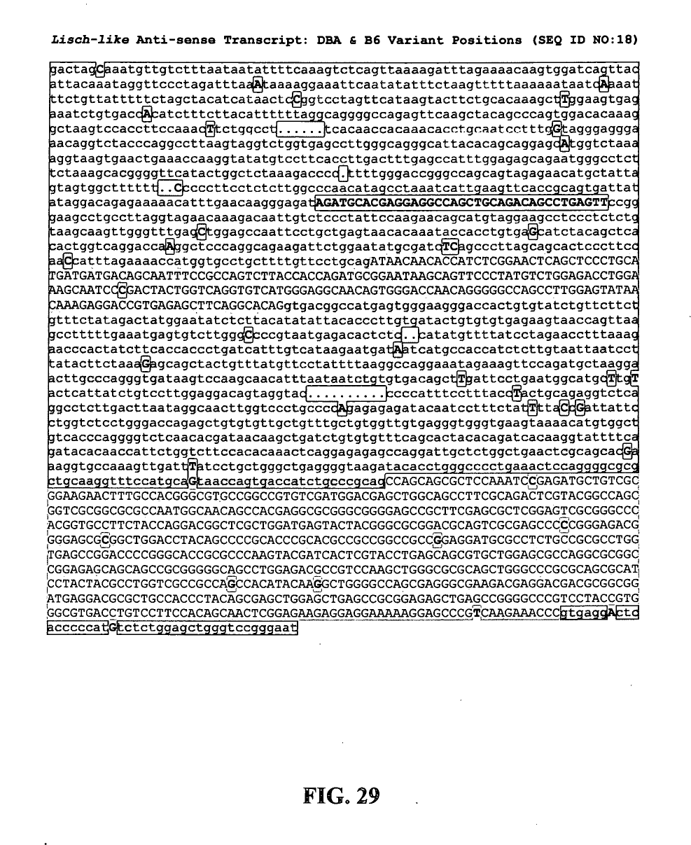

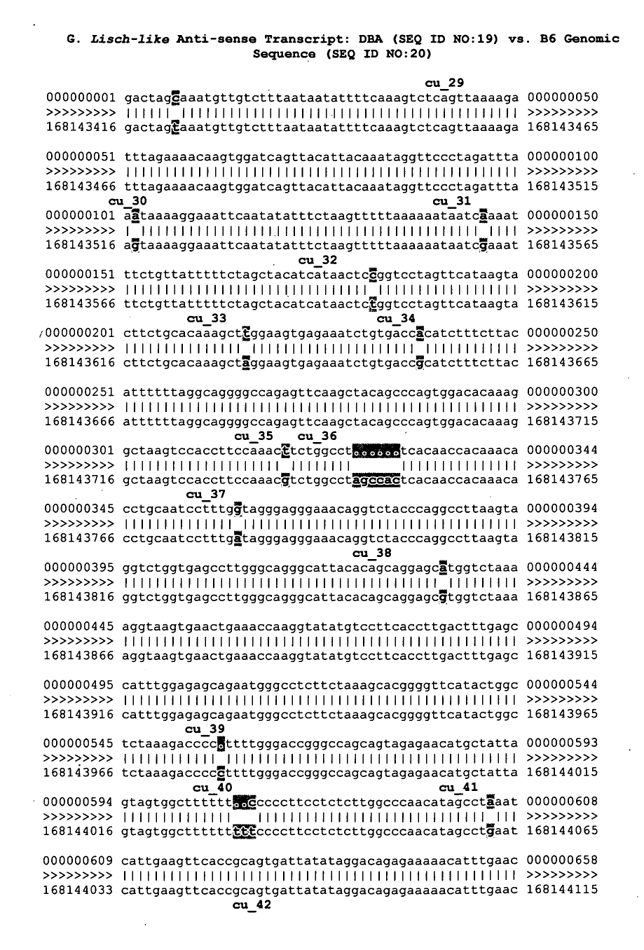

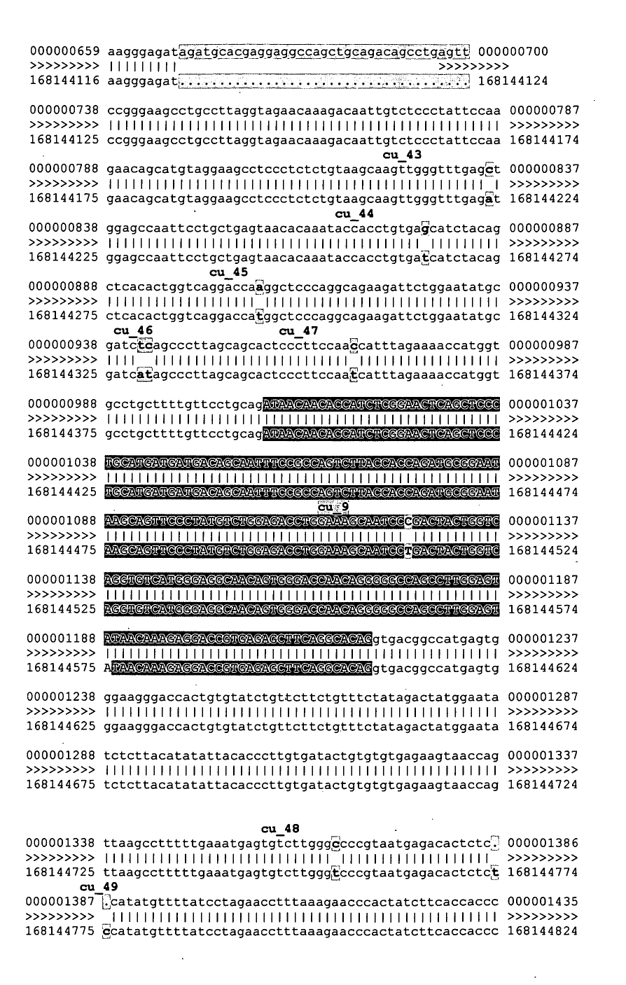

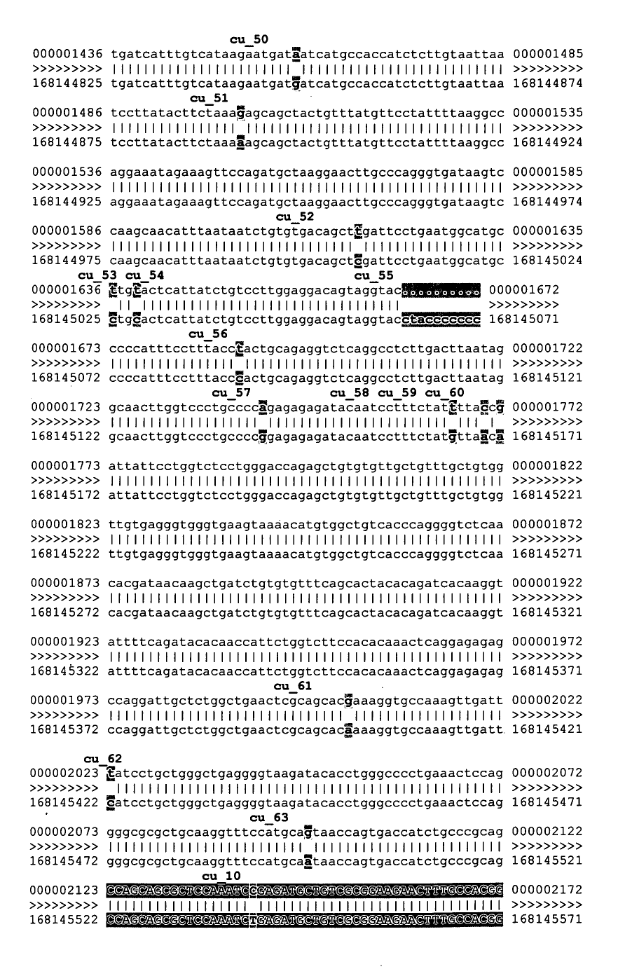

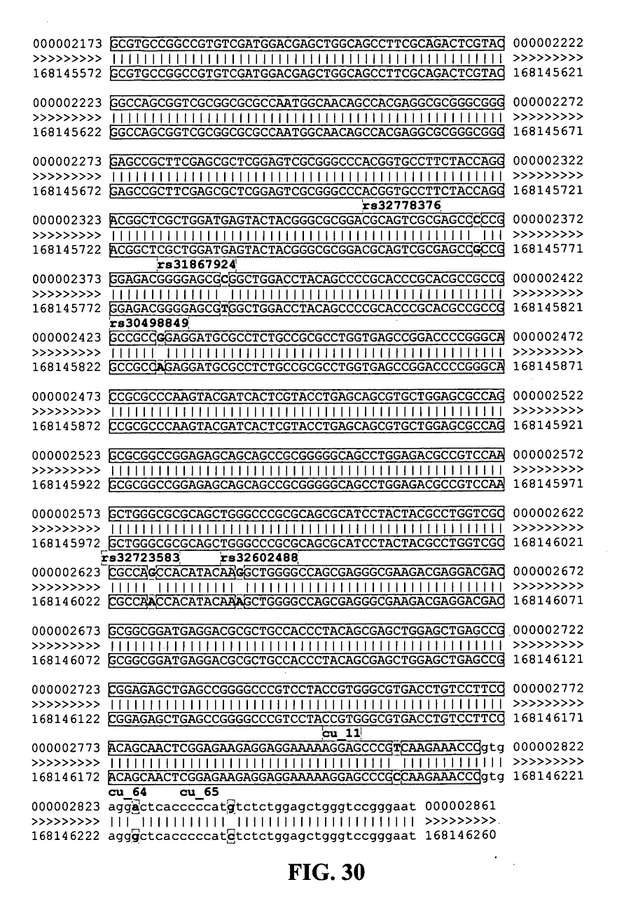

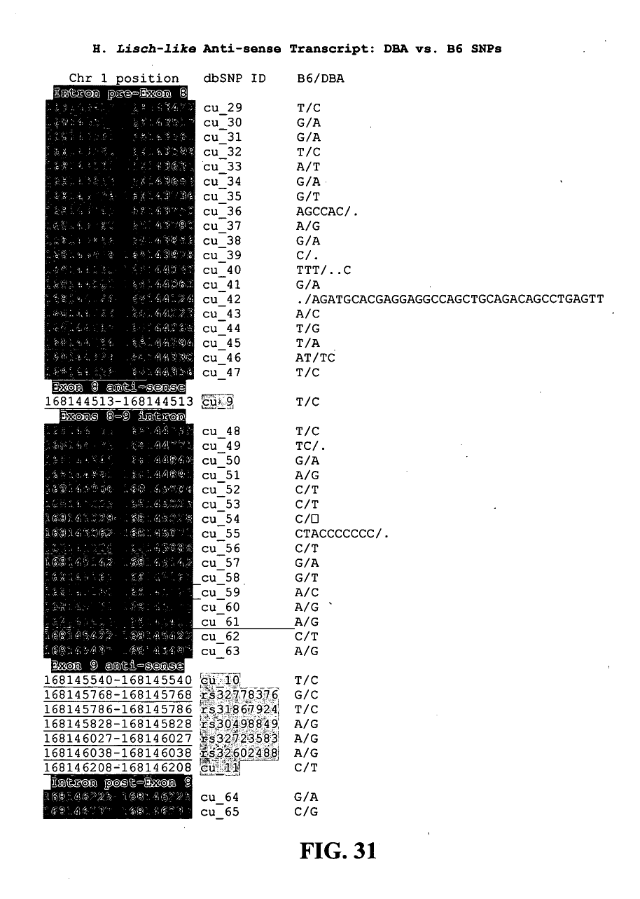

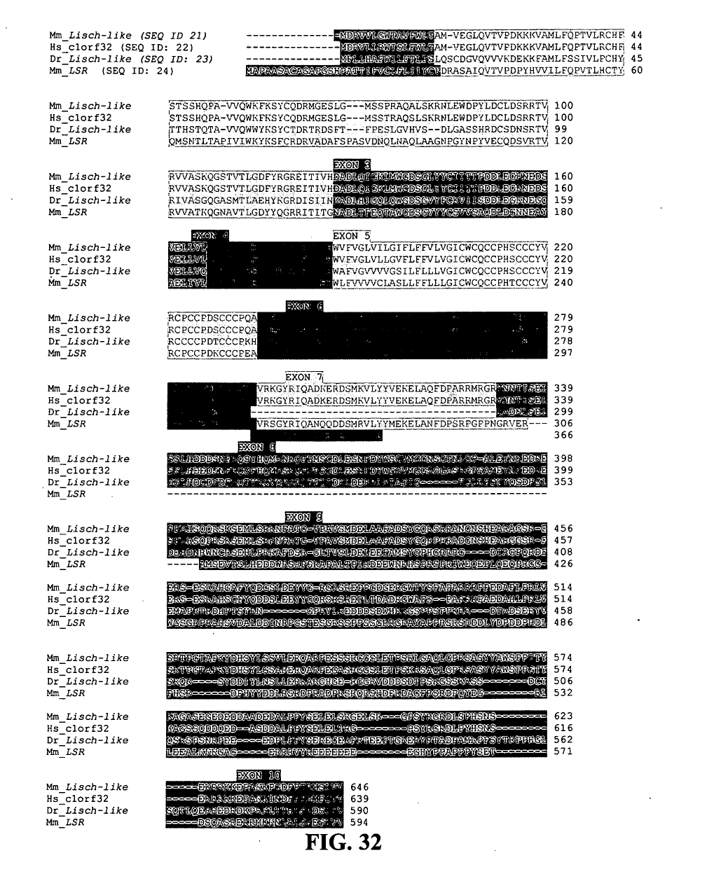

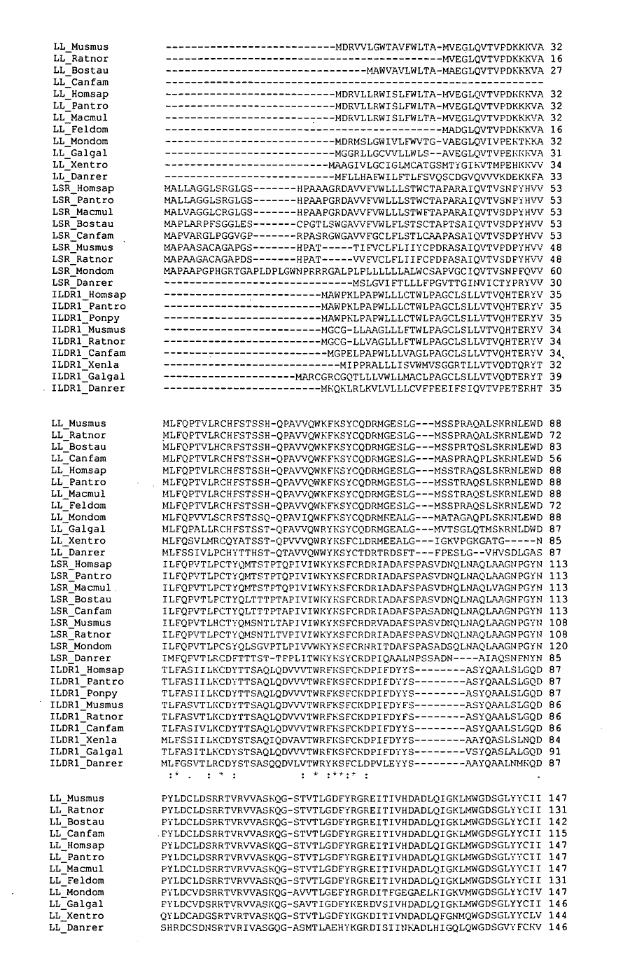

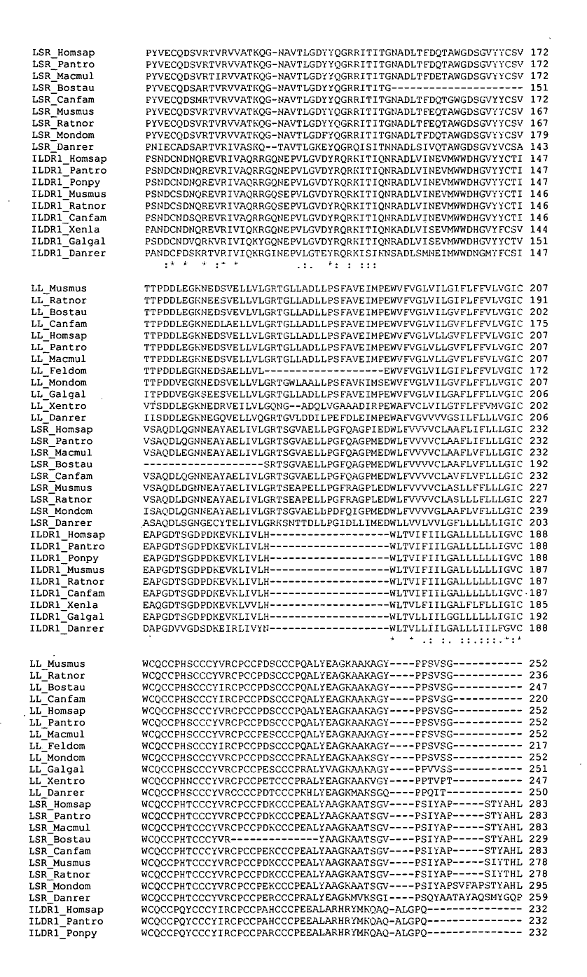

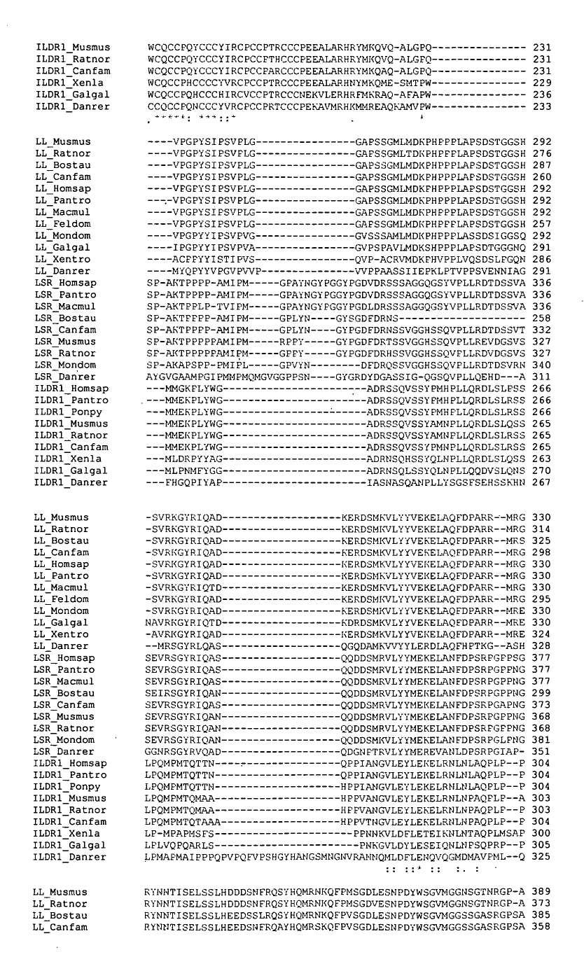

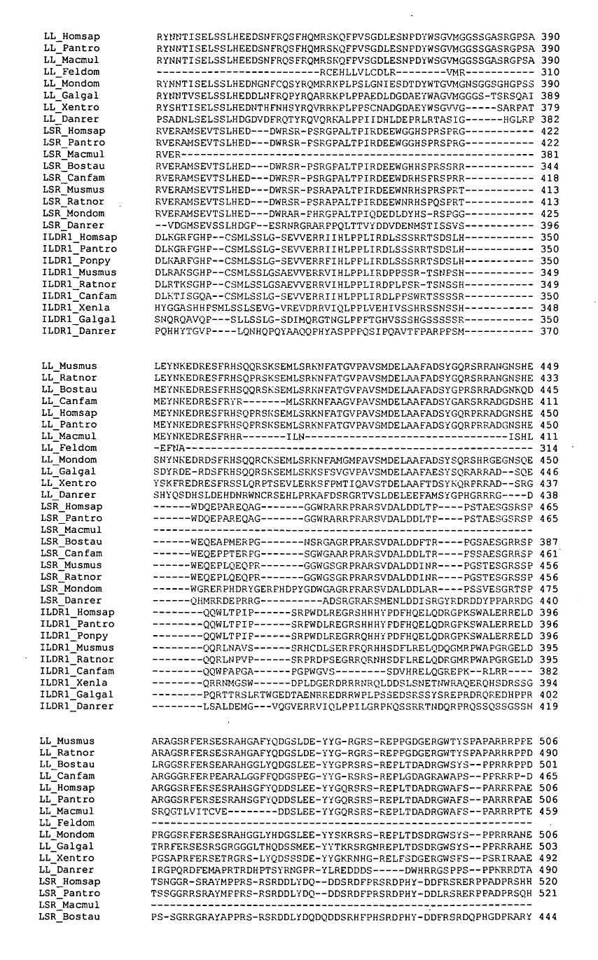

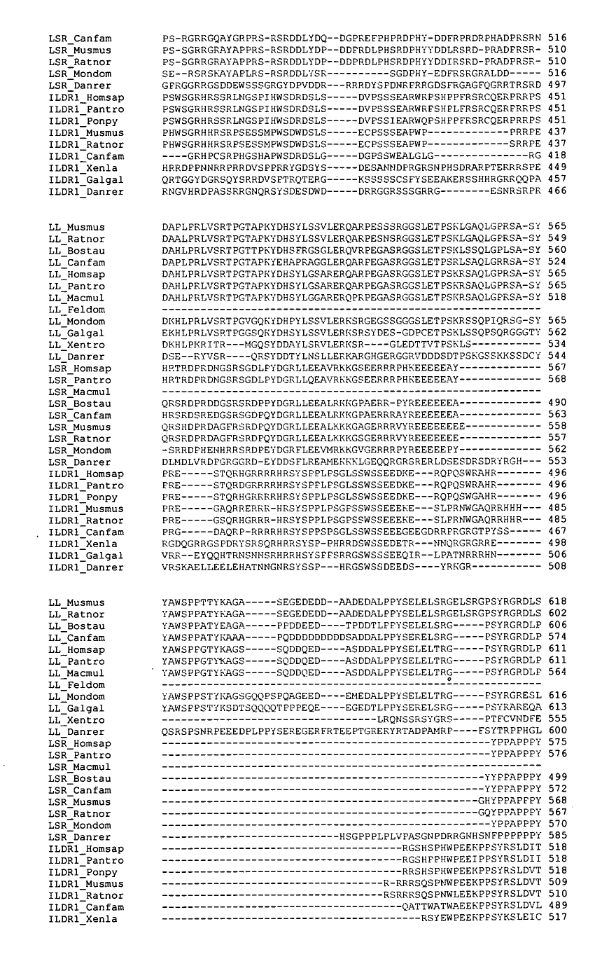

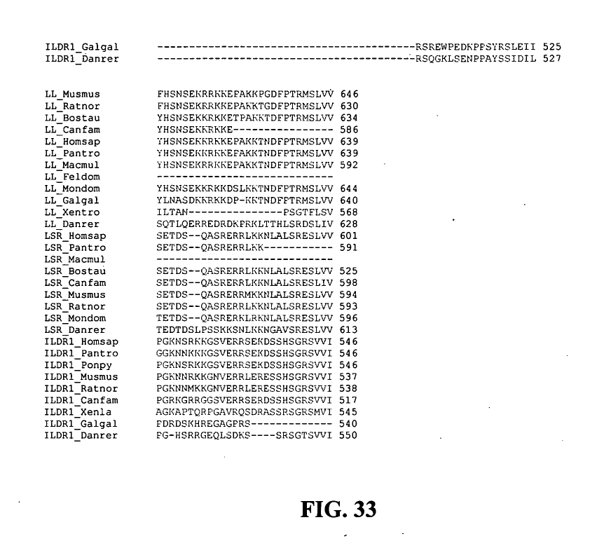

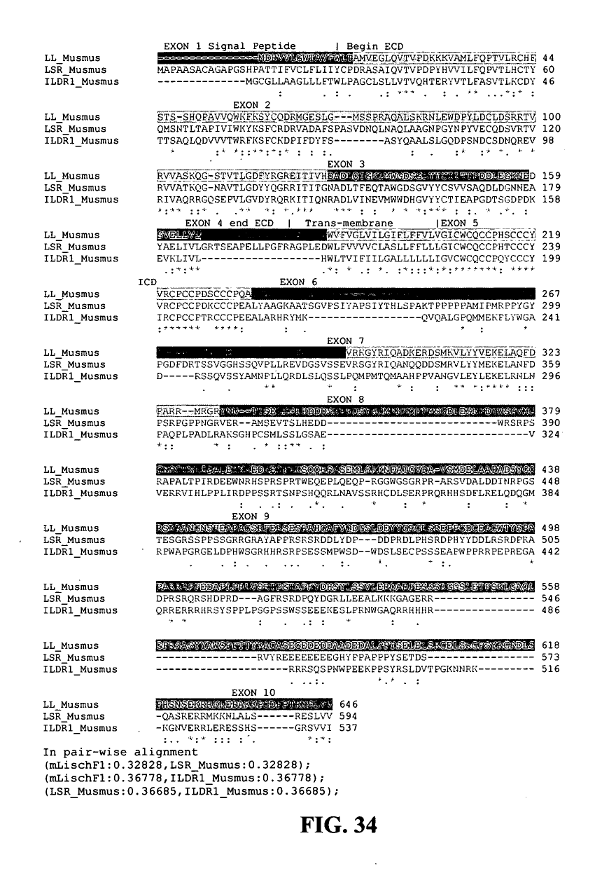

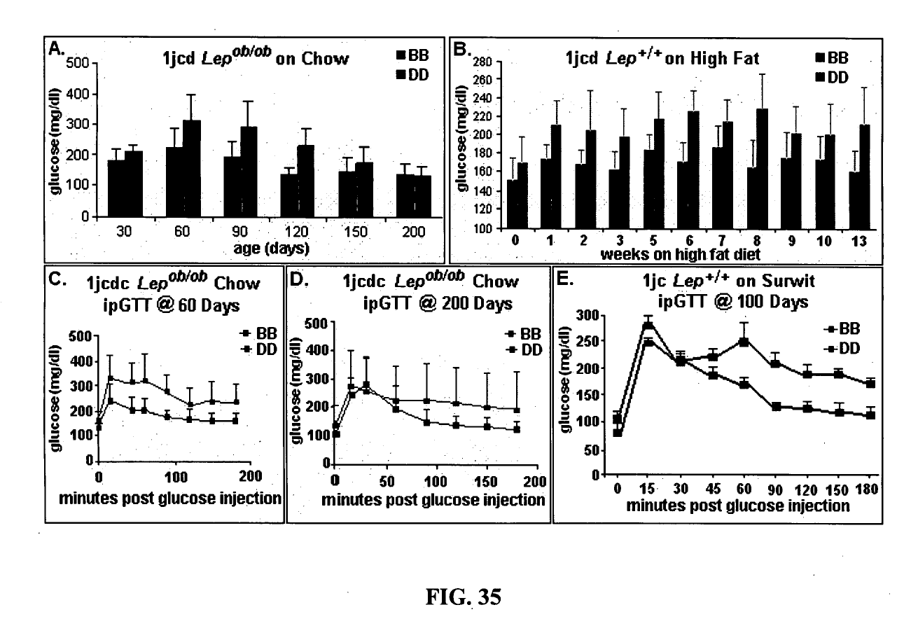

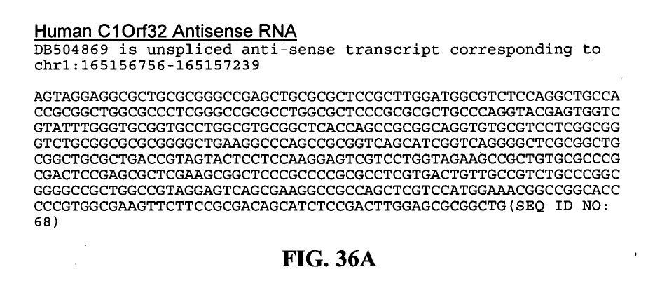

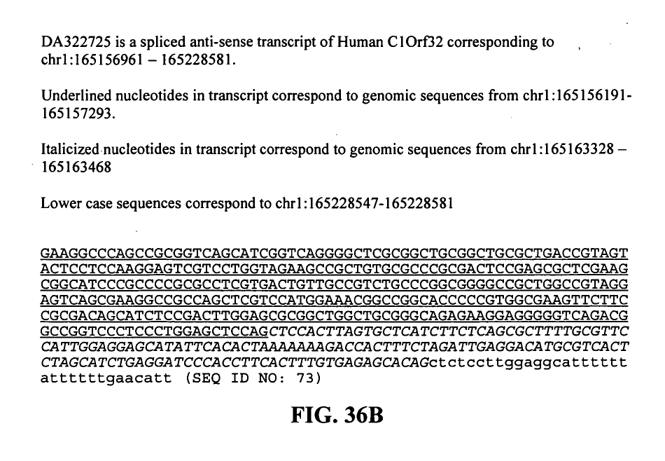

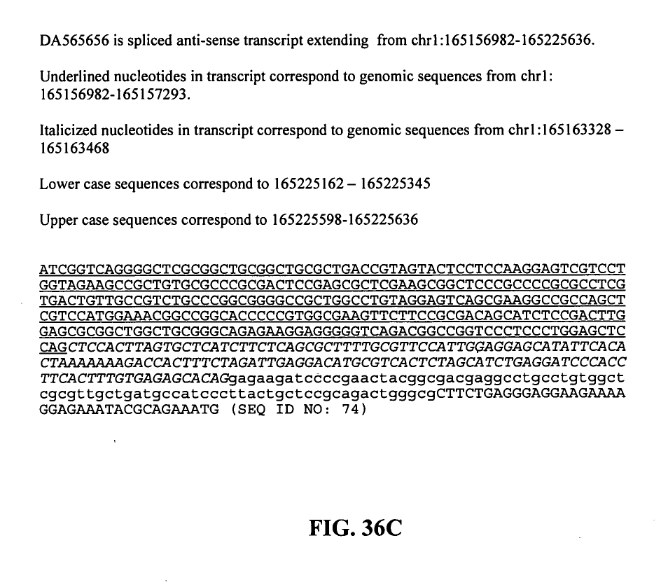

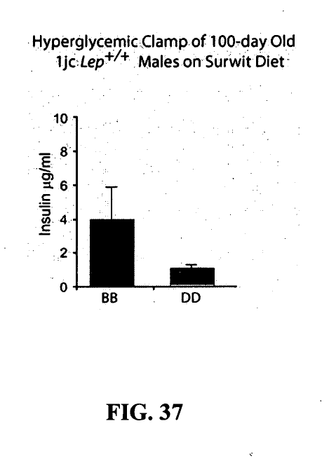

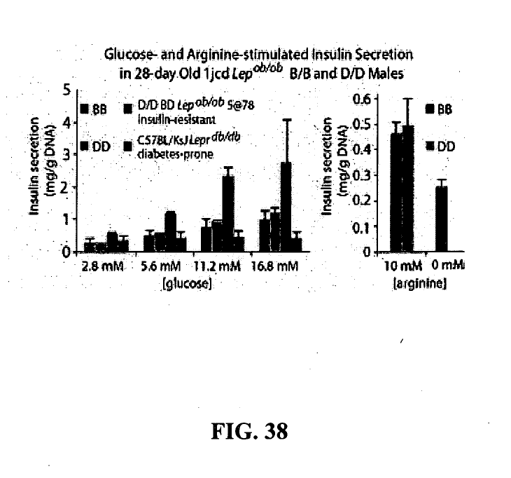

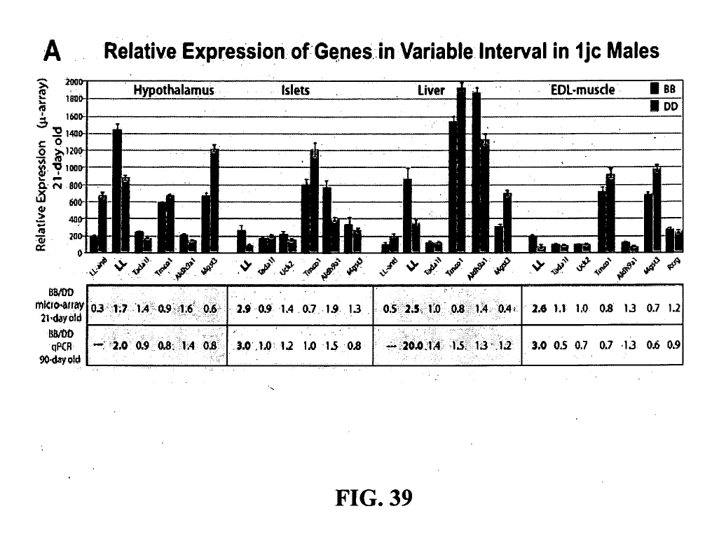

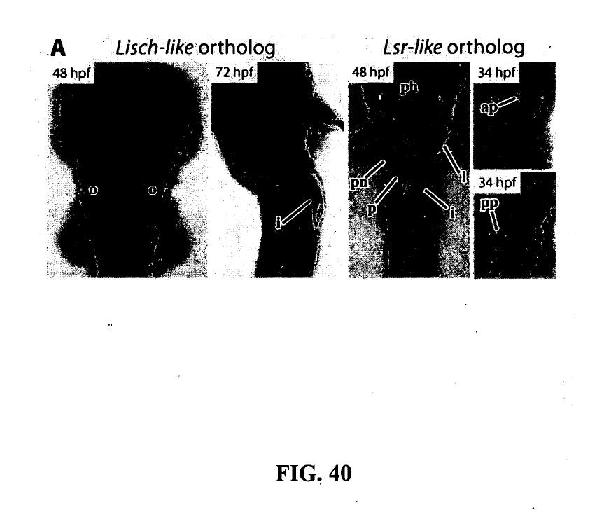

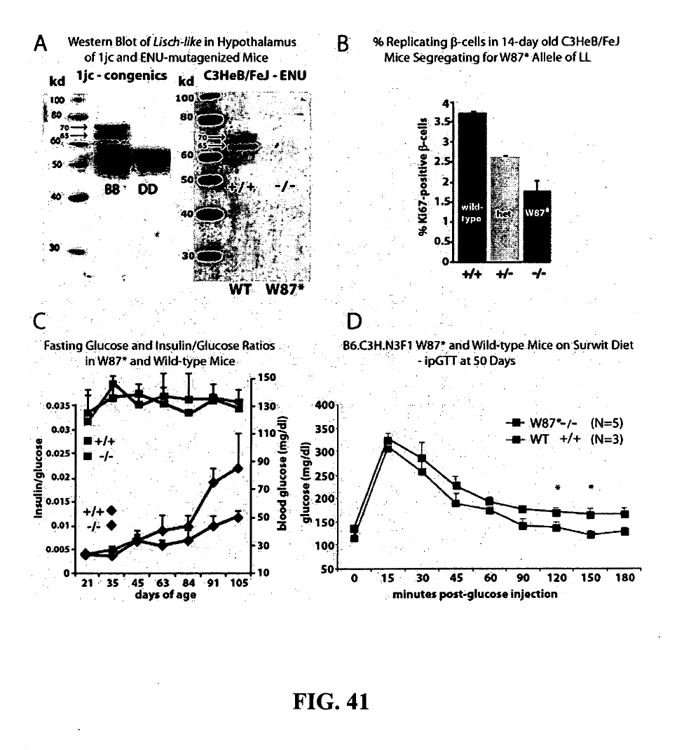

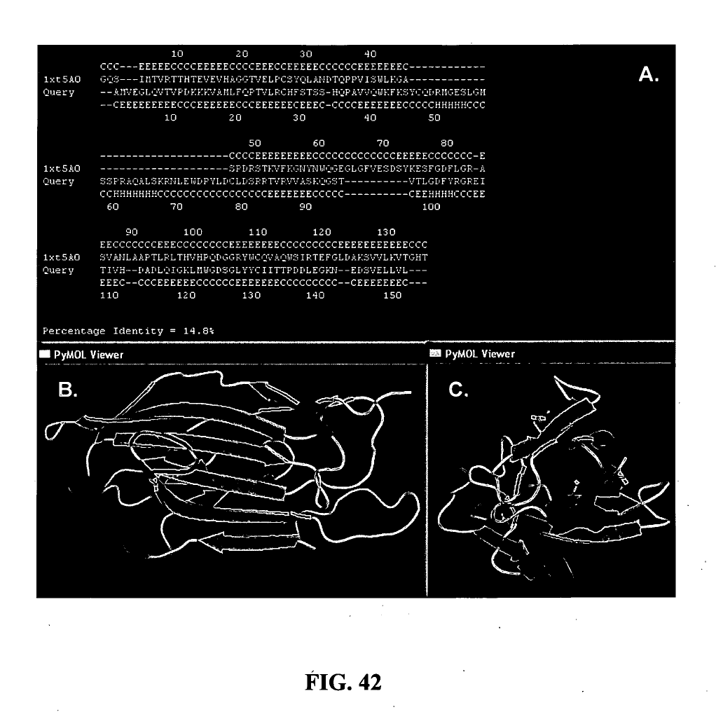

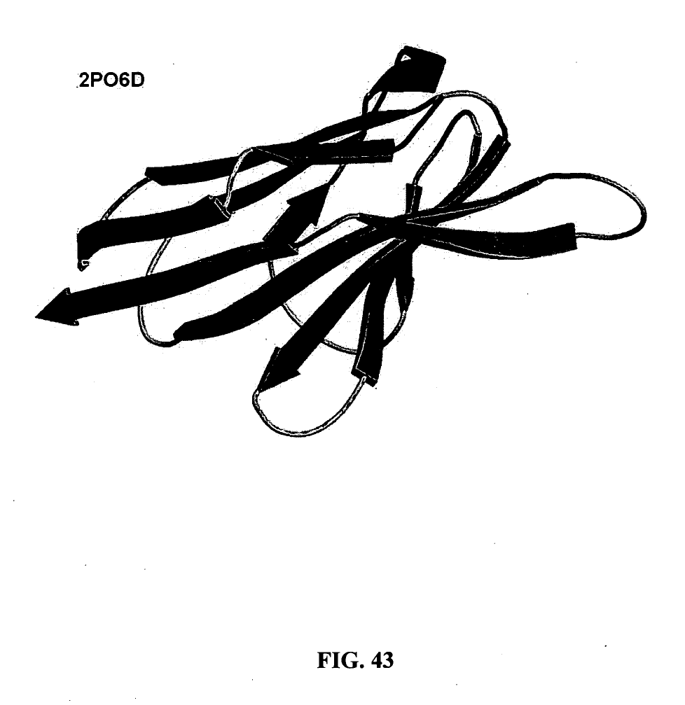

A repository of ENU-generated (ethylnitrosourea) mutant sperm DNAs from 18,000 C3HeB/FeJ G1 males was screened for mutations in Lisch-like (Ingenium) (Augustin et al, 2005, Mamm Genome 16:405-413). Non-synonymous mutations were detected in four separate samples and a nonsense (amber; stop) mutation at threonine-87 was detected in a fifth sample. Sperm containing the nonsense mutation were used for in vitro fertilization (IVF) using wild-type oocytes of the same strain as the mutant carrier. Point mutations were introduced into spermatogonia at a rate of about 20 per genome. Therefore, since the entire passenger mutations are freely segregating, the probability is low that, in a group of homozygous F3 animals, the observed phenotype, will be influenced by a recessive bystander mutation. FIG. 19 shows the positions and changes from wild-type of the five variants available to us. Functional consequences of the missense mutations are estimated using computational approaches described herein. These additional animals can be analyzed if there is indication that they can reveal structure-function relationships in LL. The ENU-generated repository of mutations can be screened further to identify additional mutations.

### Example 16

### Production of Conditional Knock-Out of Ll

To generate a hypomorphic allele of LL on a B6 background, a strategy was used which has been used successfully for creating knockout alleles of the leptin receptor: the insertion of a Pgk-NPT cassette in inverse orientation to the targeted gene (McMinn et al, 2004, Mamm Genome 15:677-685; Coppari et al, 2005, Cell Metab 1:63-72). The Pgk-NPT cassette has been reported to have cryptic splice sites that interfere with splicing of the locus that has been targeted. For other genes, such as Fgf8 and Rx, (Meyers 1998, Nat Genet. 18:136-141; Voronina et al, 2005, Genesis 41:160-164), allelic series were generated with a similar strategy.

In order allow a wide array Cre constructs with which to rescue the allele, a Pgk-NPT cassette flanked by loxP sites was used for the LL targeting construct instead of the cassette flanked by frt sites. A B6 BAC clone, RP23 169c19 (˜200 kbp), was identified that contains the entire Ll coding sequence. A 5 kbp Sac I fragment containing exon 1 was sub-cloned from the BAC. Insertion of a loxP-flanked geneticin resistance cassette, Pgk-neo, after the exon was achieved by at a BbvCI restriction site. Germline transmission was achieved in C57BL/6J mice, but the resulting LL allele did not show any alterations in expression resulting from the insertion of the loxP flanked Pgk-NPT cassette after exon 1 of the Ll locus. The Pgk-NPT cassettes between the loxP- and frt-flanked cassettes differ in that the loxP flanked cassette is shorter by ˜120 bp within the 5′ end of the Pgk promoter. The sequence differences at the ends of the two Pgk-NPT cassettes can be involved in their abilities to interfere (or not) with splicing of transcripts. As discussed herein, an ENU-generated exon 2 stop mutation segregating on C3HeB/FeJ, which is a diabetes resistant strain, is being created, and this animal can be used for preliminary analysis of the biology of Ll. Another conditional knockout allele can be made on the B6 background.

### Example 17

### Targeted Mutations of L1 in C57BL/6 Mice

Other methods can be useful for the creation of C57BL/6J mice segregating for transgenic constructs to examine the functions of L1. Such gene targeting vectors that can produce various levels of gene inactivation can be used to test whether inactivation of the L1 gene in C57BL/6 mice can confer diabetes susceptibility. A vector was designed for conditional mutagenesis that can be used for both ubiquitous and conditional inactivation. FIG. 20 shows different designs for conditional inactivation or activation of the mouse L1 gene. FIG. 20A shows genomic structure of the targeted L1 allele for (A) conditional inactivation or (B) activation. Exon 1 of the L1 gene (black rectangle), the PGKneo triple polyA cassette (white rectangle), loxP sites (black triangle) and FRT sites (white triangle) are depicted.

### Example 18

### Global Inactivation

To ablate Ll gene function completely upon cre-mediated recombination, the loxP sites will be position around the promoter region and exon 1 of the Ll gene while maintaining functionality of the Ll locus in the absence of cre. In the targeting vector a single loxP site 2 kb upstream will be insert of the transcriptional initiation site. A neomycin selection marker cassette flanked by FRT sites and one loxP site will be inserted downstream of exon 1. Since interference of the Pgk promoter of the neomycin cassette with transcription of the Ll allele cannot be excluded, as a precaution, the neomycin selection marker will be flanked with FRT (FLP recombinase target) sites to allow its removal in targeted ES cells by transient expression of the Flpe recombinase (Farley et al, 2000, Genesis 28:106-110) or by crossing mice carrying the targeted allele with FLPeR (Flipper) mice (Buchholz et al, 1998, Nat Biotechnol 16:657-662). For global inactivation, the sequence flanked by loxP sites will be excised in vitro, using transfections of ES cells carrying the gene-targeted allele (Bruning, 1998, Mol Cell 2:559-569), or by intercrossing mice carrying the Ll^{flox} allele with “deleter” cre transgenics. Relevant cre lines are available (Okamoto et all, 2004, J Clin Invest 114:214-223; Bruning et al, 1998, Mol Cell 2:559-569; Han et al, 2006, Cell Metab 3:257-266; Nandi et al, 2004, Physiol Rev 84:623-647; Xuan et al, 2002, J Clin Invest 110:1011-1019).

### Example 19

### Conditional Inactivation

L1 gene will be inactivated conditionally to determine whether Ll ablation in β cells, and/or liver, in the C57BL/6 background will affect their ability to proliferate, thus conferring diabetes susceptibility to this resistant mouse strain in vivo. Global inactivation of the Ll gene can result in lethality at late embryonic or peri-natal/early post-natal stages. This conditional inactivation will be achieved by inactiving the conditional Ll^{flox} allele at various developmental stages during endocrine pancreas differentiation using crosses of homozygous Ll^{flox/flox} mice with Neurogenin 3-cre, Pdx-cre and Insulin-cre. Each cre transgenic will cause Ll inactivation at a different stage in pancreas development, and will thus provide insight into the developmental role of Ll in this process. Importantly, the transgenic cre lines are maintained on an isogenic C57BL/6 background, allowing a determination of the phenotypic consequences of Ll inactivation in a normally diabetes-resistant background.

### Example 20

### Methods

Animal Husbandry. Mice were housed in a barrier facility in ventilated Plexiglas cages under pathogen-free conditions with a 12 hour light/dark cycle and 22±1° C. room temperature. Mice were weaned at 21 days and given ad libitum access to 9% Kcal fat Picolab Rodent Chow 20 (Purina Mills, Richmond, Ind.) and water. The high fat diet protocol used in some animals is described herein. After a 4-hour morning fast, mice were sacrificed by carbon dioxide asphyxiation and phenotyped for weight, naso-anal length, and glycosuria. Blood was collected by cardiac puncture into an anticoagulant cocktail containing 10 μl of 1 mM EDTA and 1.5 mg protein/ml aprotinin (Sigma A-6279). Plasma and red blood cell pellets were used to measure plasma glucose, insulin, and HbA1c as previously described (Chung et al, 1997, Genomics 41: 332-344). Tissues (skeletal muscle, pancreas/pancreatic islets, liver, brain, hypothalamus, kidney, spleen, heart, visceral fat, retroperitoneal fat) were collected and immediately frozen in liquid N₂, and stored at −80° C. for further studies. Pancreata were dissected under stereoscope, weighed, and fixed in Z-fix (zinc-formalin fixative, Anantech Ltd, Mich.).

Genotyping. Liver tissue or tail tips were used for genomic DNA isolation according to standard procedures (Amar et al, 1995, Embo J 4: 3695-3700). A mutation-specific assay was used to confirm that phenotypically obese animals were Lep^{ob}/Le^{ob} and lean animals +/+ or heterozygous at the Lep locus (Chung et al 1997, Diabetes 46: 1509-1511). Animals were genotyped for microsatellite markers as previously described (Chung et al, 1997, Genomics 41: 332-344). Primers for Map Pairs (microsatellites) were purchased from Research Genetics or Invitrogen (Carlsbad, Calif.).

Mapping T2D-Related Phenotypes in B6xDBA F2/F3 Progeny.

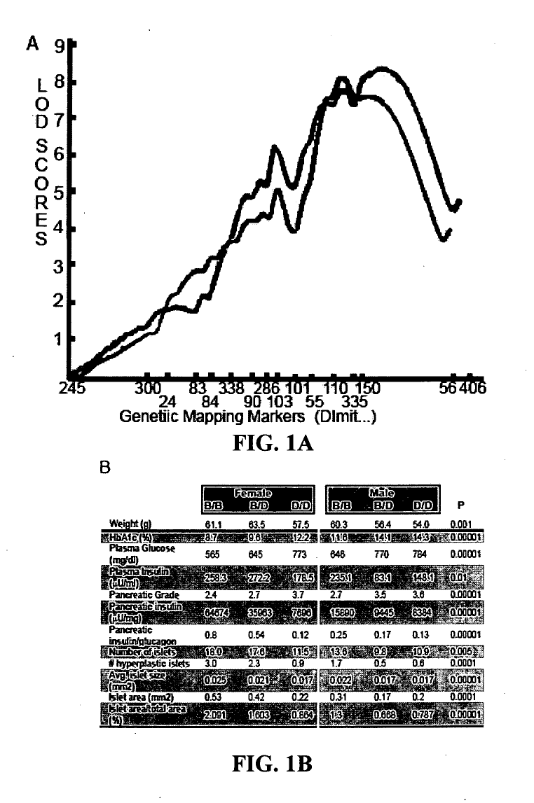

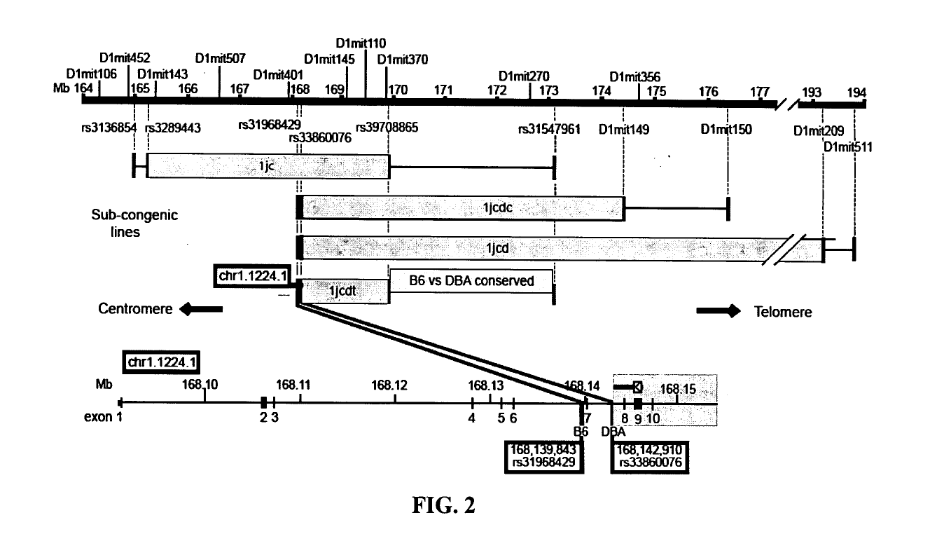

To identify genes mediating differential susceptibility to diabetes in the context of obesity, C57BL/6J (resistant) and DBA/2J (susceptible) inbred strains were used that are discordant for type 2 diabetes when made obese genetically. Maps were created using MapMarkerQTL on a dataset representing 404 obese F2 and F3 progeny of a B6/DBA cross segregating for Lep^{ob} at 120-150 days of age. The QTL for T2DM was most significantly associated with fasting blood glucose, glycosylated hemoglobin, and islet histology in male mice to a region of Chr1, with peak statistical significance at D1Mit 110 at 169.6 Mb from the centromere (p<10⁻⁸) (FIG. 1). Other QTLs were identified on other chromosomes (for example Chr5@78cM), but none had as great an effect on the phenotype or demonstrated consistent effects on all aspects of the phenotype. Interactions for QTLs were tested for and a modest interaction between the locus on chromosome 1 and a second locus at D4Mit286 (p=0.008) was identified.

B6.DBA Congenic Lines: Creation and Fine Mapping.

B6.DBA congenic mice were generated by intercrossing Lep^{ob}/Lep⁺ C57BL/6J X DBA/2J mice from Jackson Laboratory to generate F1 progeny, followed by backcrossing to the recurrent C57BL/6J strain using a “speed congenic” approach in subsequent generations (Visscher, 1999, Genet Res 74: 81-85). At the eighth backcross, a genome scan was performed in breeders using polymorphic markers at 20 cM intervals. In the mouse line that was continued, non-contiguous markers outside the interval were homozygous B6. Over the next two generations, there were two recombination events, one that eliminated the telomeric DBA interval (line 1jc) and one that preserved approximately half of the originally defined interval (line 1jcd). The 1jcd mouse was bred repeatedly to B6 mice, giving rise, by meiotic recombination, to two additional subcongenic lines (1jcdt and 1 jcdc) (FIG. 2). Preservation of the phenotypes in the original B6xDBA and DBAxB6 F2/F3 progeny was assessed by longitudinal and end-point measurements of fasting glucose, insulin, glycosylated hemoglobin and islet morphology. At N12, Lep^{ob/+} mice B6/DBA (B/D) for the congenic interval were intercrossed to produce N12F1 progeny. Obese progeny were used for fine mapping and phenotyping experiments. Lep^{ob/+} animals D/D for the congenic interval were recurrently intercrossed or crossed to B6 Lep^{ob/+} animals to generate ob/ob animals with D/D and B/D genotypes for the Chr 1 interval, respectively.

Studies of Glucose Homeostasis.

For longitudinal phenotyping studies, mice were fasted for 4 hours and restrained for blood collection by a trained individual. Blood was collected by capillary tail bleed in unanesthetized animals into heparinized tubes and stored at −80° C. Glucose was measured with an Ascensia glucometer (Bayer) or FreeStyle Flash Blood Glucose Monitor (Abbott); insulin and HbA1c were measured by ELISA (ALPCO) and affinity chromatography (Mega Diagnostics), respectively, as described herein. Urine ketones were measured using urine dipsticks (Chemistrip uGK, Roche). For intraperitoneal glucose tolerance tests (ipGTT), mice were fasted overnight and 0.5 g/kg body weight of 50% dextrose was administered at time 0. Plasma glucose was measured by capillary trail bleed using a glucometer at 15-30 min intervals for 3 hours. Terminal phenotypic characterization consisted of measurements of fasting glucose, insulin, glycosuria, and HbA1c as previously described (Chung, et al, 1997, Genomics 41: 332-344). To control for stress-induced hyperglycemia at the time of sacrifice, tail blood glucose was also measured one day prior to sacrifice with a glucometer.

High Fat and “Surwit” Diet Studies.

High fat chow pellets (#D 12492i: 60% kcal from fat, 20% kcal from protein, 20% kcal from carbohydrates) and “Surwit” (Wencel et al, 1995, Physiol Behav 57: 1215-1220). (#D12331i; 58% kcal from fat, 16.4% kcal from protein, 25.5% kcal from carbohydrates) were purchased from Research Diets (New Brunswick, N.J.). These diets were used as described herein.

Lisch-Like Antibodies.

Mouse polyclonal antibodies for LL were generated in rabbit and guinea pig, against the predicted ECD (residues 22-186) or against the predicted ICD (residues 298-401) of the protein. Peptides for injection were obtained by protein expression of mouse mRNA in human embryonic kidney 293 cells (HEK-293T). Peptide sequencing was used to confirm expression of the correct product. The following amino acid sequences were used as antigens for LL:

(SEQ ID NO: 6)
YRIQADKERDSMKVLYYVEKELAQFDPARRMRGRYNNTISELSSLHDDDS
NFRQSYHQMRNKQFPMSGDLESNPDYWSGVMGGNSGTNRGPALEYNKEDR
ESFR
(predicted Intracellular Domain, AA#298-401,
isoform 1, Fig. 23A)
(SEQ ID NO: 7)
QVTVPDKKKVAMLFQPTVLRCHFSTSSHQPAVVQWKFKSYCQDRMGESLG
MSSPRAQALSKRNLEWDPYLDCLDSRRTVRVVASKQGSTVTLGDFYRGRE
ITIVHDADLQIGKLMWGDSGLYYCIITTPDDLEGKNEDSVELLVLGRTGL
LADLLPSFAVEIMPE
(predicted Extracellular Domain, (AA#22-186,
isoform1, Fig. 23B).

FIG. 22 shows that the ICD and ECD rabbit antibodies detected the appropriate fusion proteins, with only minor cross-reactivity.

Immunohistochemistochemical and Morphometric Analysis of Pancreatic Islets.

Pancreatic tissues were dissected under stereoscope to avoid contamination with adipose tissue, and tissue weight was obtained. For IHC, pancreata were fixed in zinc-formalin fixative (Anantech Ltd, Mich.), embedded in paraffin blocks and sectioned. 4 μm sections were mounted on charged glass slides, deparaffinized and stained. Table 7 provides detailed information about specific experimental conditions used for insulin, glucagon, somatostatin, pancreatic polypeptide, Ki67, and Lisch-like immunostaining.

Islet Morphometry.

Non-overlapping images of longitudinal pancreatic sections were acquired using ImagePro software. Images were analyzed using ImageProPlus software version 5.0 (Media Cybernetics, Md.) in order to calculate insulin-positive area, insulin-positive area as % total area, and number of islets (defined by an area containing a minimum of 8 contiguous insulin-positive cells). For β-cell replication studies, Ki67⁺ insulin⁺ and Ki67⁻insulin⁺ cells were manually counted. Replication of β-cells was expressed as % (Ki67⁺ +insulin⁺)/total insulin-positive. For replication studies, ˜100 islets were examined per animal from several different non-overlapping sections through the pancreas. ImageProPlus or Image J (1.37 V; NIH) were used to determine the relative area of each section occupied by β-cells for each representative longitudinal pancreatic section (50 μm apart) that had been immunochemically stained for insulin as previously described (Finegood et al, 2001, Diabetes 50: 1021-1029). Five to seven sections from different regions of the pancreas were analyzed. Glucagon, somatostatin, and pancreatic polypeptide-stained slides were analyzed in the same way to determine the respective relative masses of these cell types. Apoptosis rates were assessed using the DeadEnd Fluormetric TUNEL System G3250 (Promega) TUNEL assay and cleaved Caspase-3 (Asp175) Antibody 96615 (Cell Signaling Technology).

Pancreatic Islet Isolation.

Pancreatic perfusion and islet collection were performed as previously described (Guillam et al, 2000, Diabetes 49: 1485-1491). Each pancreas was perfused via the bile duct with 1.5 mg/mL collagenase P (Roche Molecular Biochemicals, Mannheim, Germany) and incubated at 37° C. for 17 minutes. Following disaggregation of pancreatic tissue, pancreata were rinsed with M199 medium containing 10% NCS. Islets were collected by density-gradient centrifugation in Histopaque (Sigma-Aldrich, St. Louis, Mo.) (Guillam et al, 2000, Diabetes 49: 1485-1491), and washed several times with M199 medium. For glucose-stimulated insulin release studies (Lacy and Kostianovsky 1967, Diabetes 16: 35-39; Gotoh et al, 1985, Transplantation 40: 437-438), islets were incubated overnight in RPMI medium (Gibco Life Technologies, Rockville, Md.)

Glucose-Stimulated Insulin Secretion (GSIS).

The GSIS procedure has been described previously (Eizirik et al, 1989, Endocrinology 125: 752-759). Islets were hand-picked into tissue culture dishes containing cold Kreb's buffer (118.5 mM NaCl, 2.54 mM CaCl₂, 1.19 mM KH₂PO₄, 1.19 mM MgSO₄, 10 mM HEPES, pH 7.4), and 2% BSA (Sigma-Aldrich), 5.5 mM glucose and incubated overnight at 37° C. Islets were hand-picked and incubated another 15 min. in Kreb's buffer+BSA, containing 11.2 mM glucose. Hand-picked islets are then resuspended in Kreb's buffer plus BSA, supplemented with 2.8 mM glucose and shaken at 37° C. for 15 mM. The pellet was spun down gently and resuspended in triplicate (5-10 islets each) in 500 μl Kreb's buffer, supplemented with glucose at final concentrations of 2.8 mM, 5.6 mM, 11.2 mM or 16.8 mM, or supplemented with 10 mM arginine and incubated for 1 h in a water bath at 37° C. with constant shaking (300 rpm). After 1 h incubation, islets were gently pelleted and the supernatant collected and assayed for insulin by ELISA. Islet pellets were dissolved in high salt buffer (2.15M NaCl, 0.01M NaH₂PO₄, 0.04M Na₂HPO₄, EDTA 0.672 g/L, pH 7.4) and sonicated at 4-5 W for 30 s and DNA concentration was measured using a TKO100 fluorometer (Hoefer) with Hoechst #33258 dye (Polysciences). Results were expressed as concentration of secreted insulin/[DNA]/h.

Testing for Predicted Transcripts in cDNA Pools.

Putative transcripts, identified from public annotation and local sequencing, were validated by PCR-amplification from tissue-specific cDNA pools prepared from male and female B6 mice. Two cDNA pools were used: 1. An inclusive cDNA pool was prepared from E7 and E20 fetuses and P1 pups and included the following tissues of 60-day old mice: eyes, large intestine, skin, tongue, spinal cord, kidney, testes/ovaries, pancreatic islets, whole brain, hypothalamus, skeletal muscle, and liver. This pool was used for transcript validation. 2. A diabetes-relevant cDNA pool, from 90-day old mice, was comprised of only the following tissues and organs: pancreatic islets, whole brain, hypothalamus, skeletal muscle, liver, and adipose tissue. This pool was used to quantify transcripts identified by computational approaches and the microarrays. Nominal intron-spanning primers were generated using the Primer3 program. Amplification was first performed on the diabetes-relevant pool at an annealing temperature of 60° C. If we detected no PCR-product, we performed gradient temperature PCR on the same pool using eight different annealing temperatures from 58-68° C. Gradient temperature PCR was then used to amplify the inclusive cDNA pool. If no product was detected in this pool, a 2nd set of intron-spanning primers was used before we interpreted negative amplification as failure to substantiate a predicted transcript. Positive amplification products of predicted sizes, and those that did not match the expected sizes, were gel-purified and sequenced for confirmation. The final set of primer-pairs is listed in Real-time qPCR.

Microarray Gene Expression Analysis.

RNA extraction, purification, labeling, hybridization and analysis were performed as described (Weisberg S P, McCann D, Desai M, Rosenbaum M, Leibel R L, et al. (2003) Obesity is associated with macrophage accumulation in adipose tissue. J Clin Invest 112: 1796-1808). 10 BB and 10 DD 21-day old Lep^{ob/ob} 1jc males were dissected and RNA was extracted from hypothalamus, liver, isolated islets, EDL muscle, and soleus muscle. Individually labeled RNA (by mouse and organ) was interrogated with Affymetrix MOE430A expression arrays. For all transcripts in the region of interest, where possible, only probes that spanned multiple exons and clearly represented each of the 14 genes in the interval were used. If >1 probe met these conditions, we used only, the probe that gave the strongest signal. Organs were grouped into two groups by genotype and were compared using a two tailed T-test. The Affymetrix probe IDs selected for this analysis are shown in the table below.

| | | | | | |
|---|---|---|---|---|---|
| Gene | LL anti | LL | Tada1l | Pogk | C030014K22Rik |
| Probe | 1436894_at | 1436293_x_at | 1424427_at | 1459896_at | 1440242_at |
| Gene | Uck2 | Tmco1 | Aldh9a1 | Mgst3 | Lrrc52 |
| Probe | 1448604_at | 1423759_a_at | 1437398_a_at | 1448300_at | 1432913_at |
| Gene | Rxrg | Lmx1a | Pbx1 | | |
| Probe | 1418782_at | 1421554_a | 1449542_at | | |

Real-Time qPCR.

Effects of the DBA/2J congenic interval on the levels of confirmed transcripts expressed in diabetes-relevant organs were assessed on an organ-specific basis. For these studies, separate pools were made from 90 day old Lep^{ob/ob} 1jc D/D and B/B mice for each of the diabetes relevant organs. Each individual organ pool was generated on 2 occasions from 5 mice. (Table 5).

For the analysis in Table 8, human RNA was purchased from Clontech (Human RNA Master Panel II; Clontech catalog number 636643), and human pancreatic islet RNA from a non-diabetic patient. The mouse cDNA was purchased from Clontech (mouse panels I and III (catalog numbers 636745 and 636757) and consisted material collected from 8 to 12 week old BALB/c (adult organs) or Swiss Webster embryos (aged embryos).

RNA was extracted from organs with acid-phenol reagent (TRIzol, Invitrogen Corp.). 2 μg of RNA were reverse-transcribed using SuperScript III reverse transcriptase (cDNA First Synthesis Kit, Invitrogen) with random hexamer priming cDNA was diluted 4-fold using nuclease-free water (Quiagen, Inc.). 2 μl of diluted cDNA were amplified by PCR in a LightCycler (Roche Applied Science). A standard curve for each transcript was generated using cDNA diluted 1:1, 1:10, and 1:100. The number of mRNA molecules was assessed in each sample using the slope and intercepts of PCR product appearance during the exponential phase of the PCR reactions optimized for transcript-specific product using specific primers. Each sample was run in triplicate in the same LightCycler run. Using LightCycler Software (Roche), the crossing point (CP) was calculated for each sample. The CP is the first maximum of the second derivative of the fluorescence curve, and is equivalent to the number of cycles at which the fluorescence first exceeds background. In the exponential phase, the relationship between CP values and the initial concentration of the transcripts is linear. Relative concentration ratios, normalized to actin, were calculated as follows:

R=η_{gene}^{(ΔCPgene (sample-ref))}/η_{actin}^{(ΔCPhg (sample-ref))}

In this expression, ΔCPgene is the CP of the gene in the sample minus the CP of the gene in the relevant reference; ΔCPhg is the CP of the housekeeping gene in the sample minus the CP of the housekeeping gene in the reference (“ref”) sample; and the efficiency (where 2 is perfectly efficient) as determined by the negative slope of the plot generated when CP is plotted as a function of the log of initial concentration determined in the standard curve. Each CP listed is the mean of CP values of the triplicates for each sample. Results are summarized in Tables 5 and 8. The primers used are shown in the table below:

| Gene | Forward Primer (5′-3′) | Reverse Primer (5′-3′) |
|---|---|---|
| Actin | AGCCATGTACGTAGCCATCC | CTCTCAGCTGTGGTGGTGAA |
| | (SEQ ID NO: 81) | (SEQ ID NO: 82) |
| Lisch-like | ATCTGCTGGTGCCAATGCTG | GCCGTACGAGTCTGCGAAGG |
| | (SEQ ID NO: 83) | (SEQ ID NO: 84) |
| Tada1l | TGGGCCAACCTGAAGTTGTGGT | GCCCTTGGGTTTTCCAGGCT |
| | (SEQ ID NO: 85) | (SEQ ID NO: 86) |
| Pogk | CTGAATTTGACCCTGAAAGAAGA | ACTTCCCGGTAGAGGGC |
| | GC | (SEQ ID NO: 88) |
| | (SEQ ID NO: 87) | |
| FMO13 | AGGTTTAACCATGCCAATTATGGA | CTCTGGGGCTTTTCACAAACT |
| | C | (SEQ ID NO: 90) |
| | (SEQ ID NO: 89) | |
| FMO9 | TGGAGCTTGGTCGTGTAG | CCACGGTGCCATCATCAA |
| | (SEQ ID NO: 91) | (SEQ ID NO: 92) |
| FMO12 | AATTCTGGAGCAGATGTGGC | CATGGTCCCAAACTCGATTC |
| | (SEQ ID NO: 93) | (SEQ ID NO: 94) |
| C030014K22 | ATCGTCCTGCGCTACAAGACCC | GGGTCACAGTCTCTGTCGTGTTCC |
| | (SEQ ID NO: 95) | (SEQ ID NO: 96) |
| Uck2 | GGGAGCGTGCGTCGGT | AGGACTCGGTAGAAGCTATCCTGG |
| | (SEQ ID NO: 97) | C |
| | | (SEQ ID NO: 98) |
| Tmco1 | GCAGACACGCTGCTCATCGT | CGCGAACATGGATTTCATCCGTAC |
| | (SEQ ID NO: 99) | C |
| | | (SEQ ID NO: 100) |
| Aldh9a1 | ACGGGAAGTCCATATTTGAGGCCC | GGAGGCGCACCCGCTTT |
| | (SEQ ID NO: 101) | (SEQ ID NO: 102) |
| Mgst3 | GGCGCACGAAGGTGAGCC | CCTCGATACCGCTTGCTAGGGT |
| | (SEQ ID NO: 103) | (SEQ ID NO: 104) |
| Lrrc52 | ACCGGATTGCACATCATCGACCA | CCCCGCTCGACGTTCGGA |
| | (SEQ ID NO: 105) | (SEQ ID NO: 106) |
| Rxrg | CAGTAGCCTTGCCCACGGG | ACCTGGTAAGGGCTTGATGTCCT |
| | (SEQ ID NO: 107) | (SEQ ID NO: 108) |
| Lmx1a | CTTCGAGGCCATTGCGCCC | GGGTCGCTTATGGTCCTTGCCG |
| | (SEQ ID NO: 109) | (SEQ ID NO: 110) |

Cloning and Sequencing of Lisch-like Isoforms.

Full-length Ll cDNAs were amplified from either B6 islets (isolated by us) or from Clontech MTC Panels 1 #636745 and 3 #636757, containing pooled multiple tissue cDNAs from 8-12 week old BALB/c mice and from Swiss Webster embryos. In a final volume of 50 μl, 0.5 μl LA Taq (TaKaRa) was added to a cocktail containing TaKaRa GC Buffer II, 400 μm each dNTP, 1 μl cDNA and 1 μl each primer (300 ng/μl).

| | |
|---|---|
| Exon1_Forward | 5′-GCAGCCCAATCGGACTCTA-3′ |
| primer | (SEQ ID NO: 111) |
| Exon10_Reverse | 5′-ACATCCTGGTTGGAAAGTCG-3′ |
| primer | (SEQ ID NO: 112) |

Samples were cycled in an MJ Tetrad Thermalcycler (BioRad; www.bio-rad.com) using a Touchdown protocol of a 2 min. extension and decreasing annealing temperature from 60° C. to 55° C. for 10 cycles, followed by 25 cycles with an annealing temperature of 55° C. Each sample was TOPO TA cloned (Invitrogen) and plated. From all three libraries, a total of 140 colonies were picked and grown overnight in LB buffer. Inserts were amplified by colony PCR and sized by gel-fractionation. Inserts representing each unique size were then sequenced. The isoforms and the exons deleted (Δ): iso1 (intact 10 exons); iso2, Δ6; iso3, Δ4,5,6; iso4, Δ4; iso5, Δ5,6; iso6, Δ9; iso7, Δ,5,6,7,8,9.

Computational Methods for Evaluating Effect of nsSNPs.

Five methods to compute the likelihood of seeing a functional change due to single amino acid substitutions resulting from the identified nsSNPs were used (see Table 5). SNAP, PolyPhen, and SIFT predict changes in protein function due to a single amino acid substitution. SNAP (Bromberg and Rost 2007, Nucleic Acids Res 35: 3823-3835) is a neural-network based method that considers protein features predicted from sequence (e.g., residue solvent accessibility and chain flexibility). Scores from −9 to +9 are estimates of accuracy of prediction, computed using a testing set of ˜80,000 mutants. A low negative score indicates confidence in prediction of neutrality (functional change absent), whereas a high positive score indicates confidence in prediction of non-neutrality (functional change present). Accuracy was computed separately for neutrals using the equation below:

$${Accuracy}_{neutral} = \frac{number of correct neutral predictions}{total number of neutral predictions}$$

PolyPhen considers structural and functional information and alignments. Predictions are sorted into 4 classes: benign, possibly damaging, probably damaging, and unknown.

SIFT predictions: SIFT (Ng and Henikoff 2003, Nucleic Acids Res 31: 3812-3814) is a statistical method that only considers alignments. Scores range from 0 to 1. Scores >0.05 indicate neutrality of a substitution.

PAM250 matrix substitutions: PAM matrix (Schwartz and Dayhoff 1978, Science 199: 395-403) (Percent Accepted Mutations) is derived from observing how often amino acids interchange throughout evolution (by evaluating alignments of proteins in a family). The lowest score is −8 (substitution of this type very rarely occurs, e.g. W->C) and the highest is 17 (same residue found in almost all proteins in alignment, e.g. W->W).

Percentage in Alignment (PROFacc):

The score is reported as the difference in observed percentages of wild-type and mutated residues in alignments against a non-redundant database (at 80% sequence identity) composed of UniProt (Bairoch A, Apweiler R, Wu C H, Barker W C, Boeckmann B, et al. (2005) The Universal Protein Resource (UniProt). Nucleic Acids Res 33: D154-159) and PDB (Berman et al, 2000, Nucleic Acids Res 28: 235-242). Scores range from −100 (if the mutant is observed at all times) to +100 (if the wt is observed at all times); 0 if the mutant is observed as often as the wt. Scores near 0 favor the likelihood of a mutation being neutral.

TABLE 5
Amino Acid Variants and Transcript Ratios in Confirmed Genes
in the Chr1 (“variable”) Interval 168.1-169.9 Mb

| | Amino acid | | |
|---|---|---|---|
| | changes | | Transcript ratio |
| Confirmed Genes | B6 > DBA | name/gene family/annotation | BB/DD > 2 |
| chr1.1224.1 | T572A | Lisch-like | Liver >10x |
| | A632V | (lipolysis-stimulated remnant | Adipose 2x |
| | | receptor-related) | Brain 2x |
| | | | Islets 2x |
| | | | Muscle 2x |
| Tada1l | none | SPT3-associated factor 42 | Same |
| Pogk | none | pogo transposable element with | Same |
| | | KRAB domain | |
| LOC226601 | K282E | flavin-containing monooxygenase | inclusive only |
| (FMO13) | | family; FMO-like | |
| 4831428F09Rik | Q5R | flavin-containing monooxygenase | inclusive only |
| (FMO9) | | family; FMO-like | |
| LOC226604 | V239I | flavin-containing monooxygenase | Inclusive only |
| (FMO12) | | family; FMO-like | |
| C030014K22Rik | none | unknown | Same |
| Uck2 | none | uridine monophosphate kinase | Same |
| Tmco1 | none | membrane protein of unk. function | Same |
| Aldh9a1 | none | Aldehyde dehydrogenase 9, subfamily A1 | Same |
| Mgst3 | none | microsomal glutathione-S-transferase 3 | Same |
| Lrrc52 | none | Leucine-rich repeat (LRR) protein of | Same |
| | | unk. function | |
| Rxrg | none | retinoid X receptor, gamma | Brain 2x |
| Lmx1a | none | LIM homeobox transcription factor 1, α | Brain 0.5x |

Genes in the “variable interval” (FIG. 2) were confirmed by PCR-amplification in cDNA pools described in Methods: “Testing for Predicted Transcripts in cDNA Pools”. Known genes are in bold type and predicted transcripts in normal type. “Inclusive only” indicates that the transcript was detected only in the cDNA pool that included whole embryos, 1 day pups, and other tissues, but not in the cDNA pool prepared only from “diabetes-relevant” organs (see Methods). Amino acid changes are shown using one-letter symbols flanking the position in isoform 1. Nucleotide substitutions were confirmed by bidirectional sequencing in both C57BL/6J and DBA. Transcript ratios were determined by qRT-PCR analysis, using a Roche LightCycler 2.0, normalized to actin, in the 1jc congenic line. Each of the 11 transcripts that were confirmed and detected in the “diabetes-relevant” organ pool was quantified individually in each of 5 diabetes-related organ-specific pools (liver, islets, brain, adipose tissue, skeletal muscle) prepared from 5 D/D and B/B 1jc Lep^{ob/ob} 90 day old mice.). “Same” indicates no detectable difference in expression B/B vs. D/D in any of the diabetes-relevant single organ pools.

TABLE 6
Similarity, by Domain, Between the Mouse
Lisch-like Isoform 1 and Related Proteins

| | amino acid | full- | | | |
|---|---|---|---|---|---|
| | residues | length | Ig-like | TM | |
| Protein | (#) | protein | domain | domain | ICD |
| H. sapiens C1orf32 | 639 | 90 | 98 | 98 | 87 |
| D. rerio LI-paralog | 629 | 36 | 51 | 70 | 26 |
| M. musculus Lsr | 594 | 34 | 47 | 70 | 25 |

Similarity scores for pairwise alignments were determined by clustalW analysis on the EMBL-EBI server using their default settings between the full-length LL protein (isoform 1) and the largest isoform of each of three full-length Lisch-related proteins. For each of three domains (Ig-like, TM, and ICD), pairwise alignments were performed between Lisch-like and three Lisch-related proteins. Similarity scores are also shown Mouse Ll sequence is identical to the N-scan predicted sequence chr1.1224.1.; Amino acid residues (#) refers to the largest isoform.

TABLE 7
Procedures Used for Immunohistochemical Staining

| | | | Primary | | Secondary |
|---|---|---|---|---|---|
| | | Primary | antibody | Secondary | antibody |
| Antigen | Method | antibody | dilution | antibody | dilution |
| Insulin | LM | Guinea pig | 1:10,000 | Anti-guinea | 1:200 (30′, |
| | | anti-swine | (O/N, | pig IgG | RT) |
| | | (DAKO, | 4° C.) or | (Vectastain) | |
| | | CA) | 1:6,000 | | |
| | | | (1 hr, RT) | | |
| Insulin | FM | Guinea pig | 1:4,000 | Anti-guinea | 1:200 (3 |
| | | anti-swine | (O/N, | pig IgG | hrs, RT) |
| | | (DAKO, | 4° C.) | Texas red | |
| | | CA) | | (Vectastain) | |
| GSPP | LM | Rabbit anti- | 1:10,000 | Anti-rabbit | 1:200 (30′, |
| | | human PP, | (O/N, | IgG | RT) |
| | | S, G | 4° C.) or | (Vectastain) | |
| | | (DAKO, | 1:6,000 | | |
| | | CA) | (1 hr, RT) | | |
| GSPP | FM | Rabbit anti- | 1:400 | Anti-rabbit | 1:200 (3 |
| | | human | (O/N, | IgG FITC | hrs, RT) |
| | | PP, S, G | 4° C.) | | |
| | | (DAKO | | | |
| | | CA) | | | |
| Ki67 | LM | Rabbit | 1:800 | Anti-rabbit | 1:200 (30′, |
| | | polyclonal | (30’ at | IgG | RT) |
| | | (Novocastra, | 37C + 30′ | | |
| | | England) | at RT) | | |
| | | | or 1:1000 | | |
| | | | (O/N | | |
| | | | 4° C.) | | |
| Lisch- | FM | Rabbit anti- | 1:500 | Anti-rabbit | 1:200 (3 |
| like | | mouse | (O/N, | IgG FITC | hrs, RT) |
| | | polyclonal | 4° C.) | | |
| BrdU | LM | Mouse | 1:100 | Anti-mouse | 1:250 (10′, |
| | | monoclonal | (O/N, | IgG | RT) |
| | | antibody | 4° C.) | | |
| | | (Sigma) | | | |

GSPP, [(glucagon, somatostatin, pancreatic polypeptide)]; BrdU, 5-bromo2′ -deoxy-uridine; LM, light microscopy; FM, fluorescent microscopy; G, glucagon; S, somatostatin; PP, pancreatic polypeptide; O/N, overnight incubation; RT, room temperature.

TABLE 8
Relative Expression of Ll in Tissues of Human Adults and 12-week Old Mice

| | | | White | | | | | 11-day |
|---|---|---|---|---|---|---|---|---|
| | | | adipose | | | Skeletal | | whole |
| Tissue | Brain | Hypothalamus | tissue | Pancreas | Islets | muscle | Liver | embryo |
| Human | 430 | ND | 47.6 | 12 | 75.6 | 9.9 | 3.8 | ND |
| Mouse | 45.5 | 58.6 | 1.9 | 4 | 3.9 | 0.5 | 13.8 | 4.2 |

Expression of Ll was measured by qPCR on cDNA from the respective organs or embryo. Sources of cDNA are described in Methods. Values represent ratios (×10⁻³) of L1/actin expression. Results are mean of triplicate assays. ND=not done.

DBA BAC Shotgun Sequencing.

BAC 95f9 DNA (5 μg) was fragmented to 1-5 kb using a nebuliser supplied with the TOPO Shotgun Subcloning kit (Invitrogen) and checked for size and quantity on an agarose gel. The shotgun library was constructed with 2 μg of sheared DNA. Blunt-end repair, dephosphorylation, ligation into PCR 4Blunt-TOPO vector, and transformation into TOP10 Electrocompetent E. coli were performed with the TOPO Shotgun Subcloning kit, following the manufacturer's protocol. Phenol:chloroform extraction of the dephosphorylated DNA was replaced with Qiagen QIAquick PCR Purification spin columns (QIAGEN). Recombinant colonies were selected by blue/white screening and incubated in LB medium supplemented with 50 μg/ml ampicillin for 20 h at 37° C. in 96-well deepwell plates. Plasmid miniprep was conducted in 96-well plates using QIAGEN Turbo Miniprep kits on a QIAGEN BioRobot 9600. DNA sequencing was performed on a 3730x1 Genetic Analyzer (Applied Biosystems) using BigDye® Terminator v3.1 Cycle Sequencing Kits with M13 forward and reverse sequencing primers.

Statistical Analyses.

ANOVA and ANCOVA were used to assess effects of genotype in congenic interval. Comparisons at individual time points, or pairs of means were performed using Student's t-test. P values are 2-tailed. The Statistica package (StatSoft) was used for ANOVAE; Excel (Microsoft) for t-testing.

Western Blot.

Hypothalamic extracts were prepared using M-PER Mammalian Protein Extraction Reagent (Pierce Biotechnology). Hypothalamic extracts (85 mg for B/B and D/D congenics and 175 mg for wild-type and mutant ENU mice) were resolved by 8% SDS-PAGE, transferred to nitrocellulose membrane (Invitrogen). A set of polyclonal rabbit antibodies (Covance Research Products) was generated against the predicted ICD, spanning residues 298-401 (exons 7,8) and verified that the α-ICD rabbit antibodies detected the appropriate fusion proteins, with only minor cross-reactivity in cultured cells. The blot was hybridized with anti-LL anti-sera at a dilution of 1:5,000 in TBS/0.05% Tween/5% milk (TBSTM) or with blocked anti-LL anti-sera diluted 1:10,000 in TBSTM. To prepare blocked anti-sera, liver sections from C3HeB/FeJ wild-type mice were fixed overnight in phosphate buffered paraformaldehyde at 4° C. and rinsed in PBS. Sections equivalent to one-third of a liver were fragmented and mixed with 1 ml anti-sera diluted 1/1000 in PBS/0.1% Triton. Liver fragments were spun out and the supernatant was used to probe filters from ENU mice. We detected bound antibody with horseradish peroxidase-coupled antibody against rabbit IgG (Amersham Biosciences) at a dilution of 1:5,000 using the SuperSignal West Pico Chemiluminescent Substrate kit (Pierce Biotechnology).

Immunohistochemical and Immunofluorescnce Analysis of Pancreatic Islets. For β-Cell Replication Studies:

Pancreata were fixed overnight in 10% formalin, embedded the specimens in paraffin, and consecutive 5 μm-thick sections were mounted on slides. For immunofluoresence and diaminobenzidine (DAB) staining of Ki67 and for insulin immunoreactivity, tissue sections were de-waxed in xylene, hydrated through a descending ethanol series and subjected to an antigen retrieval step using a heated citrate buffer solution. Several longitudinal sections >100 μm apart were used to assess β-cell replication and double staining for the nuclear proliferation marker Ki67 and insulin. Sections were incubated with Novocastra rabbit polyclonal anti-Ki67 antibody (Leica Microsystems) diluted 1:200 and an insulin polyclonal guinea pig anti-swine antibody (Vector Lab) diluted 1:2000 overnight at 4° C.

For Immunofluorescence Detection:

Sections were washed in PBS and incubated with secondary anti-guinea pig IgG (1:200) and fluorescein isothiocyanate-conjugated rabbit secondary antibody (1:200) (Vector Labs) for 1 hr and counterstained with DAPI before the addition of mounting medium. Non-overlapping images of longitudinal pancreatic sections were acquired using a Nikon Eclipse microscope and images imported into ImageJ (1.37 V, NIH) to count insulin-positive and Ki67-insulin-positive cells. β-cell replication is expressed as % Ki67-positive+insulin-positive/total insulin-positive cells. For diaminobenzidine staining, sections were incubated with secondary biotinylated rabbit and guinea pig IgG for 1 hr and then subjected to an avidin:biotyinylated enzyme complex (ABC Kit; Vector Labs) with DAB as substrate. Sections were counterstained with hematoxylin. Images of pancreatic sections were acquired using SpotAdvanced version 5 software (Diagnostic Instruments) and analyzed using imageProPlus software to calculate the % of \tilde{β}cell area occupied by Ki67 positive cells. 30-50 islets per animal from several non-overlapping sections through the pancreas were examined.

### Example 21

### Morpholino-Mediated Knockdown of Ll Paralog in Zebra Fish

Zebra fish Strains and Embryo Culture.

Zebra fish and embryos were raised, maintained and staged according to standard procedures (Westerfield M (2000) The Zebrafish Book: A Guide for the Laboratory Use of Zebrafish (Danio rerio). Eugene, Oreg.: University of Oregon Press). The AB* (Eugene, Oreg.) line and Tg(gut GFP)s854 transgenic line (gutGFP; [{Field, 2003 #156}]) were used in natural matings to obtain embryos. The gutGFP line was provided by Didier Stainier. Embryos examined at stages later than 24 hpf were maintained in embryo medium containing 0.003% phenylthiourea to inhibit pigmentation.

Morpholino Injections.

Morpholino antisense oligonucleotides were purchased from Gene Tools, LLC, and injected into 1-2 cell stage embryos at concentrations from 7-20 ng/embryo as previously described (Nasevicius and Ekker 2000, Nat Genet. 26: 216-220). Morpholino sequences are shown 5′-3′ with intronic sequences in lower case. Position, at right, is from the March 2006, Zv6 assembly. (SEQ ID NO: 60) lsr-like sp1: atgttgagtgtacTTGAGCTGGCTC@chr15:38,994,445-38,994,469 (Position from the July 2007, Zv7 assembly—chr15:23,943,253-23,943,277) (SEQ ID NO:61) lsr-like sp2: gaatgaaacacacTTCCTCCAGCAT@chr15:38,994,596-38,994,620 (Position from the July 2007, Zv7 assembly—chr15:23,943,404-23,943,428) (SEQ ID NO: 62) Ll-ATGAGCGTGTAACAAAAACATGATCCAG@chr9:31,645,414-31,645,438 (Position from the July 2007, Zv7 assembly -chr9:28,374,771-28,374,795) (SEQ ID NO: 63) Ll-splice: CAACTTTGCActgtgccaaagaaag@chr9:31,641,215-31,641,239 (Position from the July 2007, Zv7 assembly—chr9:28,370,572-28,370,596) Gene Tools, LLC standard control oligo.

RT-PCR.

Following manufacturer's (Invitrogen) protocol, total RNA was extracted from morpholino-injected and uninjected sibling embryos at 29 hours post-fertilization with TRIZOL and cDNA was synthesized with SuperScript II Reverse Transcriptase. Splice blocking by the Lsr-like morpholinos was analyzed using the primer-pair:

(SEQ ID NO: 64)
TGCCTATGCAAATGGGAGTTGGTG
@ chr15:38, 994, 385-38, 994, 408
and
(SEQ ID NO: 65)
TTGGCAACCTCTCGCTCCATGTAA
@ chr15:38, 994, 894-38, 994, 917

ef1α was amplified using the primer-pair:

(SEQ ID NO: 66)
5′-CAAGGGCTCCTTCAAGTACGCCTG-3′
and
(SEQ ID NO: 67)
5′- GGAAGAATGGCATCAAGGGCA-3′

Ll ortholog primer-pair:

(SEQ ID NO: 75)
5′-GCAAACTAACCCGCACTAAACTGG-3′
and
(SEQ ID NO: 76)
5′-AGGGACTCAGGAAAGGTGAAGGAA-3′

Immunofluorescence and RNA In Situ Hybridization.

Lsr-like ortholog was amplified from a wild-type, 24 hpf cDNA using the primer pair:

(SEQ ID NO: 77)
5′-CACGGACTTTCTCTACATACTTTTG-3′
and
(SEQ ID NO: 78)
5′-TTCATCCACATCATCGTACACT-3′

Lisch-like ortholog was amplified using the primer pair:

(SEQ ID NO: 79)
5′-TTTCACTGCAAAGTTGTGATGGCG-3′
and
(SEQ ID NO: 80)
5′-ATGTCATCCAGCACACCTGTCC-3′

The products were cloned into the PSTBlue-1 vector (Novagen) and used for antisense probe synthesis with T7 RNA polymerase after XhoI linearization (Lsr-like) and SP6 polymerase following BamHI linearization (Lisch-like). Whole-mount in situ hybridization was performed as described (Thisse C, and Thisse, B. (1998) High resolution whole-mount in situ hybridization. Zebrafish Sci Monit 5: 8-9). For immunofluorescence, embryos were fixed at room temperature (rt) in 4% paraformaldehyde for 2 h. After fixation, yolks were manually removed and embryos were permeabilized in acetone at −20° C. for 7 min. Embryos were washed briefly in PBS+0.1% Triton X100 (PBSTx) and incubated for 1 h in antibody hybridization buffer (PBSTx with 2% DMSO, 2% BSA and 2% sheep serum). Guinea pig anti-insulin antibody (Biomeda V2024) was diluted 1:1000 in antibody hybridization buffer and incubated with embryos for 2 h at rt. Following antibody hybridization, embryos were washed extensively with PBSTx and incubated with Cy3-labelled donkey anti-guinea pig secondary antibody diluted 1:500 in antibody hybridization buffer for 2 h at rt. Embryos were washed extensively with PBSTx and cleared in 80% glycerol/20% PBS. Images of optical sections were captured using a confocal microscope and 2-D projections were generated from optical sections using MetaMorph software.

### Example 22

### Predicted Secondary Structure of Lisch-Like (LL)

The sequence of Lisch-like (LL) exons 2 and 3 was analyzed using GenTHREADER at the PSIPRED Protein Structure Prediction Server. GenThreader assigns confidence levels to matches between the query sequence (here, LL exons 2 and 3) and known protein structures. Three proteins of known structure matched at high confidence to the sequence of LL exons 2 and 3. At the lowest p-value (0.0003) was the V-type immunoglobulin-like domain of chitin-binding protein 3 of Branchiosoma floridae (UniProtKB/TrEMBL Q819N0; PDB 1XT5AO). FIG. 42A shows the sequence alignment and the alignment between the known secondary structure of 1XT5AO and the predicted secondary structure for LL. Both proteins show a set of 9 Beta sheets (the EEEE runs), where the LL structure has an additional large helix-containing loop FIGS. 42B and C show two views of 1XT5AO. FIG. 42B shows a wall of the Ig-like sandwich, comprised of 5 anti-parallel sheets. FIG. 43C is a rotated view looking between the two sheets to reveal a ligand-binding pocket, where fatty acids or small polysaccharides are predicted bind.

Submission of the entire LL sequence to the Robetta Structure Prediction Server, using a Hidden Markov Model search, returned as reference parent at the highest confidence, a lipid-antigen binding immune system protein (PDB; 2P06). The structure of this protein is shown in FIG. 43.

### REFERENCES

1. Cowie, C. 2003. Prevalence of diabetes and impaired fasting glucose in adults -United States, 1999-2000. MMWR 52:833-837.
2. Kaufman, F. R. 2002. Type 2 diabetes mellitus in children and youth: a new epidemic. J Pediatr Endocrinol Metab 15 Suppl 2:737-744.
3. Saltiel, A. R. 2001. New perspectives into the molecular pathogenesis and treatment of type 2 diabetes. Cell 104:517-529.
4. DeFronzo, R. A., Bonadonna, R. C., and Ferrannini, E. 1992. Pathogenesis of NIDDM. A balanced overview. Diabetes Care 15:318-368.
5. Mora, S., and Pessin, J. E. 2002. An adipocentric view of signaling and intracellular trafficking. Diabetes Metab Res Rev 18:345-356.
6. Boden, G., and Shulman, G. I. 2002. Free fatty acids in obesity and type 2 diabetes: defining their role in the development of insulin resistance and beta-cell dysfunction. Eur J Clin Invest 32 Suppl 3:14-23.
7. Kahn, S. E., Hull, R. L., and Utzschneider, K. M. 2006. Mechanisms linking obesity to insulin resistance and type 2 diabetes. Nature 444:840-846.
8. Haffner, S. M. 2006. Relationship of metabolic risk factors and development of cardiovascular disease and diabetes. Obesity (Silver Spring) 14 Suppl 3:121 S-127S.
9. Hossain, P., Kawar, B., and El Nahas, M. 2007. Obesity and diabetes in the developing world—a growing challenge. N Engl J Med 356:213-215.
10. Kloppel, G., Lohr, M., Habich, K., Oberholzer, M., and Heitz, P. U. 1985. Islet pathology and the pathogenesis of type 1 and type 2 diabetes mellitus revisited. Sury Synth Pathol Res 4:110-125.
11. Butler, A. E., Janson, J., Bonner-Weir, S., Ritzel, R., Rizza, R. A., and Butler, P. C.
2003. Beta-cell deficit and increased beta-cell apoptosis in humans with type 2 diabetes. Diabetes 52:102-110.
12. Miralles, F., and Portha, B. 2001. Early development of beta-cells is impaired in the GK rat model of type 2 diabetes. Diabetes 50 Suppl 1:S84-88.
13. Leiter, E. H. 1989. The genetics of diabetes susceptibility in mice. Faseb J 3:2231-2241
14. Zucker, L. M., and Antoniades, H. N. 1972. Insulin and obesity in the Zucker genetically obese rat “fatty”. Endocrinology 90:1320-1330.
15. Frayling, T. M., Evans, J. C., Bulman, M. P., Pearson, E., Allen, L., Owen, K., Bingham, C., Hannemann, M., Shepherd, M., Ellard, S., et al. 2001. beta-cell genes and diabetes: molecular and clinical characterization of mutations in transcription factors. Diabetes 50 Suppl 1:S94-100.
16. Bonner-Weir, S. 2000. Perspective: Postnatal pancreatic beta cell growth. Endocrinology 141:1926-1929.
17. Dor, Y., Brown, J., Martinez, O. O., and Melton, D. A. 2004. Adult pancreatic beta-cells are formed by self-duplication rather than stem-cell differentiation. Nature 429:41-46.
18. Finegood, D. T., Scaglia, L., and Bonner-Weir, S. 1995. Dynamics of beta-cell mass in the growing rat pancreas. Estimation with a simple mathematical model. Diabetes 44:249-256.
19. Teta, M., Long, S. Y., Wartschow, L. M., Rankin, M. M., and Kushner, J. A. 2005. Very slow turnover of beta-cells in aged adult mice. Diabetes 54:2557-2567.
20. Hales, C. N., and Barker, D. J. 2001. The thrifty phenotype hypothesis. Br Med Bull 60:5-20.
21. Barnett, A. H., Eff, C., Leslie, R. D., and Pyke, D. A. 1981. Diabetes in identical twins. A study of 200 pairs. Diabetologia 20:87-93.
22. Lo, S. S., Tun, R. Y., Hawa, M., and Leslie, R. D. 1991. Studies of diabetic twins. Diabetes Metab Rev 7:223-238.
23. Kahn, C. R., Vicent, D., and Doria, A. 1996. Genetics of non-insulin-dependent (type-II) diabetes mellitus. Annu Rev Med 47:509-531.
24. Medici, F., Hawa, M., Ianari, A., Pyke, D. A., and Leslie, R. D. 1999. Concordance rate for type II diabetes mellitus in monozygotic twins: actuarial analysis. Diabetologia 42:146-150.
25. Jun, H., Bae, H. Y., Lee, B. R., Koh, K. S., Kim, Y. S., Lee, K. W., Kim, H., and Yoon, J. 1999. Pathogenesis of non-insulin-dependent (type II) diabetes mellitus (NIDDM)—genetic predisposition and metabolic abnormalities. Adv Drug Deliv Rev 35:157-177.
26. Permutt, M. A., Wasson, J., and Cox, N. 2005. Genetic epidemiology of diabetes. J Clin Invest 115:1431-1439.
27. Florez, J. C., Hirschhorn, J., and Altshuler, D. 2003. The inherited basis of diabetes mellitus: implications for the genetic analysis of complex traits. Annu Rev Genomics Hum Genet. 4:257-291.
28. Khanim, F., Kirk, J., Latif, F., and Barrett, T. G. 2001. WFS1/wolframin mutations, Wolfram syndrome, and associated diseases. Hum Mutat 17:357-367.
29. Cox, N. J., Xiang, K. S., Fajans, S. S., and Bell, G. I. 1992. Mapping diabetes-susceptibility genes. Lessons learned from search for DNA marker for maturity-onset diabetes of the young. Diabetes 41:401-407.
30. Pimenta, W., Korytkowski, M., Mitrakou, A., Jenssen, T., Yki-Jarvinen, H., Evron, W., Dailey, G., and Gerich, J. 1995. Pancreatic beta-cell dysfunction as the primary genetic lesion in NIDDM. Evidence from studies in normal glucose-tolerant individuals with a first-degree NIDDM relative. Jama 273:1855-1861.
31. Gelding, S. V., Andres, C., Niththyananthan, R., Gray, I. P., Mather, H., and Johnston, D. G. 1995. Increased secretion of 32,33 split proinsulin after intravenous glucose in glucose-tolerant first-degree relatives of patients with non-insulin dependent diabetes of European, but not Asian, origin. Clin Endocrinol (Oxf) 42:255-264.
32. Knowler, W. C., Saad, M. F., Pettitt, D. J., Nelson, R. G., and Bennett, P. H. 1993. Determinants of diabetes mellitus in the Pima Indians. Diabetes Care 16:216-227.
33. Hanley, A. J., Williams, K., Gonzalez, C., D'Agostino, R. B., Jr., Wagenknecht, L. E., Stern, M. P., and Haffner, S. M. 2003. Prediction of type 2 diabetes using simple measures of insulin resistance: combined results from the San Antonio Heart Study, the Mexico City Diabetes Study, and the Insulin Resistance Atherosclerosis Study. Diabetes 52:463-469.
34. Elbein, S. C., Hoffman, M. D., Teng, K., Leppert, M. F., and Hasstedt, S. J. 1999. A genome-wide search for type 2 diabetes susceptibility genes in Utah Caucasians. Diabetes 48:1175-1182.
35. HsuEh, W. C., St Jean, P. L., Mitchell, B. D., Pollin, T. I., Knowler, W. C., Ehm, M. G., Bell, C. J., Sakul, H., Wagner, M. J., Burns, D. K., et al. 2003. Genome-wide and fine-mapping linkage studies of type 2 diabetes and glucose traits in the Old Order Amish: evidence for a new diabetes locus on chromosome 14q11 and confirmation of a locus on chromosome 1q21-q24. Diabetes 52:550-557.
36. St. Jean, P. 2000. Association between diabetes, obesity, glucose and insulin levels in the Old Amish and SNP's on 1q21-23. American Journal of Human Genetics 67.
37. Wiltshire, S., Hattersley, A. T., Hitman, G. A., Walker, M., Levy, J. C., Sampson, M., O'Rahilly, S., Frayling, T. M., Bell, J. I., Lathrop, G. M., et al. 2001. A genomewide scan for loci predisposing to type 2 diabetes in a U. K. population (the Diabetes UK Warren 2 Repository): analysis of 573 pedigrees provides independent replication of a susceptibility locus on chromosome 1q. Am J Hum Genet. 69:553-569.
38. Vionnet, N., Hani El, H., Dupont, S., Gallina, S., Francke, S., Dotte, S., De Matos, F., Durand, E., Lepretre, F., Lecoeur, C., et al. 2000. Genomewide search for type 2 diabetes-susceptibility genes in French whites: evidence for a novel susceptibility locus for early-onset diabetes on chromosome 3q27-qter and independent replication of a type 2-diabetes locus on chromosome 1q21-q24. Am J Hum Genet. 67:1470-1480.
39. Meigs, J. B., Panhuysen, C. I., Myers, R. H., Wilson, P. W., and Cupples, L. A. 2002. A genome-wide scan for loci linked to plasma levels of glucose and HbA(1c) in a community-based sample of Caucasian pedigrees: The Framingham Offspring Study. Diabetes 51:833-840.
40. Hanson, R. L., Ehm, M. G., Pettitt, D. J., Prochazka, M., Thompson, D. B., Timberlake, D., Foroud, T., Kobes, S., Baier, L., Burns, D. K., et al. 1998. An autosomal genomic scan for loci linked to type II diabetes mellitus and body-mass index in Pima Indians. Am J Hum Genet. 63:1130-1138.
41. Xiang, K., Wang, Y., Zheng, T., Jia, W., Li, J., Chen, L., Shen, K., Wu, S., Lin, X., Zhang, G., et al. 2004. Genome-wide search for type 2 diabetes/impaired glucose homeostasis susceptibility genes in the Chinese: significant linkage to chromosome 6q21-q23 and chromosome 1q21-q24. Diabetes 53:228-234.
42. Coleman, D. L. 1982. Diabetes-obesity syndromes in mice. Diabetes 31:1-6.
43. Leibel, R. L., Chung, W. K., and Chua, S. C., Jr. 1997. The molecular genetics of rodent single gene obesities. J Biol Chem 272:31937-31940.
44. Clee, S. M., and Attie, A. D. 2006. The Genetic Landscape of Type 2 Diabetes in Mice. Endocr Rev.
45. Friedman, J. M., Leibel, R. L., Siegel, D. S., Walsh, J., and Bahary, N. 1991. Molecular mapping of the mouse ob mutation. Genomics 11:1054-1062.
46. Chua, S. C., Jr., Chung, W. K., Wu-Peng, X. S., Zhang, Y., Liu, S. M., Tartaglia, L., and Leibel, R. L. 1996. Phenotypes of mouse diabetes and rat fatty due to mutations in the OB (leptin) receptor. Science 271:994-996.
47. Flint, J., Valdar, W., Shifman, S., and Mott, R. 2005. Strategies for mapping and cloning quantitative trait genes in rodents. Nat Rev Genet. 6:271-286.
48. Todd, J. A. 1999. From genome to aetiology in a multifactorial disease, type 1 diabetes. Bioessays 21:164-174.
49. York, B., Lei, K., and West, D. B. 1996. Sensitivity to dietary obesity linked to a locus on chromosome 15 in a CAST/Ei×C57BL/6J F2 intercross. Mamm Genome 7:677-681.
50. Mitsos, L. M., Cardon, L. R., Fortin, A., Ryan, L., LaCourse, R., North, R. J., and Gros, P. 2000. Genetic control of susceptibility to infection with Mycobacterium tuberculosis in mice. Genes Immun 1:467-477.
51. Welch, C. L., Bretschger, S., Latib, N., Bezouevski, M., Guo, Y., Pleskac, N., Liang, C. P., Barlow, C., Dansky, H., Breslow, J. L., et al. 2001. Localization of atherosclerosis susceptibility loci to chromosomes 4 and 6 using the Ldlr knockout mouse model. Proc Natl Acad Sci USA 98:7946-7951.
52. Legare, M. E., Bartlett, F. S., 2nd, and Frankel, W. N. 2000. A major effect QTL determined by multiple genes in epileptic EL mice. Genome Res 10:42-48.
53. Joober, R., Zarate, J. M., Rouleau, G. A., Skamene, E., and Boksa, P. 2002. Provisional mapping of quantitative trait loci modulating the acoustic startle response and prepulse inhibition of acoustic startle. Neuropsychopharmacology 27:765-781.
54. Clee, S. M., Yandell, B. S., Schueler, K. M., Rabaglia, M. E., Richards, O. C., Raines, S. M., Kabara, E. A., Klass, D. M., Mui, E. T., Stapleton, D. S., et al. 2006. Positional cloning of Sorcsl, a type 2 diabetes quantitative trait locus. Nat Genet. 38:688-693.
55. Freeman, H., Shimomura, K., Horner, E., Cox, R. D., and Ashcroft, F. M. 2006. Nicotinamide nucleotide transhydrogenase: a key role in insulin secretion. Cell Metab 3:35-45.
56. Freeman, H. C., Hugill, A., Dear, N. T., Ashcroft, F. M., and Cox, R. D. 2006. Deletion of nicotinamide nucleotide transhydrogenase: a new quantitive trait locus accounting for glucose intolerance in C57BL/6J mice. Diabetes 55:2153-2156.
57. Chung, W. K., Zheng, M., Chua, M., Kershaw, E., Power-Kehoe, L., Tsuji, M., Wu-Peng, X. S., Williams, J., Chua, S. C., Jr., and Leibel, R. L. 1997. Genetic modifiers of Leprfa associated with variability in insulin production and susceptibility to NIDDM. Genomics 41:332-344.
58. Gauguier, D., Froguel, P., Parent, V., Bernard, C., Bihoreau, M. T., Portha, B., James, M. R., Penicaud, L., Lathrop, M., and Ktorza, A. 1996. Chromosomal mapping of genetic loci associated with non-insulin dependent diabetes in the GK rat. Nat Genet. 12:38-43.
59. Lapidot, M., and Pilpel, Y. 2006. Genome-wide natural antisense transcription: coupling its regulation to its different regulatory mechanisms. EMBO Rep 7:1216-1222.
60. Costa, F. F. 2005. Non-coding RNAs: new players in eukaryotic biology. Gene 357:83-94.
61. Yen, F. T., Masson, M., Clossais-Besnard, N., Andre, P., Grosset, J. M., Bougueleret, L., Dumas, J. B., Guerassimenko, O., and Bihain, B. E. 1999. Molecular cloning of a lipolysis-stimulated remnant receptor expressed in the liver. J Biol Chem 274:13390-13398.
62. Mesli, S., Javorschi, S., Berard, A. M., Landry, M., Priddle, H., Kivlichan, D., Smith, A. J., Yen, F. T., Bihain, B. E., and Darmon, M. 2004. Distribution of the lipolysis stimulated receptor in adult and embryonic murine tissues and lethality of LSR−/− embryos at 12.5 to 14.5 days of gestation. Eur J Biochem 271:3103-3114.
63. Yen, F. T., Mann, C. J., Guermani, L. M., Hannouche, N. F., Hubert, N., Hornick, C. A., Bordeau, V. N., Agnani, G., and Bihain, B. E. 1994. Identification of a lipolysis-stimulated receptor that is distinct from the LDL receptor and the LDL receptor-related protein. Biochemistry 33:1172-1180.
64. Coleman, D. L., and Hummel, K. P. 1973. The influence of genetic background on the expression of the obese (0b) gene in the mouse. Diabetologia 9:287-293.
65. Visscher, P. M. 1999. Speed congenics: accelerated genome recovery using genetic markers. Genet Res 74:81-85.
66. Wade, C. M., Kulbokas, E. J., 3rd, Kirby, A. W., Zody, M. C., Mullikin, J. C., Lander, E. S., Lindblad-Toh, K., and Daly, M. J. 2002. The mosaic structure of variation in the laboratory mouse genome. Nature 420:574-578.
67. Prentki, M., and Nolan, C. J. 2006. Islet beta cell failure in type 2 diabetes. J Clin Invest 116:1802-1812.
68. Kido, Y., Burks, D. J., Withers, D., Bruning, J. C., Kahn, C. R., White, M. F., and Accili, D. 2000. Tissue-specific insulin resistance in mice with mutations in the insulin receptor, IRS-1, and IRS-2. J Clin Invest 105:199-205.
69. Okamoto, H., Nakae, J., Kitamura, T., Park, B. C., Dragatsis, I., and Accili, D. 2004. Transgenic rescue of insulin receptor-deficient mice. J Clin Invest 114:214-223.
70. Stanton, K. J., Sidner, R. A., Miller, G. A., Cummings, O. W., Schmidt, C. M., Howard, T. J., and Wiebke, E. A. 2003. Analysis of Ki-67 antigen expression, DNA proliferative fraction, and survival in resected cancer of the pancreas. Am J Surg 186:486-492.
71. Bonner-Weir, S. 2000. Life and death of the pancreatic beta cells. Trends Endocrinol Metab 11:375-378.
72. Bonner-Weir, S. 2001. beta-cell turnover: its assessment and implications. Diabetes 50 Suppl 1:S20-24.
73. Covey, S. D., Wideman, R. D., McDonald, C., Unniappan, S., Huynh, F., Asadi, A., Speck, M., Webber, T., Chua, S. C., and Kieffer, T. J. 2006. The pancreatic beta cell is a key site for mediating the effects of leptin on glucose homeostasis. Cell Metab 4:291-302.
74. Scaglia, L., Cahill, C. J., Finegood, D. T., and Bonner-Weir, S. 1997. Apoptosis participates in the remodeling of the endocrine pancreas in the neonatal rat. Endocrinology 138:1736-1741.
75. Wang, J., Li, S., Zhang, Y., Zheng, H., Xu, Z., Ye, J., Yu, J., and Wong, G. K. 2003. Vertebrate gene predictions and the problem of large genes. Nat Rev Genet. 4:741-749.
76. Lindblad-Toh, K., Lander, E. S., McPherson, J. D., Waterston, R. H., Rodgers, J., and Birney, E. 2001. Progress in sequencing the mouse genome. Genesis 31:137-141.
77. Bromberg, Y. a. R., B. 2006. SNAP: prediction of functional effects of non-synonymous polymorphisms.
78. Ramensky, V., Bork, P., and Sunyaev, S. 2002. Human non-synonymous SNPs: server and survey. Nucleic Acids Res 30:3894-3900.
79. Ng, P. C., and Henikoff, S. 2003. SIFT: Predicting amino acid changes that affect protein function. Nucleic Acids Res 31:3812-3814.
80. Dayhoff, M. 1978. Atlas of Protein Sequence and Structure. Washington, D.C.: National Biochemical Research Foundation. 6 pp.
81. Rost, B., and Sander, C. 1994. Combining evolutionary information and neural networks to predict protein secondary structure. Proteins 19:55-72.
82. German, M. S., Wang, J., Fernald, A. A., Espinosa, R., 3rd, Le Beau, M. M., and Bell, G. I. 1994. Localization of the genes encoding two transcription factors, LMX1 and CDX3, regulating insulin gene expression to human chromosomes 1 and 13. Genomics 24:403-404.
83. Hsieh, C. H., Liang, K. H., Hung, Y. J., Huang, L. C., Pei, D., Liao, Y. T., Kuo, S. W., Bey, M. S., Chen, J. L., and Chen, E. Y. 2006. Analysis of epistasis for diabetic nephropathy among type 2 diabetic patients. Hum Mol Genet. 15:2701-2708.
84. Numata, K., Okada, Y., Saito, R., Kiyosawa, H., Kanai, A., and Tomita, M. 2006. Comparative analysis of cis-encoded antisense RNAs in eukaryotes. Gene.
85. Shalgi, R., Lapidot, M., Shamir, R., and Pilpel, Y. 2005. A catalog of stability-associated sequence elements in 3′ UTRs of yeast mRNAs. Genome Biol 6:R86.
86. Xie, X., Lu, J., Kulbokas, E. J., Golub, T. R., Mootha, V., Lindblad-Toh, K., Lander, E. S., and Kellis, M. 2005. Systematic discovery of regulatory motifs in human promoters and 3′ UTRs by comparison of several mammals. Nature 434:338-345.
87. Schulz, H.2003. Towards a Comprehensive Description of the Human Retinal Transcriptome: Identification and Characterization of Differentially Expressed Genes. Wurzberg.
88. Vandenbroucke, II, Vandesompele, J., Paepe, A. D., and Messiaen, L. 2001. Quantification of splice variants using real-time PCR. Nucleic Acids Res 29:E68-68.
89. Draper, B. W., Morcos, P. A., and Kimmel, C. B. 2001 Inhibition of zebrafish fgf8 pre-mRNA splicing with morpholino oligos: a quantifiable method for gene knockdown. Genesis 30:154-156.
90. Gnugge, L., Meyer, D., and Driever, W. 2004. Pancreas development in zebrafish. Methods Cell Biol 76:531-551.
91. Kim, H. J., Sumanas, S., Palencia-Desai, S., Dong, Y., Chen, J. N., and Lin, S. 2006. Genetic analysis of early endocrine pancreas formation in zebrafish. Mol Endocrinol 20:194-203.
92. Field, H. A. e. a. 2003a. Formation of the digestive system in zebrafish. II. Pancreas morphogenesis. Dev Biol 261:197-208.
93. Sherwood, N. M., and Wu, S. 2005. Developmental role of GnRH and PACAP in a zebrafish model. Gen Comp Endocrinol 142:74-80.
94. McGonnell, I. M., and Fowkes, R. C. 2006. Fishing for gene function—endocrine modelling in the zebrafish. J Endocrinol 189:425-439.
95. Field, H. A., Ober, E. A., Roeser, T., and Stainier, D. Y. 2003. Formation of the digestive system in zebrafish. I. Liver morphogenesis. Dev Biol 253:279-290.
96. Zecchin, E., Mavropoulos, A., Devos, N., Filippi, A., Tiso, N., Meyer, D., Peers, B., Bortolussi, M., and Argenton, F. 2004. Evolutionary conserved role of ptfla in the specification of exocrine pancreatic fates. Dev Biol 268:174-184.
97. Lin, J. W., Biankin, A. V., Horb, M. E., Ghosh, B., Prasad, N. B., Yee, N. S., Pack, M. A., and Leach, S. D. 2004. Differential requirement for ptfla in endocrine and exocrine lineages of developing zebrafish pancreas. Dev Biol 274:491-503.
98. Yee, N. S., Yusuff, S., and Pack, M. 2001. Zebrafish pdxl morphant displays defects in pancreas development and digestive organ chirality, and potentially identifies a multipotent pancreas progenitor cell. Genesis 30:137-140.
99. Ehm, M. G., Karnoub, M. C., Sakul, H., Gottschalk, K., Holt, D. C., Weber, J. L., Vaske, D., Briley, D., Briley, L., Kopf, J., et al. 2000. Genomewide search for type 2 diabetes susceptibility genes in four American populations. Am J Hum Genet. 66:1871-1881.
100. Langefeld, C. D., Wagenknecht, L. E., Rotter, J. I., Williams, A. H., Hokanson, J. E., Saad, M. F., Bowden, D. W., Haffner, S., Norris, J. M., Rich, S. S., et al. 2004. Linkage of the metabolic syndrome to 1q23-q31 in Hispanic families: the Insulin Resistance Atherosclerosis Study Family Study. Diabetes 53:1170-1174.
101. McCarthy, M., Shuldiner, A R, Bogardus, C, Hanson, R L, Elbein, S. 2004. Positional Cloning of a Type 2 Diabetes Susceptibility Gene on Chromosome 1q: A collaborative effort by the Chromosome 1q Diabetes Positional Cloning Consotrium.
102. Zeggini, E., Damcott, C. M., Hanson, R. L., Karim, M. A., Rayner, N. W., Groves, C. J., Baier, L. J., Hale, T. C., Hattersley, A. T., Hitman, G. A., et al. 2006. Variation within the gene encoding the upstream stimulatory factor 1 does not influence susceptibility to type 2 diabetes in samples from populations with replicated evidence of linkage to chromosome 1q. Diabetes 55:2541-2548.
103. Sladek, R., Rocheleau, G., Rung, J., Dina, C., Shen, L., Serre, D., Boutin, P., Vincent, D., Belisle, A., Hadjadj, S., et al. 2007. A genome-wide association study identifies novel risk loci for type 2 diabetes. Nature.
104. Xia, J., Scherer, S. W., Cohen, P. T., Majer, M., Xi, T., Norman, R. A., Knowler, W. C., Bogardus, C., and Prochazka, M. 1998. A common variant in PPP1R3 associated with insulin resistance and type 2 diabetes. Diabetes 47:1519-1524.
105. Xia, J., Bogardus, C., and Prochazka, M. 1999. A type 2 diabetes-associated polymorphic ARE motif affecting expression of PPP1R3 is involved in RNA-protein interactions. Mol Genet Metab 68:48-55.
106. Stanger, B. Z., Tanaka, A. J., and Melton, D. A. 2007. Organ size is limited by the number of embryonic progenitor cells in the pancreas but not the liver. Nature 445:886-891.
107. Leahy, J. L., and Vandekerkhove, K. M. 1990. Insulin-like growth factor-I at physiological concentrations is a potent inhibitor of insulin secretion. Endocrinology 126:1593-1598.
108. Garcia-Ocana, A., Takane, K. K., Syed, M. A., Philbrick, W. M., Vasavada, R. C., and Stewart, A. F. 2000. Hepatocyte growth factor overexpression in the islet of transgenic mice increases beta cell proliferation, enhances islet mass, and induces mild hypoglycemia. J Biol Chem 275:1226-1232.
109. Hauge, H., Patzke, S., Delabie, J., and Aasheim, H. C. 2004. Characterization of a novel immunoglobulin-like domain containing receptor. Biochem Biophys Res Commun 323:970-978.
110. Jin, J., Smith, F. D., Stark, C., Wells, C. D., Fawcett, J. P., Kulkarni, S., Metalnikov, P., O'Donnell, P., Taylor, P., Taylor, L., et al. 2004. Proteomic, functional, and domain-based analysis of in vivo 14-3-3 binding proteins involved in cytoskeletal regulation and cellular organization. Curr Biol 14:1436-1450.
111. Onuma, H., Osawa, H., Yamada, K., Ogura, T., Tanabe, F., Granner, D. K., and Makino, H.2002. Identification of the insulin-regulated interaction of phosphodiesterase 3B with 14-3-3 beta protein. Diabetes 51:3362-3367.
112. Xiang, K. e. a. 2002. Genome wide scan for type 2 diabetes susceptibilty loci in Chinese. Diabetes 51:1066-P.
113. Pozuelo Rubio, M., Geraghty, K. M., Wong, B. H., Wood, N. T., Campbell, D. G., Morrice, N., and Mackintosh, C. 2004. 14-3-3-affinity purification of over 200 human phosphoproteins reveals new links to regulation of cellular metabolism, proliferation and trafficking. Biochem J 379:395-408.
114. Meek, S. E., Lane, W. S., and Piwnica-Worms, H.2004. Comprehensive proteomic analysis of interphase and mitotic 14-3-3-binding proteins. J Biol Chem 279:32046-32054.
115. Hermeking, H., and Benzinger, A. 2006. 14-3-3 proteins in cell cycle regulation. Semin Cancer Biol 16:183-192.
116. Moffat, J., Grueneberg, D. A., Yang, X., Kim, S. Y., Kloepfer, A. M., Hinkle, G., Piqani, B., Eisenhaure, T. M., Luo, B., Grenier, J. K., et al. 2006. A lentiviral RNAi library for human and mouse genes applied to an arrayed viral high-content screen. Cell 124:1283-1298.
117. Khvorova, A., Reynolds, A., and Jayasena, S. D. 2003. Functional siRNAs and miRNAs exhibit strand bias. Cell 115:209-216.
118. Schwarz, D. S., Hutvagner, G., Du, T., Xu, Z., Aronin, N., and Zamore, P. D. 2003. Asymmetry in the assembly of the RNAi enzyme complex. Cell 115:199-208.
119. Antinozzi, P. A., Garcia-Diaz, A., Hu, C., and Rothman, J. E. 2006. Functional mapping of disease susceptibility loci using cell biology. Proc Natl Acad Sci USA 103:3698-3703.
120. Augustin, M., Sedlmeier, R., Peters, T., Huffstadt, U., Kochmann, E., Simon, D., Schoniger, M., Garke-Canerthaler, S., Laufs, J., Canhaus, M., et al. 2005. Efficient and fast targeted production of murine models based on ENU mutagenesis. Mamm Genome 16:405-413.
121. McMinn, J. E., Liu, S. M., Dragatsis, I., Dietrich, P., Ludwig, T., Eiden, S., and Chua, S. C., Jr. 2004. An allelic series for the leptin receptor gene generated by CRE and FLP recombinase. Mamm Genome 15:677-685.
122. Coppari, R., Ichinose, M., Lee, C. E., Pullen, A. E., Kenny, C. D., McGovern, R. A., Tang, V., Liu, S. M., Ludwig, T., Chua, S. C., Jr., et al. 2005. The hypothalamic arcuate nucleus: a key site for mediating leptin's effects on glucose homeostasis and locomotor activity. Cell Metab 1:63-72.
123. Meyers, E. N., Lewandoski, M., and Martin, G. R. 1998. An Fgf8 mutant allelic series generated by Cre- and Flp-mediated recombination. Nat Genet. 18:136-141.
124. Voronina, V. A., Kozlov, S., Mathers, P. H., and Lewandoski, M. 2005. Conditional alleles for activation and inactivation of the mouse Rx homeobox gene. Genesis 41:160-164.
125. Farley, F. W., Soriano, P., Steffen, L. S., and Dymecki, S. M. 2000. Widespread recombinase expression using FLPeR (flipper) mice. Genesis 28:106-110.
126. Buchholz, F., Angrand, P. O., and Stewart, A. F. 1998. Improved properties of FLP recombinase evolved by cycling mutagenesis. Nat Biotechnol 16:657-662.
127. Bruning, J. C., Michael, M. D., Winnay, J. N., Hayashi, T., Horsch, D., Accili, D., Goodyear, L. J., and Kahn, C. R. 1998. A muscle-specific insulin receptor knockout exhibits features of the metabolic syndrome of NIDDM without altering glucose tolerance. Mol Cell 2:559-569.
128. Han, S., Liang, C. P., DeVries-Seimon, T., Ranalletta, M., Welch, C. L., Collins-Fletcher, K., Accili, D., Tabas, I., and Tall, A. R. 2006. Macrophage insulin receptor deficiency increases ER stress-induced apoptosis and necrotic core formation in advanced atherosclerotic lesions. Cell Metab 3:257-266.
129. Nandi, A., Kitamura, Y., Kahn, C. R., and Accili, D. 2004. Mouse models of insulin resistance. Physiol Rev 84:623-647.
130. Xuan, S., Kitamura, T., Nakae, J., Politi, K., Kido, Y., Fisher, P. E., Morroni, M., Cinti, S., White, M. F., Herrera, P. L., et al. 2002. Defective insulin secretion in pancreatic beta cells lacking type 1 IGF receptor. J Clin Invest 110:1011-1019.
131. Accili, D., Frapier, C., Mosthaf, L., McKeon, C., Elbein, S. C., Permutt, M. A., Ramos, E., Lander, E., Ullrich, A., and Taylor, S. I. 1989. A mutation in the insulin receptor gene that impairs transport of the receptor to the plasma membrane and causes insulin-resistant diabetes. Embo J 8:2509-2517.
132. Barbetti, F., Raben, N., Kadowaki, T., Cama, A., Accili, D., Gabbay, K. H., Merenich, J. A., Taylor, S. I., and Roth, J. 1990. Two unrelated patients with familial hyperproinsulinemia due to a mutation substituting histidine for arginine at position 65 in the proinsulin molecule: identification of the mutation by direct sequencing of genomic deoxyribonucleic acid amplified by polymerase chain reaction. J Clin Endocrinol Metab 71:164-169.
133. Kadowaki, T., Kadowaki, H., Accili, D., and Taylor, S. I. 1990. Substitution of lysine for asparagine at position 15 in the alpha-subunit of the human insulin receptor. A mutation that impairs transport of receptors to the cell surface and decreases the affinity of insulin binding. J Biol Chem 265:19143-19150.
134. Kadowaki, T., Kadowaki, H., Accili, D., Yazaki, Y., and Taylor, S. I. 1991. Substitution of arginine for histidine at position 209 in the alpha-subunit of the human insulin receptor. A mutation that impairs receptor dimerization and transport of receptors to the cell surface. J Biol Chem 266:21224-21231.
135. Accili, D., Kadowaki, T., Kadowaki, H., Mosthaf, L., Ullrich, A., and Taylor, S. I. 1992 Immunoglobulin heavy chain-binding protein binds to misfolded mutant insulin receptors with mutations in the extracellular domain. J Biol Chem 267:586-590.
136. Rother, K. I., Imai, Y., Caruso, M., Beguinot, F., Formisano, P., and Accili, D. 1998. Evidence that IRS-2 phosphorylation is required for insulin action in hepatocytes. J Biol Chem 273:17491-17497.
137. Santagata, S., Boggon, T. J., Baird, C. L., Gomez, C. A., Zhao, J., Shan, W. S., Myszka, D. G., and Shapiro, L. 2001. G-protein signaling through tubby proteins. Science 292:2041-2050.
138. Boggon, T. J., Shan, W. S., Santagata, S., Myers, S. C., and Shapiro, L. 1999. Implication of tubby proteins as transcription factors by structure-based functional analysis. Science 286:2119-2125.
139. Horton, J. D., Goldstein, J. L., and Brown, M. S. 2002. SREBPs: activators of the complete program of cholesterol and fatty acid synthesis in the liver. J Clin Invest 109:1125-1131.
140. Nakae, J., Kitamura, T., Silver, D. L., and Accili, D. 2001. The forkhead transcription factor Foxo1 (Fkhr) confers insulin sensitivity onto glucose-6-phosphatase expression. J Clin Invest 108:1359-1367.
141. Nakae, J., Barr, V., and Accili, D. 2000. Differential regulation of gene expression by insulin and IGF-1 receptors correlates with phosphorylation of a single amino acid residue in the forkhead transcription factor FKHR. Embo J 19:989-996.
142. Accili, D., Mosthaf, L., Ullrich, A., and Taylor, S. I. 1991. A mutation in the extracellular domain of the insulin receptor impairs the ability of insulin to stimulate receptor autophosphorylation. J Biol Chem 266:434-439.
143. Nakae, J., Park, B. C., and Accili, D. 1999. Insulin stimulates phosphorylation of the forkhead transcription factor FKHR on serine 253 through a Wortmannin-sensitive pathway. J Biol Chem 274:15982-15985.
144. Perrotti, N., Accili, D., Marcus-Samuels, B., Rees-Jones, R. W., and Taylor, S. I.
1987. Insulin stimulates phosphorylation of a 120-kDa glycoprotein substrate (pp 120) for the receptor-associated protein kinase in intact H-35 hepatoma cells. Proc Natl Acad Sci USA 84:3137-3140.
145. Accili, D., Perrotti, N., Rees-Jones, R., and Taylor, S. I. 1986. Tissue distribution and subcellular localization of an endogenous substrate (pp 120) for the insulin receptor-associated tyrosine kinase. Endocrinology 119:1274-1280.
146. Efrat, S., Linde, S., Kofod, H., Spector, D., Delannoy, M., Grant, S., Hanahan, D., and Baekkeskov, S. 1988. Beta-cell lines derived from transgenic mice expressing a hybrid insulin gene-oncogene. Proc Natl Acad Sci USA 85:9037-9041.
147. Okamoto, H., Hribal, M. L., Lin, H. V., Bennett, W. R., Ward, A., and Accili, D. 2006. Role of the forkhead protein FoxO1 in beta cell compensation to insulin resistance. J Clin Invest 116:775-782.
148. Buteau, J., Spatz, M. L., and Accili, D. 2006. Transcription factor FoxO1 mediates glucagon-like peptide-1 effects on pancreatic beta-cell mass. Diabetes 55:1190-1196.
149. Kitamura, Y. I., Kitamura, T., Kruse, J. P., Raum, J. C., Stein, R., Gu, W., and Accili, D. 2005. FoxO1 protects against pancreatic beta cell failure through NeuroD and MafA induction. Cell Metab 2:153-163.
150. Kitamura, T., Nakae, J., Kitamura, Y., Kido, Y., Biggs, W. H., 3rd, Wright, C. V., White, M. F., Arden, K. C., and Accili, D. 2002. The forkhead transcription factor Foxo1 links insulin signaling to Pdx1 regulation of pancreatic beta cell growth. J Clin Invest 110:1839-1847.
151. Matsumoto, M., Han, S., Kitamura, T., and Accili, D. 2006. Dual role of transcription factor FoxO1 in controlling hepatic insulin sensitivity and lipid metabolism. J Clin Invest 116:2464-2472.
152. Kim, J. J., Park, B. C., Kido, Y., and Accili, D. 2001. Mitogenic and metabolic effects of type I IGF receptor overexpression in insulin receptor-deficient hepatocytes. Endocrinology 142:3354-3360.
153. Park, B. C., Kido, Y., and Accili, D. 1999. Differential signaling of insulin and IGF-1 receptors to glycogen synthesis in murine hepatocytes. Biochemistry 38:7517-7523.
154. Liu, S. M., Leibel, R. L., and Chua, S. C., Jr. 1998. Partial duplication in the leprdb-Pas mutation is a result of unequal crossing over. Mamm Genome 9:780-781.
155. Chua, S. C., Jr., Koutras, I. K., Han, L., Liu, S. M., Kay, J., Young, S. J., Chung, W. K., and Leibel, R. L. 1997. Fine structure of the murine leptin receptor gene: splice site suppression is required to form two alternatively spliced transcripts. Genomics 45:264-270.
156. Taylor, S. I. 1992. Lilly Lecture: molecular mechanisms of insulin resistance. Lessons from patients with mutations in the insulin-receptor gene. Diabetes 41:1473-1490.
157. Foti, D., Chiefari, E., Fedele, M., Iuliano, R., Brunetti, L., Paonessa, F., Manfioletti, G., Barbetti, F., Brunetti, A., Croce, C. M., et al. 2005. Lack of the architectural factor HMGA1 causes insulin resistance and diabetes in humans and mice. Nat Med 11:765-773.
158. McKeon, C., Accili, D., Chen, H., Pham, T., and Walker, G. E. 1997. A conserved region in the first intron of the insulin receptor gene binds nuclear proteins during adipocyte differentiation. Biochem Biophys Res Commun 240:701-706.
159. McKeon, C., Moncada, V., Pham, T., Salvatore, P., Kadowaki, T., Accili, D., and Taylor, S. I. 1990. Structural and functional analysis of the insulin receptor promoter. Mol Endocrinol 4:647-656.
160. Coudert, A. E., Pibouin, L., Vi-Fane, B., Thomas, B. L., Macdougall, M., Choudhury, A., Robert, B., Sharpe, P. T., Berdal, A., and Lezot, F. 2005. Expression and regulation of the Msxl natural antisense transcript during development. Nucleic Acids Res 33:5208-5218.
161. Werner, A., and Berdal, A. 2005. Natural antisense transcripts: sound or silence? Physiol Genomics 23:125-131.
162. Blin-Wakkach, C., Lezot, F., Ghoul-Mazgar, S., Hotton, D., Monteiro, S., Teillaud, C., Pibouin, L., Orestes-Cardoso, S., Papagerakis, P., Macdougall, M., et al. 2001. Endogenous Msx1 antisense transcript: in vivo and in vitro evidences, structure, and potential involvement in skeleton development in mammals. Proc Natl Acad Sci U S a 98:7336-7341.
163. Okamoto, H., Obici, S., Accili, D., and Rossetti, L. 2005. Restoration of liver insulin signaling in Insr knockout mice fails to normalize hepatic insulin action. J Clin Invest 115:1314-1322.
164. Cinti, S., Eberbach, S., Castellucci, M., and Accili, D. 1998. Lack of insulin receptors affects the formation of white adipose tissue in mice. A morphometric and ultrastructural analysis. Diabetologia 41:171-177.
165. Postic, C., and Magnuson, M. A. 2000. DNA excision in liver by an albumin-Cre transgene occurs progressively with age. Genesis 26:149-150.
166. Nakae, J., Biggs, W. H., 3rd, Kitamura, T., Cavenee, W. K., Wright, C. V., Arden, K. C., and Accili, D. 2002. Regulation of insulin action and pancreatic beta-cell function by mutated alleles of the gene encoding forkhead transcription factor Foxo1. Nat. Genet. 32:245-253.
167. Accili, D. 2004. Lilly lecture 2003: the struggle for mastery in insulin action: from triumvirate to republic. Diabetes 53:1633-1642.
168. Fairhurst, A. M., Wandstrat, A. E., and Wakeland, E. K. 2006. Systemic lupus erythematosus: multiple immunological phenotypes in a complex genetic disease. Adv Immunol 92:1-69.
169. Hingorani, S. R., Petricoin, E. F., Maitra, A., Rajapakse, V., King, C., Jacobetz, M. A., Ross, S., Conrads, T. P., Veenstra, T. D., Hitt, B. A., et al. 2003. Preinvasive and invasive ductal pancreatic cancer and its early detection in the mouse. Cancer Cell 4:437-450.
170. Ventura, A., Kirsch, D. G., McLaughlin, M. E., Tuveson, D. A., Grimm, J., Lintault, L., Newman, J., Reczek, E. E., Weissleder, R., and Jacks, T. 2007. Restoration of p53 function leads to tumour regression in vivo. Nature 445:661-665.

What is claimed is:

1. A method for identifying an agent which modulates expression of a murine Ll RNA , the method comprising:
a) contacting a murine cell with an agent, wherein the cell contains an L1 gene;
b) determining expression of Ll RNA in the cell in the presence and absence of the agent; and
c) comparing expression of Ll RNA in the cell in the presence and absence of the agent, wherein a change in the expression of the Ll RNA in the presence of the agent is indicative of an agent which modulates the level of expression of the RNA.

2. A method for identifying an agent which modulates expression of a human C1orf32 RNA , the method comprising:
a) contacting a human cell with an agent, wherein the cell contains a C1orf32 gene;
b) determining expression of the C1orf32 RNA in the cell in the presence and the absence of the agent; and
c) comparing expression of the C1orf32 RNA in the cell in the presence and the absence of the agent, wherein a change in the expression of the C1orf32 RNA in the presence of the agent is indicative of an agent which modulates the level of expression of the RNA.

3. A method for identifying an agent which modulates expression of an mRNA encoding a murine LL protein, or a fragment or an isoform thereof, the method comprising:
a) contacting a cell with an agent;
b) determining expression of the mRNA in the presence and the absence of the agent, and
c) comparing the expression of the mRNA in the presence or the absence of the agent, wherein a change in the expression of the mRNA encoding LL protein in the presence of the agent is indicative of an agent which modulates the expression of the mRNA.

4. A method for identifying an agent which modulates expression level of an mRNA encoding the protein encoded by human C1ORF32 gene, the method comprising:
a) contacting a cell expressing the C1ORF32 gene with an agent;
b) determining expression levels of mRNA encoded by C1ORF32 in the presence and the absence of the agent; and
c) comparing the expression level of the mRNA in the presence and the absence of the agent, wherein a change in the level of expression of the mRNA encoding C1ORF32 in the presence of the agent is indicative of an agent which modulates the expression level of the mRNA.

5. A method for identifying an agent which modulates expression of murine Ll RNA , the method comprising:
a) contacting a cell expressing L1 RNA with an agent;
b) determining expression of an antisense RNA in the presence and the absence of the agent, wherein the antisense RNA comprises the sequence shown in SEQ ID NO: 18, 19 or 20; and
c) comparing the expression of the antisense RNA in the presence and the absence of the agent, wherein a change in the expression of the antisense RNA is indicative of an agent which modulates the expression of the Ll RNA.

6. A method for identifying an agent which modulates expression of C1orf32 RNA, the method comprising:
a) contacting a cell expressing C1ORF32 RNA with an agent;
b) determining expression of an antisense RNA in the presence and the absence of the agent, wherein the antisense RNA comprises the sequence shown in SEQ ID NO: 68, 73 or 74; and
c) comparing the expression of the antisense RNA in the presence and the absence of the agent, wherein an a change in the expression of the antisense RNA is indicative of an agent which modulates the of expression of the C1orf32 RNA.

7. The method of any of claims 1-6, wherein determining the expression comprises determining stability of RNA, determining level of RNA expression, determining level of expression of a type of C1ORF32 or LL RNA isoform or any combination thereof.

8. A method for identifying an agent which modulates expression of an LL murine protein, the method comprising:
a) contacting a cell expressing the LL protein with an agent;
b) determining expression of the LL protein in the presence and the absence of the agent; and
c) comparing the expression of the LL protein in the presence or the absence of the agent, wherein a change in the expression of the LL protein in the presence of the agent is indicative of an agent which modulates the expression of the LL protein.

9. A method for identifying an agent which modulates expression of human C1ORF32 protein, the method comprising:
a) contacting a cell expressing human C1ORF32, with an agent;
b) determining expression of the human C1ORF32 protein in the presence and absence of the agent; and
c) comparing the expression of the human C1ORF32 protein in the presence and absence of the agent, wherein a change in the expression of the C1ORF32 protein in the presence of the agent is indicative of an agent which modulates the expression of the human C1ORF32 protein.

10. The method of claim 8 or 9, wherein the LL protein or the C1ORF32 protein comprises a label.

11. The method of claim 10, wherein the label comprises a fluorescent label.

12. The method of claim 11, wherein the fluorescent label comprises a Green, Yellow, Cyanne, Cherry, Fluorescent Protein or any variant thereof.

13. The method of any one of claims 1-9, wherein the change is an increase.

14. The method of any one of claims 1-9, wherein the change is a decrease.

15. The method of any one of claims 1-9, wherein the change is transient.

16. The method of any one of claims 1-9, wherein the change is in localization, stability, modification, processing, posttranslational modification, or any combination thereof.

17. The method of any one of claims 1, 2, 5 and 6, wherein the Ll RNA or the C1orf32 RNA is endogenous.

18. The method of any of claim 3, 4, 8 or 9, wherein the LL RNA or protein or the C1ORF3 RNA or protein is endogenous.

19. The method of any one of claims 1-9, wherein the cell is transfected with a nucleic acid comprising the nucleic acid of any of SEQ ID NO: 10-13, 15-20 or a nucleic acid which is at least 75% homologous to any of SEQ ID NO: 10-13, 15-20.

20. The method of claim 19, wherein the cell comprises a fluorescently labeled C1ORF32.

21. The method of any of claims 1-9, wherein the cell is transfected with a nucleic acid comprising the nucleic acid of C1orf32 cDNA sequence or genomic sequence, with regulatory elements or a nucleic acid which is at least 75% homologous to same.

22. The method of any of claims 1-9, wherein the cell is derived from a diabetes-relevant tissue.

23. The method of claim 22, wherein the tissue comprises liver, pancreatic islet, skeletal muscle, brain, adipose tissue, or combination thereof.

24. The method of claim 22, wherein the cell comprises a pancreatic cell, a β-cell or an islet of Langerhans cell.

25. The method of any of claims 1-9, wherein the cell comprises an insulin producing beta cell, a hepatocyte cell, or a hypothalamic cell.

26. The method of claim 22, wherein the cell comprises a murine cell, a rat cell, or a human cell.

27. The method of any of claims 1-9, wherein the method is performed in vivo or in vitro.

28. An isolated peptide consisting essentially of the amino acid sequence of SEQ ID NO: 6 or an isolated peptide which is at least 75% identical to SEQ ID NO: 6.

29. An isolated peptide consisting essentially of the amino acid sequence of SEQ ID NO: 7 or an isolated peptide which is at least 75% identical to SEQ ID NO: 7.

30. An isolated peptide consisting essentially of the amino acid sequence of SEQ ID NO: 8 or an isolated peptide which is at least 75% identical to SEQ ID NO: 8.

31. An isolated peptide consisting essentially of the amino acid sequence of SEQ ID NO: 9 or an isolated peptide which is at least 75% identical to SEQ ID NO: 9.

32. An isolated peptide consisting essentially of the amino acid sequence of SEQ ID NO: 70 or an isolated peptide which is at least 75% identical to SEQ ID NO: 70.

33. An isolated peptide consisting essentially of the amino acid sequence of SEQ ID NO: 71 or an isolated peptide which is at least 75% identical to SEQ ID NO: 71.

34. An isolated peptide consisting essentially of the amino acid sequence of SEQ ID NO:72 or an isolated peptide which is at least 75% identical to SEQ ID NO: 72.

35. An isolated peptide consisting essentially of the amino acid sequence of SEQ ID NO: 69 or an isolated peptide which is at least 75% identical to SEQ ID NO: 69.

36. A mixture comprising at least two peptides of any of claims 28-35.

37. An antibody which specifically binds to a polypeptide comprising the peptide of any of claims 28-35.

38. An antibody which specifically binds to the peptide of any of claims 28-35.

39. The antibody of claim 37 or 38, wherein the antibody is a polyclonal antibody.

40. The antibody of claim 37 or 38, wherein the antibody is a monoclonal antibody.

41. The antibody of claim 37 or 38, wherein the antibody is a soluble antibody fragment.

42. An isolated nucleic acid consisting essentially of SEQ ID NO: 18, 19 or 20 or an isolated nucleic acid which is at least 75% homologous to the nucleic acid of SEQ ID NO: 18, 19 or 20.

43. An isolated nucleic acid consisting essentially of SEQ ID NO: 68, 73 or 74 or an isolated nucleic acid which is at least 75% homologous to the nucleic acid of SEQ ID NO: 68, 73, or 74.

44. A composition comprising the nucleic acid of claim 42 or 43.

45. A method for detecting a predisposition to type 2 diabetes in a subject, the method comprising determining expression of C1orf32 RNA or C1ORF32 protein in a sample obtained from a subject, wherein decreased expression, compared to expression in a control sample from a subject known not to have type 2 diabetes, indicates that the subject is susceptible to type II diabetes.

46. The method of claim 45, wherein determining comprises measuring expression level of C1orf32 RNA or C1ORF32 protein in the sample, or determining C1ORF32 protein localization or determining post-translational modification of C1ORF32 protein.

47. The method of claim 45, wherein determining expression level of C1ORF32 protein comprises immunohistochemistry or Western blotting using an antibody which specifically binds to C1ORF32 protein.

48. The method of claim 45, wherein the sample from the subject and the control sample are from a diabetes-relevant tissue or cell.

49. The method of claim 45, wherein the diabetes-relevant tissue or cell comprises liver , pancreatic islet, skeletal muscle, brain, adipose tissue, adipose cell, or any combination thereof.

50. The method of claim 45, wherein determining comprises quantifying RNA encoding the C1Orf32 polypeptide, a variant thereof, a fragment thereof, or any combination thereof.

51. A method for manipulating beta cell mass to treat a biological condition in a subject, comprising contacting a beta cell precursor with an agent which increases expression of C1orf32 mRNA or C1ORF32 protein, thereby manipulating beta cell mass in the subject.

52. A method for treating a biological condition associated with reduced beta cell mass in a subject, comprising administering to the subject an agent which increases expression of C1orf32 mRNA or C1ORF32, so as to increase beta cell mass in the subject thereby treating the biological condition.

53. A method for treating a biological condition associated with reduced levels of C1orf32 mRNA or C1ORF32 in a subject, comprising administering an agent which increases expression of C1orf32 mRNA or C1ORF32, thereby treating the biological condition.

54. The method of any of claims 51-53 , wherein the biological condition is type II diabetes.

55. The method of any of claims 51-53, wherein the expression of C1orf32 mRNA or C1ORF32 protein is increased in pancreas, in skeletal muscle, in adipose tissue, in brain hypothalamus, or any combination thereof.

56. The method of any of claims 51-53, wherein the expression of C1orf32 mRNA or C1ORF32 protein is increased in beta cells.

57. A method for increasing expression of C1orf32 RNA or C1ORF32 protein in a pancreatic cell, the method comprising contacting the cell with an agent which increases the levels of the C1orf32 RNA or C1ORF32 protein.

58. The method of claim 57, wherein the pancreatic cell is a β-cell or an islet of Langerhans cell.

59. A method of modulating beta cell development, the method comprising contacting a pancreatic cell with an agent which increases the levels of C1orf32 mRNA or C1ORF32 protein.

60. A method for increasing beta cell mass, beta cell numbers or beta cell proliferation, the method comprising contacting a pancreatic cell with an agent which increases expression of C1orf32 mRNA or C1ORF32 protein.

61. The method of claim 59 or 60, wherein the method is performed in vivo.

62. The method of claim 59 or 60, wherein the method is performed ex vivo.

63. A method for treating a pre-diabetic or a diabetic subject, the method comprising administering to the subject a therapeutically effective amount of an agent which increases the expression of C1orf32 mRNA or C1ORF32 protein.

64. The method of any of claim 51, 52, 53 or 63, wherein the subject is suspected to have or has type2 diabetes (T2DM).

65. A method for treating a subject suffering from a disease or disorder associated with defects in beta cell mass, beta cell proliferation or beta cell activity, the method comprising:
(a) isolating a pancreatic (beta cell) cell from a donor,
(b) introducing a nucleic acid which comprises a nucleic acid sequence encoding C1ORF32 polypeptide into the pancreatic cell;
(c) transferring the pancreatic cell of (b) in the subject, wherein the pancreatic cell grows, and differentiates into insulin producing beta cell.

66. The method of claim 65, wherein the donor is the subject.

67. The method of claim 65, optionally comprising a step of ex vivo expanding of the pancreatic cell of step (b).

68. The method of claim 67, wherein the step of expanding is performed in the presence of growth factors.

69. The method of any one of claim 51, 52, 53, 57, 59, 60 or 63, wherein the agent is a nucleic acid which comprises a nucleic acid sequence encoding a C1ORF32 protein, a C1ORF32 polypeptide, a C1ORF32 isoform, or a C1ORF32 functional fragment.

70. The method of any one of claim 51, 52, 53, 57, 59, 60 or 63, wherein the agent is a C1ORF32 protein, a C1ORF32 polypeptide, a C1ORF32 isoform, or a C1ORF32 functional fragment.

71. A method for manipulating beta cell mass to treat a biological condition in a subject, comprising contacting a beta cell precursor with a peptide having SEQ ID NO:1-9 or 69-72, or a C1ORF32 protein, a C1ORF32 polypeptide, a C1ORF32 isoform, or a C1ORF32 functional fragment, or any combination thereof, thereby manipulating beta cell mass in the subject.

72. A method for treating a biological condition associated with reduced beta cell mass in a subject, comprising administering to the subject a peptide having SEQ ID NO:1-9 or 69-72, or a C1ORF32 protein, a C1ORF32 polypeptide, a C1ORF32 isoform, or a C1ORF32 functional fragment, or any combination thereof, so as to increase beta cell mass in the subject thereby treating the biological condition.

73. The method of any of claims 71-72 , wherein the biological condition is type II diabetes, obesity, a dyslipidemia, or any combination thereof.

74. A method for treating a pre-diabetic or a diabetic subject, the method comprising administering to the subject a therapeutically effective amount of a peptide having SEQ ID NO:1-9 or 69-72, or a C1ORF32 protein, a C1ORF32 polypeptide, a C1ORF32 isoform, or a C1ORF32 functional fragment, or any combination thereof.

75. A peptide having SEQ ID NO:1-9 or 69-72, or a C1ORF32 protein, a C1ORF32 polypeptide, a C1ORF32 isoform, or a C1ORF32 functional fragment for use in treating a pre-diabetic or a diabetic condition in a subject.

76. A peptide having SEQ ID NO:1-9 or 69-72, or a C1ORF32 protein, a C1ORF32 polypeptide, a C1ORF32 isoform, or a C1ORF32 functional fragment for use in treating a biological condition associated with reduced beta cell mass in a subject.

77. A peptide having SEQ ID NO:1-9 or 69-72, or a C1ORF32 protein, a C1ORF32 polypeptide, a C1ORF32 isoform, or a C1ORF32 functional fragment for use in manipulating beta cell mass to treat a biological condition in a subject.

78. An antibody that specifically binds to a peptide having SEQ ID NO:1-9 or 69-72, or a C1ORF32 protein, a C1ORF32 polypeptide, a C1ORF32 isoform, or any fragment thereof.

79. A method for diagnosing type 2 diabetes in a subject, the method comprising (a) detecting expression of a peptide having SEQ ID NO:1-9 or 69-72, or a C1ORF32 protein or a C1ORF32 polypeptide in a sample blood or tissue from a subject, wherein the antibody of claim 78 is used to determine expression, and (b) comparing expression the expression in (a) to the expression of a peptide having SEQ ID NO:1-9 or 69-72, or a C1ORF32 protein or a C1ORF32 polypeptide in a control sample.
